(19) United States
(12) Patent Application Publication
(10) Pub. No.: US 2025/0188085 A1
(43) Pub. Date: Jun. 12, 2025

(54) COMPOSITIONS AND METHODS FOR INHIBITION OF RAS

(71) Applicants: THERAS, INC., South San Francisco, CA (US); Leidos Biomedical Research, Inc., Frederick, MD (US); Lawrence Livermore National Security, LLC, Livermore, CA (US)

(72) Inventors: Bin Wang, Dallas, TX (US); Rui Xu, Dallas, TX (US); Eli Wallace, Loveland, CO (US); Zuhui Zhang, Millbrae, CA (US); Anna Elzbieta Maciag, Frederick, MD (US); David Michael Turner, Frederick, MD (US); Dhirendra Kumar Simanshu, Frederick, MD (US); Albert Hay Wah Chan, Frederick, MD (US); Felice Lightstone, Fremont, CA (US); Yue Yang, Foster City, CA (US)

(21) Appl. No.: 18/726,865

(22) PCT Filed: Jan. 5, 2023
(86) PCT No.: PCT/US2023/010172

§ 371 Date: Jul. 5, 2024

Related U.S. Application Data
(60) Provisional application No. 63/297,132, filed on Jan. 6, 2022.

(51) Int. Cl. C07D 487/04; A61K 31/517; A61K 31/519; C07D 417/14; C07D 519/00

(52) U.S. Cl. CPC C07D 487/04; A61K 31/517; A61K 31/519; C07D 417/14; C07D 519/00

(57) ABSTRACT

Provided herein are compounds, or salts, esters, tautomers, prodrugs, zwitterionic forms, or stereoisomers thereof, as well as pharmaceutical compositions comprising the same. Also provided herein are methods of using the same in modulating (e.g., inhibiting) KRAS (e.g., KRAS having a G12C mutation) and treating diseases or disorders such as cancers in subjects in need thereof.

### RELATED APPLICATIONS

This application claims priority to and benefit of U.S. Application No. 63/297,132, filed Jan. 6, 2022, the entire contents of which are hereby incorporated by reference.

### STATEMENT AS TO RIGHTS TO INVENTIONS MADE UNDER FEDERALLY SPONSORED RESEARCH AND DEVELOPMENT

This invention was made with government support under (1) Contract No.: 75N91019D00024 awarded by the National Institutes of Health and (2) Contract No. DE-AC52-07NA27344 awarded by the United States Department of Energy. The government has certain rights in the invention.

### BACKGROUND

RAS mutations occur in approximately 30% of human cancers, including the majority of pancreatic ductal adenocarcinoma (PDAC), half of colorectal cancers, and a third of all lung cancers. With the highest RAS mutation frequencies seen with the top three causes of cancer deaths in the United States (lung, colorectal, and pancreatic cancer), the development of anti-RAS therapies is a major priority and a major challenge for cancer research. RAS proteins did not appear to present suitable pockets to which drugs could bind, except for the GDP/GTP binding site. Unfortunately, RAS proteins bind to these nucleotides with very high (picomolar) affinities, making the development of effective nucleotide analogs virtually impossible. Attempts to block pathways downstream of RAS with a hope to provide clinical benefit for patients suffering from RAS-driven cancers have been generally disappointing.

The three RAS genes (HRAS, NRAS, and KRAS) encode four 188-189 amino acid proteins that share 82%-90% amino acid sequence identity and near-identical structural and biochemical properties. However, they are differentially expressed, and mutated with different frequencies in cancer. KRAS is the most frequently mutated oncogene in cancer and KRAS mutation is commonly associated with poor prognosis and resistance to therapy. Significant cancer type preferences exist among the RAS genes. KRAS mutations predominate in lung, colorectal, and pancreatic cancer, whereas NRAS mutations predominate in cutaneous melanomas and acute myelogenous leukemia, and HRAS mutations are found in bladder and head and neck squamous cell carcinomas.

An estimated over 600,000 Americans will die from cancer in 2021, corresponding to more than 1600 deaths per day (Cancer Facts and Figures 2021). The greatest number of deaths are from cancers of the lung, prostate, and colorectum in men, and cancers of the lung, breast, and colorectum in women. Almost one-quarter of all cancer deaths are due to lung cancer, 82% of which is directly caused by cigarette smoking. The 5-year survival rate for lung cancer patients is only about 20%.

KRAS is mutationally activated in lung cancer, and Glycine-to-Cysteine (G12C) mutations account for the majority of codon 12 mutations associated with cigarette smoking. A significant percentage of colorectal cancers are also driven by KRAS G12C mutations.

Early clinical data for allele-specific covalent KRAS G12C inhibitors show some effectiveness, at least in lung cancer. Those KRAS G12C inhibitors (e.g., Amgen Inc.'s sotorasib and Mirati Therapeutics. Inc.'s adagrasib) target inactive (GDP)-bound protein, and their effectiveness is enabled by high (comparable to wild type (WT) KRAS) intrinsic GTP hydrolysis rates of KRAS G12C mutant. Clinical data for these agents have shown that though most patients with KRAS G12C mutant non-small cell lung cancer (NSCLC) experience clinical benefit from selective KRAS G12C inhibition, patients with colorectal cancer bearing the same mutation rarely respond.

The cause of limited efficacy of KRAS G12C (GDP-bound) inhibitors in colorectal cancers has been investigated. Unlike NSCLC cell lines, KRAS G12C colorectal cancer models have high basal receptor tyrosine kinase (RTK) activation and are responsive to growth factor stimulation. In colorectal cancer lines, KRAS G12C inhibition induces higher phospho-ERK rebound than in NSCLC cells. Also, it has been reported that KRAS G12C (GDP-bound) inhibitors induce transcription of new KRAS G12C that is in GTP-bound conformation, and insensitive to KRAS G12C inactive state inhibitors.

Therefore, KRAS G12C inhibitors targeting active GTP-bound protein could have therapeutic advantage over KRAS G12C-GDP inhibitors.

### SUMMARY

In an aspect, the present disclosure provides compositions comprising compounds according to one of Formulas I and II:

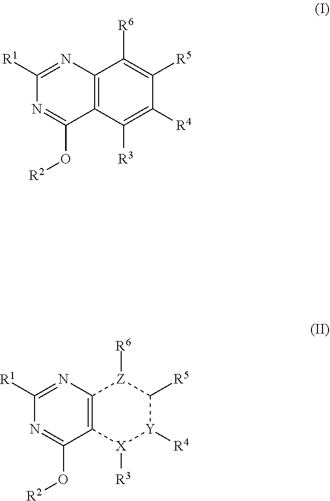

or a salt (e.g., pharmaceutically acceptable salt), ester, tautomer, prodrug, zwitterionic form, or stereoisomer thereof, wherein R¹, R², R³, R⁴, R⁵, R⁶, X, Y, and Z are as provided herein. In some embodiments, a compound provided herein, or a salt (e.g., pharmaceutically acceptable salt), ester, tautomer, zwitterionic form, or stereoisomer thereof, can modulate the activity of a KRAS protein, such as a KRAS protein having a G12C mutation. In some embodiments, a compound provided herein, or a salt (e.g., pharmaceutically acceptable salt), ester, tautomer, zwitterionic form, or stereoisomer thereof, includes an electrophilic moiety E, as provided herein. In some embodiments, a compound provided herein, or a salt (e.g., pharmaceutically acceptable salt), ester, tautomer, zwitterionic form, or stereoisomer thereof, is capable of interacting covalently with a cysteine at the 12 position of the KRAS protein (e.g., a G12C mutation). In some embodiments, a compound provided herein, or a salt (e.g., pharmaceutically acceptable salt), ester, tautomer, zwitterionic form, or stereoisomer thereof, is capable of binding a KRAS protein in an active (GTP-bound) conformation. In some embodiments, a compound provided herein, or a salt (e.g., pharmaceutically acceptable salt), ester, tautomer, zwitterionic form, or stereoisomer thereof, is capable of binding a KRAS protein in an inactive (GDP-bound) conformation.

In another aspect, the present disclosure provides a pharmaceutical composition comprising a compound provided herein (e.g., a compound according to any one of Formulas I, IA, IA1, IA2, IA3, IB, II, IIA, IIB, and IIC), or a salt (e.g., pharmaceutically acceptable salt), ester, tautomer, prodrug, zwitterionic form, or stereoisomer thereof, together with a pharmaceutically acceptable carrier.

In a further aspect, the present disclosure provides a method of inhibition of KRAS activity in a human or animal subject for the treatment of a disease such as cancer, including pancreatic cancer (e.g., pancreatic ductal adenocarcinoma (PDAC)), colorectal cancer, and lung cancer, using, e.g., a compound provided herein (e.g., a compound according to any one of Formulas I, IA, IA1, IA2, IA3, IB, II, IIA, IIB, and IIC), or a salt (e.g., pharmaceutically acceptable salt), ester, tautomer, prodrug, zwitterionic form, or stereoisomer thereof, or a pharmaceutical composition comprising the same.

In another aspect, the present disclosure provides a use of a compound provided herein (e.g., a compound according to any one of Formulas I, IA, IA1, IA2, IA3, IB, II, IIA, IIB, and IIC), or a salt (e.g., pharmaceutically acceptable salt), ester, tautomer, zwitterionic form, or stereoisomer thereof, in the manufacture of a medicament for the treatment of a disease, disorder, or condition (e.g., a cancer) ameliorated, treated, inhibited, or reduced by inhibition of KRAS, including KRAS having a G12C mutation. In some embodiments, the disease, disorder, or condition is pancreatic cancer (e.g., pancreatic ductal adenocarcinoma (PDAC)), colorectal cancer, or lung cancer.

In a further aspect, the present disclosure provides a compound as provided herein (e.g., a compound according to any one of Formulas I, IA, IA1, IA2, IA3, IB, II, IIA, IIB, and IIC), or a salt (e.g., pharmaceutically acceptable salt), ester, tautomer, zwitterionic form, or stereoisomer thereof, for use as a medicament. In some embodiments, the medicament is used in the treatment of a disease, disorder, or condition (e.g., a cancer). In some embodiments, the disease, disorder, or condition is pancreatic cancer (e.g., pancreatic ductal adenocarcinoma (PDAC)), colorectal cancer, or lung cancer.

### DETAILED DESCRIPTION

The present disclosure provides compounds and methods of using such compounds and compositions comprising the same in inhibiting RAS (e.g., KRAS)-mediated cell signaling, such as in the treatment or prophylaxis of a disease, disorder, or condition in which KRAS plays an active role. The present disclosure provides compounds (e.g., compounds of any one of Formulas I, IA, IA1, IA2, IA3, IB, II, IIA, IIB, and IIC) and forms thereof (e.g., a salt (e.g., pharmaceutically acceptable salt), ester, tautomer, zwitterionic form, or stereoisomer thereof) that may possess useful KRAS inhibitory activity and may be used in inhibiting KRAS activity in a cell and/or in the treatment or prophylaxis of a disease, disorder, or condition in which KRAS plays an active role. In particular, certain compounds and forms thereof provided herein may possess useful inhibitory activity of KRAS having a G12C mutation, which KRAS protein is in an active (GTP-bound) or inactive (GDP-bound) conformation. Certain compounds and forms thereof provided herein may be capable of inhibiting both active and inactive forms of KRAS. The present disclosure also provides pharmaceutical compositions comprising one or more compounds or forms thereof provided herein together with a pharmaceutically acceptable carrier, as well as methods of making and using the compounds and compositions. The present disclosure also provides methods for inhibiting KRAS, including KRAS having a G12C mutation, which KRAS is in an active or inactive conformation. In an aspect, the present disclosure provides a method for treating a disorder mediated by KRAS including a KRAS having a G12C mutation in a subject in need of such treatment, which method comprises administering to the subject a therapeutically effective amount of a compound or composition provided herein. Also provided herein is the use of certain compounds provided herein in the manufacture of a medicament for the treatment of a disease, disorder, or condition ameliorated, treated, inhibited, or reduced by inhibition of KRAS, including KRAS having a G12C mutation. In some embodiments, the disease, disorder, or condition is a cancer (e.g., as described herein).

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched hydrocarbon having at least 2 carbon atoms and at least one triple bond and having the number of carbon atoms indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as described herein. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms (e.g., C₁₋₂₀ alkyl). In certain embodiments, said alkyl will comprise from 1 to 10 carbon atoms (e.g., C₁₋₁₀ alkyl). In further embodiments, said alkyl will comprise from 1 to 8 carbon atoms (e.g., C₁₋₈ alkyl). In further embodiments, said alkyl will comprise from 1 to 6 carbon atoms (e.g., C₁₋₆ alkyl). In further embodiments, said alkyl will comprise from 1 to 3 carbon atoms (e.g., C₁₋₃ alkyl). Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino, and the like.

“Alkylthio,” as used herein, alone or in combination, refers to an alkyl thioether (R—S—) radical wherein the term alkyl is as described herein and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described herein attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O)NH₂, C₁₋₄alkylamido, and di(C₁₋₄alkyl)amido. The term “C₁₋₄alkylamido”, as used herein, refers to —C(O)NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form a heterocycloalkyl, which is unsubstituted or substituted. An “amino” group may be a primary amine (e.g., —NH₂), secondary or di-substituted amine (e.g., —NHR where R is not hydrogen), or tertiary or tri-substituted amine (e.g., —NRR′ where neither R nor R′ is hydrogen).

“Aryl,” as used herein, alone or in combination, means a carbocyclic aromatic system containing one, two, or three rings wherein such polycyclic ring systems are fused together. The term “aryl” embraces aromatic groups such as phenyl, naphthyl, anthracenyl, and phenanthryl. An aryl moiety may include, for example, between 5 to 20 carbon atoms, such as between 5 to 12 carbon atoms, such as 5 or 6 carbon atoms.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′ group, with R and R′ as defined herein.

“N-carbamyl” as used herein, alone or in combination, refers to a ROC(O)NR′— group, with R and R′ as defined herein.

“Carbonyl,” as used herein, when alone includes formyl [—C(O)H] and in combination is a —C(O)— group.

“Carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic, or tricyclic alkyl group wherein each cyclic moiety contains from 3 to 12 carbon atom ring members and which may optionally be a benzo fused ring system which is unsubstituted or substituted as defined herein. A carbocycle may comprise a bridged ring system and/or a spiro ring system (e.g., a system including two rings sharing a single carbon atom). The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl, and the like. “Bicyclic” and “tricyclic” as used herein are intended to include both fused ring systems, such as decahydronaphthalene and octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen,” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as described herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

“Heteroalkyl,” as used herein, alone or in combination, refers to a stable straight or branched hydrocarbon chain, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3 to 15 membered aromatic monocyclic ring, or a fused monocyclic, bicyclic, or tricyclic ring system in which at least one of the fused rings is aromatic, which ring or ring system contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. A heterocycle may comprise a bridged ring system and/or a spiro ring system (e.g., a system including two rings sharing a single atom, such as a single carbon atom). “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to ═N(OH) and ═N—O—.

“Lower amino,” as used herein, alone or in combination, refers to —NRR′, wherein R and R′ are independently selected from hydrogen and lower alkyl, either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo,” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), S(O)ₘ, wherein m is 0, 1, or 2. Unless specifically prohibited, a ring is unsubstituted or substituted. Two or more rings may be fused together (e.g., they may share a bond and two common atoms). Two or more rings may be linked together in a spiro arrangement such that only a single atom is shared between two rings. Two or more rings may also or alternatively be configured in a bridged arrangement such that three or more atoms are shared between two or more rings.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to —S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

“Thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl-C(S) H and in combination is a —C(S)— group.

“N-thiocarbamyl” refers to an ROC(S) NR′— group, with R and R′ as defined herein.

“O-thiocarbamyl” refers to a —OC(S) NRR′ group with R and R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As described herein, groups may be substituted or unsubstituted (e.g., “optionally substituted”). Unless otherwise specified, any group may be substituted with one or more substituents, such as one or more substituents provided herein. Examples of substituents that may substitute a group include, but are not limited to, one or more substituents independently selected from the following groups or a particular designated set of groups, alone or in combination: alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), alkynyl (e.g., C₂₋₂₀ alkynyl, such as C₂₋₁₀ alkynyl, such as C₂₋₆ alkynyl), alkanoyl (e.g., C₁₋₂₀ alkanoyl, such as C₁₋₁₀ alkanoyl, such as C₁₋₆ alkanoyl), heteroalkyl (e.g., a heteroalkyl moiety including 1-20 carbon atoms and 1-6 heteroatoms, such as a heteroalkyl moiety including 1-6 carbon atoms and 1-3 heteroatoms), haloalkyl (e.g., a halo-substituted C₁₋₂₀ alkyl, such as a halo-substituted C₁₋₁₀ alkyl, a halo-substituted C₁₋₆ alkyl), haloalkenyl (e.g., a halo-substituted C₂₋₂₀ alkenyl, such as a halo-substituted C₂₋₆ alkenyl), haloalkynyl (e.g., a halo-substituted C₂₋₂₀ alkynyl, such as a halo-substituted C₂₋₆ alkynyl), perhaloalkyl (e.g., C₁₋₂₀ perhaloalkyl, such as C₁₋₆ perhaloalkyl, such as C₁₋₃ perhaloalkyl), perhaloalkoxy (e.g., C₁₋₂₀ perhaloalkoxy, such as C₁₋₆ perhaloalkoxy), phenyl, aryl (e.g., C₅₋₂₀ aryl, such as C₅₋₁₀ aryl, such as C₅₋₆ aryl), aryloxy (e.g., C₅₋₂₀ aryloxy, such as C₅₋₁₀ aryloxy, such as C₅₋₆ aryloxy), alkoxy (e.g., C₁₋₂₀ alkoxy, such as C₁₋₁₀ alkoxy, such as C₁₋₆ alkoxy), haloalkoxy (e.g., C₁₋₂₀ haloalkoxy, such as C₁₋₁₀ haloalkoxy, such as C₁₋₆ haloalkoxy), oxo, acyloxy (e.g., an acyloxy group including 1-20 carbon atoms, such as 1-10 carbon atoms, such as 1-6 carbon atoms), carbonyl (e.g., C(O) or C═O), carboxyl (e.g., C(O)O), alkylcarbonyl (e.g., C₁₋₂₀ alkylcarbonyl, such as C₁₋₁₀ alkylcarbonyl, such as C₁₋₆ alkylcarbonyl, such as C₁₋₃ alkylcarbonyl), carboxyester (e.g., C(O)OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), carboxamido, cyano (e.g., CN), hydrogen, halogen (e.g., iodine, bromine, chlorine, or fluorine), hydroxy, amino (e.g., NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), alkylamino (e.g., NR′R″ where R′ is alkyl (e.g., C₁₋₂₀ alkyl, such as C₁₋₁₀ alkyl, such as C₁₋₆ alkyl, such as C₁₋₃ alkyl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), arylamino (e.g., NR′R″ where R′ is aryl (e.g., C₅₋₂₀ aryl, such as C₅₋₁₀ aryl, such as C₅₋₆ aryl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), amido (e.g., C(O)NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), nitro (e.g., NO₂), thiol (e.g., SH), alkylthio (e.g., C₁₋₂₀ alkyl substituted with a thiol group, such as C₁₋₁₀ alkyl substituted with a thiol group, such as C₁₋₆ alkyl substituted with a thiol group, such as C₁₋₃ alkyl substituted with a thiol group), haloalkylthio (e.g., C₁₋₂₀ haloalkylthio, such as C₁₋₁₀ haloalkylthio, such as C₁₋₆ haloalkylthio, such as C₁₋₃ haloalkylthio), perhaloalkylthio (e.g., C₁₋₂₀ perhaloalkylthio, such as C₁₋₁₀ perhaloalkylthio, such as C₁₋₆ perhaloalkylthio, such as C₁₋₃ perhaloalkylthio), arylthiol (e.g., C₅₋₂₀ arylthiol, such as C₅₋₁₀ arylthiol, such as C₅₋₆ arylthiol), sulfonate (e.g., S(O)₂OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), sulfonic acid (e.g., S(O)₂OH), trisubstituted silyl (e.g., SiR′R″R* where R′, R″, and R* are independently selected from, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein; in some cases, a trisubstituted silyl can be trimethylsilyl), N₃, SCH₃, C(O)CH₃, CO₂CH₃, CO₂H, pyridinyl, thiophene, furanyl, carbamate, and urea. Additional groups may also be contemplated. Where structurally feasible, two substituents may be joined together to form a fused five-, six-, or seven-membered carbocyclic or heterocyclic ring consisting of zero to three heteroatoms (e.g., N, O, S, etc.), for example forming methylenedioxy or ethylenedioxy. An unsubstituted or substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “unsubstituted or substituted with.”

The terms R, R′, R″, R*, etc., appearing by themselves and without a number designation, unless otherwise defined, refer to a moiety selected from hydrogen, alkyl, cycloalkyl, heteroalkyl, aryl, heteroaryl and heterocycloalkyl, any of which is unsubstituted or substituted (e.g., as described herein). Such R and R′ groups should be understood to be unsubstituted or substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers may exist in the compounds disclosed herein. These centers are designated by the symbols “R” or “S,” depending on the configuration of substituents around the chiral carbon atom. It should be understood that the present disclosure encompasses all stereochemical isomeric forms, including diastereomeric, enantiomeric, atropisomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“KRAS inhibitor” is used herein to refer to a compound that exhibits inhibitory activity of RAS (e.g., KRAS)-mediated signal transduction. Such activity may be assessed and demonstrated by various methods, including a showing of a decrease in the levels of signaling transduction molecules downstream in the RAS pathway (e.g., pMEK, pERK, or pAKT), and/or a decrease in binding of RAS complex to downstream signaling molecules such as Raf. A KRAS inhibitor may refer to a compound that exhibits an IC₅₀ with respect to KRAS activity of no more than about 100 μM and more typically not more than about 50 μM, as measured in the assays described generally herein, such as level of covalent modification to Cysteine (Cys) 12 in KRAS G12C as measured using a matrix assisted laser desorption ionization-time of flight mass spectrometry (MALDI-TOF MS) assay, and/or a KRAS G12C protein-effector protein interaction disruption assay. “IC₅₀” is that concentration of inhibitor which reduces the activity of an enzyme (e.g., KRAS) to half-maximal level. Certain compounds disclosed herein have been discovered to exhibit inhibition against KRAS. In certain embodiments, compounds exhibit an IC₅₀ with respect to KRAS (e.g., KRAS having a G12C mutation) of no more than about 50 μM; in further embodiments, compounds exhibit an IC₅₀ with respect to KRAS (e.g., KRAS having a G12C mutation) of no more than about 10 μM; in yet further embodiments, compounds exhibit an IC₅₀ with respect to KRAS (e.g., KRAS having a G12C mutation) of not more than about 1 μM; in yet further embodiments, compounds exhibit an IC₅₀ with respect to KRAS (e.g., KRAS having a G12C mutation) of not more than about 200 nanomolar (nM), as measured in the KRAS assay described herein. In some embodiments, compounds exhibit an IC₅₀ with respect to KRAS (e.g., KRAS having a G12C mutation) of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In certain embodiments, compounds exhibit an IC₅₀ with respect to KRAS (e.g., KRAS having a G12C mutation) of less than about 1 μM, such as less than about 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, a KRAS inhibitor has inhibitory activity against KRAS having a G12C mutation that exceeds its inhibitory activity against KRAS having another mutation, such as a G12D mutation, a G12V mutation, a G12S mutation, and/or a G12R mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12C mutation relative to KRAS having a G12D mutation, a G12V mutation, a G12S mutation, and/or a G12R mutation. In some embodiments, a KRAS inhibitor provided herein has inhibitory activity against both active (GTP-bound) KRAS having a G12C mutation and inactive (GDP-bound) KRAS having a G12C mutation. In some embodiments, a KRAS inhibitor provided herein has greater inhibitory activity against active KRAS having a G12C mutation than against an inactive KRAS having a G12C mutation. In some embodiments, a KRAS inhibitor provided herein has lower inhibitory activity against active KRAS having a G12C mutation than against an inactive KRAS having a G12C mutation. In some embodiments, a KRAS inhibitor provided herein has similar inhibitory activity against active and inactive KRAS having a G12C mutation. In some embodiments, a KRAS inhibitor provided herein has inhibitory activity against a K-RAS4a splice variant. In some embodiments, a KRAS inhibitor provided herein has inhibitory activity against a K-RAS4b splice variant. In some embodiments, a KRAS inhibitor provided herein has inhibitory activity against both K-RAS4a and K-RAS4b splice variants.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). A therapeutically effective amount may decrease KRAS activity by at least 1% compared to a control, such as at least 5%, 10%, 15%, 20%, 25%, 30%, 35%, 40%, 45%, 50%, 55%, 60%, 65%, 70%, 75%, 80%, 85%, 90%, 95%, or more.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, disease, disorder, or condition, including any objective or subjective parameter such as abatement; remission; diminishing of symptoms or making the injury, pathology, disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, hamsters, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts (also referred to herein as “pharmaceutically acceptable salts”). The present disclosure includes compounds provided herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt” or “pharmaceutically acceptable salt” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, NN-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

“KRAS G12C-positive cancer” refers to a cancer characterized by a KRAS G12C mutation.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a KRAS G12C inhibitor, as defined herein, and an additional agent, which additional agent may be an agent configured to treat a disease, disorder, or condition or a symptom thereof. The effect can be, for example, slowing the symptomatic progression of a proliferative disease, such as cancer, particularly lung cancer, or symptoms thereof. Analogously, a “synergistically effective amount” refers to the amount needed to obtain a synergistic effect.

“A,” “an,” or “a (n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Compounds

In an aspect, the present disclosure provides a compound according to Formula I:

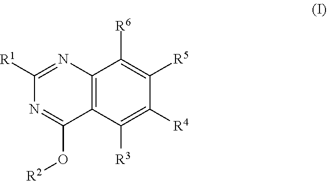

or a salt (e.g., pharmaceutically acceptable salt), ester, tautomer, prodrug, zwitterionic form, or stereoisomer thereof, wherein:

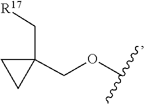

R¹ is selected from —OR⁷, and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is substituted with one or more E and 0-4 R⁸;
R³ is selected from H, —OR¹⁰, and C₁₋₆alkyl, wherein any C₁₋₆alkyl is unsubstituted or substituted with one or more R¹¹;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is independently selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶,
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
each E is independently selected from

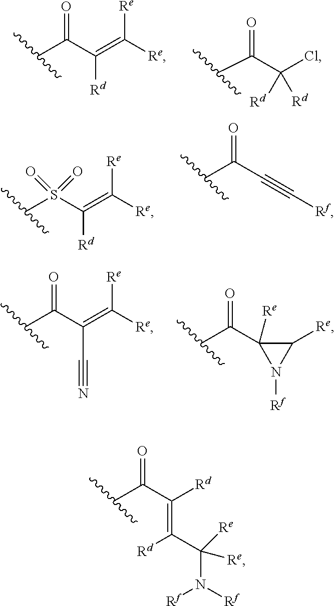

and —CN;
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} and R^{e} is independently selected from halogen, C₁₋₆ alkyl, and H; and
each R^{f} is independently selected from C₁₋₆ alkyl and H.

In some embodiments, the present disclosure provides a compound of Formula I, or a salt (e.g., a pharmaceutically acceptable salt) thereof.

In some embodiments, for a compound according to Formula I, R² is a 4-6 membered heterocycle containing one nitrogen atom, wherein the heterocycle is substituted with one or more E and 0-4 R⁸. In some embodiments, R² is a heterocycle selected from azetidine, pyrrolidine, and piperidine, wherein the heterocycle is substituted with one or more E and 0-4 R⁸. In some embodiments, R² is an azetidine that is substituted with one or more E and 0-4 R⁸. In some embodiments, R² is a pyrrolidine that is substituted with one or more E and 0-4 R⁸. In some embodiments, In some embodiments, R² is a piperidine that is substituted with one or more E and 0-4 R⁸. In some embodiments, R² is substituted with one E and 0-4 R⁸. In some embodiments, R² is substituted with one E and 1-4 R⁸. In some embodiments, the 1-4 R⁸ are each independently selected from halogen and C₁₋₆alkyl, wherein any C₁₋₆alkyl is unsubstituted. In some embodiments, the 1-4 R⁸ are each independently selected from halogen and C₁₋₆alkyl, wherein any C₁₋₆alkyl is substituted with one or more R¹¹. In some embodiments, R² is substituted with one E and 0 R⁸. In some embodiments, R² is substituted with one E and 1 R⁸, wherein the R⁸ is a halogen. In some embodiments, R² is substituted with one E and 1 R⁸, wherein the R⁸ is C₁₋₆alkyl that is unsubstituted or substituted with one or more R¹¹. In some embodiments, R² is substituted with one E and 1 R⁸, wherein the R⁸ is C₁₋₆alkyl that is unsubstituted. In some embodiments, R² is substituted with one E and 1 R⁸, wherein the R⁸ is C₁₋₆alkyl that is substituted with an R¹¹ that is —OR¹², such as —OCH₃. In some embodiments, each E is independently selected from:

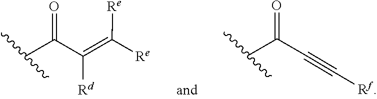

In some embodiments, R² is substituted with one E having the structure:

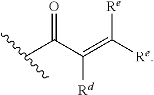

In some embodiments, each R^{d} and R^{e} is H.

In some embodiments, for a compound according to Formula I, R² is a heterocycle selected from azetidine, pyrrolidine, and piperidine, wherein the heterocycle is substituted with one or more E and 0-4 R⁸; R³ is H; R¹ is selected from:

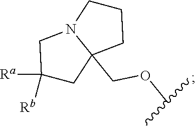

and R⁵ is selected from:

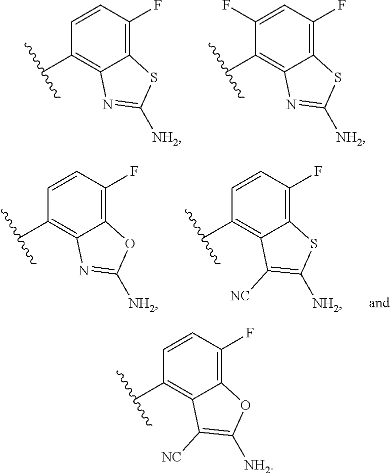

In some embodiments, each E is independently selected from:

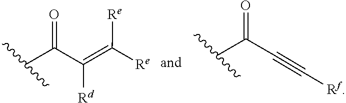

In some embodiments, R² is substituted with one E having the structure:

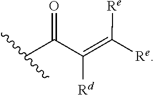

In some embodiments, each R^{d} and R^{e} is H. In some embodiments, R⁴ is selected from C₁₋₆alkyl, wherein any C₁₋₆alkyl is unsubstituted or substituted with one or more R¹¹. In some embodiments, R⁴ is selected from C₁₋₆alkyl that is substituted with one or more halogens. In some embodiments, R⁴ is —CF₃. In some embodiments, R⁶ is selected from halogen and H. In some embodiments, R⁶ is H. In some embodiments, the compound is a compound according to Formula IA:

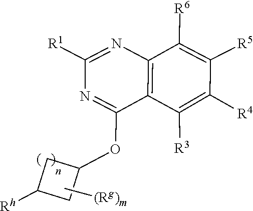

or a salt (e.g., pharmaceutically acceptable salt), ester, tautomer, prodrug, zwitterionic form, or stereoisomer thereof, wherein:

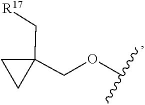

R¹ is selected from —OR⁷, and a 4-6 membered heterocycle comprising a nitrogen atom, wherein the heterocycle is unsubstituted or substituted with one or more R¹⁵;
R³ is selected from H, —OR¹⁰, and C₁₋₆alkyl, wherein any C₁₋₆alkyl is unsubstituted or substituted with one or more R¹¹;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
n is 1, 2, or 3;
m is 0, 1, 2, 3, or 4;
R^{h} and each R^{g} is independently selected from E, halogen, C₁₋₆alkyl, and —OR¹⁰, wherein any C₁₋₆alkyl is unsubstituted or substituted with one or more R¹¹, and R^{h} or at least one R⁸ is E;
each E is independently selected from

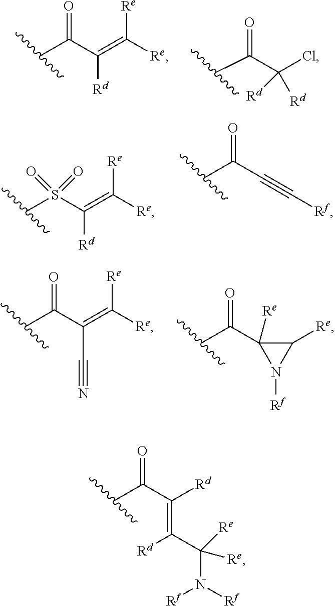

and —CN;
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} and R^{e} is independently selected from halogen, C₁₋₆ alkyl, and H; and
each R^{f} is independently selected from C₁₋₆ alkyl and H.

In some embodiments, the present disclosure provides a compound of Formula IA, or a salt (e.g., a pharmaceutically acceptable salt) thereof.

In some embodiments, the compound is a compound according to Formula IA1:

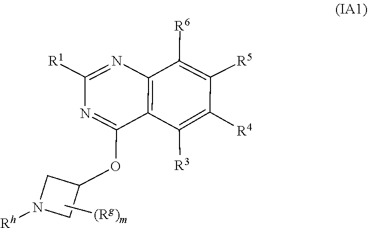

or a salt (e.g., pharmaceutically acceptable salt), ester, tautomer, prodrug, zwitterionic form, or stereoisomer thereof. In some embodiments, the compound is a compound according to Formula IA1, or a salt (e.g., a pharmaceutically acceptable salt) thereof.

In some embodiments, the compound is a compound according to Formula IA2:

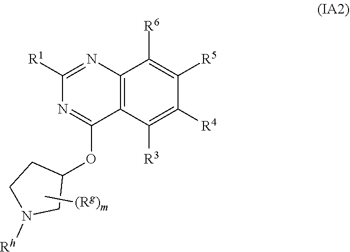

or a salt (e.g., pharmaceutically acceptable salt), ester, tautomer, prodrug, zwitterionic form, or stereoisomer thereof. In some embodiments, the compound is a compound according to Formula IA2, or a salt (e.g., a pharmaceutically acceptable salt) thereof.

In some embodiments, the compound is a compound according to Formula IA3:

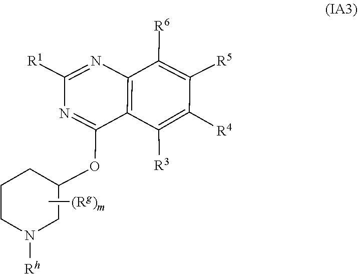

or a salt (e.g., pharmaceutically acceptable salt), ester, tautomer, prodrug, zwitterionic form, or stereoisomer thereof. In some embodiments, the compound is a compound according to Formula IA3, or a salt (e.g., a pharmaceutically acceptable salt) thereof.

In some embodiments, for a compound according to any one of Formulas IA, IA1, IA2, and IA3, R^{h} is E. In some embodiments, R^{h} is:

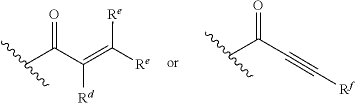

In some embodiments, R^{h} is:

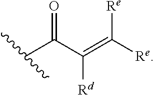

In some embodiments, each R^{d} and R^{e} is H.

In some embodiments, for a compound according to any one of Formulas IA, IA1, IA2, and IA3, R^{h} and each R^{g} is independently selected from E, halogen, and C₁₋₆alkyl, wherein any C₁₋₆alkyl is unsubstituted or substituted with one or more R¹¹, and R^{h} or at least one R⁸ is E. In some embodiments, R^{h} is E; and each R⁸ is independently selected from halogen and C₁₋₆alkyl, wherein any C₁₋₆alkyl is unsubstituted or substituted with one or more R¹¹.

In some embodiments, for a compound according to any one of Formulas IA, IA1, IA2, and IA3, m is 0. In some embodiments, m is 1 or 2. In some embodiments, m is 1 or 2, and each R⁸ is independently selected from halogen and C₁₋₆alkyl, wherein any C₁₋₆alkyl is unsubstituted. In some embodiments, m is 1 or 2, and each R⁸ is independently selected from halogen and C₁₋₆alkyl, wherein any C₁₋₆alkyl is unsubstituted or substituted with one or more R¹¹.

In some embodiments, for a compound according to any one of Formulas I, IA, IA1, IA2, and IA3, R¹ is —OR⁷, wherein R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁷ is a heterocycle or an alkylheterocycle, wherein any heterocycle contains 4-8 members and is substituted with one or more R^{a} or R^{b}. In some embodiments, R⁷ is a heterocycle that is unsubstituted or substituted with one or more R^{a} or R^{b}. In some embodiments, R⁷ is an alkylheterocycle that is unsubstituted or substituted with one or more R^{a} or R^{b}. In some embodiments, R⁷ is —CH₂ (heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃).

In some embodiments, for a compound according to any one of Formulas I, IA, IA1, IA2, and IA3, R¹ is selected from:

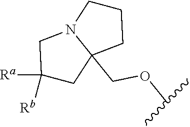

wherein R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

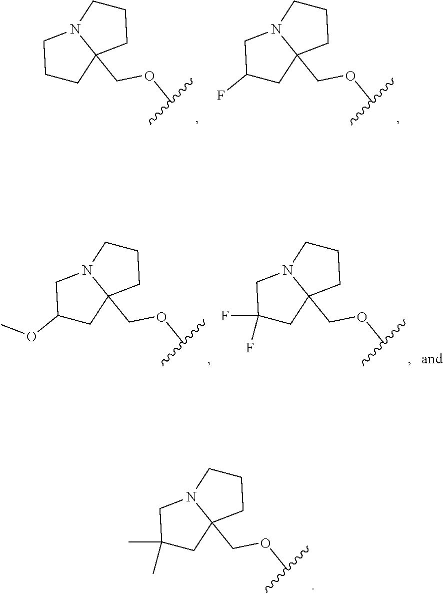 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

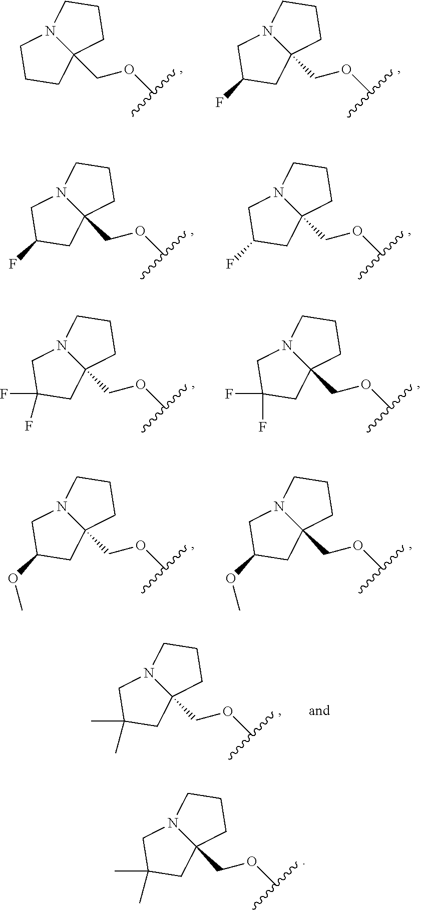

In some embodiments, for a compound according to any one of Formulas I, IA, IA1, IA2, and IA3, R¹ is selected from:

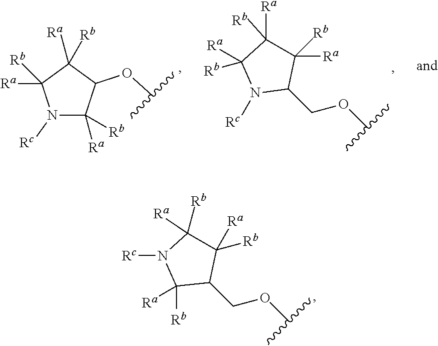

wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H, wherein an R^{a} and R^{e} join together to form a 3-6 membered heterocycle. In some embodiments, each R^{a} and R^{b} is independently selected from halogen, C₁₋₆ alkyl, —OR¹², and H; and R^{e} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆ alkyl (e.g., methyl). In some embodiments, two R^{a} groups, two R^{b} groups, or an R^{a} and an R^{b} are C₁₋₆ alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{e} join together to form a 3-6 membered heterocycle. In some embodiments, R¹ is selected from:

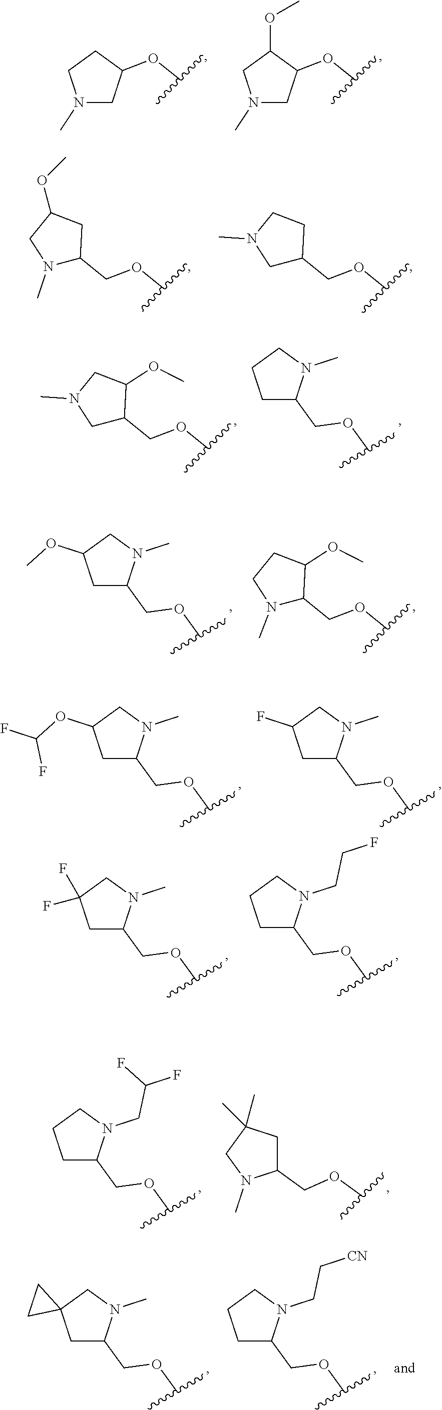

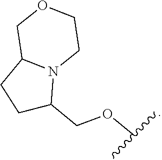 [SMILES: *OCC1CCC2COCCN12]

In some embodiments, R¹ is selected from:

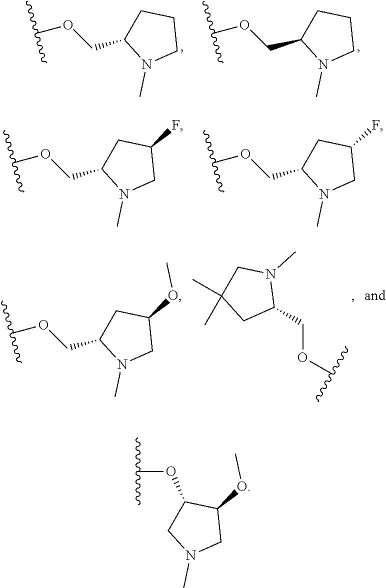

In some embodiments, for a compound according to any one of Formulas I, IA, IA1, IA2, and IA3, R¹ is

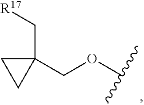

In some embodiments, R¹ is selected from:

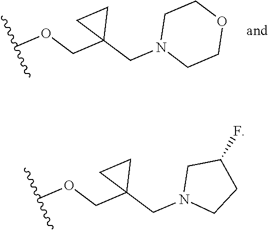 [SMILES: *OCC1(CN2CCOCC2)CC1 ; *OCC1(CN2CC[C@@H](F)C2)CC1]

In some embodiments, for a compound according to any one of Formulas I, IA, IA1, IA2, and IA3, R¹ is a 4-6 membered heterocycle comprising a nitrogen atom, wherein the heterocycle is unsubstituted or substituted with one or more R¹⁵. In some embodiments, R¹ is a 4-membered heterocycle comprising a nitrogen atom, wherein the heterocycle is unsubstituted or substituted with one or more R¹⁵. In some embodiments, R¹ is selected from:

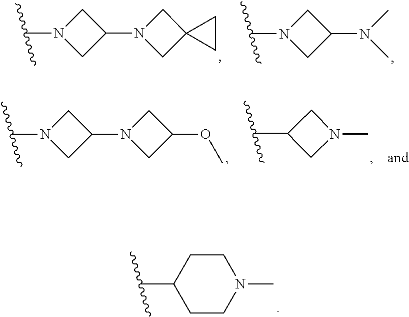

In some embodiments, for a compound according to any one of Formulas I, IA, IA1, IA2, and IA3, R³ is H. In some embodiments, R³ is selected from —OR¹⁰ and C₁₋₆alkyl, wherein any C₁₋₆alkyl is unsubstituted or substituted with one or more R¹¹.

In some embodiments, for a compound according to any one of Formulas I, IA, IA1, IA2, and IA3, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is —CN. In some embodiments, R⁴ is a halogen (e.g., F or C₁). In some embodiments, R⁴ is C₁₋₆alkyl that is unsubstituted, such as methyl or ethyl. In some embodiments, R⁴ is C₁₋₆alkyl that is substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴) 2. In some embodiments, R⁴ is —CH₂CN.

In some embodiments, for a compound according to any one of Formulas I, IA, IA1, IA2, and IA3, R⁵ is:

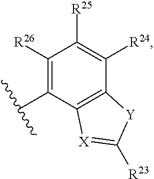

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²) 2. In some embodiments, X is C—CN, Y is S, and R²³ is —NH₂.

In some embodiments, for a compound according to any one of Formulas I, IA, IA1, IA2, and IA3, R⁵ is selected from:

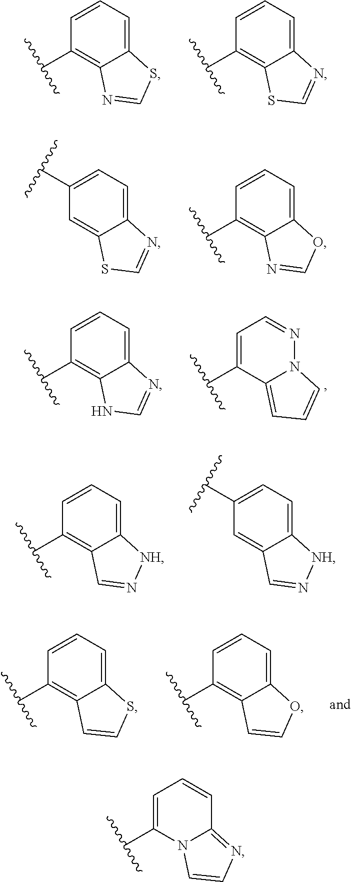

any of which is substituted with one or more R⁹.

In some embodiments, for a compound according to any one of Formulas I, IA, IA1, IA2, and IA3, R⁵ is selected from:

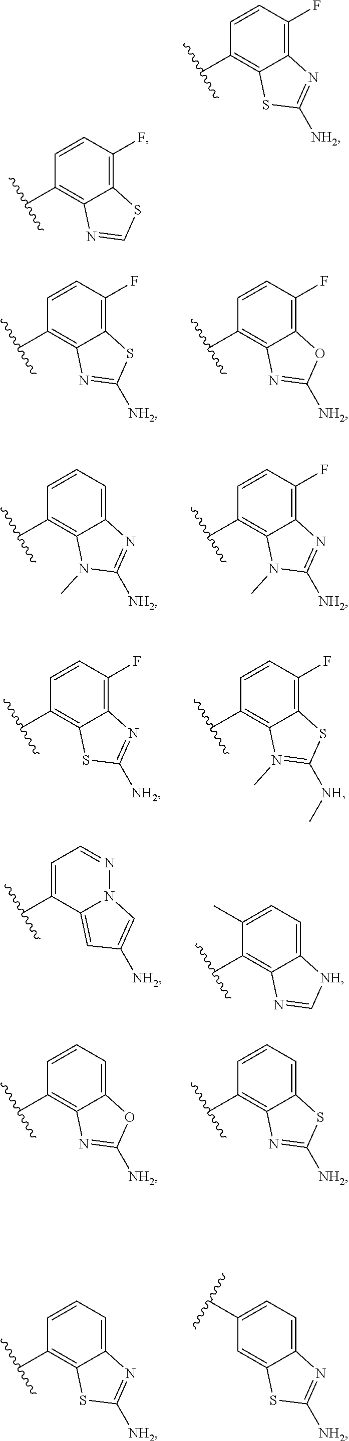

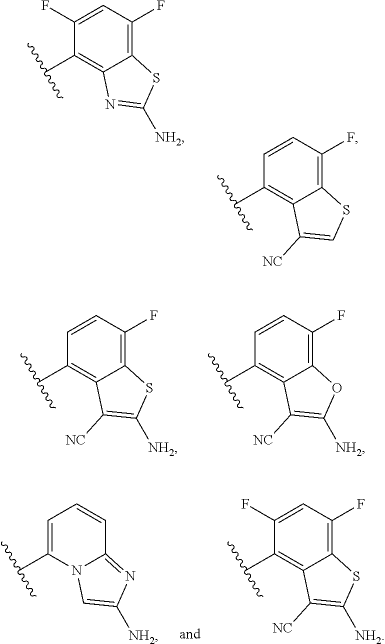 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12 ; *c1c(F)cc(F)c2sc(N)c(C#N)c12]

In some embodiments, for a compound according to any one of Formulas I, IA, IA1, IA2, and IA3, R⁵ is selected from:

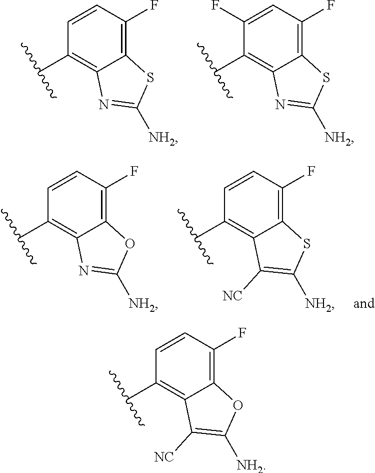

In some embodiments, for a compound according to any one of Formulas I, IA, IA1, IA2, and IA3, R⁶ is selected from halogen, —CN, and H. In some embodiments, R⁶ is a halogen (e.g., Cl or F). In some embodiments, R⁶ is —CN. In some embodiments, R⁶ is H.

In some embodiments, the present disclosure provides a compound according to Formula IB:

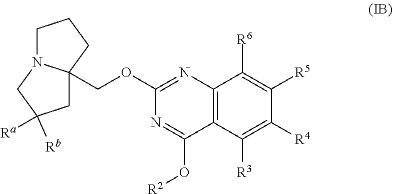

or a salt (e.g., pharmaceutically acceptable salt), ester, tautomer, prodrug, zwitterionic form, or stereoisomer thereof, wherein:
R² is a 4-6 membered heterocycle containing one or more nitrogen atoms, wherein the heterocycle is substituted with one or more E and 0-4 R⁸;
R³ is selected from H, —OR¹⁰, and C₁₋₆alkyl, wherein any C₁₋₆alkyl is unsubstituted or substituted with one or more R¹¹;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
each R⁸ is independently selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each E is independently selected from

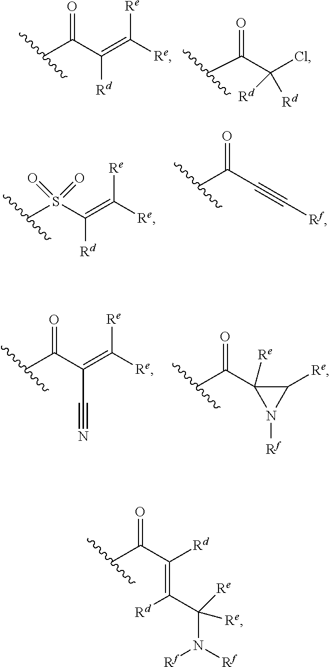

and —CN;
R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³,
each R^{d} and R^{e} is independently selected from halogen, C₁₋₆ alkyl, and H; and
each R^{f} is independently selected from C₁₋₆ alkyl and H.

In some embodiments, the present disclosure provides a compound of Formula IB, or a salt (e.g., a pharmaceutically acceptable salt) thereof.

In some embodiments, for a compound according to Formula IB, R^{a} is a halogen. In some embodiments, R^{a} is a —OR¹². In some embodiments, R^{b} is H. In some embodiments, R^{a} is a halogen and R^{b} is H. In some embodiments, R^{a} is —OR¹² and R^{b} is H.

In some embodiments, for a compound according to Formula IB, R² is a 4-6 membered heterocycle containing one nitrogen atom, wherein the heterocycle is substituted with one or more E and 0-4 R⁸. In some embodiments, R² is a heterocycle selected from azetidine, pyrrolidine, and piperidine, wherein the heterocycle is substituted with one or more E and 0-4 R⁸. In some embodiments, R² is an azetidine that is substituted with one or more E and 0-4 R⁸. In some embodiments, R² is a pyrrolidine that is substituted with one or more E and 0-4 R⁸. In some embodiments, In some embodiments, R² is a piperidine that is substituted with one or more E and 0-4 R⁸. In some embodiments, R² is substituted with one E and 0-4 R⁸. In some embodiments, R² is substituted with one E and 1-4 R⁸. In some embodiments, the 1-4 R⁸ are each independently selected from halogen and C₁₋₆alkyl, wherein any C₁₋₆alkyl is unsubstituted. In some embodiments, the 1-4 R⁸ are each independently selected from halogen and C₁₋₆alkyl, wherein any C₁₋₆alkyl is substituted with one or more R¹¹. In some embodiments, R² is substituted with one E and 0 R⁸. In some embodiments, R² is substituted with one E and 1 R⁸, wherein the R⁸ is a halogen. In some embodiments, R² is substituted with one E and 1 R⁸, wherein the R⁸ is C₁₋₆alkyl that is unsubstituted or substituted with one or more R¹¹. In some embodiments, R² is substituted with one E and 1 R⁸, wherein the R⁸ is C₁₋₆alkyl that is unsubstituted. In some embodiments, R² is substituted with one E and 1 R⁸, wherein the R⁸ is C₁₋₆alkyl that is substituted with an R¹¹ that is —OR¹², such as —OCH₃. In some embodiments, each E is independently selected from:

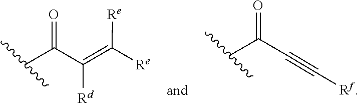

In some embodiments, R² is substituted with one E having the structure:

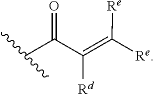

In some embodiments, each R^{d} and R^{e} is H.

In some embodiments, for a compound according to Formula IB, R³ is H. In some embodiments, R³ is selected from —OR¹⁰ and C₁₋₆alkyl, wherein any C₁₋₆alkyl is unsubstituted or substituted with one or more R¹¹.

In some embodiments, for a compound according to Formula IB, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is —CN. In some embodiments, R⁴ is a halogen (e.g., F or C₁). In some embodiments, R⁴ is C₁₋₆alkyl that is unsubstituted, such as methyl or ethyl. In some embodiments, R⁴ is C₁₋₆alkyl that is substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴) 2. In some embodiments, R⁴ is —CH₂CN.

In some embodiments, for a compound according to Formula IB, R⁵ is selected from:

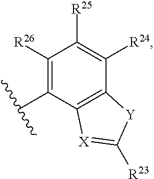

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂.

In some embodiments, for a compound according to Formula IB, R⁵ is selected from:

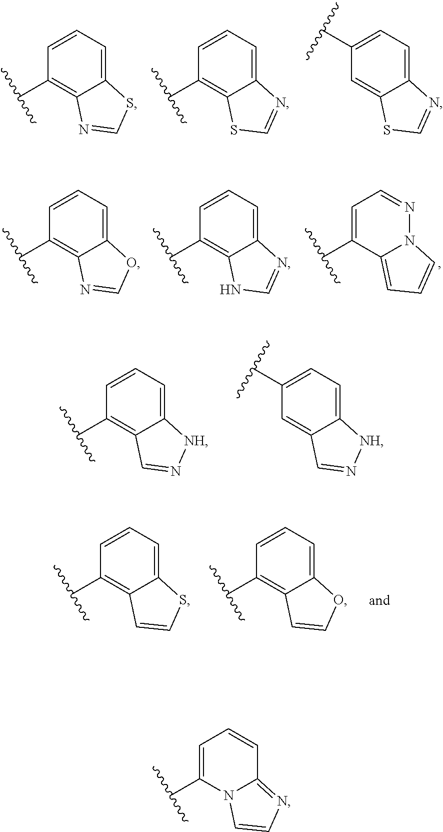

any of which is substituted with one or more R⁹.

In some embodiments, for a compound according to Formula IB, R⁵ is selected from:

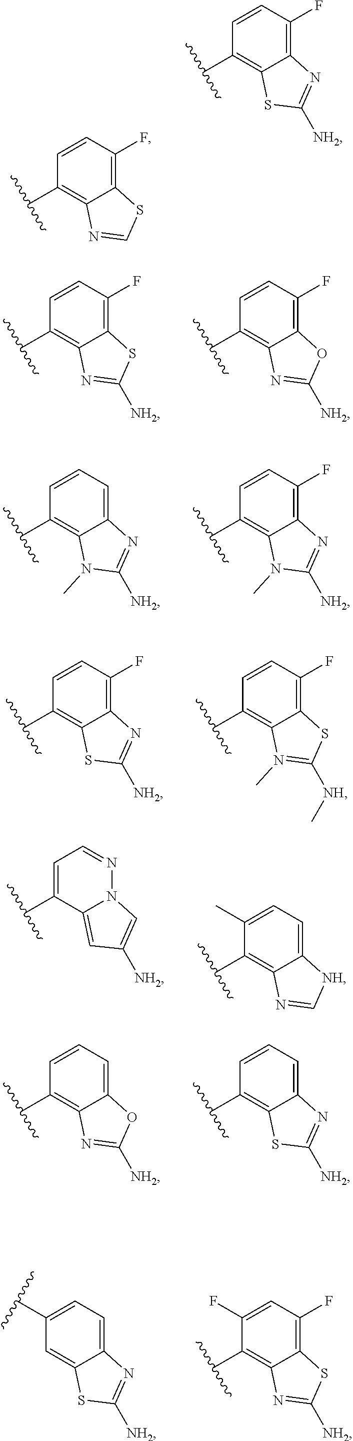

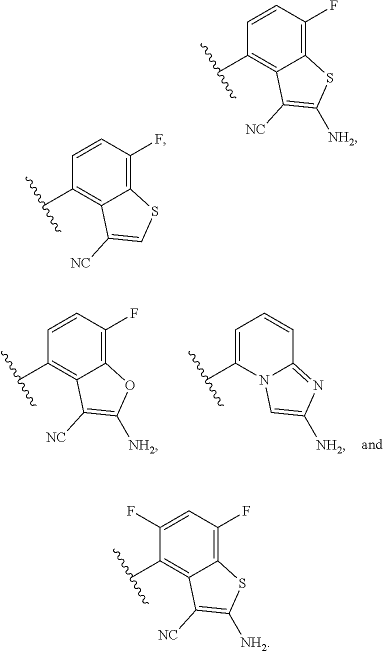 [SMILES: *c1ccc(F)c2sc(N)c(C#N)c12 ; *c1ccc(F)c2scc(C#N)c12 ; *c1ccc(F)c2oc(N)c(C#N)c12 ; *c1cccc2nc(N)cn12 ; *c1c(F)cc(F)c2sc(N)c(C#N)c12]

In some embodiments, for a compound according to Formula IB, R⁵ is selected from:

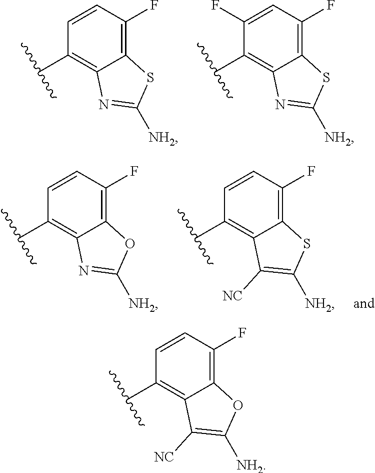

In some embodiments, for a compound according to Formula IB, R⁶ is selected from halogen, —CN, and H. In some embodiments, R⁶ is a halogen (e.g., Cl or F). In some embodiments, R⁶ is —CN. In some embodiments, R⁶ is H.

In another aspect, the present disclosure provides a compound according to Formula II:

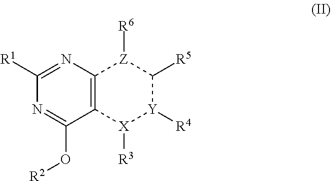

or a salt (e.g., pharmaceutically acceptable salt), ester, tautomer, prodrug, zwitterionic form, or stereoisomer thereof, wherein:
each dashed line represents a single or double bond;
X, Y, and Z are selected from N and C, wherein one and only one of X, Y, and Z is N;
R¹ is selected from —OR⁷,

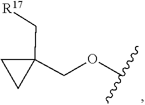

and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is substituted with one or more E and 0-4 R⁸;
when X is C, R³ is selected from H, —OR¹⁰, and C₁₋₆alkyl, wherein any C₁₋₆alkyl is unsubstituted or substituted with one or more R¹¹, and when X is N, R³ is absent;
when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
R⁵ is a bicyclic heteroaryl substituted with one or more R⁹;
when Z is C, R⁶ is selected from halogen, —OR¹², —CN, and H, and when Z is N, R⁶ is absent;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is selected from halogen and C₁₋₆alkyl, wherein any C₁₋₆alkyl is unsubstituted or substituted with one or more R¹;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
each E is independently selected from

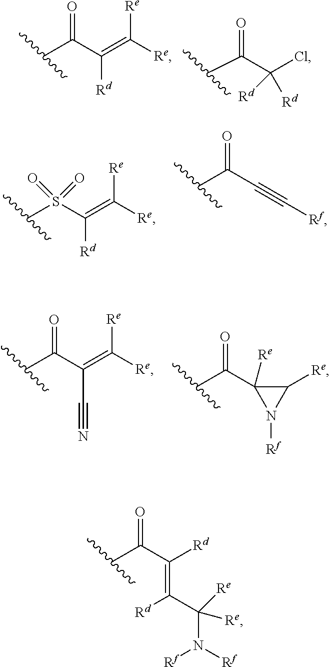

and —CN;
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} and R^{e} is independently selected from halogen, C₁₋₆ alkyl, and H; and
each R^{f} is independently selected from C₁₋₆ alkyl and H.

In some embodiments, the present disclosure provides a compound of Formula II, or a salt (e.g., a pharmaceutically acceptable salt) thereof.

In some embodiments, for a compound according to Formula II, the ring comprising X, Y, and Z is aromatic.

In some embodiments, for a compound according to Formula II, X is N, and Y and Z are C. In some embodiments, the compound is a compound according to Formula IIA:

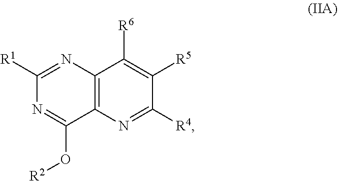

or a salt (e.g., pharmaceutically acceptable salt), ester, tautomer, prodrug, zwitterionic form, or stereoisomer thereof. In some embodiments, the compound is a compound according to Formula IIA, or a salt (e.g., a pharmaceutically acceptable salt) thereof. In some embodiments, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is halogen (e.g., Cl or F). In some embodiments, R⁴ is —CN. In some embodiments, R⁴ is —OR¹². In some embodiments, R⁴ is C₁₋₆alkyl that is unsubstituted or substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴) 2. In some embodiments, R⁴ is —CH₂CN. In some embodiments, R⁶ is selected from halogen, —CN, and H. In some embodiments, R⁶ is halogen (e.g., Cl or F). In some embodiments, R⁶ is —CN. In some embodiments, R⁶ is H.

In some embodiments, for a compound according to Formula II, Y is N, and X and Z are C. In some embodiments, the compound is a compound according to Formula IIB:

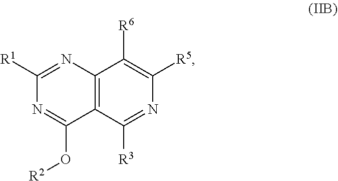

or a salt (e.g., pharmaceutically acceptable salt), ester, tautomer, prodrug, zwitterionic form, or stereoisomer thereof. In some embodiments, the compound is a compound according to Formula IIB, or a salt (e.g., a pharmaceutically acceptable salt) thereof. In some embodiments, R³ is H. In some embodiments, R³ is —OR¹⁰. In some embodiments, R³ is C₁₋₆alkyl that is unsubstituted or substituted with one or more R¹¹. In some embodiments, R⁶ is selected from halogen, —CN, and H. In some embodiments, R⁶ is a halogen (e.g., Cl or F). In some embodiments, R⁶ is —CN. In some embodiments, R⁶ is H.

In some embodiments, for a compound according to Formula II, Z is N, and X and Y are C. In some embodiments, the compound is a compound according to Formula IIC:

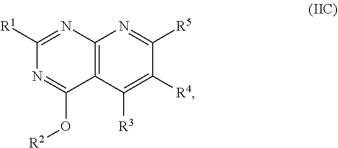

or a salt (e.g., pharmaceutically acceptable salt), ester, tautomer, prodrug, zwitterionic form, or stereoisomer thereof. In some embodiments, the compound is a compound according to Formula IIC, or a salt (e.g., a pharmaceutically acceptable salt) thereof. In some embodiments, R³ is H. In some embodiments, R³ is —OR¹⁰. In some embodiments, R³ is C₁₋₆alkyl that is unsubstituted or substituted with one or more R¹¹. In some embodiments, R⁴ is H. In some embodiments, R⁴ is selected from halogen, —CN, —OR¹², and C₁₋₆alkyl, wherein any C₁₋₆alkyl is unsubstituted or substituted with one or more R¹¹. In some embodiments, R⁴ is halogen (e.g., Cl or F). In some embodiments, R⁴ is —CN. In some embodiments, R⁴ is —OR¹². In some embodiments, R⁴ is C₁₋₆alkyl that is unsubstituted or substituted with one or more R¹¹. In some embodiments, R⁴ is C₁₋₆alkyl that is substituted with one or more halogens or —CN. In some embodiments, R⁴ is C₁₋₆alkyl that is substituted with one or more halogens, such as one or more fluorines. In some embodiments, R⁴ is —CF₃. In some embodiments, R⁴ is —CHF₂. In some embodiments, R⁴ is selected from —CF₂H, —CF₃, —CH₂CN, and —CH₂CH₃. In some embodiments, R⁴ is selected from —CH₃, —CH₂CH₃, —CF₂H, —CF₃, —CF₂CH₃, and —CH₂CN. In some embodiments, R⁴ is C₁₋₆alkyl that is substituted with one or more R¹³, wherein each R¹³ is independently selected from —OR¹⁴, —CN, and —N(R¹⁴) 2. In some embodiments, R⁴ is —CH₂CN.

In some embodiments, for a compound according to any one of Formulas II, IIA, IIB, and IIC, R² is a 4-6 membered heterocycle containing one nitrogen atom, wherein the heterocycle is substituted with one or more E and 0-4 R⁸. In some embodiments, R² is a heterocycle selected from azetidine, pyrrolidine, and piperidine, wherein the heterocycle is substituted with one or more E and 0-4 R⁸. In some embodiments, R² is an azetidine that is substituted with one or more E and 0-4 R⁸. In some embodiments, R² is a pyrrolidine that is substituted with one or more E and 0-4 R⁸. In some embodiments, In some embodiments, R² is a piperidine that is substituted with one or more E and 0-4 R⁸. In some embodiments, R² is substituted with one E and 0-4 R⁸. In some embodiments, R² is substituted with one E and 1-4 R⁸. In some embodiments, the 1-4 R⁸ are each independently selected from halogen and C₁₋₆alkyl, wherein any C₁₋₆alkyl is unsubstituted. In some embodiments, the 1-4 R⁸ are each independently selected from halogen and C₁₋₆alkyl, wherein any C₁₋₆alkyl is substituted with one or more R¹¹. In some embodiments, R² is substituted with one E and 0 R⁸. In some embodiments, R² is substituted with one E and 1 R⁸, wherein the R⁸ is a halogen. In some embodiments, R² is substituted with one E and 1 R⁸, wherein the R⁸ is C₁₋₆alkyl that is unsubstituted or substituted with one or more R¹¹. In some embodiments, R² is substituted with one E and 1 R⁸, wherein the R⁸ is C₁₋₆alkyl that is unsubstituted. In some embodiments, R² is substituted with one E and 1 R⁸, wherein the R⁸ is C₁₋₆alkyl that is substituted with an R¹¹ that is —OR¹², such as —OCH₃. In some embodiments, each E is independently selected from:

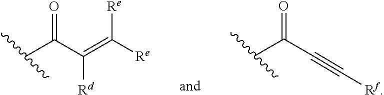

In some embodiments, R² is substituted with one E having the structure:

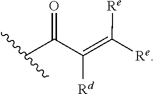

In some embodiments, each R^{d} and R^{e} is H.

In some embodiments, for a compound according to any one of Formulas II, IIA, IIB, and IIC, R¹ is —OR⁷, wherein R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁷ is a heterocycle or an alkylheterocycle, wherein any heterocycle contains 4-8 members and is substituted with one or more R^{a} or R^{b}. In some embodiments, R⁷ is a heterocycle that is unsubstituted or substituted with one or more R^{a} or R^{b}. In some embodiments, R⁷ is an alkylheterocycle that is unsubstituted or substituted with one or more R^{a} or R^{b}. In some embodiments, R⁷ is —CH₂ (heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃).

In some embodiments, for a compound according to any one of Formulas II, IIA, IIB, and IIC, R¹ is selected from:

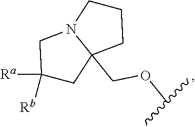

wherein R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

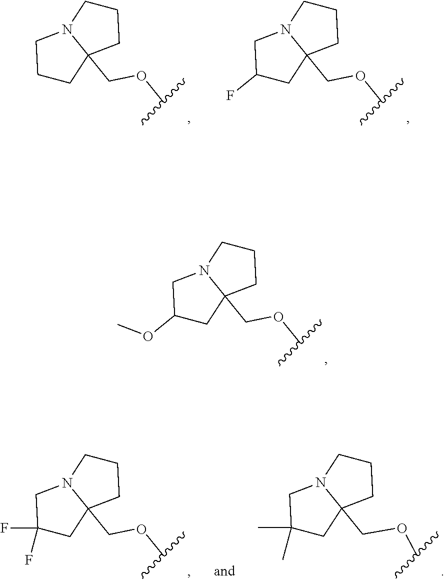 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

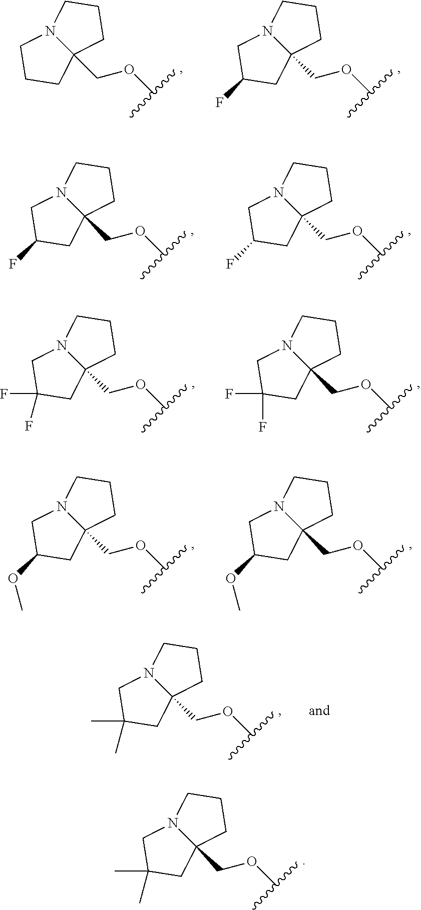

In some embodiments, for a compound according to any one of Formulas II, IIA, IIB, and IIC, R¹ is selected from:

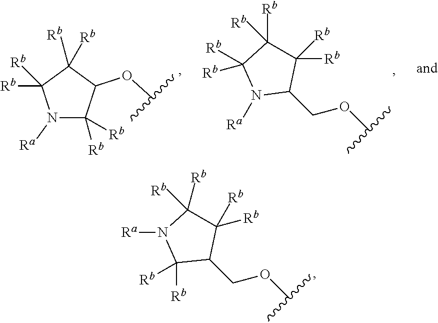

wherein each R^{a} and R^{b} is independently selected from halogen, C₁₋₆ alkyl, —OR¹², and H; and R^{e} is selected from C₁₋₆ alkyl, wherein any C₁₋₆ alkyl is unsubstituted or is substituted with one or more R¹³, and wherein an R^{a} and R^{b} or R^{c} optionally join together to form a 3-6 membered carbocycle or heterocycle. In some embodiments, each R^{a} and R^{b} is independently selected from halogen, C₁₋₆ alkyl, —OR¹², and H; and R^{e} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H, wherein an R^{a} and R^{e} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆ alkyl (e.g., methyl). In some embodiments, two R^{a} groups, two R^{b} groups, or an R^{a} and an R^{b} are C₁₋₆ alkyl (e.g., methyl). In some embodiments, R^{e} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

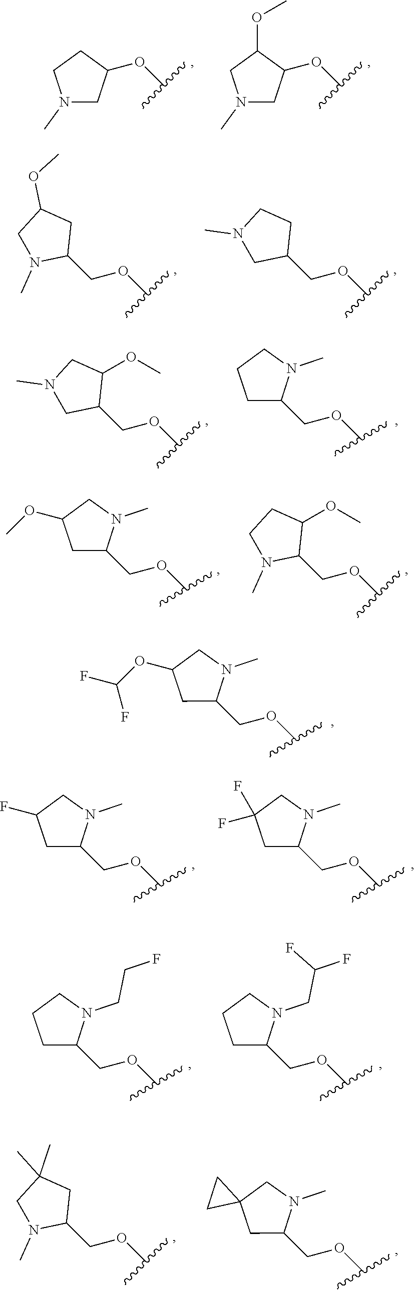

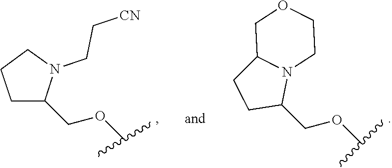 [SMILES: *OCC1CCCN1CCC#N ; *OCC1CCC2COCCN12]

In some embodiments, R¹ is selected from:

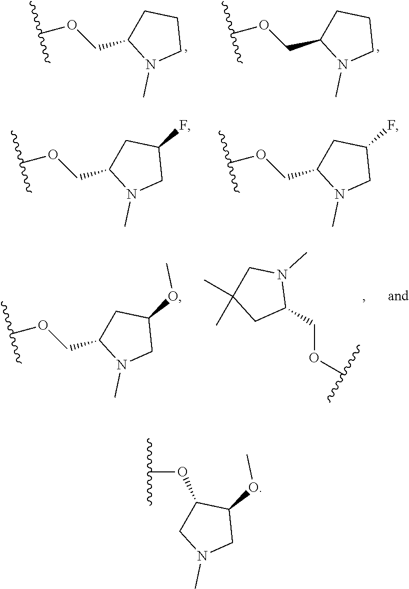

In some embodiments, for a compound according to any one of Formulas II, IIA, IIB, and IIC, R¹ is

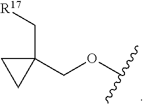

In some embodiments, R¹ is selected from:

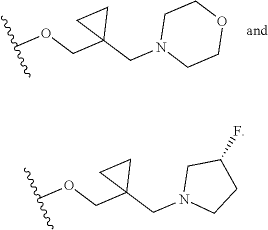 [SMILES: *OCC1(CN2CCOCC2)CC1 ; *OCC1(CN2CC[C@@H](F)C2)CC1]

In some embodiments, for a compound according to any one of Formulas II, IIA, IIB, and IIC, R¹ is a 4-6 membered heterocycle comprising a nitrogen atom, wherein the heterocycle is unsubstituted or substituted with one or more R¹⁵. In some embodiments, R¹ is a 4-membered heterocycle comprising a nitrogen atom, wherein the heterocycle is unsubstituted or substituted with one or more R¹⁵. In some embodiments, R¹ is selected from:

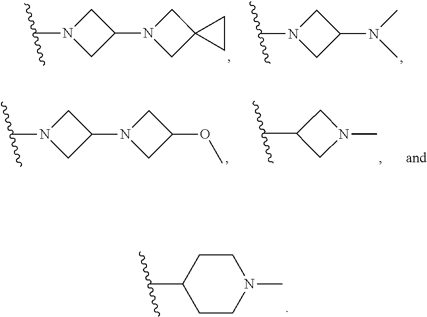

In some embodiments, for a compound according to any one of Formulas II, IIA, IIB, and IIC, R⁵ is:

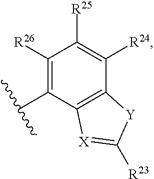

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²) 2. In some embodiments, X is C—CN, Y is S, and R²³ is —NH₂.

In some embodiments, for a compound according to any one of Formulas II, IIA, IIB, and IIC, R⁵ is selected from:

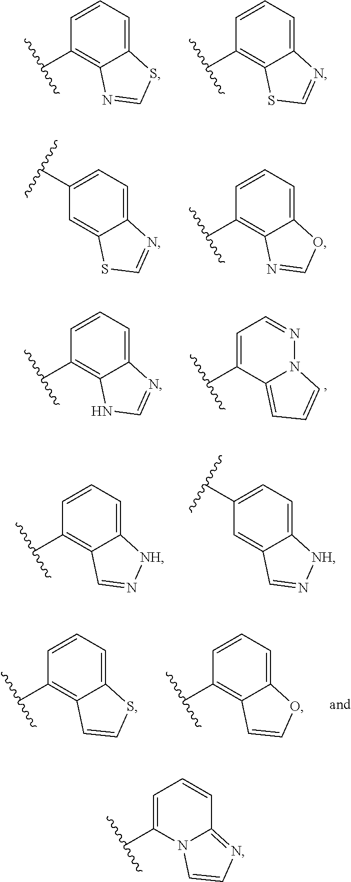

any of which is substituted with one or more R⁹.

In some embodiments, for a compound according to any one of Formulas II, IIA, IIB, and IIC, R⁵ is selected from:

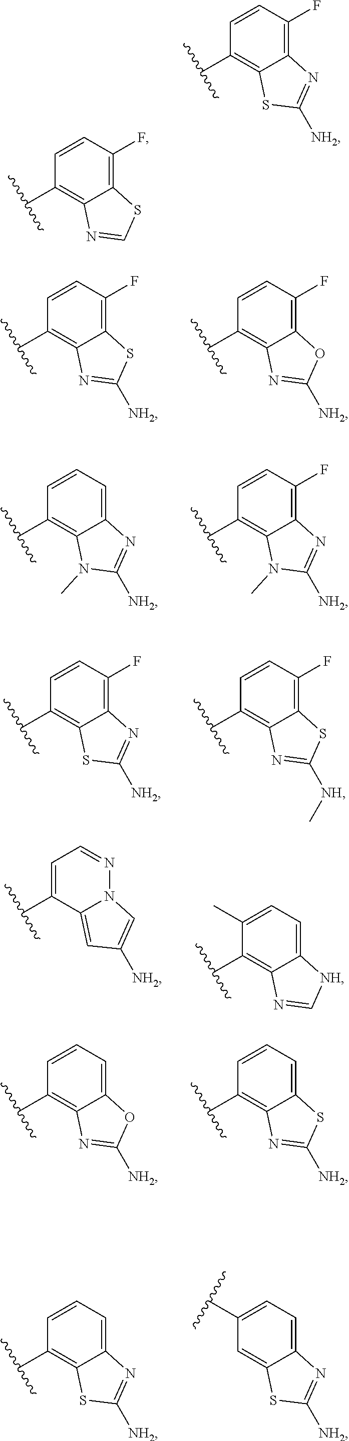

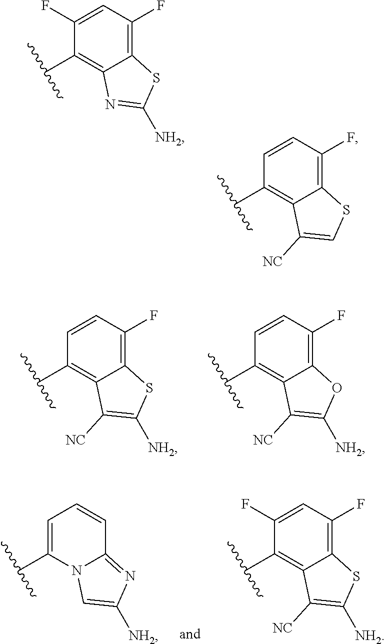 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12 ; *c1c(F)cc(F)c2sc(N)c(C#N)c12]

In some embodiments, for a compound according to any one of Formulas II, IIA, IIB, and IIC, R⁵ is selected from:

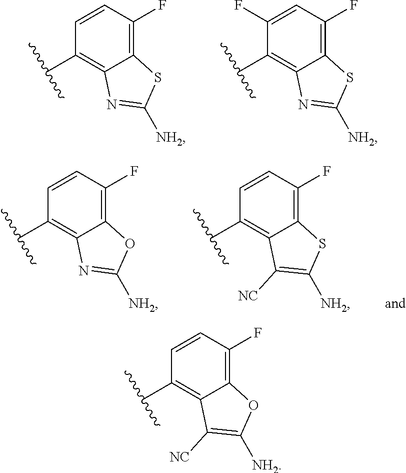

In some embodiments, for a compound according to any one of Formulas II, IIA, IIB, and IIC, R² is a heterocycle selected from azetidine, pyrrolidine, and piperidine, wherein the heterocycle is substituted with one or more E and 0-4 R⁸; R¹ is selected from:

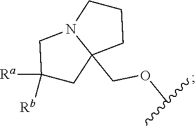

and R⁵ is selected from:

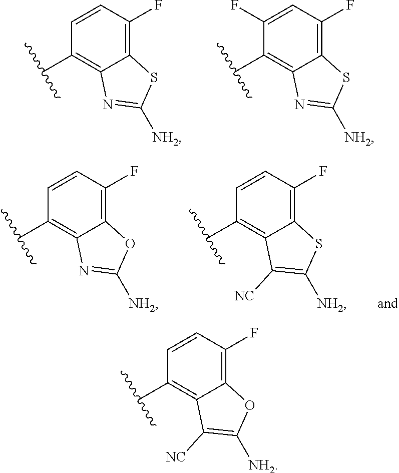

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound included in Table 2, or a salt (e.g., pharmaceutically acceptable salt), ester, tautomer, prodrug, zwitterionic form, or stereoisomer thereof. In some embodiments, the compound is a compound included in Table 2, or a salt (e.g., pharmaceutically acceptable salt) thereof. In some embodiments, the compound is a compound included in Table 2.

In some embodiments of any of the preceding aspects, the compound is a compound included in Table 3, or a salt (e.g., pharmaceutically acceptable salt), ester, tautomer, prodrug, zwitterionic form, or stereoisomer thereof. In some embodiments, the compound is a compound included in Table 3, or a salt (e.g., pharmaceutically acceptable salt) thereof. In some embodiments, the compound is a compound included in Table 3.

Also provided herein is a compound selected from Table 2 or Table 3 or any of the Examples provided herein, or a salt (e.g., pharmaceutically acceptable salt), ester, tautomer, prodrug, zwitterionic form, or stereoisomer thereof. In some embodiments, the present disclosure provides a compound selected from Table 2 or Table 3 or any of the Examples provided herein, or a salt (e.g., pharmaceutically acceptable salt) thereof.

In some embodiments of any of the preceding aspects, a compound provided herein, or a salt, ester, tautomer, zwitterionic form, or stereoisomer thereof, includes an electrophilic moiety E, as provided herein. In some embodiments of any of the preceding aspects, a compound includes multiple electrophilic moieties. In some embodiments of any of the preceding aspects, a compound provided herein, or a salt, ester, tautomer, zwitterionic form, or stereoisomer thereof, is capable of interacting covalently with a cysteine (C) at the 12 position of the KRAS protein (e.g., a G12C mutation) (e.g., via an electrophilic moiety E). In some embodiments of any of the preceding aspects, a compound provided herein, or a salt, ester, tautomer, zwitterionic form, or stereoisomer thereof, binds selectively to KRAS having a G12C mutation relative to KRAS having other residues at the 12 position of the P loop, such as glycine (G), valine (V), arginine (R), serine(S), alanine (A), and aspartic acid (D). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12C mutation relative to KRAS having other residues at the 12 position of the P loop, such as glycine (G), valine (V), arginine (R), serine(S), alanine (A), and aspartic acid (D). In some embodiments of any of the preceding aspects, a compound provided herein, or a salt, ester, tautomer, zwitterionic form, or stereoisomer thereof, binds selectively to KRAS having a G12C mutation relative to wildtype KRAS. For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12C mutation relative to wildtype KRAS. In some embodiments of any of the preceding aspects, a compound provided herein, or a salt, ester, tautomer, zwitterionic form, or stereoisomer thereof, binds selectively to KRAS having a G12C mutation relative to other forms of RAS (e.g., HRAS and NRAS). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100, or greater binding selectivity for KRAS having a G12C mutation relative to another form of RAS (e.g., HRAS or NRAS). In some embodiments of any of the preceding aspects, a compound provided herein, or a salt, ester, tautomer, zwitterionic form, or stereoisomer thereof, is capable of binding to a KRAS protein having a G12C mutation and one or more additional mutations, such as a mutation at the 13 position (to, e.g., D, A, R, S, V, or C).

In some embodiments of any of the preceding aspects, a compound provided herein, or a salt (e.g., pharmaceutically acceptable salt), ester, tautomer, zwitterionic form, or stereoisomer thereof, is capable of selectively binding a KRAS protein in an active (GTP-bound) conformation. In some embodiments of any of the preceding aspects, a compound provided herein, or a salt, ester, tautomer, zwitterionic form, or stereoisomer thereof, is capable of selectively binding a KRAS protein in an inactive (GDP-bound) conformation. In some embodiments of any of the preceding aspects, a compound provided herein, or a salt, ester, tautomer, zwitterionic form, or stereoisomer thereof, is capable of selectively binding a KRAS protein in both active (GTP-bound) and inactive (GDP-bound) conformations. In some embodiments of any of the preceding aspects, a compound provided herein, or a salt, ester, tautomer, zwitterionic form, or stereoisomer(s) thereof, has higher selectivity for a KRAS protein in its active (GTP-bound) conformation than in its inactive (GDP-bound) conformation. In some embodiments of any of the preceding aspects, a compound provided herein, or a salt, ester, tautomer, zwitterionic form, or stereoisomer(s) thereof, has higher selectivity for a KRAS protein in its inactive (GDP-bound) conformation than in its active (GTP-bound) conformation.

### Compositions

The present disclosure also provides a composition (e.g., a pharmaceutical composition) comprising a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof. For example, the present disclosure provides a pharmaceutical composition comprising a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, IA, IA1, IA2, IA3, IB, II, IIA, IIB, and IIC), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, IA, IA1, IA2, IA3, IB, II, IIA, IIB, and IIC), or a pharmaceutically acceptable salt, ester, amide, prodrug or solvate thereof (“active ingredient”), with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, IA, IA1, IA2, IA3, IB, II, IIA, IIB, and IIC in any available form (e.g., salt, ester, tautomer, prodrug, zwitterionic form, stereoisomer(s) etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, IA, IA1, IA2, IA3, IB, II, IIA, IIB, and IIC in any suitable form (e.g., salt, ester, tautomer, prodrug, zwitterionic form, stereoisomer, etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, IA, IA1, IA2, IA3, IB, II, IIA, IIB, and IIC) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, IA, IA1, IA2, IA3, IB, II, IIA, IIB, and IIC or any suitable form thereof (e.g., salt, ester, tautomer, prodrug, zwitterionic form, stereoisomer, etc.)) may be formulated for non-systematic administration, such as topical administration. This includes the application of a compound disclosed herein, or a form thereof, externally to the epidermis or the buccal cavity and the instillation of such a compound, or a form thereof, into the ear, eye and nose, such that the compound, or a form thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments, or pastes, and drops suitable for administration to the eye, ear, or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, IA, IA1, IA2, IA3, IB, II, IIA, IIB, and IIC or any suitable form thereof (e.g., salt, ester, tautomer, prodrug, zwitterionic form, stereoisomer, etc.)) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, IA, IA1, IA2, IA3, IB, II, IIA, IIB, and IIC) or forms thereof (e.g., salt, ester, tautomer, prodrug, zwitterionic form, stereoisomer, etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### Methods

The present disclosure also provides a method of modulating (e.g., inhibiting) KRAS (e.g., KRAS having a G12C mutation) comprising contacting KRAS with a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof. For example, the present disclosure may provide a method of altering a cell phenotype, cell proliferation, KRAS activity, biochemical output produced by active or inactive KRAS, expression of KRAS, and/or binding of KRAS with a natural binding partner. Any such feature may be monitored and may be altered upon contacting KRAS with a compound provided herein, or a form thereof (e.g., salt, ester, tautomer, prodrug, zwitterionic form, or stereoisomer). A method of modulating (e.g., inhibiting) KRAS (e.g., KRAS having a G12C mutation) may be a mode of treatment of a disease, disorder, or condition (e.g., a cancer), a biological assay, a cellular assay, a biochemical assay, etc. In some embodiments, a method of modulating (e.g., inhibiting) KRAS (e.g., KRAS having a G12C mutation) comprises contacting a KRAS protein with a compound provided herein, or a salt, ester, tautomer, zwitterionic form, or stereoisomer thereof, where the KRAS protein is in the active (GTP-bound) conformation. In some embodiments, a method of modulating (e.g., inhibiting) KRAS (e.g., KRAS having a G12C mutation) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwitterionic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a G12C mutation.

The present disclosure also provides methods of treating a disease, disorder, or condition in a subject in need thereof using a compound provided herein, (e.g., a compound of any one of Formulas I, IA, IA1, IA2, IA3, IB, II, IIA, IIB, and IIC), or a salt, ester, tautomer, prodrug, zwitterionic form, or stereoisomer thereof. For example, the present disclosure provides a method comprising providing (e.g., administering) to a subject (e.g., patient) in need thereof an effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof. The present disclosure also provides methods of treating a disease, disorder, or condition in a subject in need thereof using a pharmaceutical composition comprising a compound provided herein, (e.g., a compound of any one of Formulas I, IA, IA1, IA2, IA3, IB, II, IIA, IIB, and IIC), or a salt, ester, tautomer, prodrug, zwitterionic form, or stereoisomer thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof. In some embodiments, the subject is known to have (e.g., has previously been diagnosed with) a disease, disorder, or condition such as a cancer. The disease, disorder, or condition may be a KRAS-mediated disease, such as a cancer characterized by a G12C mutation in KRAS. In some embodiments, the compound administered to the subject in need thereof according to the methods described herein is a compound described in an embodiment, example, figure, or table herein, or a stereoisomer or pharmaceutically acceptable salt thereof.

The present disclosure also provides a compound as provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for use as a medicament, such as a medicament for the treatment of a disease, disorder, or condition (e.g., a cancer). The present disclosure also provides a compound as provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for the treatment of a disease, disorder, or condition (e.g., a cancer) in a subject in need thereof.

The present disclosure also provides the use of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for treating a disease, disorder, or condition (e.g., a cancer) in a subject in need thereof.

The present disclosure also provides a method of inhibiting KRAS (e.g., KRAS having a G12C mutation) (e.g., in a subject in need thereof) comprising contacting KRAS with a compound as provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient. In some embodiments, a method of inhibiting KRAS (e.g., KRAS having a G12C mutation) comprises contacting a KRAS protein with a compound provided herein, or a salt, ester, tautomer, zwitterionic form, or stereoisomer thereof, where the KRAS protein is in the active (GTP-bound) conformation. In some embodiments, a method of inhibiting KRAS (e.g., KRAS having a G12C mutation) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwitterionic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a G12C mutation.

The present disclosure also provides a compound as provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for use in inhibiting KRAS (e.g., KRAS having a G12C mutation) (e.g., in a subject in need thereof). The present disclosure also provides a compound as provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for inhibiting KRAS (e.g., KRAS having a G12C mutation) in a subject in need thereof.

The present disclosure also provides the use of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, for inhibiting KRAS (e.g., KRAS having a G12C mutation) in a subject in need thereof.

The present disclosure also provides the use of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for inhibiting KRAS (e.g., KRAS having a G12C mutation) in a subject in need thereof.

The present disclosure also provides a method comprising administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof to a subject (e.g., patient) (e.g., a subject in need thereof), thereby ameliorating, reducing, eliminating, ceasing, or improving one or more symptoms of the subject, such as one or more symptoms of a disease, disorder, or condition (e.g., a cancer). In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, slows or prevents growth of a tumor. In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer thereof, results in shrinkage of a tumor (e.g., tumor regression). In some embodiments, the subject has a cancer characterized by a mutant KRAS (e.g., KRAS having a G12C mutation).

In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer(s) thereof, slows or prevents growth of a tumor. In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer(s) thereof, results in shrinkage of a tumor (e.g., tumor regression). In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer(s) thereof, results in at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer(s) thereof, stabilizes a tumor. In some embodiments, administering a therapeutically effective amount of a compound provided herein (e.g., a compound of any one of Formulas I, IA, IA1, IA2, IA3, IB, II, IIA, IIB, and IIC), or a salt, ester, tautomer, prodrug, zwitterionic form, or stereoisomer(s) thereof, stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, the subject has a cancer characterized by a mutant KRAS (e.g., KRAS protein having a G12C mutation).

In some embodiments of any of the methods, uses, and medicaments provided herein, the disease, disorder, or condition is a cancer. In some embodiments, the cancer is pancreatic cancer (e.g., pancreatic ductal adenocarcinoma), lung cancer (e.g., non-small cell lung cancer (NSCLC)), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, or ovarian cancer. In some embodiments, the cancer is pancreatic cancer (e.g., pancreatic ductal adenocarcinoma), lung cancer (e.g., non-small cell lung cancer adenocarcinoma), or colorectal cancer (CRC). In some embodiments, the cancer is pancreatic cancer (e.g., pancreatic ductal adenocarcinoma). In some embodiments, the cancer is lung cancer (e.g., non-small cell lung cancer adenocarcinoma). In some embodiments, the cancer is colorectal cancer (CRC). In some embodiments, the cancer is or comprises a solid tumor.

In some embodiments of any of the methods, uses, and medicaments provided herein, the disease, disorder, or condition is a RASopathy (e.g., a genetic syndrome caused by a germline mutation in a gene that encodes a component or regulator of the RAS/MAPK pathway). In some embodiments, the RASopathy is selected from the group consisting of neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome.

In some embodiments of any of the methods, uses, and medicaments provided herein, the disease, disorder, or condition is related to KRAS, such as a disorder associated with a mutation of KRAS or dysregulation of KRAS. In some embodiments, the disease, disorder, or condition is related to the KRAS gene, such as a disease, disorder, or condition associated with a mutation of the KRAS gene or dysregulation of the KRAS gene. Mutation or dysregulation of KRAS or KRAS may include mutation or dysregulation of human K-Ras4a and/or human K-Ras4b. In some embodiments, the disease, disorder, or condition is related to the KRAS (e.g., human K-Ras4a or K-Ras4b) signaling pathway activity, such as a disease, disorder, or condition related to aberrant KRAS signaling pathway activity. In some embodiments, the disease, disorder, or condition is related to mutation or dysregulation of human K-Ras4b. In some embodiments, the disease, disorder, or condition is related to aberrant K-Ras4b signaling pathway activity. In some embodiments, the disease, disorder, or condition is related to mutation or dysregulation of human K-Ras4a. In some embodiments, the disease, disorder, or condition is related to aberrant K-Ras4a signaling pathway activity.

### Administration and Combination Therapy

The compounds provided herein (e.g., compounds of any one of Formulas I, IA, IA1, IA2, IA3, IB, II, IIA, IIB, and IIC or any suitable form thereof (e.g., salt, ester, tautomer, prodrug, zwitterionic form, stereoisomer, etc.)), or compositions (e.g., pharmaceutical compositions) comprising the same, can be administered in various modes (e.g., orally, topically, or by injection). The amount of active ingredient (e.g., compound provided herein or a form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, IA, IA1, IA2, IA3, IB, II, IIA, IIB, and IIC), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, IA, IA1, IA2, IA3, IB, II, IIA, IIB, and IIC), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, IA, IA1, IA2, IA3, IB, II, IIA, IIB, and IIC), or a form thereof, may be administered in combination with an additional agent that may be effective in the treatment of a disease, disorder, or condition such as a cancer. Generally, the combination of a compound provided herein (e.g., a compound of any one of Formulas I, IA, IA1, IA2, IA3, IB, II, IIA, IIB, and IIC), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer, etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MEDI4736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-1BB (tumor necrosis factor receptor superfamily member 9), TIM3 (T-cell immunoglobulin and mucin-domain containing-3), or 0X40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). Additional examples of agents that may be useful in combination with a compound provided herein, or a form thereof (e.g., salt, ester, tautomer, prodrug, zwitterionic form, stereoisomer, etc.), include, but are not limited to, amsacrine; Bacillus Calmette-Guerin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,r′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Two or more therapeutic agents, one of which is a compound provided herein (e.g., a compound of any one of Formulas I, IA, IA1, IA2, IA3, IB, II, IIA, IIB, and IIC) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer, etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, IA, IA1, IA2, IA3, IB, II, IIA, IIB, and IIC), or a form thereof (e.g., salt, ester, tautomer, prodrug, zwitterionic form, stereoisomer, etc.), and at least one additional agent for use in the treatment of a disease, disorder, or condition (e.g., a cancer).

In some embodiments, a method provided herein is used to treat a disease, disorder, or condition (e.g., a cancer) comprising administering to a subject in need thereof a therapeutically effective amount of a compound of any one of Formulas I, IA, IA1, IA2, IA3, IB, II, IIA, IIB, and IIC or a pharmaceutically acceptable salt thereof, wherein the disease, disorder, or condition is a cancer that has developed a resistance to one or more chemotherapeutic drugs and/or ionizing radiation. In some embodiments, a method provided herein is used to treat a disease, disorder, or condition (e.g., a cancer) comprising administering to a subject in need thereof a therapeutically effective amount of a compound of any one of Formulas I, IA, IA1, IA2, IA3, IB, II, IIA, IIB, and IIC or a pharmaceutically acceptable salt thereof, in combination with an additional agent, wherein the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma, and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML)) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. Additional diseases and disorders that may be treated by the methods disclosed herein include, but are not limited to, diseases or disorders related to KRAS, such as diseases or disorders associated with a mutation of KRAS or dysregulation of KRAS, and diseases or disorders related to the KRAS gene, such as diseases or disorders associated with a mutation of the KRAS gene or dysregulation of the KRAS gene.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### Exemplary Embodiments

1. A compound according to Formula I:

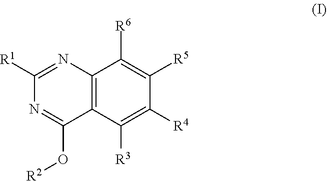

or a salt (e.g., pharmaceutically acceptable salt), ester, tautomer, prodrug, zwitterionic form, or stereoisomer thereof, wherein:
   R¹ is selected from —OR⁷;
   R² is a 4-6 membered heterocycle containing one or more nitrogen atoms, wherein the heterocycle is substituted with one or more E and 0-4 R⁸;
   R³ is selected from H, —OR¹⁰, and C₁₋₆alkyl, wherein any C₁₋₆alkyl is unsubstituted or substituted with one or more R¹¹;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁸ is independently selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹⁰ is independently selected from C₁₋₆ alkyl, C₂₋₆ alkenyl, and H;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H;
   each E is independently selected from

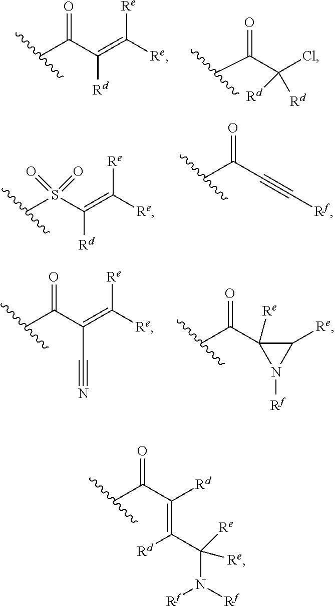

and —CN;
R^{a} and R^{b} are each independently selected from halogen, C₁₋₆ alkyl, —OR¹², and H, wherein any C₁₋₆alkyl is unsubstituted or is substituted with one or more R¹³;
each R^{d} and R^{e} is independently selected from halogen, C₁₋₆ alkyl, and H; and each R^{f} is independently selected from C₁₋₆ alkyl and H.
2. The compound of embodiment 1, wherein R² is a 4-6 membered heterocycle containing one nitrogen atom, wherein the heterocycle is substituted with one or more E and 0-4 R⁸.
3. The compound of embodiment 2, wherein R² is a heterocycle selected from azetidine, pyrrolidine, and piperidine, wherein the heterocycle is substituted with one or more E and 0-4 R⁸.
4. The compound of any one of embodiments 1-3, wherein R² is substituted with one E and 0-4 R⁸.
5. The compound of embodiment 4, wherein R² is substituted with one E and 1-4 R⁸.
6. The compound of embodiment 5, wherein the 1-4 R⁸ are each independently selected from halogen and C₁₋₆alkyl, wherein any C₁₋₆alkyl is unsubstituted.
7. The compound of any one of embodiments 1-6, wherein each E is independently selected from:

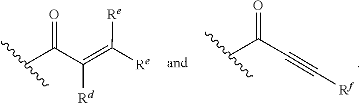

8. The compound of any one of embodiments 1-7, wherein R¹ is selected from:

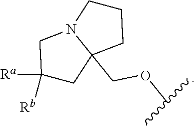

9. The compound of embodiment 8, wherein R^{a} is selected from halogen, C₁₋₆alkyl, —OR¹², and H, wherein any C₁₋₆alkyl is unsubstituted or is substituted with one or more R¹³.
10. The compound of embodiment 8 or 9, wherein R^{b} is H.
11. The compound of embodiment 8, wherein R¹ is selected from:

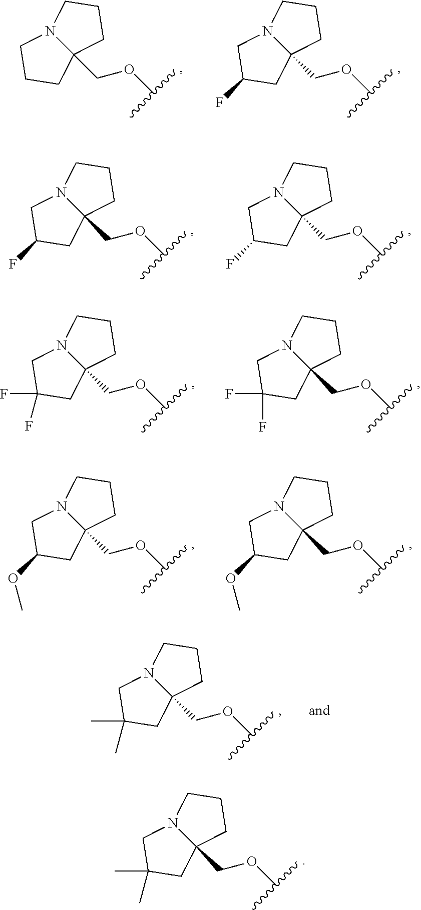

12. The compound of any one of embodiments 1-7, wherein R¹ is selected from:

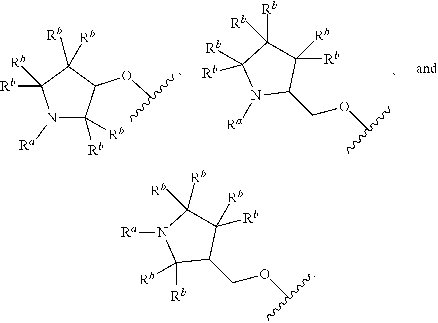

13. The compound of embodiment 12, wherein R^{a} is C₁₋₆alkyl that is unsubstituted or is substituted with one or more R¹³.
14. The compound of embodiment 12 or 13, wherein R¹ is selected from:

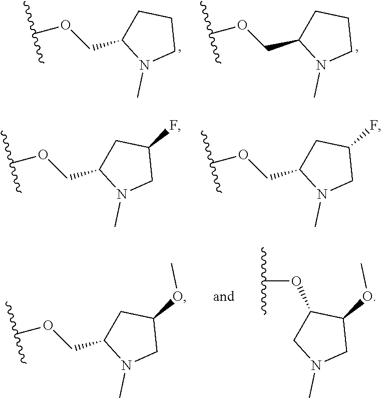

15. The compound of any one of embodiments 1-7, wherein R¹ is selected from:

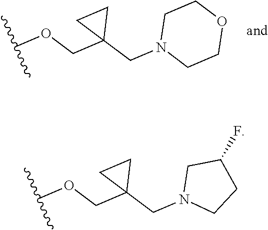 [SMILES: *OCC1(CN2CCOCC2)CC1 ; *OCC1(CN2CC[C@@H](F)C2)CC1]

16. The compound of any one of embodiments 1-15, wherein R³ is H.
17. The compound of any one of embodiments 1-16, wherein R⁴ is H.
18. The compound of any one of embodiments 1-16, wherein R⁴ is selected from halogen, —CN, —OR¹², and C₁₋₆alkyl, wherein any C₁₋₆alkyl is unsubstituted or substituted with one or more R¹¹.
19. The compound of embodiment 18, wherein R⁴ is —CN.
20. The compound of embodiment 18, wherein R⁴ is a halogen.
21. The compound of embodiment 18, wherein R⁴ is C₁₋₆alkyl that is substituted with one or more R¹¹.
22. The compound of any one of embodiments 1-21, wherein R⁵ is selected from:

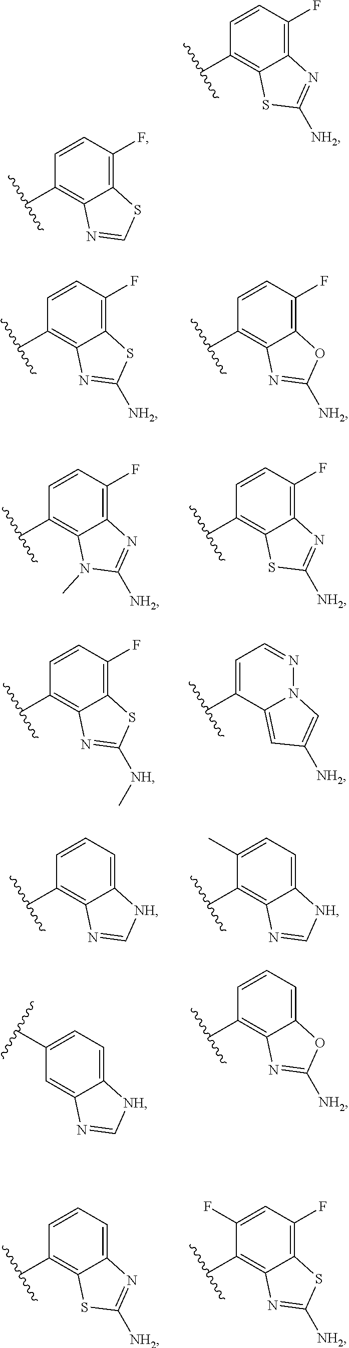

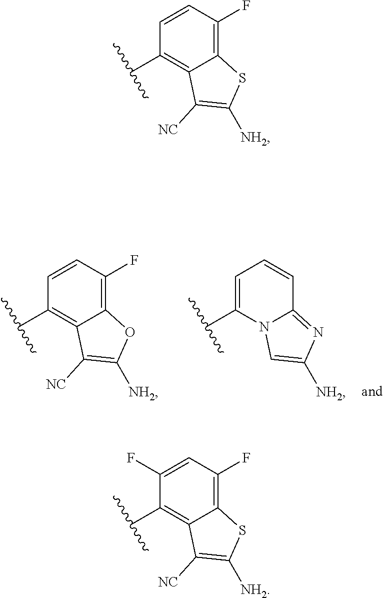 [SMILES: *c1ccc(F)c2sc(N)c(C#N)c12 ; *c1ccc(F)c2oc(N)c(C#N)c12 ; *c1cccc2nc(N)cn12 ; *c1c(F)cc(F)c2sc(N)c(C#N)c12]

23. The compound of embodiment 22, wherein R⁵ is selected from:

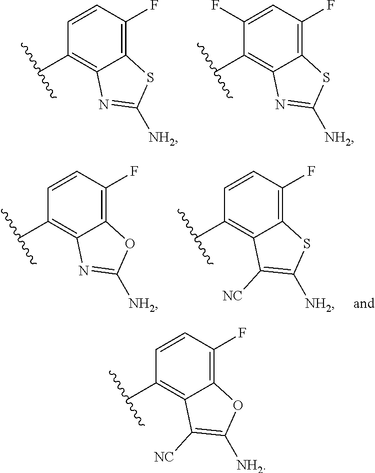

24. The compound of any one of embodiments 1-23, wherein R⁶ is selected from halogen, —CN, and H.
25. The compound of embodiment 24, wherein R⁶ is a halogen.
26. The compound of embodiment 1, wherein R² is a heterocycle selected from azetidine, pyrrolidine, and piperidine, wherein the heterocycle is substituted with one or more E and 0-4 R⁸; R³ is H; R¹ is selected from:

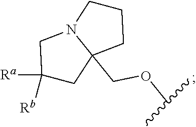

and R⁵ is selected from:

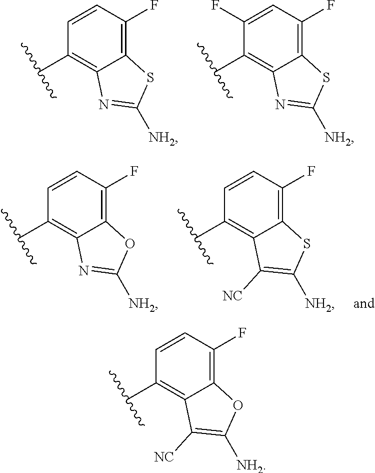

27. A compound according to Formula IA:

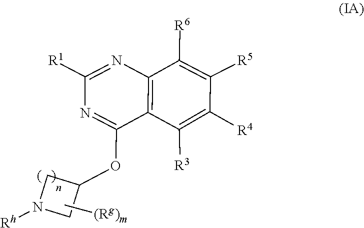

or a salt (e.g., pharmaceutically acceptable salt), ester, tautomer, prodrug, zwitterionic form, or stereoisomer thereof, wherein:
   R¹ is selected from —OR⁷;
   R³ is selected from H, —OR¹⁰, and C₁₋₆alkyl, wherein any C₁₋₆alkyl is unsubstituted or substituted with one or more R¹¹;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁹ is independently selected from halogen, N(R¹²)₂, —CN, and C₁₋₆alkyl, wherein any C₁₋₆alkyl is unsubstituted or substituted with one or more R¹³;
   each R¹⁰ is independently selected from C₁₋₆ alkyl, C₂₋₆ alkenyl, and H;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H;
   n is 1, 2, or 3;
   m is 0, 1, 2, 3, or 4;
   R^{h} and each R^{g} is independently selected from E, halogen, C₁₋₆alkyl, and —OR¹⁰, wherein any C₁₋₆alkyl is unsubstituted or substituted with one or more R¹¹, and R^{h} or at least one R$ is E;
   each E is independently selected from

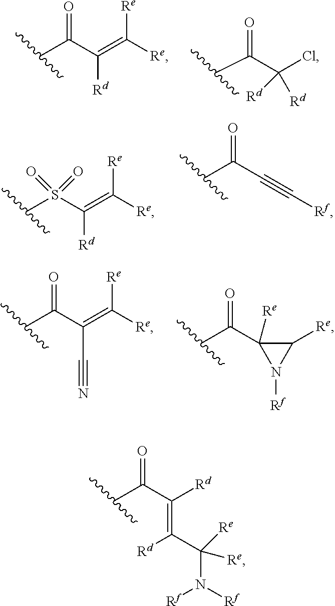

and —CN;
R^{a} and R^{b} are each independently selected from halogen, C₁₋₆ alkyl, —OR¹², and H, wherein any C₁₋₆alkyl is unsubstituted or is substituted with one or more R¹³;
each R^{d} and R^{e} are independently selected from halogen, C₁₋₆ alkyl, and H; and
each R^{f} is independently selected from C₁₋₆ alkyl and H.
28. The compound of embodiment 27, wherein the compound is a compound according to Formula IA1:

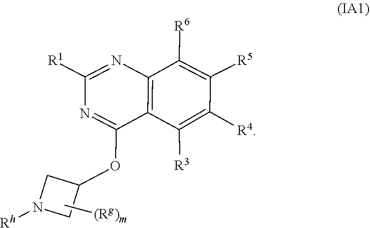

29. The compound of embodiment 27, wherein the compound is a compound according to Formula IA2:

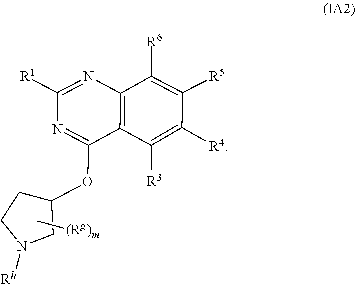

30. The compound of embodiment 27, wherein the compound is a compound according to Formula IA3:

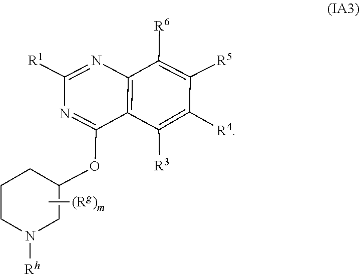

31. The compound of any one of embodiments 27-30, wherein R^{h} is E.
32. The compound of embodiment 31, wherein R^{h} is:

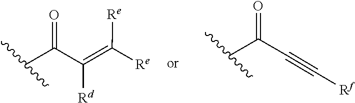

33. The compound of any one of embodiments 27-32, wherein m is 1 or 2, and each R⁸ is independently selected from halogen and C₁₋₆alkyl, wherein any C₁₋₆alkyl is unsubstituted.
34. The compound of any one of embodiments 27-33, wherein R¹ is selected from:

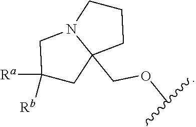

35. The compound of embodiment 34, wherein R^{a} is selected from halogen, C₁₋₆alkyl, —OR¹², and H, wherein any C₁₋₆alkyl is unsubstituted or is substituted with one or more R¹³.
36. The compound of embodiment 34 or 35, wherein R^{b} is H.
37. The compound of embodiment 34, wherein R¹ is selected from:

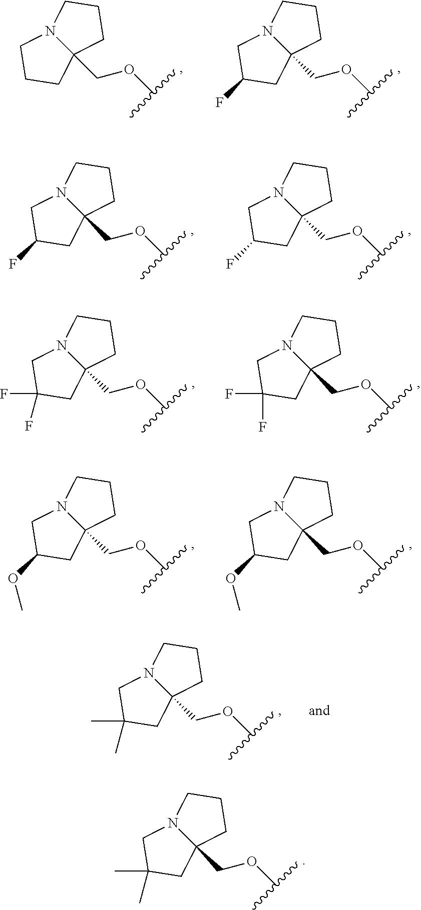

38. The compound of any one of embodiments 27-33, wherein R¹ is selected from:

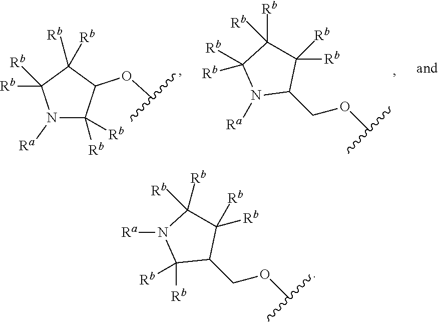

39. The compound of embodiment 38, wherein R^{a} is C₁₋₆alkyl that is unsubstituted or is substituted with one or more R¹³.
40. The compound of embodiment 38 or 39, wherein R¹ is selected from:

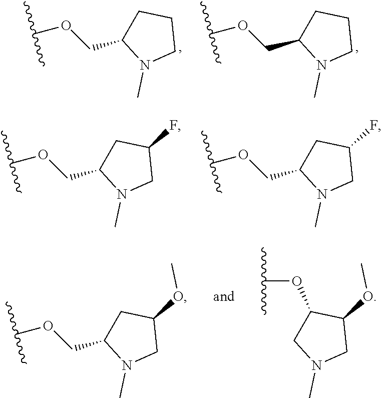

41. The compound of any one of embodiments 27-33, wherein R¹ is selected from:

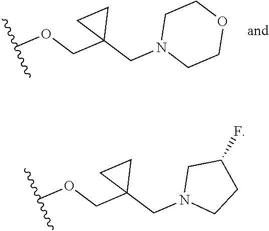 [SMILES: *OCC1(CN2CCOCC2)CC1 ; *OCC1(CN2CC[C@@H](F)C2)CC1]

42. The compound of any one of embodiments 27-41, wherein R³ is H.
43. The compound of any one of embodiments 27-42, wherein R⁴ is H.
44. The compound of any one of embodiments 27-42, wherein R⁴ is selected from halogen, —CN, —OR¹², and C₁₋₆alkyl, wherein any C₁₋₆alkyl is unsubstituted or substituted with one or more R¹¹.
45. The compound of embodiment 44, wherein R⁴ is —CN.
46. The compound of embodiment 44, wherein R⁴ is a halogen.
47. The compound of embodiment 44, wherein R⁴ is C₁₋₆alkyl that is substituted with one or more R¹¹.
48. The compound of any one of embodiments 27-48, wherein R⁵ is selected from:

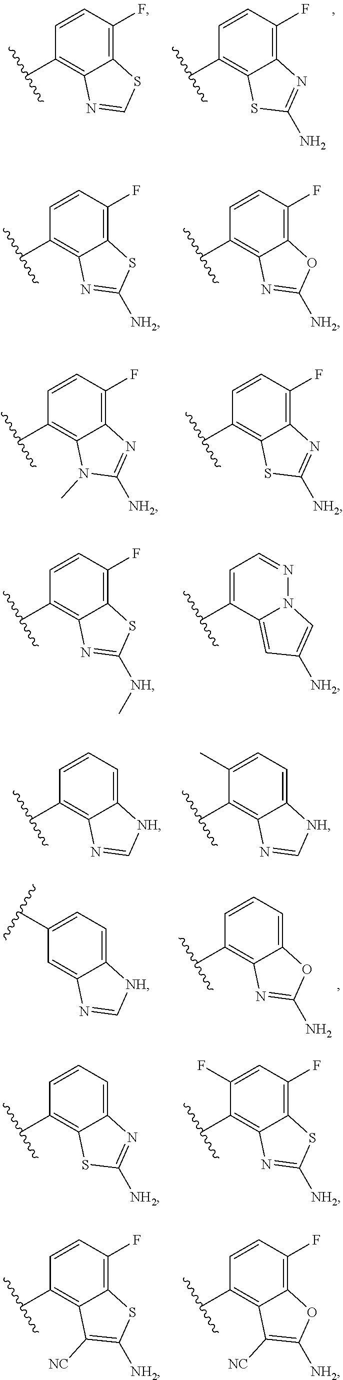

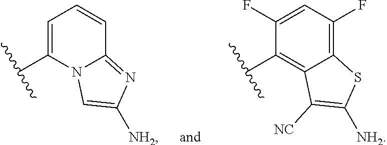 [SMILES: *c1cccc2nc(N)cn12 ; "and" ; *c1c(F)cc(F)c2sc(N)c(C#N)c12]

49. The compound of embodiment 48, wherein R⁵ is selected from:

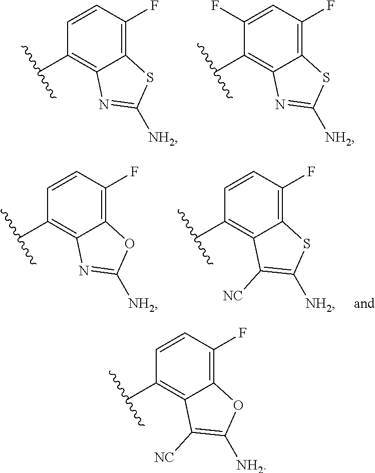

50. The compound of any one of embodiments 27-49, wherein R⁶ is selected from halogen, —CN, and H.
51. The compound of embodiment 50, wherein R⁶ is a halogen.
52. A compound according to Formula IB:

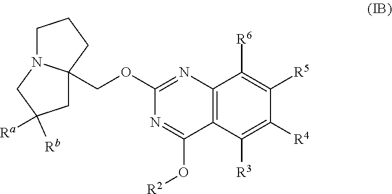

or a salt (e.g., pharmaceutically acceptable salt), ester, tautomer, prodrug, zwitterionic form, or stereoisomer thereof, wherein:
   R² is a 4-6 membered heterocycle containing one or more nitrogen atoms, wherein the heterocycle is substituted with one or more E and 0-4 R⁸;
   R³ is selected from H, —OR¹⁰, and C₁₋₆alkyl, wherein any C₁₋₆alkyl is unsubstituted or substituted with one or more R¹¹;
   R⁴ is selected from H, halogen, —CN, —OR¹², and C₁₋₆alkyl, wherein any C₁₋₆alkyl is unsubstituted or substituted with one or more R¹¹;
   R⁵ is a bicyclic heteroaryl substituted with one or more R⁹;
   R⁶ is selected from halogen, —OR¹², —CN, and H;
   each R⁸ is independently selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹⁰ is independently selected from C₁₋₆ alkyl, C₂₋₆ alkenyl, and H;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H;
   each E is independently selected from

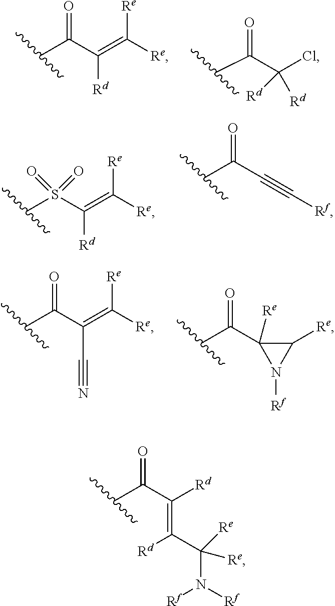

and —CN;
R^{a} and R^{b} are each independently selected from halogen, C₁₋₆ alkyl, —OR¹², and H, wherein any C₁₋₆alkyl is unsubstituted or is substituted with one or more R¹³;
each R^{d} and R^{e} are independently selected from halogen, C₁₋₆ alkyl, and H; and
each R^{f} is independently selected from C₁₋₆ alkyl and H.
53. The compound of embodiment 52, wherein R^{a} is a halogen.
54. The compound of embodiment 52, wherein R^{a} is a —OR¹².
55. The compound of any one of embodiments 52-54, wherein R^{b} is H.
56. The compound of any one of embodiments 52-55, wherein R² is a 4-6 membered heterocycle containing one nitrogen atom, wherein the heterocycle is substituted with one or more E and 0-4 R⁸.
57. The compound of embodiment 56, wherein R² is a heterocycle selected from azetidine, pyrrolidine, and piperidine, wherein the heterocycle is substituted with one or more E and 0-4 R⁸.
58. The compound of any one of embodiments 52-57, wherein R² is substituted with one E and 0-4 R⁸.
59. The compound of embodiment 58, wherein R² is substituted with one E and 1-4 R⁸.
60. The compound of embodiment 59, wherein the 1-4 R⁸ are each independently selected from halogen and C₁₋₆alkyl, wherein any C₁₋₆alkyl is unsubstituted.
61. The compound of any one of embodiments 52-60, wherein each E is independently selected from:

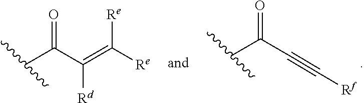

62. The compound of any one of embodiments 52-61, wherein R³ is H.
63. The compound of any one of embodiments 52-62, wherein R⁴ is H.
64. The compound of any one of embodiments 52-62, wherein R⁴ is selected from halogen, —CN, —OR¹², and C₁₋₆alkyl, wherein any C₁₋₆alkyl is unsubstituted or substituted with one or more R¹¹.
65. The compound of embodiment 64, wherein R⁴ is —CN.
66. The compound of embodiment 64, wherein R⁴ is a halogen.
67. The compound of embodiment 64, wherein R⁴ is C₁₋₆alkyl that is substituted with one or more R¹¹.
68. The compound of any one of embodiments 52-67, wherein R⁵ is selected from:

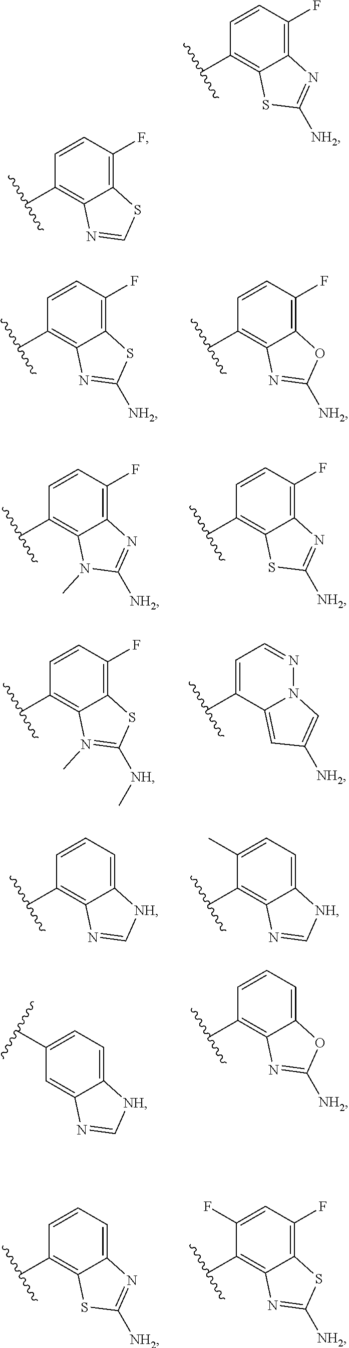

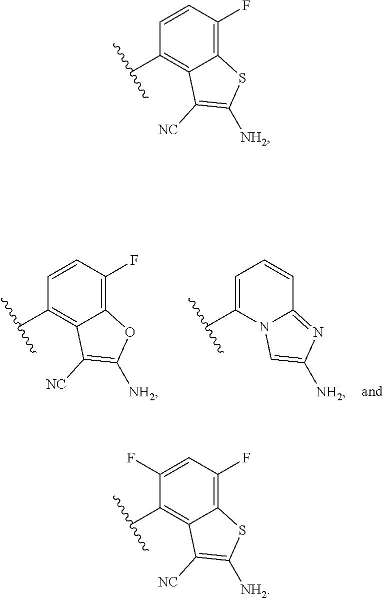 [SMILES: *c1ccc(F)c2sc(N)c(C#N)c12 ; *c1ccc(F)c2oc(N)c(C#N)c12 ; *c1cccc2nc(N)cn12 ; *c1c(F)cc(F)c2sc(N)c(C#N)c12]

69. The compound of embodiment 68, wherein R⁵ is selected from:

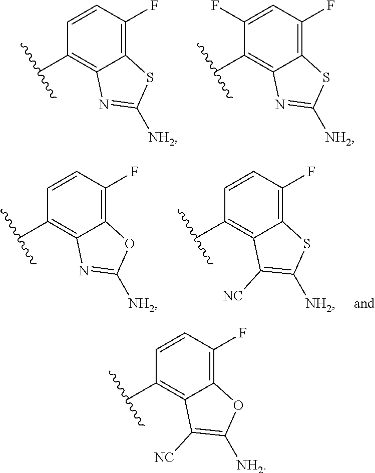

70. The compound of any one of embodiments 52-69, wherein R⁶ is selected from halogen, —CN, and H.
71. The compound of embodiment 70, wherein R⁶ is a halogen.
72. A compound according to Formula II:

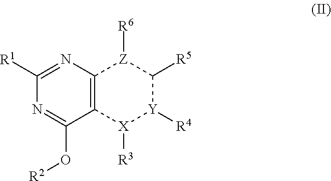

or a salt (e.g., pharmaceutically acceptable salt), ester, tautomer, prodrug, zwitterionic form, or stereoisomer thereof, wherein:
   each dashed line represents a single, double, or mixed bond;
   X, Y, and Z are selected from N and C, wherein one and only one of X, Y, and Z is N; R¹ is selected from —OR⁷;
   R² is a 4-6 membered heterocycle containing one or more nitrogen atoms, wherein the heterocycle is substituted with one or more E and 0-4 R⁸;
   when X is C, R³ is selected from H, —OR¹⁰, and C₁₋₆alkyl, wherein any C₁₋₆alkyl is unsubstituted or substituted with one or more R¹¹, and when X is N, R³ is absent;
   when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
   R⁵ is a bicyclic heteroaryl substituted with one or more R⁹;
   when Z is C, R⁶ is selected from halogen, —OR¹², —CN, and H, and when Z is N, R⁶ is absent;
   R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
   each R⁸ is selected from halogen and C₁₋₆alkyl, wherein any C₁₋₆alkyl is unsubstituted or substituted with one or more R¹¹;
   each R⁹ is independently selected from halogen, N(R¹²)₂, —CN, and C₁₋₆alkyl, wherein any C₁₋₆alkyl is unsubstituted or substituted with one or more R¹³;
   each R¹⁰ is independently selected from C₁₋₆ alkyl, C₂₋₆ alkenyl, and H;
   each R¹¹ is independently selected from halogen, —OH, and —CN;
   each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆ alkyl, C₂₋₆ alkenyl, and H;
   each E is independently selected from

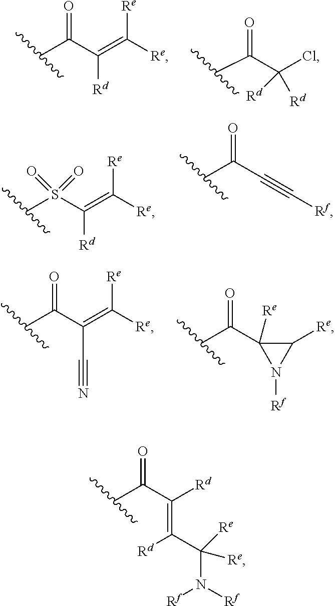

and —CN;
R^{a} and R^{b} are each independently selected from halogen, C₁₋₆ alkyl, —OR¹², and H, wherein any C₁₋₆alkyl is unsubstituted or is substituted with one or more R¹³;
each R^{d} and R^{e} are independently selected from halogen, C₁₋₆ alkyl, and H; and
each R^{f} is independently selected from C₁₋₆ alkyl and H.
73. The compound of embodiment 72, wherein X is N, and wherein Y and Z are C.
74. The compound of embodiment 73, wherein the compound is a compound according to Formula IIA:

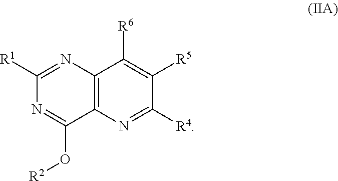

75. The compound of embodiment 73 or 74, wherein R⁴ is H.
76. The compound of embodiment 73 or 74, wherein R⁴ is selected from halogen, —CN, —OR¹², and C₁₋₆alkyl, wherein any C₁₋₆alkyl is unsubstituted or substituted with one or more R¹¹.
77. The compound of any one of embodiments 73-76, wherein R⁶ is selected from halogen, —CN, and H.
78. The compound of embodiment 72, wherein Y is N, and wherein X and Z are C.
79. The compound of embodiment 78, wherein the compound is a compound according to Formula IIB

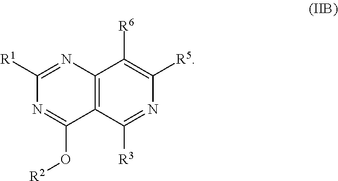

80. The compound of embodiment 78 or 79, wherein R³ is H.
81. The compound of any one of embodiments 78-80, wherein R⁶ is a halogen.
82. The compound of embodiment 72, wherein Z is N, and wherein X and Y are C.
83. The compound of embodiment 82, wherein the compound is a compound according to Formula IIC:

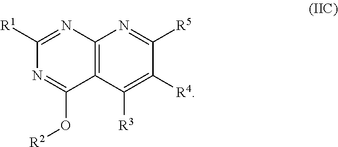

84. The compound of embodiment 82 or 83, wherein R³ is H.
85. The compound of any one of embodiments 82-84, wherein R⁴ is H.
86. The compound of any one of embodiments 82-84, wherein R⁴ is selected from halogen, —CN, —OR¹², and C₁₋₆alkyl, wherein any C₁₋₆alkyl is unsubstituted or substituted with one or more R¹¹.
87. The compound of any one of embodiments 72-86, wherein R² is a 4-6 membered heterocycle containing one nitrogen atom, wherein the heterocycle is substituted with one or more E and 0-4 R⁸.
88. The compound of embodiment 87, wherein R² is a heterocycle selected from azetidine, pyrrolidine, and piperidine, wherein the heterocycle is substituted with one or more E and 0-4 R⁸.
89. The compound of any one of embodiments 72-88, wherein R² is substituted with one E and 0-4 R⁸.
90. The compound of embodiment 89, wherein R² is substituted with one E and 1-4 R⁸.
91. The compound of embodiment 90, wherein the 1-4 R⁸ are each independently selected from halogen and C₁₋₆alkyl, wherein any C₁₋₆alkyl is unsubstituted.
92. The compound of any one of embodiments 72-91, wherein each E is independently selected from:

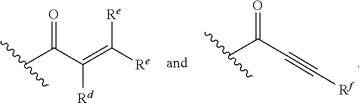

93. The compound of any one of embodiments 72-92, wherein R¹ is selected from:

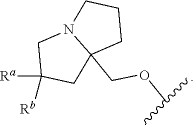

94. The compound of embodiment 93, wherein R^{a} is selected from halogen, C₁₋₆alkyl, —OR¹², and H, wherein any C₁₋₆alkyl is unsubstituted or is substituted with one or more R¹³.
95. The compound of embodiment 93 or 94, wherein R^{b} is H.
96. The compound of embodiment 93, wherein R¹ is selected from:

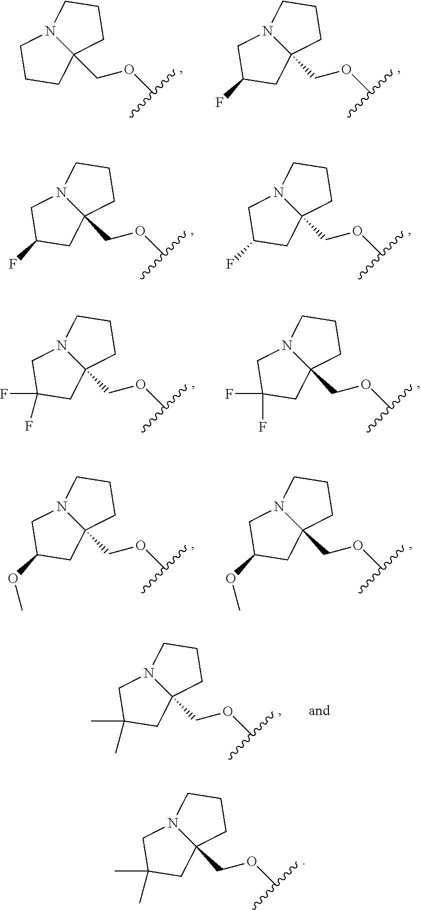

97. The compound of any one of embodiments 72-92, wherein R¹ is selected from:

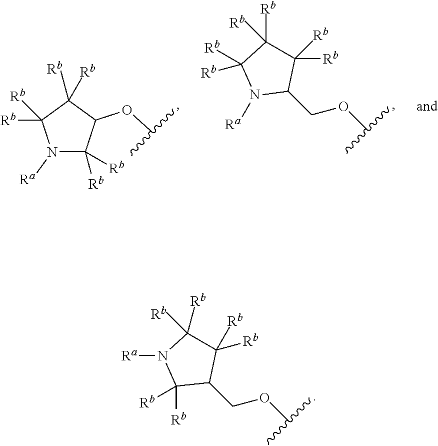

98. The compound of embodiment 97, wherein R^{a} is C₁₋₆alkyl that is unsubstituted or is substituted with one or more R¹³.
99. The compound of embodiment 97 or 98, wherein R¹ is selected from:

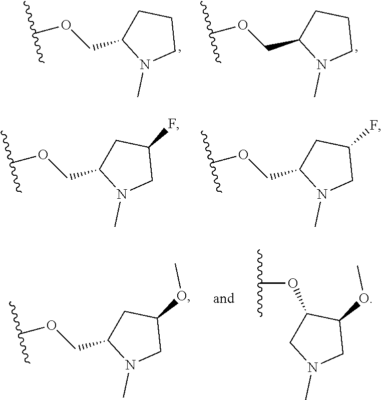

100. The compound of any one of embodiments 72-92, wherein R¹ is selected from:

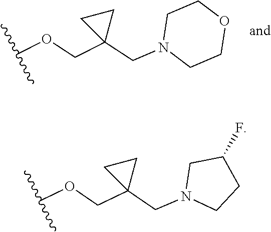 [SMILES: *OCC1(CN2CCOCC2)CC1 ; *OCC1(CN2CC[C@@H](F)C2)CC1]

101. The compound of any one of embodiments 72-100, wherein R⁵ is selected from:

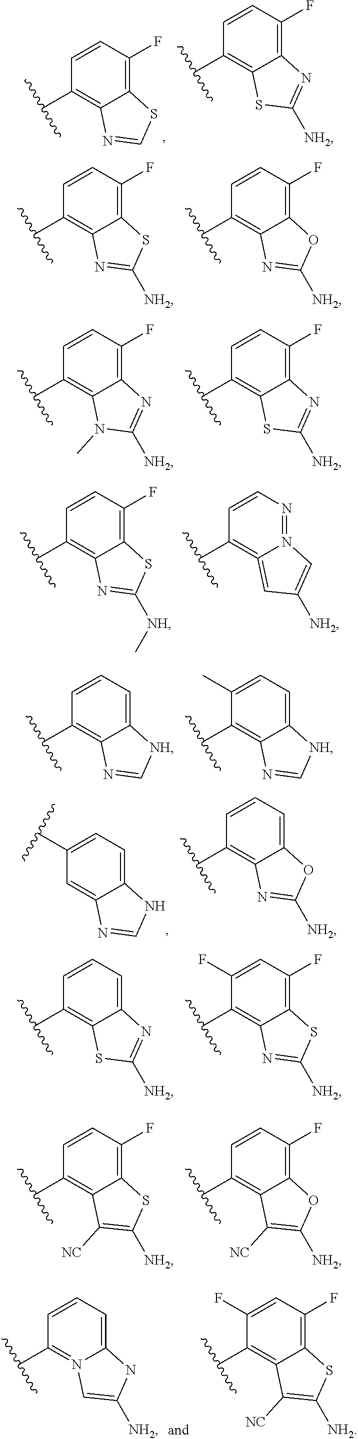

102. The compound of embodiment 101, wherein R⁵ is selected from:

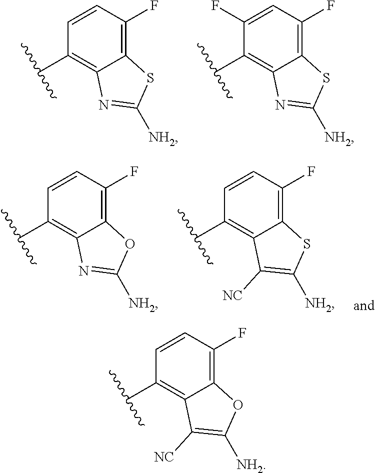

103. A compound shown in Table 2, or a salt (e.g., pharmaceutically acceptable salt), ester, tautomer, prodrug, zwitterionic form, or stereoisomer thereof.
104. A pharmaceutical composition comprising a compound of any one of embodiments 1-103, or a salt (e.g., pharmaceutically acceptable salt), ester, tautomer, prodrug, zwitterionic form, or stereoisomer thereof, and a pharmaceutically acceptable excipient.
105. A compound of any one of embodiments 1-103, or a salt (e.g., pharmaceutically acceptable salt), ester, tautomer, prodrug, zwitterionic form, or stereoisomer thereof, for use as a medicament.
106. The compound of embodiment 105, wherein the medicament is useful in the prevention or treatment of a disease, disorder, or condition ameliorated by the inhibition of KRAS having a G12C mutation.
107. The compound of embodiment 105 or 106, wherein the medicament is useful in the prevention or treatment of a cancer.
108. The compound of embodiment 107, wherein the cancer is selected from the group consisting of pancreatic cancer, colorectal cancer, and lung cancer.
109. A compound of any one of embodiments 1-103, or a salt (e.g., pharmaceutically acceptable salt), ester, tautomer, prodrug, zwitterionic form, or stereoisomer thereof, for use in the treatment of a disease, disorder, or condition.
110. The compound of embodiment 109, wherein the disease, disorder, or condition is a cancer.
111. The compound of embodiment 110, wherein the cancer is selected from the group consisting of pancreatic cancer, colorectal cancer, and lung cancer.
112. The compound of any one of embodiments 109-111, wherein the compound is used in the treatment of a disease, disorder, or condition in a subject in need thereof.
113. A compound of any one of embodiments 1-103, or a salt (e.g., pharmaceutically acceptable salt), ester, tautomer, prodrug, zwitterionic form, or stereoisomer thereof, for use in the manufacture of a medicament.
114. The compound of embodiment 113, wherein the medicament is useful in the prevention or treatment of a disease, disorder, or condition ameliorated by the inhibition of KRAS having a G12C mutation.
115. The compound of embodiment 113 or 114, wherein the medicament is useful in the treatment of a cancer.
116. The compound of embodiment 115, wherein the cancer is selected from the group consisting of pancreatic cancer, colorectal cancer, and lung cancer.
117. A method, comprising administering a therapeutically effective amount of a compound of any one of embodiments 1-103, or a salt (e.g., pharmaceutically acceptable salt), ester, tautomer, prodrug, zwitterionic form, or stereoisomer thereof, to a subject in need thereof.
118. The method of embodiment 117, wherein the subject has a disease, disorder, or condition ameliorated by the inhibition of KRAS having a G12C mutation.
119. The method of embodiment 117 or 118, wherein the subject has a cancer.
120. The method of embodiment 119, wherein the subject was previously diagnosed with the cancer.
121. The method of embodiment 119, wherein the subject has previously undergone a treatment regimen for the cancer.
122. The method of embodiment 119, wherein the subject has previously entered remission from the cancer.
123. The method of any one of embodiments 119-122, wherein the cancer is selected from the group consisting of pancreatic cancer, colorectal cancer, and lung cancer.
124. The method of any one of embodiments 117-123, wherein the compound, or the salt, ester, tautomer, prodrug, zwitterionic form, or stereoisomer thereof, is administered in combination with an additional therapeutic agent.
125. The use of a compound of any one of embodiments 1-103 or a salt (e.g., pharmaceutically acceptable salt), ester, tautomer, prodrug, zwitterionic form, or stereoisomer thereof, for the manufacture of a medicament for the treatment of a cancer.
126. The use of embodiment 125, wherein the cancer is selected from the group consisting of pancreatic cancer, colorectal cancer, and lung cancer.
127. A method, comprising contacting a KRAS protein with a compound of any one of embodiments 1-103, or a salt (e.g., pharmaceutically acceptable salt), ester, tautomer, prodrug, zwitterionic form, or stereoisomer thereof.
128. The method of embodiment 127, wherein contacting the KRAS protein with the compound modulates KRAS.
129. The method of embodiment 127 or 128, wherein the KRAS protein has a G12C mutation.
130. The method of any one of embodiments 127-129, wherein the KRAS protein is in an active state.
131. The method of any one of embodiments 127-129, wherein the KRAS protein is in an inactive state.

### Examples

Selected abbreviations used in the preceding sections and the Examples are summarized in Table 1.

TABLE 1
Abbreviations.

| Abbreviation | Term |
|---|---|
| MeCN | acetonitrile |
| cm | centimeter |
| ° C. | degrees Celsius |
| ° K | degrees Kelvin |
| BINAP | [1,1′-Binaphthalene]-2,2′-diyl)bis(diphenylphosphane) |
| DCM | dichloromethane |
| DIEA | N,N-diisopropylethylamine (Hünig's base) |
| DMF | dimethylformamide |
| DMSO | dimethyl sulfoxide |
| ESI | electrospray ion-mass spectrometry |
| EtOH | ethanol |
| EtOAc, EA | ethyl acetate |
| g | gram |
| Hz | hertz |
| HATU | Hexafluorophosphate azabenzotriazole tetramethyl uronium |
| HPLC | high performance liquid chromatography |
| HTRF | homogenous time-resolved fluorescence |
| h | hour |
| kDa | kilodalton |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NMP | N-methyl-2-pyrrolidone |
| PE:EA | Petroleum ether:ethyl acetate |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SPR | surface plasmon resonance |
| THF | tetrahydrofuran |
| TMS | tetramethylsilane |
| TLC | thin layer chromatography |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera. One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g., hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend™ 400 spectrometer (operating at 400 MHZ), Bruker Ascend 700 MHz Advance Neo Spectrometer (Bruker-Biospin) or Bruker Advance ultrashield 300/54 (operating at 300 MHz) at 298° K following standard operating procedure suggested by manufacturer. Reference frequency was set using TMS as an internal standard. Chemical shift values (8) are reported in parts per million (ppm) with splitting patterns abbreviated to: s (singlet), br. s (broad singlet), d (doublet), dd (double doublet), t (triplet), and m (multiplet). The coupling constant (J) is given in Hz. Typical deuterated solvents were utilized as indicated in the individual examples.

LCMS analysis were typically performed using one of the following conditions:

(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min. using a poroshell 120 EC-C18 50 mm×3.0 mm×2.7 μm capillary column; Flow Rate: 0.7 mL/min. Mass spectra (MS) were measured by electrospray ion-mass spectroscopy (ESI). All temperatures are in degrees Celsius (° C.) unless otherwise noted.

(2) LCMS spectra were taken on an Agilent Technologies 1290-6420 Triple Quadrupole spectrometer: The mobile phase for the LC was acetonitrile (A) with 0.05% formic acid, and water (B) with 0.05% formic acid, and the eluent gradient was from 5-95% A in 5.0 min, using a ZORBAX SB—C18 50 mm×2.1 mm×1.8 μm capillary column; Flow Rate: 0.3 mL/min. Mass spectra (MS) were measured by electrospray ion-mass spectroscopy (ESI). All temperatures are in degrees Celsius unless otherwise noted.

(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 μm RapidResol column with a mobile phase of water (0.1% formic acid)/MeCN (0.1% formic acid) and a gradient of 5-95% MeCN over 10 minutes at a flow rate of 1 mL/min. Accurate mass data was obtained using a Thermo Fisher extractive plus EMR orbitrap LCMS system. Exact mass values were calculated by ChemCalc.

(4) LCMS spectra were taken on an alliance Waters 2695 coupled to a dual absorbance detector waters 2487 and a waters micro mass ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

Typically, analytical HPLC mass spectrometry conditions were as follows:

LC1: Agilent Technologies 1260 Infinity coupled, Column: poroshell 120 EC-C18 150 mm×4.6 mm×4 μm; Temperature: 40° C.; Eluent: 5:95 v/v acetonitrile/water+0.02% trifluoroacetic acid in 20 min; Flow Rate: 1.2 mL/min; Detection: VWD, 190-600 nm.

LC2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μM OBD™ 19×250 mm column with a mobile phase of water/MeCN or water (0.1% TFA)/MeCN (0.1% TFA).

Preparative HPLC were carried out with one of the following two conditions:

Condition 1: GILSON Preparative HPLC System; Column: Ultimate XB—C18, 21.2 mm×250 mm, 5 um; Mobile phase: Water with 0.1% trifluoroacetic acid; MeCN with 0.1% trifluoroacetic acid; Method: 15 minutes gradient elution; Initial organic: 10% to 30%; Final organic: 60% to 80%; UV1: 240; UV2:230; Flow: 15 mL/min.

Condition 2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 uM OBD™ 19×250 mm column with a mobile phase of water/MeCN or water (0.1% TFA)/MeCN (0.1% TFA).

Compound names were generated with ChemDraw Professional.

The compounds provided herein, or alternate forms thereof (e.g., salt, ester, tautomer, prodrug, zwitterionic form, stereoisomer, etc.) may be prepared according to various methods including those set forth in the following examples.

### Synthetic Example 1: Synthesis of 1-(3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy) azetidin-1-yl) prop-2-en-1-one (Compound 7)

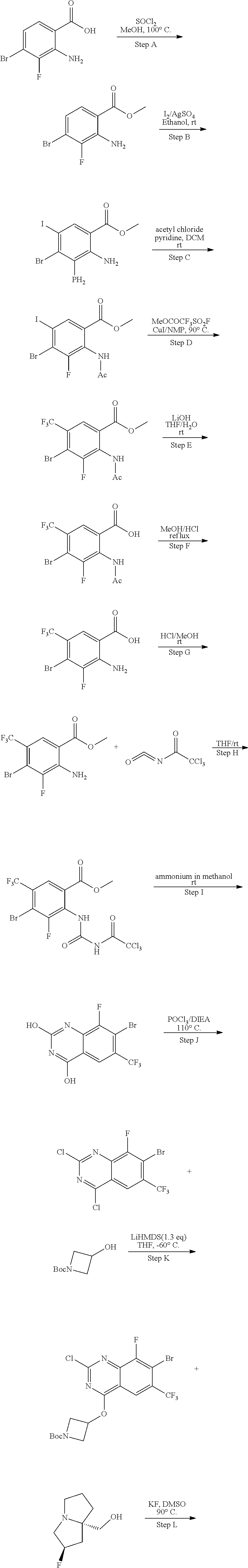

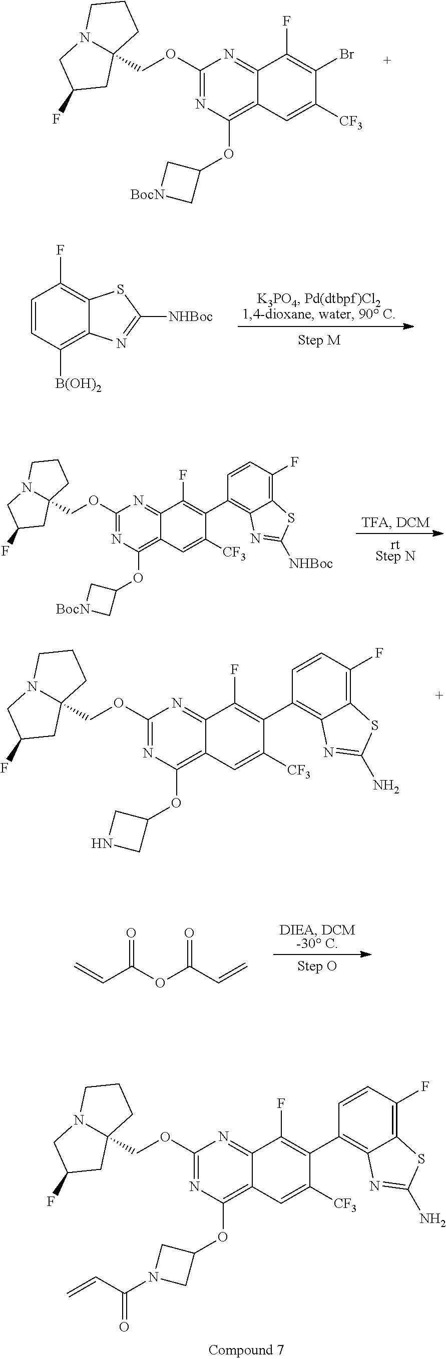

Step A: Preparation of methyl 2-amino-4-bromo-3-fluorobenzoate: To a stirring solution of 2-amino-4-bromo-3-fluorobenzoic acid (5.0 g, 21.4 mmol) in MeOH (30 mL) was added dropwise thionyl chloride (15.6 ml, 21 mmol) at 0° C. under argon. The resulting mixture was heated to 100° C. for 16 hours. The solvent was evaporated, and the residue was dissolved in ethyl acetate (100 mL). The organic layer was washed with a saturated aqueous NaHCO₃ solution then dried over Na₂SO₄, filtered, and concentrated under vacuum. The resulting crude material was purified by silica gel column chromatography using EtOAc in hexanes (0% to 20%) as eluent to give methyl 2-amino-4-bromo-3-fluorobenzoate (5.0 g, 94%) as a solid. LCMS ESI (+) m/z 249 (M+H). ¹HNMR (300 MHz, CDCl₃) δ 7.53 (dd, J=8.8, 1.8 Hz, 1H), 6.78 (dd, J=8.8, 6.3 Hz, 1H), 5.93 (s, 1H), 3.90 (s, 1H).

Step B: Preparation of methyl 2-amino-4-bromo-3-fluoro-5-iodobenzoate: To a mixture of iodine (7.16 g, 28 mmol) and silver sulfate (5.3 g, 17 mmol) in EtOH (200 mL), methyl 2-amino-4-bromo-3-fluorobenzoate (5.0 g, 20 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution, brine and the resulting organic solution was dried over Na₂SO₄, filtered, and concentrated under vacuum to give methyl 2-amino-4-bromo-3-fluoro-5-iodobenzoate: the title compound (6.66 g, 88% yield) as a yellow solid. LCMS ESI (+) m/z 373 (M+H). ¹HNMR (300 MHz, CDCl₃) δ 8.14 (d, J=1.9 Hz, 1H), 5.94 (s, 2H), 3.91 (s, 3H).

Step C: Preparation of methyl 2-acetamido-4-bromo-3-fluoro-5-iodobenzoate: The methyl 2-amino-4-bromo-3-fluoro-5-iodobenzoate (3.50 g, 9.4 mmol) and pyridine (2.3 ml, 28 mmol) were dissolved in DCM at 0° C. Acetyl chloride (0.79 ml, 11 mmol) was added and the reaction was warmed to ambient temperature and stirred at this temperature for 16 hours. The reaction mixture was concentrated under vacuum and the residue obtained was purified by silica gel column chromatography using ethyl acetate in hexanes (0% to 30%) as eluent to give methyl 2-acetamido-4-bromo-3-fluoro-5-iodobenzoate (2.7 g, 69%) as solid. LCMS ESI (+) m/z 417 (M+H). ¹HNMR (300 MHz, CDCl₃) δ 8.87 (s, 1H), 8.25 (s, 1H), 3.95 (s, 3H), 2.26 (s, 3H).

Step D: Preparation of methyl 2-acetamido-4-bromo-3-fluoro-5-(trifluoromethyl)benzoate: To a stirred solution of methyl 4-bromo-2-acetamido-3-fluoro-5-iodobenzoate (1.0 g, 2.4 mmol) and methyl fluorosulfonyldifluoroacetate (0.92 g, 0.72 mmol) in NMP (22.0 mL) at ambient temperature, CuI (0.14 g, 0.73 mmol) was added and the resulting mixture was stirred at 80° C. for 16 hours. After cooling to ambient temperature, the mixture was quenched with water and extracted with ethyl acetate. The organic layer was dried over Na₂SO₄, filtered, and concentrated and the crude material was purified by silica gel column chromatography using ethyl acetate in hexanes (0% to 20%) as eluent to give methyl 2-acetamido-4-bromo-3-fluoro-5-(trifluoromethyl)benzoate (0.64 g, 74%) as solid. LCMS ESI (+) m/z 358 (M+H). ¹HNMR (300 MHz, CDCl₃) δ 9.23 (s, 1H), 8.10 (s, 1H), 3.93 (s, 3H), 2.28 (s, 3H).

Step E: Preparation of 2-Acetamido-4-bromo-3-fluoro-5-(trifluoromethyl)benzoic acid: Methyl 4-bromo-2-acetamido-3-fluoro-5-(trifluoromethyl)benzoate (3.4 g, 9.49 mmol) was dissolved in THF (56 ml) and water (14 ml) at ambient temperature, then LiOH (0.91 g, 38 mmol) was added. The resulting mixture was stirred at 80° C. for 2 hours. The reaction was diluted with water, acidified with 2M HCl to adjust to a pH˜ 4 and then extracted with ethyl acetate (2×25 mL). The organic layer was washed with brine, dried over Na₂SO₄, filtered and concentrated in vacuum to afford 4-bromo-2-acetamido-3-fluoro-5-(trifluoromethyl)benzoic acid as solid (3.0 g, 92%). ¹HNMR (300 MHz, CD₃OD) δ 8.10 (s, 1H), 8.01 (s, 1H), 2.21 (s, 3H).

Step F: Preparation of 2-Amino-4-bromo-3-fluoro-5-(trifluoromethyl)benzoic acid: The 4-Bromo-2-acetamido-3-fluoro-5-(trifluoromethyl)benzoic acid (0.50 g, 1.45 mmol) was dissolved in a 3 M solution of HCl in MeOH (0.064 mL, 1.74 mmol) and refluxed at 80° C. for 2 hours. After cooling to ambient temperature, the reaction mixture was concentrated under vacuum to provide 2-Amino-4-bromo-3-fluoro-5-(trifluoromethyl)benzoic acid as solid (0.40 g, 91%). LCMS ESI (−) m/z 300 (M−H). ¹HNMR (300 MHz, CD₃OD) δ 7.86 (s, 1H).

Step G: Preparation of 2-amino-4-bromo-3-fluoro-5-(trifluoromethyl)benzoate: A mixture of methyl 2-acetamido-4-bromo-3-fluoro-5-(trifluoromethyl)benzoate (1.20 g, 3.35 mmol) in 3 M HCl in MeOH was heated at 60° C. for 2 hours. After cooling to ambient temperature, the solvent was evaporated, and the crude product was partitioned between EtOAc and saturated NaHCO₃. The organic layer was separated, dried over sodium sulfate, filtered, and evaporated to provide 2-amino-4-bromo-3-fluoro-5-(trifluoromethyl)benzoate (1.00 g, 94%) as an oil. LCMS ESI (+) m/z 316.9 (M+H). ¹H NMR (300 MHz, CDCl₃) δ 8.01 (s, 1H), 6.28 (s, 2H), 3.94 (s, 3H).

Step H: Preparation of methyl 4-bromo-3-fluoro-2-(3-(2,2,2-trichloroacetyl)ureido)-5-(trifluoromethyl)benzoate: To a mixture of methyl 2-amino-4-bromo-3-fluoro-5-(trifluoromethyl)benzoate (0.80 g, 2.53 mmol) in THF (4.2 mL) was added trichloroethanecarbonyl isocyanate (0.45 mL, 3.79 mmol) at ambient temperature. After 15 minutes, the reaction mixture was evaporated, followed by the addition of MTBE and the solid formed was collected and washed with MTBE to provide methyl 4-bromo-3-fluoro-2-(3-(2,2,2-trichloroacetyl)ureido)-5-(trifluoromethyl)benzoate (0.71 g, 56%). LCMS ESI (+) m/z 529.99 (M+Na). ¹H NMR (300 MHz, CDCl₃) δ 10.91 (s, 1H), 8.65 (s, 1H), 8.23 (s, 1H), 4.03 (s, 3H).

Step I: Preparation of 7-bromo-8-fluoro-6-(trifluoromethyl) quinazoline-2,4-diol: To a solution of methyl 4-bromo-3-fluoro-2-(3-(2,2,2-trichloroacetyl)ureido)-5-(trifluoromethyl)benzoate (0.71 g, 1.40 mmol) in methanol (7.0 mL) was added 7 M solution of ammonia in methanol (0.46 mL, 3.23 mmol) at ambient temperature and stirred at ambient temperature for 1 hour. The mixture was concentrated under reduced pressure to provide a solid which was co-evaporated with ether to provide 7-bromo-8-fluoro-6-(trifluoromethyl) quinazoline-2,4-diol (0.60 g, 100%) as solid. LCMS ESI (+) m/z 260.0 (M+H). ¹H NMR (300 MHz, DMSO-d₆) δ 8.41 (s, 1H), 8.28 (s, 1H), 7.99 (s, 1H).

Step J: Preparation of 7-bromo-2,4-dichloro-8-fluoro-6-(trifluoromethyl) quinazoline: To a stirring solution of phosphorus oxychloride (0.97 mL, 10.5 mmol) and Hunig's base (0.40 mL, 2.29 mmol) was added 7-bromo-8-fluoro-6-(trifluoromethyl) quinazoline-2,4-diol (0.15 g, 0.46 mmol) at 0° C. After addition, the resulting mixture was stirred at 110° C. for 1 hour. After cooling down to ambient temperature, the mixture was evaporated and co-evaporated with chloroform to give 7-bromo-2,4-dichloro-8-fluoro-6-(trifluoromethyl) quinazoline which used as such for the next step. ¹H NMR (300 MHz, CDCl₃) δ 8.45 (s, 1H).

Step K: Preparation of tert-butyl 3-[7-bromo-2-chloro-8-fluoro-6-(trifluoromethyl) quinazolin-4-yl]oxyazetidine-1-carboxylate: To a solution of tert-butyl 3-hydroxyazetidine-1-carboxylate (190 mg, 1.10 mmol) in THF (1.5 mL) was added LiHMDS (1M in THF) (0.71 mL, 0.71 mmol) dropwise at −70° C. under Ar and then stirred at −70° C. for 1 hour. 7-bromo-2,4-dichloro-8-fluoro-6-(trifluoromethyl) quinazoline (200 mg, 0.55 mmol) in THF (1 mL) was added at −70° C. After stirring for 1.5 hours at −70° C., the mixture was quenched with NH₄Cl aqueous solution and extracted with EtOAc (20 mL×3). The combined organics were dried (sodium sulfate), filtered and concentrated to dry under vacuum and the residue was purified by preparative-TLC (15% EtOAc in petroleum ether) to give tert-butyl 3-[7-bromo-2-chloro-8-fluoro-6-(trifluoromethyl) quinazolin-4-yl]oxyazetidine-1-carboxylate (81 mg, 29%). LCMS ESI (+) m/z 500.0 (M+H).

Step L: Preparation of tert-butyl 3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate: To s solution of [(2R,8S)-2-fluoro-1,2,3,5,6,7-hexahydropyrrolizin-8-yl]methanol (69 mg, 0.43 mmol) in DMSO (1.5 mL) was added tert-butyl 3-[7-bromo-2-chloro-8-fluoro-6-(trifluoromethyl) quinazolin-4-yl]oxyazetidine-1-carboxylate (145 mg, 0.29 mmol) and KF (135 mg, 2.32 mmol) at ambient temperature. The mixture was sealed and then stirred at 90° C. for 10 hours. After cooling to ambient temperature, the mixture was diluted with brine, extracted with EtOAc and washed with Brine. The organics were separated, dried (sodium sulfate), filtered and concentrated under vacuum. The residue was purified by preparative-TLC (50% EtOAc in petroleum ether) to give tert-butyl 3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate (47 mg, 26%). LCMS ESI (+) m/z 623.1 (M+H).

Step M: Preparation of tert-butyl 3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate: To a solution of [2-(tert-butoxycarbonylamino)-7-fluoro-1,3-benzothiazol-4-yl]boronic acid (31 mg, 0.098 mmol) in 1,4-Dioxane (2 mL)/Water (0.50 mL) was added tert-butyl 3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate (47 mg, 0.075 mmol), Pd(dtbpf)Cl₂ (3.9 mg, 0.0060 mmol) and K₃PO₄ (48 mg, 0.23 mmol). The mixture was bubbled with Ar for 1˜2 minutes and then sealed. After that, the mixture was stirred at 90° C. for 1 hour. After cooling to ambient temperature, water and ethyl acetate were added. The organic layer was separated, dried (sodium sulfate), filtered and concentrated under reduced pressure. The residue was purified by preparative-TLC (50% EtOAc in petroleum ether) to give tert-butyl 3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate (26 mg, 37%). LCMS ESI (+) m/z 811.2 (M+H).

Step N: Preparation of 4-[4-(azetidin-3-yloxy)-8-fluoro-2-[[(2R,8S)-2-fluoro-1,2,3,5,6,7-hexahydropyrrolizin-8-yl]methoxy]-6-(trifluoromethyl) quinazolin-7-yl]-7-fluoro-1,3-benzothiazol-2-amine: To a solution of tert-butyl 3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidine-1-carboxylate (26 mg, 0.032 mmol) in DCM (1 mL) was added trifluoroacetic acid (0.50 mL, 6.49 mmol) at room temperature and stirred for 1 hour. After concentrated under vacuum, the residue was lyophilized under vacuum to give 4-[4-(azetidin-3-yloxy)-8-fluoro-2-[[(2R,8S)-2-fluoro-1,2,3,5,6,7-hexahydropyrrolizin-8-yl]methoxy]-6-(trifluoromethyl) quinazolin-7-yl]-7-fluoro-1,3-benzothiazol-2-amine (30 mg, 100%). LCMS ESI (+) m/z 611.2 (M+H).

Step O: Preparation of 1-(3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy) azetidin-1-yl) prop-2-en-1-one: To a solution of 4-[4-(azetidin-3-yloxy)-8-fluoro-2-[[(2R,8S)-2-fluoro-1,2,3,5,6,7-hexahydropyrrolizin-8-yl]methoxy]-6-(trifluoromethyl) quinazolin-7-yl]-7-fluoro-1,3-benzothiazol-2-amine (30 mg, 0.049 mmol) in DCM (3 mL) was added TEA (0.027 mL, 0.20 mmol) and then to this was added dropwise a solution of prop-2-enoyl prop-2-enoate (4.3 mg, 0.034 mmol) in DCM (0.50 mL) at −40° C. under Ar. The mixture was stirred for 30 minutes at −40° C. Water and DCM were added. The organic layer was separated, dried over Na₂SO₄, filtered, and concentrated to dry and the residue was purified by reverse phase preparative-HPLC to give 1-[3-[7-(2-amino-7-fluoro-1,3-benzothiazol-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]oxyazetidin-1-yl]prop-2-en-1-one (3.7 mg, 11%). LCMS ESI (+) m/z 665.1 (M+H). ¹HNMR (400 MHZ, CD₃OD) δ 8.41 (s, 1H), 7.12-7.21 (m, 1H), 6.99 (t, J=8.8 Hz, 1H), 6.21-6.41 (m, 2H), 5.68-5.80 (m, 2H), 5.47 (dd, J=52.4, 11.6 Hz, 1H), 4.68 (s, 2H), 4.49-4.55 (m, 2H), 4.26-4.31 (m, 1H), 3.98-3.13 (m, 3H), 3.79-3.90 (m, 1H), 3.51-3.58 (m, 1H), 2.31-2.75 (m, 6H).

### Synthetic Example 2: Synthesis of 1-((R)-3-(((S)-7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 11) and 1-((R)-3-(((R)-7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 12)

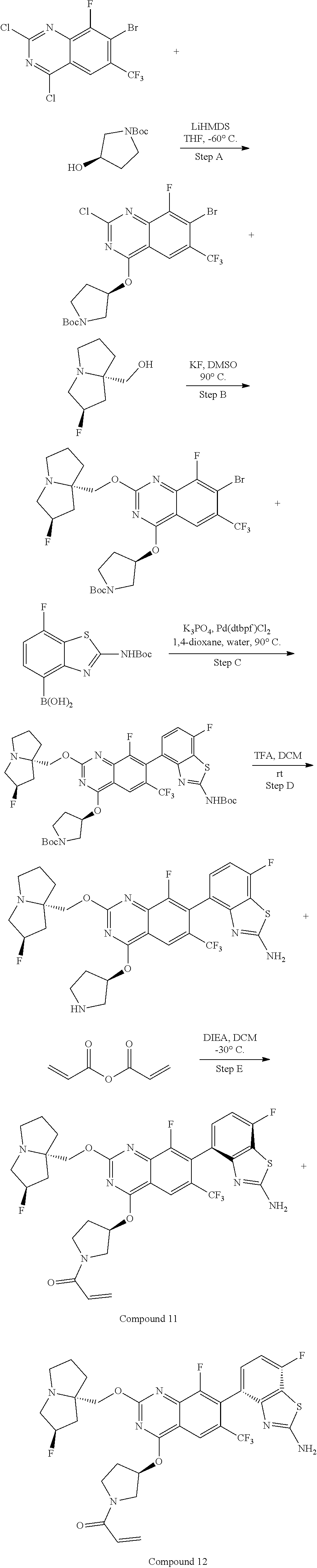

Step A: Preparation of tert-butyl (3R)-3-[7-bromo-2-chloro-8-fluoro-6-(trifluoromethyl) quinazolin-4-yl]oxopyrrolidine-1-carboxylate: To a solution of tert-butyl (3R)-3-hydroxypyrrolidine-1-carboxylate (309 mg, 1.65 mmol) in THF (1.5 mL) was added LiHMDS (1M in THF) (1.07 mL, 1.07 mmol) dropwise at −70° C. under Ar and then stirred at −70° C. for 1 hour. 7-bromo-2,4-dichloro-8-fluoro-6-(trifluoromethyl) quinazoline (300 mg, 0.82 mmol) in THF (1 mL) was added at −70° C. After stirring for 1.5 hours at −70° C., the mixture was quenched with NH₄Cl aqueous solution and extracted with EtOAc (20 mL×3). The combined organics were dried (sodium sulfate), filtered, and concentrated to dry under vacuum and the residue was purified by preparative-TLC (15% EtOAc in petroleum ether) to give tert-butyl (3R)-3-[7-bromo-2-chloro-8-fluoro-6-(trifluoromethyl) quinazolin-4-yl]oxypyrrolidine-1-carboxylate (183 mg, 43%). LCMS ESI (+) m/z 514.0 (M+H).

Step B: Preparation of tert-butyl (3R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate: To a solution of [(2R,8S)-2-fluoro-1,2,3,5,6,7-hexahydropyrrolizin-8-yl]methanol (85 mg, 0.53 mmol) in DMSO (2 mL) was added tert-butyl (3R)-3-[7-bromo-2-chloro-8-fluoro-6-(trifluoromethyl) quinazolin-4-yl]oxypyrrolidine-1-carboxylate (183 mg, 0.36 mmol) and KF (165 mg, 2.84 mmol) at ambient temperature. The mixture was sealed and then stirred at 90° C. for 10 hours. After cooling to ambient temperature, the mixture was diluted with brine, extracted with EtOAc, and washed with Brine. The organics were separated, dried (sodium sulfate), filtered, and concentrated under vacuum. The residue was purified by preparative-TLC (50% EtOAc in petroleum ether) to give tert-butyl (3R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (80 mg, 35%). LCMS ESI (+) m/z 637.1 (M+H).

Step C: Preparation of tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate: To a solution of [2-(tert-butoxycarbonylamino)-7-fluoro-1,3-benzothiazol-4-yl]boronic acid (51 mg, 0.16 mmol) in 1,4-Dioxane (2 mL)/Water (0.50 mL) was added tert-butyl (3R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (80 mg, 0.13 mmol), Pd(dtbpf)Cl₂ (6.5 mg, 0.010 mmol) and K₃PO₄ (80 mg, 0.38 mmol). The mixture was bubbled with Ar for 1-2 minutes and then sealed. After that, the mixture was stirred at 90° C. for 1 hour. After cooling to ambient temperature, water and ethyl acetate were added. The organic layer was separated, dried (sodium sulfate), filtered, and concentrated under reduced pressure. The residue was purified by preparative-TLC (50% EtOAc in petroleum ether) to give tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (40 mg, 30%). LCMS ESI (+) m/z 825.2 (M+H).

Step D: Preparation of 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine: To a solution of tert-butyl (3R)-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (40 mg, 0.049 mmol) in DCM (2 mL) was added trifluoroacetic acid (1.0 mL, 13.0 mmol) at room temperature and stirred for 1 hour. After concentrated under vacuum, the residue was lyophilized under vacuum to give 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine (46 mg, 100%). LCMS ESI (+) m/z 625.2 (M+H).

Step E: Preparation of 1-((R)-3-(((S)-7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 11) and 1-((R)-3-(((R)-7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 12): To a solution of 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine (46 mg, 0.074 mmol) in DCM (3 mL) was added TEA (0.051 mL, 0.368 mmol) and then to this was added dropwise a solution of prop-2-enoyl prop-2-enoate (6.5 mg, 0.0516 mmol) in DCM (0.50 mL) at −40° C. under Ar. The mixture was stirred for 30 minutes at −40° C. Water and DCM were added. The organic layer was separated, dried over Na₂SO₄, filtered, and concentrated to dry and the residue was purified by reverse phase preparative-HPLC. The first compound off the column is identified as one atropisomer, 1-((R)-3-(((S)-7-(2-amino-7-fluorobenzo[d]thiazol-4-yl)-8-fluoro-2-(((2R,7aS)-2-fluorotetrahydro-1H-pyrrolizin-7a (5H)-yl) methoxy)-6-(trifluoromethyl) quinazolin-4-yl)oxy)pyrrolidin-1-yl) prop-2-en-1-one (Compound 11) (6.8 mg, 13%). LCMS ESI (+) m/z 679.3 (M+H). ¹HNMR (400 MHZ, CD₃OD) δ 8.27 (s, 1H), 7.12-7.30 (m, 1H), 7.00 (t, J=8.8 Hz, 1H), 6.58-6.81 (m, 1H), 6.29-6.41 (m, 1H), 5.88-6.15 (m, 1H), 5.74-5.89 (m, 1H), 5.31 (d, J=52 Hz, 1H), 4.28-4.48 (m, 2H), 3.75-4.21 (m, 4H), 3.15-3.31 (m, 3H), 2.98-3.11 (m, 1H), 1.85-2.65 (m, 8H). The second compound off the column is identified as the other atropisomer 1-((R)-3-(((R)-7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 12) (4.5 mg, 9%). LCMS ESI (+) m/z 679.3 (M+H). ¹HNMR (400 MHZ, CD₃OD) δ 8.27 (s, 1H), 7.12-7.30 (m, 1H), 7.00 (t, J=8.8 Hz, 1H), 6.58-6.81 (m, 1H), 6.29-6.41 (m, 1H), 5.88-6.15 (m, 1H), 5.74-5.89 (m, 1H), 5.31 (d, J=52 Hz, 1H), 4.28-4.48 (m, 2H), 3.75-4.21 (m, 4H), 3.15-3.31 (m, 3H), 2.98-3.11 (m, 1H), 1.85-2.65 (m, 8H).

### Synthetic Example 3: Synthesis of 1-((R)-3-(((S)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 14) and 1-((R)-3-(((R)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 15)

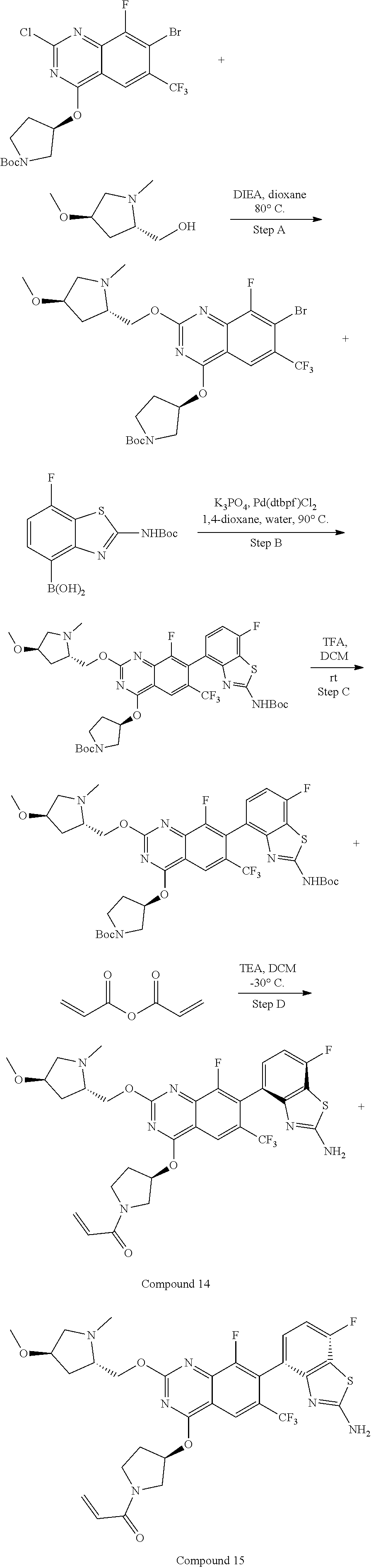

Step A: Preparation of tert-butyl (R)-3-((7-bromo-8-fluoro-2-(((2S,4R)-4-methoxy-1-methylpyrrolidin-2-yl)methoxy)-6-(trifluoromethyl) quinazolin-4-yl)oxy)pyrrolidine-1-carboxylate: To a solution of tert-butyl (3R)-3-[7-bromo-2-chloro-8-fluoro-6-(trifluoromethyl) quinazolin-4-yl]oxypyrrolidine-1-carboxylate (80 mg, 0.16 mmol) in 1, 4-dioxane (0.2 mL) was added [(2S)-4-methoxy-1-methyl-pyrrolidin-2-yl]methanol (45 mg, 0.31 mmol) and DIEA (0.083 mL, 0.47 mmol) at ambient temperature. The mixture was sealed and then stirred at 80° C. for 3 hours. After cooling to ambient temperature, the mixture was diluted with brine, extracted with EtOAc, and washed with brine. The organics were separated, dried (sodium sulfate), filtered, and concentrated under vacuum. The residue was purified by column chromatography on silica gel, eluting with 50% EtOAc in petroleum ether, to give tert-butyl (R)-3-((7-bromo-8-fluoro-2-(((2S,4R)-4-methoxy-1-methylpyrrolidin-2-yl)methoxy)-6-(trifluoromethyl) quinazolin-4-yl)oxy)pyrrolidine-1-carboxylate (70 mg, 72%). LCMS ESI (+) m/z 623.2 (M+H).

Step B: Preparation of tert-butyl (3R)-3-[7-[2-(tert-butoxycarbonylamino)-7-fluoro-1,3-benzothiazol-4-yl]-8-fluoro-2-[[(2S,4R)-4-methoxy-1-methyl-pyrrolidin-2-yl]methoxy]-6-(trifluoromethyl) quinazolin-4-yl]oxypyrrolidine-1-carboxylate: To a solution of tert-butyl (3R)-3-[7-bromo-8-fluoro-2-[[(2S,4R)-4-methoxy-1-methyl-pyrrolidin-2-yl]methoxy]-6-(trifluoromethyl) quinazolin-4-yl]oxypyrrolidine-1-carboxylate (70 mg, 0.112 mmol) in 1,4-Dioxane (10 mL)/Water (2 mL) was added [2-(tert-butoxycarbonylamino)-7-fluoro-1,3-benzothiazol-4-yl]boronic acid (46 mg, 0.15 mmol), potassium phosphate (48 mg, 0.23 mmol) and 1,1′-Bis (di-t-butylphosphino) ferrocene palladium dichloride (7.3 mg, 0.011 mmol) at room temperature. The mixture was bubbled with Ar for 1-2 minutes and then sealed. After that, the mixture was stirred at 90° C. for 1 hour. After cooling to ambient temperature, water and ethyl acetate were added. The organic layer was separated, dried (sodium sulfate), filtered, and concentrated under reduced pressure. The residue was purified by preparative-TLC (50% EtOAc in petroleum ether) to give tert-butyl (3R)-3-[7-[2-(tert-butoxycarbonylamino)-7-fluoro-1,3-benzothiazol-4-yl]-8-fluoro-2-[[(2S,4R)-4-methoxy-1-methyl-pyrrolidin-2-yl]methoxy]-6-(trifluoromethyl) quinazolin-4-yl]oxypyrrolidine-1-carboxylate (69 mg, 75%). LCMS ESI (+) m/z 811.3 (M+H).

Step C: Preparation of 7-fluoro-4-[8-fluoro-2-[[(2S,4R)-4-methoxy-1-methyl-pyrrolidin-2-yl]methoxy]-4-[(3R)-pyrrolidin-3-yl]oxy-6-(trifluoromethyl) quinazolin-7-yl]-1,3-benzothiazol-2-amine: To a solution of tert-butyl (3R)-3-[7-[2-(tert-butoxycarbonylamino)-7-fluoro-1,3-benzothiazol-4-yl]-8-fluoro-2-[[(2S,4R)-4-methoxy-1-methyl-pyrrolidin-2-yl]methoxy]-6-(trifluoromethyl) quinazolin-4-yl]oxypyrrolidine-1-carboxylate (69 mg, 0.085 mmol) in DCM (3 mL) was added trifluoroacetic acid (1.5 mL, 19.5 mmol) at room temperature and stirred for 2 hours. After concentrated under vacuum, the residue was lyophilized under vacuum to give 7-fluoro-4-[8-fluoro-2-[[(2S,4R)-4-methoxy-1-methyl-pyrrolidin-2-yl]methoxy]-4-[(3R)-pyrrolidin-3-yl]oxy-6-(trifluoromethyl) quinazolin-7-yl]-1,3-benzothiazol-2-amine (57 mg, 100%). LCMS ESI (+) m/z 611.2 (M+H).

Step D: Preparation of 1-((R)-3-(((S)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 14) and 1-((R)-3-(((R)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 15): To a solution of 7-fluoro-4-[8-fluoro-2-[[(2S,4R)-4-methoxy-1-methyl-pyrrolidin-2-yl]methoxy]-4-[(3R)-pyrrolidin-3-yl]oxy-6-(trifluoromethyl) quinazolin-7-yl]-1,3-benzothiazol-2-amine (1.00 eq, 57 mg, 0.0934 mmol) in DCM (2 mL) was added TEA (0.039 mL, 0.28 mmol) and then to this was added dropwise a solution of prop-2-enoyl prop-2-enoate (11 mg, 0.084 mmol) in DCM (0.50 mL) at −40° C. under Ar. The mixture was stirred for 30 minutes at −40° C. Water and DCM were added. The organic layer was separated, dried over Na₂SO₄, filtered, and concentrated to dry and the residue was purified by reverse phase preparative-HPLC. The crude was then purified by Preparative-HPLC. The first compound off the column is identified as one atropisomer, 1-((R)-3-(((S)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 14) (11 mg, 18%). LCMS ESI (+) m/z 665.2 (M+H). ¹HNMR (400 MHZ, CD₃OD) δ 8.30 (s, 1H), 7.22 (s, 1H), 7.00 (t, J=8.6 Hz, 1H), 6.57-6.76 (m, 1H), 6.32 (d, J=16.8 Hz, 1H), 5.97-6.08 (m, 1H), 5.73-5.85 (m, 1H), 4.93-5.06 (m, 2H), 4.70-4.80 (m, 1H), 4.07-4.26 (m, 3H), 3.77-4.02 (m, 4H), 3.34-3.40 (m, 3H), 3.11-3.21 (s, 3H), 2.41-2.66 (m, 3H), 2.14-2.25 (m, 1H). The second compound off the column is identified as the other atropisomer, 1-((R)-3-(((R)-7-(2-amino-7-fluorobenzo[d]thiazol-4-yl)-8-fluoro-2-(((2S,4R)-4-methoxy-1-methylpyrrolidin-2-yl)methoxy)-6-(trifluoromethyl) quinazolin-4-yl)oxy)pyrrolidin-1-yl) prop-2-en-1-one (Compound 15) (6.5 mg, 10%). LCMS ESI (+) m/z 665.2 (M+H). ¹HNMR (400 MHZ, CD₃OD) δ 8.30 (s, 1H), 7.22 (s, 1H), 7.00 (t, J=8.6 Hz, 1H), 6.57-6.76 (m, 1H), 6.32 (d, J=16.8 Hz, 1H), 5.97-6.08 (m, 1H), 5.73-5.85 (m, 1H), 4.93-5.06 (m, 2H), 4.70-4.80 (m, 1H), 4.07-4.26 (m, 3H), 3.77-4.02 (m, 4H), 3.34-3.40 (m, 3H), 3.11-3.21 (s, 3H), 2.41-2.66 (m, 3H), 2.14-2.25 (m, 1H).

### Synthetic Example 4: Synthesis of 1-((3S,4S)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one (Compound 28)

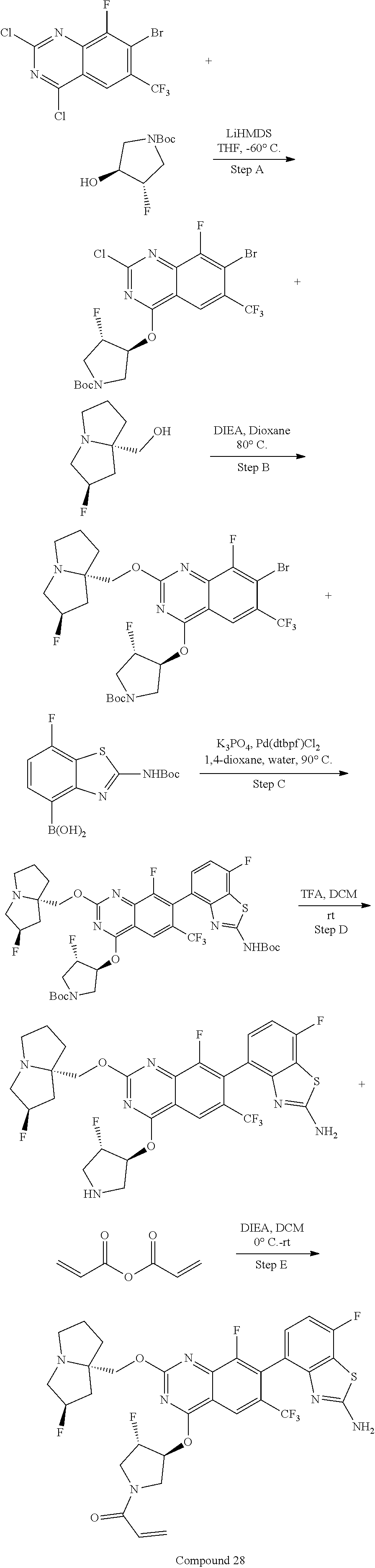

Step A: Preparation of tert-butyl (3S,4S)-3-[7-bromo-2-chloro-8-fluoro-6-(trifluoromethyl) quinazolin-4-yl]oxy-4-fluoro-pyrrolidine-1-carboxylate: To a solution of tert-butyl (3S,4S)-3-fluoro-4-hydroxy-pyrrolidine-1-carboxylate (250 mg, 1.22 mmol) in THF (1.0 mL) was added LiHMDS (1M in THF) (1.02 mL, 1.02 mmol) dropwise at −70° C. under Ar and then stirred at −70° C. for 1 hour. 7-bromo-2,4-dichloro-8-fluoro-6-(trifluoromethyl) quinazoline (370 mg, 1.02 mmol) in THF (3 mL) was added at −70° C. After stirring for 1 hour at −70° C., the mixture was quenched with NH₄Cl aqueous solution and extracted with EtOAc. The combined organics were dried (sodium sulfate), filtered, and concentrated to dry under vacuum and the residue was purified by column chromatography on silica gel, eluting with 25% EtOAc in petroleum ether, to give tert-butyl (3S,4S)-3-[7-bromo-2-chloro-8-fluoro-6-(trifluoromethyl) quinazolin-4-yl]oxy-4-fluoro-pyrrolidine-1-carboxylate (148 mg, 27%). LCMS ESI (+) m/z 532.1 (M+H).

Step B: Preparation of (3S,4S)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-4-fluoro-pyrrolidine-1-carboxylate: To a solution of tert-butyl (3S,4S)-3-[7-bromo-2-chloro-8-fluoro-6-(trifluoromethyl) quinazolin-4-yl]oxy-4-fluoro-pyrrolidine-1-carboxylate (148 mg, 0.28 mmol) in 1,4-dioxzne (1 mL) was [(2R,8S)-2-fluoro-1,2,3,5,6,7-hexahydropyrrolizin-8-yl]methanol (88 mg, 0.56 mmol) and DIEA (0.14 mL, 0.83 mmol) at ambient temperature. The mixture was sealed and then stirred at 80° C. for 8 hours. After cooling to ambient temperature, the mixture was diluted with brine, extracted with EtOAc and washed with Brine. The organics were separated, dried (sodium sulfate), filtered, and concentrated under vacuum. The residue was purified by column chromatography on silica gel, eluting with 50% EtOAc in petroleum ether, to give (3S,4S)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-4-fluoro-pyrrolidine-1-carboxylate (143 mg, 78%). LCMS ESI (+) m/z 655.1 (M+H).

Step C: Preparation of tert-butyl (3S,4S)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate: To a solution of [2-(tert-butoxycarbonylamino)-7-fluoro-1,3-benzothiazol-4-yl]boronic acid (82 mg, 0.26 mmol) in 1,4-Dioxane (2 mL)/Water (0.40 mL) was added ((7-bromo-4-(((3S,4S)-1-(tert-butoxycarbonyl)-4-fluoropyrrolidin-3-yl)oxy)-8-fluoro-2-(((2R,7aS)-2-fluorotetrahydro-1H-pyrrolizin-7a (5H)-yl) methoxy)quinazolin-6-yl)difluoromethyl)fluoronium (143 mg, 0.22 mmol), Pd(dtbpf)Cl₂ (14 mg, 0.022 mmol) and K₃PO₄ (93.4 mg, 0.44 mmol) at room temperature. The mixture was bubbled with Ar for 1-2 minutes and then sealed. After that, the mixture was stirred at 90° C. for 4 hours. After cooling to ambient temperature, water and ethyl acetate were added. The organic layer was separated, dried (sodium sulfate), filtered, and concentrated under reduced pressure. The residue was purified by preparative-TLC (50% EtOAc in petroleum ether) to give tert-butyl (3S,4S)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate (100 mg, 54%). LCMS ESI (+) m/z 843.3 (M+H).

Step D: Preparation of 7-fluoro-4-(8-fluoro-4-(((3S,4S)-4-fluoropyrrolidin-3-yl)oxy)-2-(((2R,7aS)-2-fluorotetrahydro-1H-pyrrolizin-7a (5H)-yl)methoxy)-6-(trifluoromethyl) quinazolin-7-yl)benzo[d]thiazol-2-amine: To a solution of tert-butyl (3S,4S)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate (40 mg, 0.048 mmol) in DCM (2 mL) was added trifluoroacetic acid (0.4 mL, 5.2 mmol) at room temperature and stirred for 2 hours. After concentrated under vacuum, the residue was suspended in DCM and water and adjusted pH with saturated sodium bicarbonate to pH˜9. It was extracted with DCM. Organic layer was separated, dried (sodium sulfate), filtered, concentrated under reduced pressure and dried to give 7-fluoro-4-(8-fluoro-4-(((3S,4S)-4-fluoropyrrolidin-3-yl)oxy)-2-(((2R,7aS)-2-fluorotetrahydro-1H-pyrrolizin-7a (5H)-yl)methoxy)-6-(trifluoromethyl) quinazolin-7-yl)benzo[d]thiazol-2-amine (30 mg, 98%). LCMS ESI (+) m/z 643.2 (M+H).

Step E: Preparation of 1-((3S,4S)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one: To a 7-fluoro-4-(8-fluoro-4-(((3S,4S)-4-fluoropyrrolidin-3-yl)oxy)-2-(((2R,7aS)-2-fluorotetrahydro-1H-pyrrolizin-7a (5H)-yl)methoxy)-6-(trifluoromethyl) quinazolin-7-yl)benzo[d]thiazol-2-amine (45 mg, 0.07 mmol) in DCM (3 mL) was added TEA (0.049 mL, 0.35 mmol) and then to this was added dropwise a solution of prop-2-enoyl prop-2-enoate (7.9 mg, 0.063 mmol) in DCM (0.50 mL) at 0° C. under Ar. And then it was warmed to room temperature and stirred at room temperature for 1 hour. Water and DCM were added. The organic layer was separated, dried over Na₂SO₄, filtered and concentrated to dry and the residue was purified by reverse phase preparative-HPLC. The crude was then purified by reverse phase Preparative-HPLC to give 1-((3S,4S)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one (14.5 mg, 28%). LCMS ESI (+) m/z 697.2 (M+H). ¹HNMR (400 MHZ, CD₃OD) δ 8.22-8.29 (m, 1H), 7.12-7.23 (m, 1H), 6.97 (t, J=8.8 Hz, 1H), 6.62-6.72 (m, 1H), 6.35 (d, J=16.7 Hz, 1H), 5.96-6.08 (m, 1H), 5.78-5.87 (m, 1H), 5.57 (dd, J=48.6, 20.7 Hz, 1H), 5.35 (d, J=55.2 Hz, 1H), 4.36-4.48 (m, 2H), 3.91-4.31 (m, 4H), 3.33-3.42 (m, 3H), 3.06-3.12 (m, 1H), 2.17-2.47 (m, 3H), 2.03 (m, 3H).

### Synthetic Example 5: Synthesis of 1-((2R,3R)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-2-methylpyrrolidin-1-yl) prop-2-en-1-one (Compound 29)

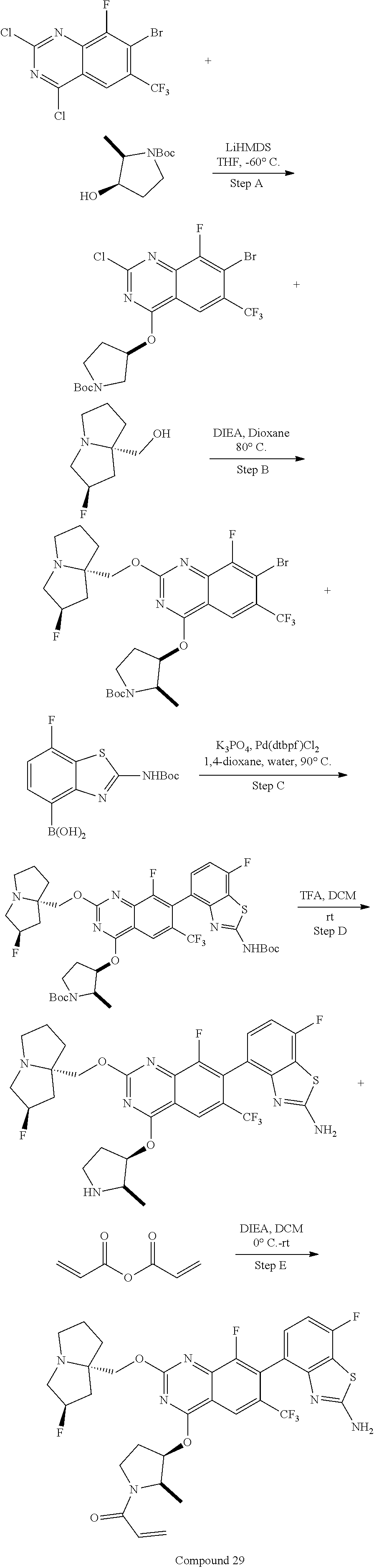

Step A: Preparation of tert-butyl cis-3-[7-bromo-2-chloro-8-fluoro-6-(trifluoromethyl) quinazolin-4-yl]oxy-2-methyl-pyrrolidine-1-carboxylate: To a solution of Cis-tert-butyl-3-hydroxy-2-methyl-pyrrolidine-1-carboxylate (200 mg, 0.99 mmol) in THF (1.0 mL) was added LiHMDS (1M in THF) (0.82 mL, 0.82 mmol) dropwise at −70° C. under Ar and then stirred at −70° C. for 1 hour. 7-bromo-2,4-dichloro-8-fluoro-6-(trifluoromethyl) quinazoline (302 mg, 0.83 mmol) in THF (1 mL) was added at −70° C. After stirring for 1 hour at −70° C., the mixture was quenched with NH₄Cl aqueous solution and extracted with EtOAc. The combined organics were dried (sodium sulfate), filtered, and concentrated to dry under vacuum and the residue was purified by column chromatography on silica gel, eluting with 25% EtOAc in petroleum ether, to give tert-butyl cis-3-[7-bromo-2-chloro-8-fluoro-6-(trifluoromethyl) quinazolin-4-yl]oxy-2-methyl-pyrrolidine-1-carboxylate (80 mg, 18%). LCMS ESI (+) m/z 473.9 (M+H).

Step B: Preparation of tert-butyl cis-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate: To a solution of tert-butyl cis-3-[7-bromo-2-chloro-8-fluoro-6-(trifluoromethyl) quinazolin-4-yl]oxy-2-methyl-pyrrolidine-1-carboxylate (80 mg, 0.15 mmol) in 1,4-dioxzne (1 mL) was [(2R,8S)-2-fluoro-1,2,3,5,6,7-hexahydropyrrolizin-8-yl]methanol (48 mg, 0.30 mmol) and DIEA (0.07 mL, 0.41 mmol) at ambient temperature. The mixture was sealed and then stirred at 80° C. for 8 hours. After cooling to ambient temperature, the mixture was diluted with brine, extracted with EtOAc and washed with brine. The organics were separated, dried (sodium sulfate), filtered and concentrated under vacuum. The residue was purified by column chromatography on silica gel, eluting with 50% EtOAc in petroleum ether, to give tert-butyl cis-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (85 mg, 86%). LCMS ESI (+) m/z 653.2 (M+H).

Step C: Preparation of tert-butyl cis-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate: To a solution of [2-(tert-butoxycarbonylamino)-7-fluoro-1,3-benzothiazol-4-yl]boronic acid (43 mg, 0.14 mmol) in 1,4-Dioxane (2 mL)/Water (0.40 mL) was added tert-butyl cis-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (75 mg, 0.12 mmol), Pd(dtbpf)Cl₂ (7.5 mg, 0.012 mmol) and K₃PO₄ (49 mg, 0.23 mmol) at room temperature. The mixture was bubbled with Ar for 1-2 minutes and then sealed. After that, the mixture was stirred at 90° C. for 4 hours. After cooling to ambient temperature, water and ethyl acetate were added. The organic layer was separated, dried (sodium sulfate), filtered and concentrated under reduced pressure. The residue was purified by preparative-TLC (50% EtOAc in petroleum ether) to give tert-butyl cis-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (80 mg, 82%). LCMS ESI (+) m/z 839.3 (M+H).

Step D: Preparation of 7-fluoro-4-(8-fluoro-2-(((2R,7aS)-2-fluorotetrahydro-1H-pyrrolizin-7a (5H)-yl)methoxy)-4-((cis-4-methylpyrrolidin-3-yl)oxy)-6-(trifluoromethyl) quinazolin-7-yl)benzo[d]thiazol-2-amine: To a solution of tert-butyl cis-3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (40 mg, 0.048 mmol) in DCM (2 mL) was added trifluoroacetic acid (0.6 mL, 7.8 mmol) at room temperature and stirred for 2 hours. After concentrated under vacuum, the residue was suspended in DCM and water and adjusted pH with saturated sodium bicarbonate to pH˜9. It was extracted with DCM. Organic layer was separated, dried (sodium sulfate), filtered, concentrated under reduced pressure and dried to give 7-fluoro-4-(8-fluoro-2-(((2R,7aS)-2-fluorotetrahydro-1H-pyrrolizin-7a (5H)-yl)methoxy)-4-((cis-4-methylpyrrolidin-3-yl)oxy)-6-(trifluoromethyl) quinazolin-7-yl)benzo[d]thiazol-2-amine (30 mg, 98%). LCMS ESI (+) m/z 639.2 (M+H).

Step E: Preparation of 1-(cis-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-2-methylpyrrolidin-1-yl) prop-2-en-1-one: To a 7-fluoro-4-(8-fluoro-2-(((2R,7aS)-2-fluorotetrahydro-1H-pyrrolizin-7a (5H)-yl)methoxy)-4-((cis-4-methylpyrrolidin-3-yl)oxy)-6-(trifluoromethyl) quinazolin-7-yl)benzo[d]thiazol-2-amine (30 mg, 0.047 mmol) in DCM (3 mL) was added TEA (0.033 mL, 0.24 mmol) and then to this was added dropwise a solution of prop-2-enoyl prop-2-enoate (5.3 mg, 0.042 mmol) in DCM (0.50 mL) at 0° C. under Ar. It was then warmed to room temperature and stirred at room temperature for 1 hour. Water and DCM were added. The organic layer was separated, dried over Na₂SO₄, filtered and concentrated to dry and the residue was purified by reverse phase preparative-HPLC to give 1-(cis-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-2-methylpyrrolidin-1-yl) prop-2-en-1-one (6.2 mg, 19%). LCMS ESI (+) m/z 693.2 (M+H). ¹HNMR (400 MHZ, CD₃OD) δ 8.30 (s, 1H), 7.16-7.25 (m, 1H), 6.97 (t, J=8.8 Hz, 1H), 6.66 (dd, J=16.8, 10.8 Hz, 1H), 6.33 (t, J=16.8 Hz, 1H), 5.74-5.92 (m, 2H), 5.32 (d, J=54.4 Hz, 1H), 4.69-4.77 (m, 1H), 4.52-4.63 (m, 1H), 4.28-4.42 (m, 2H), 3.61-3.97 (m, 2H), 3.18-3.26 (m, 2H), 2.99-3.10 (m, 1H), 1.89-2.64 (m, 8H), 1.21-1.33 (m, 3H).

### Synthetic Example 6: Synthesis of 1-((R)-3-(((S)-7-(2-amino-7-fluorobenzo[d]thiazol-4-yl)-8-fluoro-2-(((2R,7aR)-2-methoxytetrahydro-1H-pyrrolizin-7a (5H)-yl)methoxy)-6-(trifluoromethyl) quinazolin-4-yl)oxy)pyrrolidin-1-yl) prop-2-en-1-one (Compound 30) and 1-((R)-3-(((R)-7-(2-amino-7-fluorobenzo[d]thiazol-4-yl)-8-fluoro-2-(((2R,7aR)-2-methoxytetrahydro-1H-pyrrolizin-7a (5H)-yl) methoxy)-6-(trifluoromethyl) quinazolin-4-yl)oxy)pyrrolidin-1-yl) prop-2-en-1-one (Compound 31)

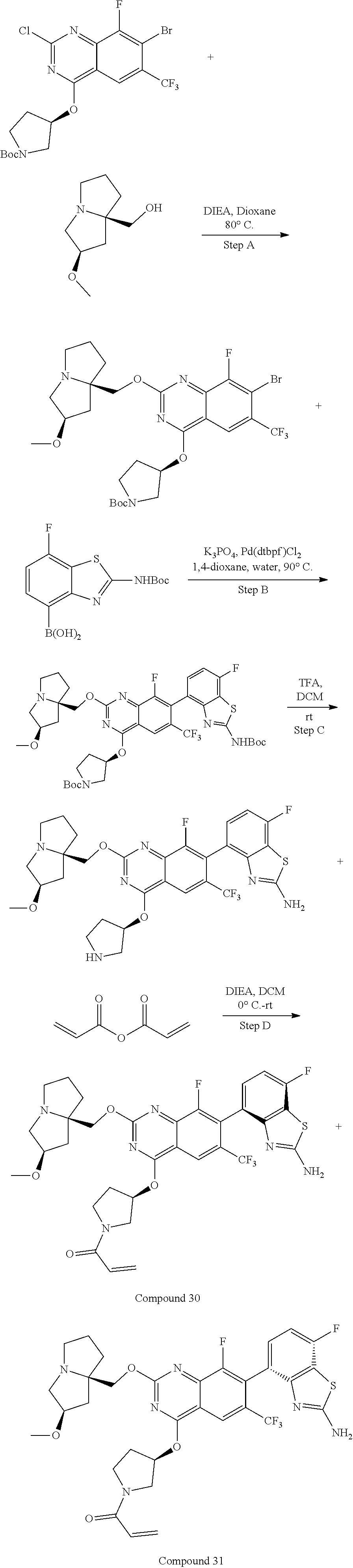

Step A: Preparation of tert-butyl (3R)-3-[7-bromo-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate: To a solution of tert-butyl (3R)-3-[7-bromo-2-chloro-8-fluoro-6-(trifluoromethyl) quinazolin-4-yl]oxypyrrolidine-1-carboxylate (100 mg, 0.19 mmol) in 1,4-dioxzne (1 mL) was added [(2R,8R)-2-methoxy-1,2,3,5,6,7-hexahydropyrrolizin-8-yl]methanol (67 mg, 0.39 mmol) and DIEA (0.10 mL, 0.58 mmol) at ambient temperature. The mixture was sealed and then stirred at 80° C. for 4 hours. After cooling to ambient temperature, the mixture was diluted with brine, extracted with EtOAc and washed with brine. The organics were separated, dried (sodium sulfate), filtered and concentrated under vacuum. The residue was purified by preparative-TLC (DCM:MeOH=20:1) to give tert-butyl (3S)-3-[7-bromo-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate (114 mg, 90%). LCMS ESI (+) m/z 649.2 (M+H).

Step B: Preparation of tert-butyl (3R)-3-[7-[2-(tert-butoxycarbonylamino)-7-fluoro-1,3-benzothiazol-4-yl]-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate: To a solution of tert-butyl (3S)-3-[7-bromo-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate (114 mg, 0.18 mmol), [2-(tert-butoxycarbonylamino)-7-fluoro-1,3-benzothiazol-4-yl]boronic acid (71 mg, 0.23 mmol) in 1,4-Dioxane (5 mL) and Water (1 mL) was added Pd(dtbpf)Cl₂ (11 mg, 0.018 mmol) and K₃PO₄ (75 mg, 0.35 mmol) at room temperature. The mixture was bubbled with Ar for 1-2 minutes and then sealed. After that, the mixture was stirred at 90° C. for 4 hours. After cooling to ambient temperature, water and ethyl acetate were added. The organic layer was separated, dried (sodium sulfate), filtered, and concentrated under reduced pressure. The residue was purified by preparative-TLC (DCM:MeOH=20:1) to give tert-butyl (3R)-3-[7-[2-(tert-butoxycarbonylamino)-7-fluoro-1,3-benzothiazol-4-yl]-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate (143 mg, 97%). LCMS ESI (+) m/z 837.4 (M+H).

Step C: Preparation of 7-fluoro-4-[8-fluoro-2-[[(2R,8R)-2-methoxy-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine: To a solution of tert-butyl (3R)-3-[7-[2-(tert-butoxycarbonylamino)-7-fluoro-1,3-benzothiazol-4-yl]-8-fluoro-2-[[(2R,8R)-2-methoxy-1,2,3,5,6,7-hexahydropyrrolizin-8-yl]methoxy]-6-(trifluoromethyl) quinazolin-4-yl]oxypyrrolidine-1-carboxylate (143 mg, 0.17 mmol) in DCM (4 mL) was added trifluoroacetic acid (1.0 mL, 13.0 mmol) at room temperature and stirred for 2 hours. After concentrated under vacuum to give 7-fluoro-4-[8-fluoro-2-[[(2R,8R)-2-methoxy-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine (104 mg, 95%). LCMS ESI (+) m/z 637.2 (M+H).

Step D: Preparation of 1-((R)-3-(((S)-7-(2-amino-7-fluorobenzo[d]thiazol-4-yl)-8-fluoro-2-(((2R,7aR)-2-methoxytetrahydro-1H-pyrrolizin-7a (5H)-yl)methoxy)-6-(trifluoromethyl) quinazolin-4-yl)oxy)pyrrolidin-1-yl) prop-2-en-1-one (Compound 30) and 1-((R)-3-(((R)-7-(2-amino-7-fluorobenzo[d]thiazol-4-yl)-8-fluoro-2-(((2R,7aR)-2-methoxytetrahydro-1H-pyrrolizin-7a (5H)-yl) methoxy)-6-(trifluoromethyl) quinazolin-4-yl)oxy)pyrrolidin-1-yl) prop-2-en-1-one (Compound 31): To a solution of 7-fluoro-4-[8-fluoro-2-[[(2R,8R)-2-methoxy-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzothiazol-2-amine (52 mg, 0.082 mmol), TEA (0.11 mL, 0.82 mmol) in DCM (5 mL) was added prop-2-enoyl prop-2-enoate (15 mg, 0.12 mmol) at 0° C. under Ar. It was then warmed to room temperature and stirred at room temperature for 1 hour. Water and DCM were added. The organic layer was separated, dried over Na₂SO₄, filtered, and concentrated to dry and the residue was purified by reverse phase preparative-HPLC. The first compound off the column is identified as one atropisomer, 1-((R)-3-(((S)-7-(2-amino-7-fluorobenzo[d]thiazol-4-yl)-8-fluoro-2-(((2R,7aR)-2-methoxytetrahydro-1H-pyrrolizin-7a (5H)-yl)methoxy)-6-(trifluoromethyl) quinazolin-4-yl)oxy)pyrrolidin-1-yl) prop-2-en-1-one (Compound 30) (7.5 mg, 13%) LCMS ESI (+) m/z 691.3 (M+H). ¹HNMR (400 MHZ, CD₃OD) δ 8.27 (s, 1H), 7.18-7.21 (m, 1H), 6.97 (t, J=8.82 Hz, 1H), 6.58-6.74 (m, 1H), 6.29-6.34 (m, 1H), 5.99-6.06 (m, 1H), 5.75-5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H). The second compound off the column is identified as the other atropisomer, 1-((R)-3-(((R)-7-(2-amino-7-fluorobenzo[d]thiazol-4-yl)-8-fluoro-2-(((2R,7aR)-2-methoxytetrahydro-1H-pyrrolizin-7a (5H)-yl) methoxy)-6-(trifluoromethyl) quinazolin-4-yl)oxy)pyrrolidin-1-yl) prop-2-en-1-one (Compound 31) (5.3 mg, 9%). LCMS ESI (+) m/z 691.3 (M+H). ¹HNMR (400 MHZ, CD₃OD) δ 8.27 (s, 1H), 7.18-7.21 (m, 1H), 6.97 (t, J=8.82 Hz, 1H), 6.58-6.74 (m, 1H), 6.29-6.34 (m, 1H), 5.99-6.06 (m, 1H), 5.75-5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H).

### Synthetic Example 7: Synthesis of 1-((3R)-3-((7-(2-amino-7-fluorobenzo[d]oxazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one (Compound 36)

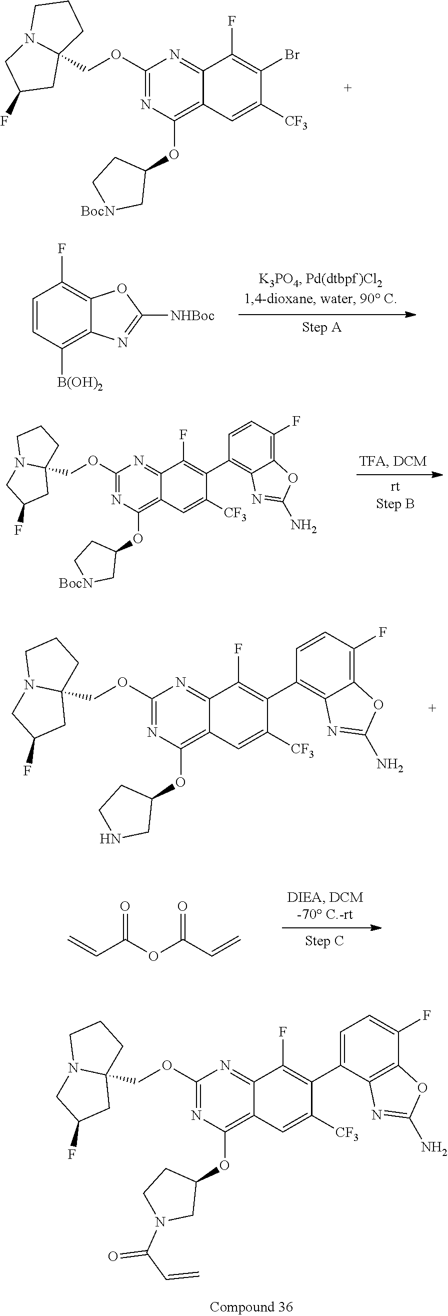

Step A: Preparation of tert-butyl (3R)-3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate: To a solution of tert-butyl (3R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (106 mg, 0.17 mmol), (2-amino-7-fluoro-1,3-benzoxazol-4-yl) boronic acid (65 mg, 0.33 mmol) in 1,4-Dioxane (5 mL) and Water (ImL) in 1,4-Dioxane (5 mL) and water (1 mL) was added Pd(dtbpf)Cl₂ (11 mg, 0.017 mmol) and K₃PO₄ (75 mg, 0.35 mmol) at room temperature. The mixture was bubbled with Ar for 1-2 minutes and then sealed. After that, the mixture was stirred at 90° C. for 4 hours. After cooling to ambient temperature, water and ethyl acetate were added. The organic layer was separated, dried (sodium sulfate), filtered, and concentrated under reduced pressure. The residue was purified by preparative-TLC (DCM:MeOH=20:1) to give tert-butyl (3R)-3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (29 mg, 24%). LCMS ESI (+) m/z 709.3 (M+H).

Step B: Preparation of 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzoxazol-2-amine: To a solution of tert-butyl (3R)-3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidine-1-carboxylate (31 mg, 0.044 mmol) in DCM (4 mL) was added was added trifluoroacetic acid (1.0 mL, 13.0 mmol) at room temperature and stirred for overnight. After concentrated under vacuum to give 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzoxazol-2-amine (24 mg, 90%). LCMS ESI (+) m/z 609.3 (M+H).

Step C: Preparation of 1-((3R)-3-((7-(2-amino-7-fluorobenzo[d]oxazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)pyrrolidin-1-yl) prop-2-en-1-one: To a solution of 7-fluoro-4-[8-fluoro-2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl) quinazolin-7-yl]-1,3-benzoxazol-2-amine (23 mg, 0.038 mmol), TEA (0.053 mL, 0.38 mmol) in DCM (4 mL) was added prop-2-enoyl prop-2-enoate (7.1 mg, 0.057 mmol) at −70° C. under Ar. It was then warmed to room temperature and stirred at room temperature for 1 hour. Water and DCM were added. The organic layer was separated, dried over Na₂SO₄, filtered, and concentrated to dry and the residue was purified by reverse phase preparative-HPLC to give 1-[(3R)-3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]oxypyrrolidin-1-yl]prop-2-en-1-one (6.2 mg, 24%). LCMS ESI (+) m/z 663.3 (M+H). ¹HNMR (400 MHZ, CD₃OD) δ 8.27 (s, 1H), 7.18-7.21 (m, 1H), 6.97 (t, J=8.82 Hz, 1H), 6.58-6.74 (m, 1H), 6.29-6.34 (m, 1H), 5.99-6.06 (m, 1H), 5.75-5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H).

### Synthetic Example 8: Synthesis of 1-((3S,4R)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one (Compound 40)

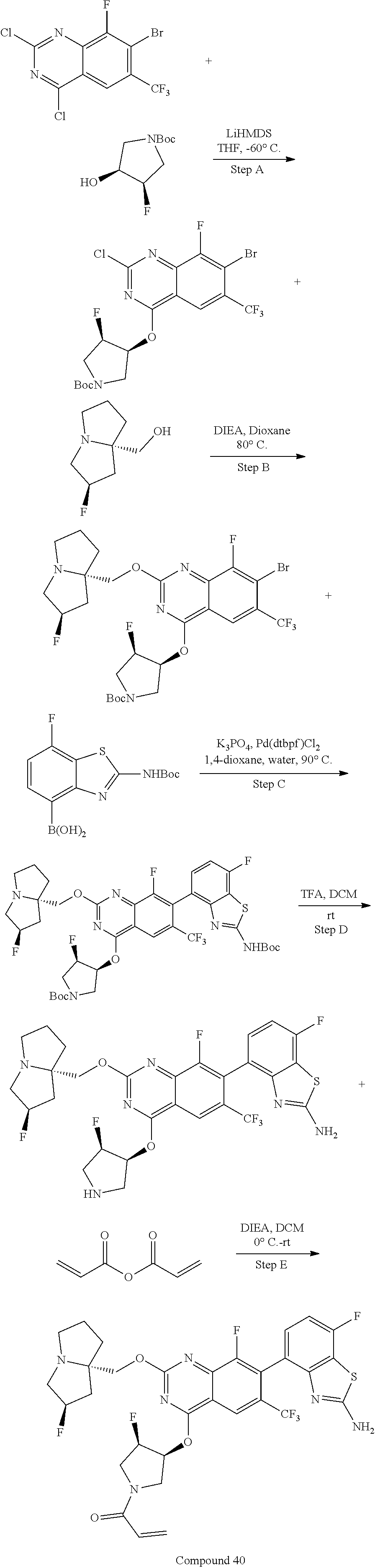

Step A: Preparation of tert-butyl (3S,4R)-3-[7-bromo-2-chloro-8-fluoro-6-(trifluoromethyl) quinazolin-4-yl]oxy-4-fluoro-pyrrolidine-1-carboxylate: To a solution of tert-butyl (3S,4R)-3-fluoro-4-hydroxy-pyrrolidine-1-carboxylate (200 mg, 0.97 mmol) in THF (1.0 mL) was added LiHMDS (1M in THF) (1.02 mL, 1.02 mmol) dropwise at −70° C. under Ar and then stirred at −70° C. for 1 hour. 7-bromo-2,4-dichloro-8-fluoro-6-(trifluoromethyl) quinazoline (295 mg, 0.81 mmol) in THF (1 mL) was added at −70° C. After stirring for 1 hour at −70° C., the mixture was quenched with NH₄Cl aqueous solution and extracted with EtOAc. The combined organics were dried (sodium sulfate), filtered and concentrated to dry under vacuum and the residue was purified by column chromatography on silica gel, eluting with 25% EtOAc in petroleum ether, to give tert-butyl (3S,4R)-3-[7-bromo-2-chloro-8-fluoro-6-(trifluoromethyl) quinazolin-4-yl]oxy-4-fluoro-pyrrolidine-1-carboxylate (64 mg, 14%). LCMS ESI (+) m/z 532.0 (M+H).

Step B: Preparation of tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-4-fluoro-pyrrolidine-1-carboxylate: To a solution of tert-butyl (3S,4R)-3-[7-bromo-2-chloro-8-fluoro-6-(trifluoromethyl) quinazolin-4-yl]oxy-4-fluoro-pyrrolidine-1-carboxylate (64 mg, 0.12 mmol) in 1,4-dioxzne (1 mL) was added [(2R,8S)-2-fluoro-1,2,3,5,6,7-hexahydropyrrolizin-8-yl]methanol (38 mg, 0.24 mmol) and DIEA (0.04 mL, 0.83 mmol) at ambient temperature. The mixture was sealed and then stirred at 80° C. for 8 hours. After cooling to ambient temperature, the mixture was diluted with brine, extracted with EtOAc and washed with brine. The organics were separated, dried (sodium sulfate), filtered and concentrated under vacuum. The residue was purified by column chromatography on silica gel, eluting with 50% EtOAc in petroleum ether, to give tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-4-fluoro-pyrrolidine-1-carboxylate (65 mg, 82%). LCMS ESI (+) m/z 655.1 (M+H).

Step C: Preparation of tert-butyl (3S,4R)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate: To a solution of [2-(tert-butoxycarbonylamino)-7-fluoro-1,3-benzothiazol-4-yl]boronic acid (34 mg, 0.11 mmol) in 1,4-Dioxane (2 mL)/Water (0.40 mL) was added tert-butyl (3S,4R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-4-fluoro-pyrrolidine-1-carboxylate (60 mg, 0.092 mmol), Pd(dtbpf)Cl₂ (6.0 mg, 0.01 mmol) and K₃PO₄ (39 mg, 0.18 mmol) at room temperature. The mixture was bubbled with Ar for 1˜2 minutes and then sealed. After that, the mixture was stirred at 90° C. for 4 hours. After cooling to ambient temperature, water and ethyl acetate were added. The organic layer was separated, dried (sodium sulfate), filtered, and concentrated under reduced pressure. The residue was purified by preparative-TLC (50% EtOAc in petroleum ether) to give tert-butyl (3S,4R)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate (51 mg, 66%). LCMS ESI (+) m/z 843.3 (M+H).

Step D: Preparation of 7-fluoro-4-(8-fluoro-4-(((3S,4R)-4-fluoropyrrolidin-3-yl)oxy)-2-(((2R,7aS)-2-fluorotetrahydro-1H-pyrrolizin-7a (5H)-yl)methoxy)-6-(trifluoromethyl) quinazolin-7-yl)benzo[d]thiazol-2-amine: To a solution of tert-butyl (3S,4R)-3-((7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidine-1-carboxylate (25 mg, 0.030 mmol) in DCM (3 mL) was added trifluoroacetic acid (1.0 mL, 13.0 mmol) at room temperature and stirred for 2 hours. After concentrated under vacuum, the residue was suspended in DCM and water and adjusted pH with saturated sodium bicarbonate to pH˜9. It was extracted with DCM. Organic layer was separated, dried (sodium sulfate), filtered, concentrated under reduced pressure and dried to give 7-fluoro-4-(8-fluoro-4-(((3S,4R)-4-fluoropyrrolidin-3-yl)oxy)-2-(((2R,7aS)-2-fluorotetrahydro-1H-pyrrolizin-7a (5H)-yl) methoxy)-6-(trifluoromethyl) quinazolin-7-yl)benzo[d]thiazol-2-amine (20 mg, 100%). LCMS ESI (+) m/z 643.2 (M+H).

Step E: Preparation of 1-((3S,4R)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one: To a solution of 7-fluoro-4-(8-fluoro-4-(((3S,4R)-4-fluoropyrrolidin-3-yl)oxy)-2-(((2R,7aS)-2-fluorotetrahydro-1H-pyrrolizin-7a (5H)-yl)methoxy)-6-(trifluoromethyl) quinazolin-7-yl)benzo[d]thiazol-2-amine (20 mg, 0.031 mmol) in DCM (3 mL) was added TEA (0.022 mL, 0.16 mmol) and then to this was added dropwise a solution of prop-2-enoyl prop-2-enoate (3.5 mg, 0.028 mmol) in DCM (0.50 mL) at 0° C. under Ar. And then it was warmed to room temperature and stirred at room temperature for 1 hour. Water and DCM were added. The organic layer was separated, dried over Na₂SO₄, filtered and concentrated to dry and the residue was purified by reverse phase preparative-HPLC to give 1-((3S,4R)-3-((7-(2-amino-7-fluorobenzo[d]thiazol-4-yl)-8-fluoro-2-(((2R,7aS)-2-fluorotetrahydro-1H-pyrrolizin-7a (5H)-yl)methoxy)-6-(trifluoromethyl) quinazolin-4-yl)oxy)-4-fluoropyrrolidin-1-yl) prop-2-en-1-one (3 mg, 14%). LCMS ESI (+) m/z 697.2 (M+H). ¹HNMR (400 MHZ, CD₃OD) δ 8.51 (s, 1H), 8.33 (s, 1H), 7.18-7.24 (m, 1H), 6.97 (t, J=8.8 Hz, 1H), 6.59-6.72 (m, 1H), 6.36 (d, J=16.4 Hz, 1H), 5.89-6.02 (m, 2H), 5.66 (dd, J=54.4, 4.0 Hz, 1H), 5.33 (d, J=54 Hz, 1H), 5.09-5.21 (m, 1H), 4.30-4.51 (m, 3H), 3.94-4.19 (m, 3H), 3.01-3.28 (m, 3H), 1.85-2.43 (m, 6H).

### Synthetic Example 9: Synthesis of 4-(4-(((R)-1-acryloylpyrrolidin-3-yl)oxy)-2-(((2R,7aS)-2-fluorotetrahydro-1H-pyrrolizin-7a (5H)-yl)methoxy)-6-(trifluoromethyl)pyrido[3,2-d]pyrimidin-7-yl)-2-amino-7-fluorobenzo[b]thiophene-3-carbonitrile (Compound 55)

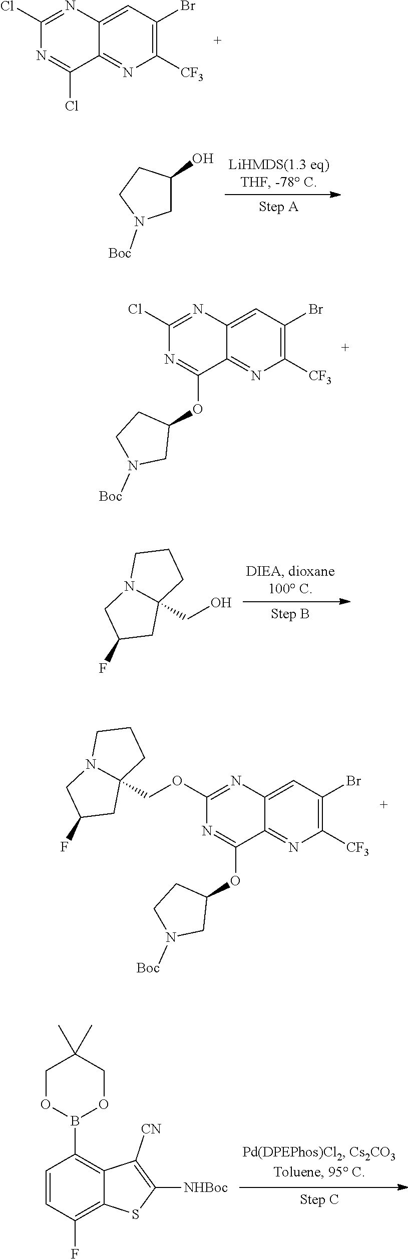

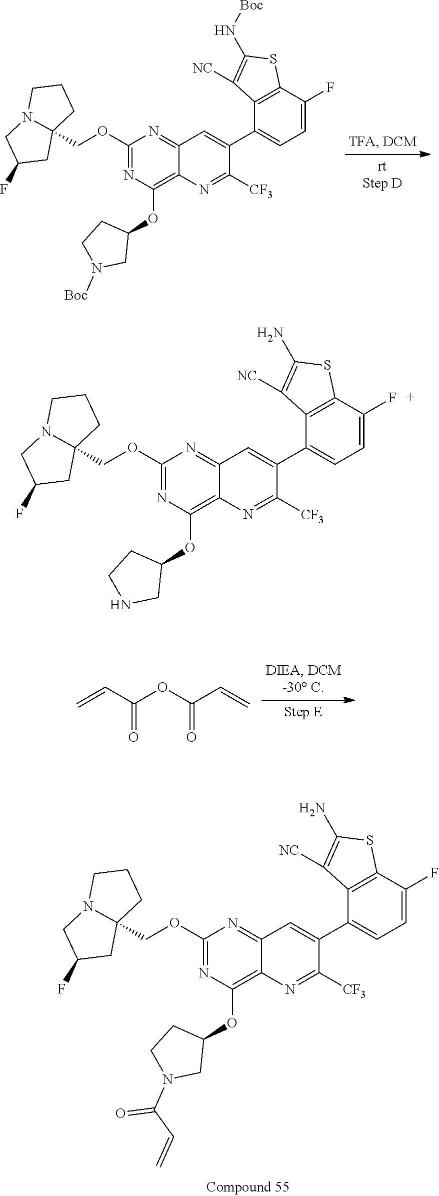

Step A: Preparation of tert-butyl (R)-3-((7-bromo-2-chloro-6-(trifluoromethyl)pyrido[3,2-d]pyrimidin-4-yl)oxy)pyrrolidine-1-carboxylate: To a solution of tert-butyl (3R)-3-hydroxypyrrolidine-1-carboxylate (378 mg, 2.02 mmol) in anhydrous THF (6 mL) was added was added LiHMDS (2.8 mL, 3.03 mmol) dropwise at −78° C. under Ar and then stirred at −78° C. for 1 hour. 7-bromo-2,4-dichloro-6-(trifluoromethyl)pyrido[3,2-d]pyrimidine (1.00 eq, 700 mg, 2.02 mmol) in THF (2 mL) was added at −78° C. After stirred for 1 hour at −78° C. water, extracted with EA (3*10 mL), the combined organic phase was washed brine, dried over anhydrous sodium sulfate, concentrated under vacuum. The residue was purified with preparative-TLC (petroleum ether/ethyl acetate=5:1) to afford tert-butyl (3R)-3-[7-bromo-2-chloro-6-(trifluoromethyl)pyrido[3,2-d]pyrimidin-4-yl]oxypyrrolidine-1-carboxylate (350 mg, 31%) as solid. LCMS ESI (+) m/z 497.1 (M+H).

Step B: Preparation of tert-butyl (R)-3-((7-bromo-2-(((2R,7aS)-2-fluorotetrahydro-1H-pyrrolizin-7a (5H)-yl)methoxy)-6-(trifluoromethyl)pyrido[3,2-d]pyrimidin-4-yl)oxy)pyrrolidine-1-carboxylate: To a solution of tert-butyl (3R)-3-[7-bromo-2-chloro-6-(trifluoromethyl)pyrido[3,2-d]pyrimidin-4-yl]oxypyrrolidine-1-carboxylate (350 mg, 0.70 mmol) in dioxane (5 mL) was added [(2R,8S)-2-fluoro-1,2,3,5,6,7-hexahydropyrrolizin-8-yl]methanol (179 mg, 1.13 mmol) and DIEA (0.63 mL, 3.52 mmol) at room temperature. The resulting reaction mixture was stirred at 80° C. for 16 hours under nitrogen atmosphere. After cooled to ambient temperature, the resulting mixture was diluted with water and extracted with EA (3*20 mL). The combined organic phase was washed brine, dried over anhydrous sodium sulfate, concentrated under vacuum. The residue was purified with preparative-TLC (petroleum ether/ethyl acetate=1:1) to afford tert-butyl (R)-3-((7-bromo-2-(((2R,7aS)-2-fluorotetrahydro-1H-pyrrolizin-7a (5H)-yl) methoxy)-6-(trifluoromethyl)pyrido[3,2-d]pyrimidin-4-yl)oxy)pyrrolidine-1-carboxylate (298 mg, 68% yield) as solid. LCMS ESI (+) m/z 620.1 (M+H).

Step C: Preparation tert-butyl (R)-3-((7-(2-((tert-butoxycarbonyl)amino)-3-cyano-7-fluorobenzo[b]thiophen-4-yl)-2-(((2R,7aS)-2-fluorotetrahydro-1H-pyrrolizin-7a (5H)-yl)methoxy)-6-(trifluoromethyl)pyrido[3,2-d]pyrimidin-4-yl)oxy)pyrrolidine-1-carboxylate: To a mixture of tert-butyl (3R)-3-[7-bromo-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (110 mg, 0.18 mmol) and tert-butyl N-[3-cyano-4-(5,5-dimethyl-1,3,2-dioxaborinan-2-yl)-7-fluoro-benzothiophen-2-yl]carbamate (143 mg, 0.36 mmol) in dioxane (2 mL) was added potassium trimethylsilanolate (0.11 mL, 0.53 mmol) and Pd(DPEPhos)Cl₂ (63 mg, 0.089 mmol) at room temperature. The resulting mixture was stirred at 95° C. for 6 hours under nitrogen atmosphere. After cooled to ambient temperature, the resulting mixture was diluted with water and extracted with EA (3*20 mL). The combined organic phase was washed brine, dried over anhydrous sodium sulfate, concentrated under vacuum. The residue was purified with preparative-TLC (petroleum ether/ethyl acetate=1:1) to afford tert-butyl (3R)-3-[7-[2-(tert-butoxycarbonylamino)-3-cyano-7-fluoro-benzothiophen-4-yl]-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (110 mg, 74% yield) as solid. LCMS ESI (+) m/z 832.1 (M+H).

Step D: Preparation of 2-amino-7-fluoro-4-(2-(((2R,7aS)-2-fluorotetrahydro-1H-pyrrolizin-7a (5H)-yl)methoxy)-4-(((R)-pyrrolidin-3-yl)oxy)-6-(trifluoromethyl)pyrido[3,2-d]pyrimidin-7-yl)benzo[b]thiophene-3-carbonitrile: To a solution of tert-butyl (3R)-3-[7-[2-(tert-butoxycarbonylamino)-3-cyano-7-fluoro-benzothiophen-4-yl]-2-[[(2R,8S)-2-fluoro-1,2,3,5,6,7-hexahydropyrrolizin-8-yl]methoxy]-6-(trifluoromethyl)pyrido[3,2-d]pyrimidin-4-yl]oxypyrrolidine-1-carboxylate (200 mg, 0.24 mmol) in DCM (5 mL) was added TFA (2.5 mL, 32.5 mmol) at room temperature under nitrogen atmosphere. The resulting solution was stirred for 16 hours at room temperature under nitrogen atmosphere. The solvent was removed and the residue was purified by RP preparative-HPLC to give 2-amino-4-[4-(3,8-diazabicyclo[3.2.1]octan-3-yl)-2-[[(2R,8S)-2-fluoro-1,2,3,5,6,7-hexahydropyrrolizin-8-yl]methoxy]pyrido[2,3-d]pyrimidin-7-yl]-7-fluoro-benzothiophene-3-carbonitrile (14 mg, 9% yield) as solid. LCMS ESI (+) m/z 632.0 (M+H). ¹H NMR (400 MHZ, CD₃OD) δ 8.32 (d, 1H), 8.16-8.20 (m, 1H), 7.22-7.25 (m, 1H), 7.02-7.06 (m, 1H), 6.15 (s, 1H), 5.58-5.64 (m, 0.5H), 5.45-5.51 (m, 0.5H), 4.62-4.69 (m, 2H), 4.29 (s, 1H), 3.59-3.85 (m, 6H), 2.05-2.62 (m, 9H).

Step E: Preparation of 4-(4-(((R)-1-acryloylpyrrolidin-3-yl)oxy)-2-(((2R,7aS)-2-fluorotetrahydro-1H-pyrrolizin-7a (5H)-yl)methoxy)-6-(trifluoromethyl)pyrido[3,2-d]pyrimidin-7-yl)-2-amino-7-fluorobenzo[b]thiophene-3-carbonitrile: To a solution of 2-amino-7-fluoro-4-[2-[[(2R,8S)-2-fluoro-1,2,3,5,6,7-hexahydropyrrolizin-8-yl]methoxy]-4-[(3R)-pyrrolidin-3-yl]oxy-6-(trifluoromethyl)pyrido[3,2-d]pyrimidin-7-yl]benzothiophene-3-carbonitrile (10 mg, 0.016 mmol) in DCM (1 mL) was added prop-2-enoyl prop-2-enoate (1.6 mg, 0.013 mmol) dropwise at −30° C. under nitrogen atmosphere. The resulting solution was stirred for 30 minutes at −30° C. under nitrogen atmosphere. The mixture was poured into water (10 mL), extracted with DCM (3*5 mL). The combined organic phase was washed brine, dried over anhydrous sodium sulfate, concentrated under vacuum. The residue was purified with preparative-TLC (DCM/MeOH=20/1) to afford 2-amino-7-fluoro-4-[2-[[(2R,8S)-2-fluoro-1,2,3,5,6,7-hexahydropyrrolizin-8-yl]methoxy]-4-[(3R)-1-prop-2-enoylpyrrolidin-3-yl]oxy-6-(trifluoromethyl)pyrido[3,2-d]pyrimidin-7-yl]benzothiophene-3-carbonitrile (2.0 mg, 18% yield) as solid. LCMS ESI (+) m/z 686.1 (M+H). ¹HNMR (400 MHz, CD₃OD) δ 8.51 (s, 1H), 8.10 (s, 1H), 7.24 (s, 1H), 7.03 (t, 1H), 6.61-6.75 (m, 2H), 6.34 (s, 0.5H), 6.30 (s, 0.5H), 6.07 (s, 0.5H), 6.05 (s, 0.5H), 5.77-5.82 (m, 1H), 5.50-5.51 (m, 1H), 4.51-4.63 (m, 2H), 4.16 (s, 1H), 3.98-4.04 (m, 2H), 3.50-3.63 (m, 4H), 2.05-2.58 (m, 9H).

### Synthetic Example 10: Synthesis of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 62), 4-((S)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 63) and 4-((R)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 64)

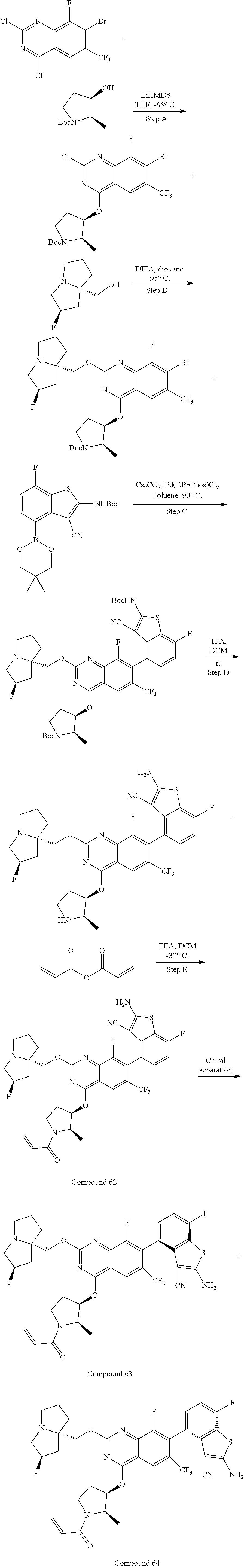

Step A: Preparation of tert-butyl (2R,3R)-3-((7-bromo-2-chloro-8-fluoro-6-(trifluoromethyl) quinazolin-4-yl)oxy)-2-methylpyrrolidine-1-carboxylate: To a solution of tert-butyl (2R,3R)-3-hydroxy-2-methyl-pyrrolidine-1-carboxylate (143 mg, 0.71 mmol) in THF (3 mL) was added LiHMDS (0.5 mL, 0.61 mmol) at −65° C. dropwise under argon. The mixture was kept at this temperature for 1 hour and then 7-bromo-2,4-dichloro-8-fluoro-6-(trifluoromethyl) quinazoline (200 mg, 0.55 mmol) in THF (3 mL) was added dropwise. Then the mixture was warmed to ambient temperature and kept at this temperature for 2 hours. The mixture was quenched with water and extracted with EA. The organics were washed with saturated brine solution. The organics were then separated and dried (MgSO₄) before concentration to dryness. The crude was then purified by preparative-TLC (EA:PE=1:3) to give tert-butyl (2R,3R)-3-((7-bromo-2-chloro-8-fluoro-6-(trifluoromethyl) quinazolin-4-yl)oxy)-2-methylpyrrolidine-1-carboxylate (115 mg, 39% yield) as solid.

Step B: Preparation of tert-butyl (2R,3R)-3-((7-bromo-8-fluoro-2-(((2R,7aS)-2-fluorotetrahydro-1H-pyrrolizin-7a (5H)-yl)methoxy)-6-(trifluoromethyl) quinazolin-4-yl)oxy)-2-methylpyrrolidine-1-carboxylate: To a stirred solution of tert-butyl (2R,3R)-3-((7-bromo-2-chloro-8-fluoro-6-(trifluoromethyl) quinazolin-4-yl)oxy)-2-methylpyrrolidine-1-carboxylate (115 mg, 0.21 mmol) in 1,4-dioxane (4 mL) were added [(2R,8S)-2-fluoro-1,2,3,5,6,7-hexahydropyrrolizin-8-yl]methanol (100 mg, 0.63 mmol) and DIEA (0.47 mL, 2.66 mmol). The mixture was heated to 95° C. and kept at this temperature for 8 hours under argon. The mixture was cooled to room temperature and concentrated to dryness. The residue was purified by preparative-TLC (EA:PE=1:1) to give tert-butyl (2R,3R)-3-((7-bromo-8-fluoro-2-(((2R,7aS)-2-fluorotetrahydro-1H-pyrrolizin-7a (5H)-yl)methoxy)-6-(trifluoromethyl) quinazolin-4-yl)oxy)-2-methylpyrrolidine-1-carboxylate (100 mg, 72.8% yield) as solid.

Step C: Preparation of tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a (5H)-yl) methoxy)-6-(trifluoromethyl) quinazolin-4-yl)oxy)-2-methylpyrrolidine-1-carboxylate: A mixture of tert-butyl (2R,3R)-3-[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (100 mg, 0.15 mmol), tert-butyl N-[3-cyano-4-(5,5-dimethyl-1,3,2-dioxaborinan-2-yl)-7-fluoro-benzothiophen-2-yl] carbamate (186 mg, 0.46 mmol), Cs₂CO₃ (100 mg, 0.31 mmol) and DPEPhosPdCl₂ (44 mg, 0.062 mmol) in dioxane (1.5 mL) under Ar was stirred at 90° C. for 2 hours. After cooled to ambient temperature, the reaction was purified by preparative-TLC (EtOAc:PE=1:1) to give tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (63 mg, 47%). LCMS ESI (+) m/z 863.4 (M+H).

Step D: Preparation of 2-amino-7-fluoro-4-(8-fluoro-2-(((2R,7aS)-2-fluorotetrahydro-1H-pyrrolizin-7a (5H)-yl)methoxy)-4-(((2R,3R)-2-methylpyrrolidin-3-yl)oxy)-6-(trifluoromethyl) quinazolin-7-yl)benzo[b]thiophene-3-carbonitrile: To a solution of tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl) quinazolin-4-yl]oxy-2-methyl-pyrrolidine-1-carboxylate (31 mg, 0.036 mmol) in DCM (1 mL) was added TFA (0.50 mL) at 25° C., the mixture was stirred at 25° C. for 1.5 hours. The reaction mixture was dissolved in EtOAc and adjust pH to ˜8 with saturated NaHCO₃ solution, the organics were concentrated to dryness and the crude product was purified by RP preparative-HPLC to give 2-amino-7-fluoro-4-[8-fluoro-2-[[(2R,8S)-2-fluoro-1,2,3,5,6,7-hexahydropyrrolizin-8-yl]methoxy]-4-[(2R,3R)-2-methylpyrrolidin-3-yl]oxy-6-(trifluoromethyl) quinazolin-7-yl]benzothiophene-3-carbonitrile (10 mg, 38%) as solid. LCMS ESI (+) m/z 663.3 (M+H).

Step E: Preparation of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 62), 4-((S)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 63) and 4-((R)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 64): To a solution of 2-amino-7-fluoro-4-[8-fluoro-2-[[(2R,8S)-2-fluoro-1,2,3,5,6,7-hexahydropyrrolizin-8-yl]methoxy]-4-[methyl-[(3R)-pyrrolidin-3-yl]amino]pyrido[4,3-d]pyrimidin-7-yl]benzothiophene-3-carbonitrile (10 mg, 0.017 mmol) in DCM (1 mL) was added Et3N (5.1 mg, 0.050 mmol). The mixture was cooled to −30° C., and prop-2-enoyl prop-2-enoate (2.1 mg, 0.017 mmol) was added at −30° C. under Ar, the mixture was stirred for 0.5 hr at −30° C. The reaction was dissolved in EtOAc) and washed with water, brine solution. The organics were separated and dried (MgSO₄) before concentration to dryness, the crude was purified by RP preparative-HPLC to give 2-amino-7-fluoro-4-[8-fluoro-2-[[(2R,8S)-2-fluoro-1,2,3,5,6,7-hexahydropyrrolizin-8-yl]methoxy]-4-[(2R,3R)-2-methyl-1-prop-2-enoyl-pyrrolidin-3-yl]oxy-6-(trifluoromethyl) quinazolin-7-yl]benzothiophene-3-carbonitrile (3.4 mg, 12% yield). ESI (+) m/z 717.3 (M+H). ¹HNMR (400 MHZ, CD₃OD) δ 8.31 (s, 1H), 7.21-7.29 (m, 1H), 7.04 (t, J=9.16 Hz, 1H), 6.67-6.77 (m, 1H), 6.27-6.40 (m, 1H), 5.75-5.94 (m, 2H), 5.34 (d, J=54.20 Hz, 1H), 4.70-4.83 (m, 1H), 4.28-4.51 (m, 2H), 3.62-3.98 (m, 2H), 3.35-2.40 (m, 1H), 3.22-3.29 (m, 2H), 3.07-3.12 (m, 1H), 2.16-2.64 (m, 5H), 1.85-2.01 (m, 3H), 1.29-1.35 (m, 3H).

The diastereomers of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 62) (464.6 mg) were separated with chiral chromatography condition [ChiralPak IH 3 cm×25 cm, 5 μm, CO₂:MeOH (0.2% 2 mM NH₃-MeOH)]=55:45, 80 mL/min]. The first compound off the column was identified as one atropisomer, 4-((S)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 63) (150.6 mg, 100% de). LCMS ESI (+) m/z 717.3 (M+H). ¹HNMR (400 MHZ, CD₃OD) δ 8.28 (s, 1H), 7.20-7.26 (m, 1H), 6.98-7.06 (m, 1H), 6.62-6.73 (m, 1H), 6.27-6.40 (m, 1H), 5.75-5.90 (m, 2H), 5.30 (d, J=54.8 Hz, 1H), 4.70-4.83 (m, 1H), 4.29-4.41 (m, 2H), 3.66-3.95 (m, 2H), 2.99-3.25 (m, 4H), 1.85-2.64 (m, 8H), 1.27-1.35 (m, 3H).

The second compound off the column was identified as the other atropisomer, 4-((R)-4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)-6-(trifluoromethyl) quinazolin-7-yl)-2-amino-7-fluorobenzo[b]thiophene-3-carbonitrile (Compound 64) (177.2 mg, 99.68% de). LCMS ESI (+) m/z 717.4 (M+H). ¹HNMR (400 MHz, CD₃OD) δ 8.28 (s, 1H), 7.20-7.26 (m, 1H), 6.98-7.06 (m, 1H), 6.62-6.73 (m, 1H), 6.27-6.40 (m, 1H), 5.75-5.90 (m, 2H), 5.30 (d, J=54.8 Hz, 1H), 4.70-4.83 (m, 1H), 4.29-4.41 (m, 2H), 3.66-3.95 (m, 2H), 2.99-3.25 (m, 4H), 1.85-2.64 (m, 8H), 1.27-1.35 (m, 3H).

### Synthetic Example 11: Synthesis of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl) methoxy)pyrido[4,3-d]pyrimidin-7-yl)-2-amino-7-fluorobenzo[b]thiophene-3-carbonitrile (Compound 73)

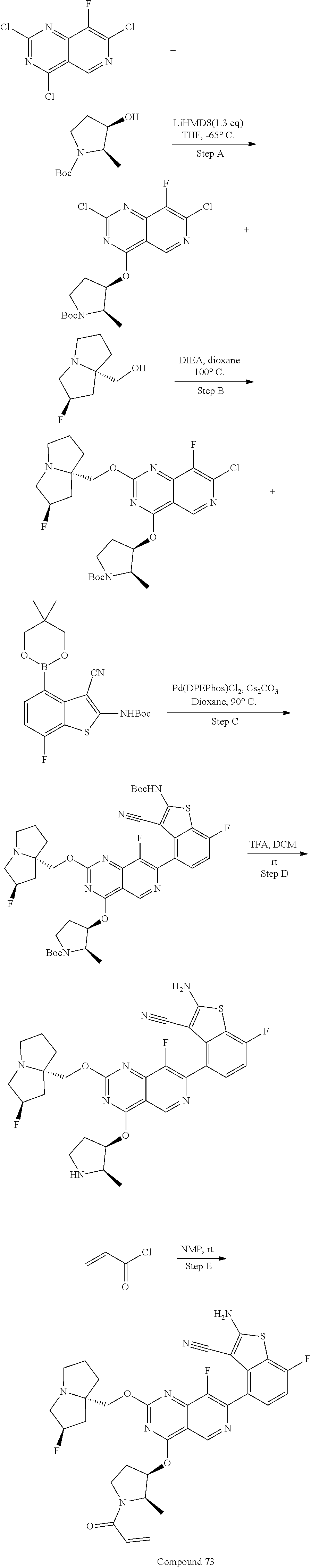

Step A: Preparation of tert-butyl (2R,3R)-3-((2,7-dichloro-8-fluoropyrido[4,3-d]pyrimidin-4-yl)oxy)-2-methylpyrrolidine-1-carboxylate: To a solution of tert-butyl (2R,3R)-3-hydroxy-2-methyl-pyrrolidine-1-carboxylate (106 mg, 0.53 mmol) in THF (3 mL) at −65° C. was added LiHMDS (0.45 mL, 0.45 mmol) dropwise under argon. The mixture was kept at this temperature for 1 hour and then 2,4,7-trichloro-8-fluoropyrido[4,3-d]pyrimidine (102 mg, 0.41 mmol) in THF (3 mL) was added dropwise. Then the mixture was warmed to ambient temperature and kept at this temperature for 2 hours. The mixture was quenched with water and extracted with EA. The organics were washed with brine solution. The organics were then separated and dried (MgSO₄) before concentration to dryness. The crude was then purified by preparative-TLC (EA:PE=1:3) to give tert-butyl (2R,3R)-3-((2,7-dichloro-8-fluoropyrido[4,3-d]pyrimidin-4-yl)oxy)-2-methylpyrrolidine-1-carboxylate (93 mg, 54% yield) as solid.

Step B: Preparation of tert-butyl (2R,3R)-3-((7-chloro-8-fluoro-2-(((2R,7aS)-2-fluorotetrahydro-1H-pyrrolizin-7a (5H)-yl)methoxy)pyrido[4,3-d]pyrimidin-4-yl)oxy)-2-methylpyrrolidine-1-carboxylate: To a stirred solution of tert-butyl (2R,3R)-3-((2,7-dichloro-8-fluoropyrido[4,3-d]pyrimidin-4-yl)oxy)-2-methylpyrrolidine-1-carboxylate (93 mg, 0.223 mmol) in 1,4-Dioxane was added [(2R,8S)-2-fluoro-1,2,3,5,6,7-hexahydropyrrolizin-8-yl]methanol (106 mg, 0.67 mmol) and DIEA (0.47 mL, 2.66 mmol). The mixture was heated to 95° C. and kept at this temperature for 8 hours under argon. The mixture was cooled to room temperature and concentrated to dryness. The residue was purified by preparative-TLC (EA:PE=1:1) to give tert-butyl (2R,3R)-3-((7-chloro-8-fluoro-2-(((2R,7aS)-2-fluorotetrahydro-1H-pyrrolizin-7a (5H)-yl)methoxy)pyrido[4,3-d]pyrimidin-4-yl)oxy)-2-methylpyrrolidine-1-carboxylate (98 mg, 81% yield) as solid.

Step C: Preparation tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a (5H)-yl) methoxy)pyrido[4,3-d]pyrimidin-4-yl)oxy)-2-methylpyrrolidine-1-carboxylate: To a stirred solution of tert-butyl (2R,3R)-3-((7-chloro-8-fluoro-2-(((2R,7aS)-2-fluorotetrahydro-1H-pyrrolizin-7a (5H)-yl) methoxy)pyrido[4,3-d]pyrimidin-4-yl)oxy)-2-methylpyrrolidine-1-carboxylate (98 mg, 0.18 mmol) in 1,4-Dioxane (4 mL) were added tert-butyl N-[3-cyano-4-(5,5-dimethyl-1,3,2-dioxaborinan-2-yl)-7-fluoro-benzothiophen-2-yl]carbamate (219 mg, 0.54 mmol), Pd(DPEPhos)Cl₂ (13 mg, 0.018 mmol) and Cs₂CO₃ (118 mg, 0.36 mmol). The mixture was heated to 95° C. and stirred at this temperature for 8 hours under argon. The mixture was cooled to room temperature and diluted with EA, then filtered. The filtrate was concentrated and the residue was purified by preparative-TLC (MeOH:DCM=1:20) to give tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a (5H)-yl)methoxy)pyrido[4,3-d]pyrimidin-4-yl)oxy)-2-methylpyrrolidine-1-carboxylate (75 mg, 52% yield) as solid. LCMS ESI (+) m/z 796.3 (M+H).

Step D: Preparation of 2-amino-7-fluoro-4-(8-fluoro-2-(((2R,7aS)-2-fluorotetrahydro-1H-pyrrolizin-7a (5H)-yl)methoxy)-4-(((2R,3R)-2-methylpyrrolidin-3-yl)oxy)pyrido[4,3-d]pyrimidin-7-yl)benzo[b]thiophene-3-carbonitrile: To a stirred solution of tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a (5H)-yl)methoxy)pyrido[4,3-d]pyrimidin-4-yl)oxy)-2-methylpyrrolidine-1-carboxylate (75 mg, 0.094 mmol) in DCM (3 mL) was added TFA (1.0 mL, 13.0 mmol). The mixture was stirred at temperature for 4 hours. The mixture was concentrated, and the residue was dissolved in EtOAc (20 mL) and the organics washed with 10 mL NaHCO₃ solution then 10 mL saturated brine solution. The organics were then separated and dried (MgSO₄) before concentration to dryness. The crude was then used in the next step without further purification. LCMS ESI (+) m/z 596.3 (M+H).

Step E: Preparation of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)pyrido[4,3-d]pyrimidin-7-yl)-2-amino-7-fluorobenzo[b]thiophene-3-carbonitrile: To a stirred solution of 2-amino-7-fluoro-4-(8-fluoro-2-(((2R,7aS)-2-fluorotetrahydro-1H-pyrrolizin-7a (5H)-yl)methoxy)-4-(((2R,3R)-2-methylpyrrolidin-3-yl)oxy)pyrido[4,3-d]pyrimidin-7-yl)benzo[b]thiophene-3-carbonitrile (30 mg, 0.050 mmol) in NMP (2 mL) was added acryloyl chloride (4.98 mg, 0.055 mmol) dropwise at ambient temperature. The mixture was stirred at ambient for 1 hour under argon. The mixture was quenched with water (20 mL) and extracted with EA. The organics were washed with brine solution. The organics were then separated and dried (MgSO₄) before concentration to dryness. The crude was then purified by RP preparative-HPLC to give 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl)methoxy)pyrido[4,3-d]pyrimidin-7-yl)-2-amino-7-fluorobenzo[b]thiophene-3-carbonitrile (5.14 mg, 14% yield) as solid. LCMS ESI (+) m/z 650.3 (M+H). ¹H NMR (400 MHz, CD₃OD) δ 9.22 (s, 1H), 7.40-7.43 (m, 1H), 7.06 (t, J=8.0 Hz, 1H), 6.62-6.69 (m, 1H), 6.28-6.33 (m, 1H), 5.77-5.90 (m, 2H), 5.30 (d, J=54.8 Hz, 1H), 4.44-4.70 (m, 3H), 3.38-3.81 (m, 5H), 3.13-3.14 (m, 1H), 2.08-2.52 (m, 8H), 1.33 (d, J=6.4 Hz, 3H).

### Synthetic Example 12: Synthesis of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl) methoxy)quinazolin-7-yl)-2-amino-7-fluorobenzo[b]thiophene-3-carbonitrile (Compound 76)

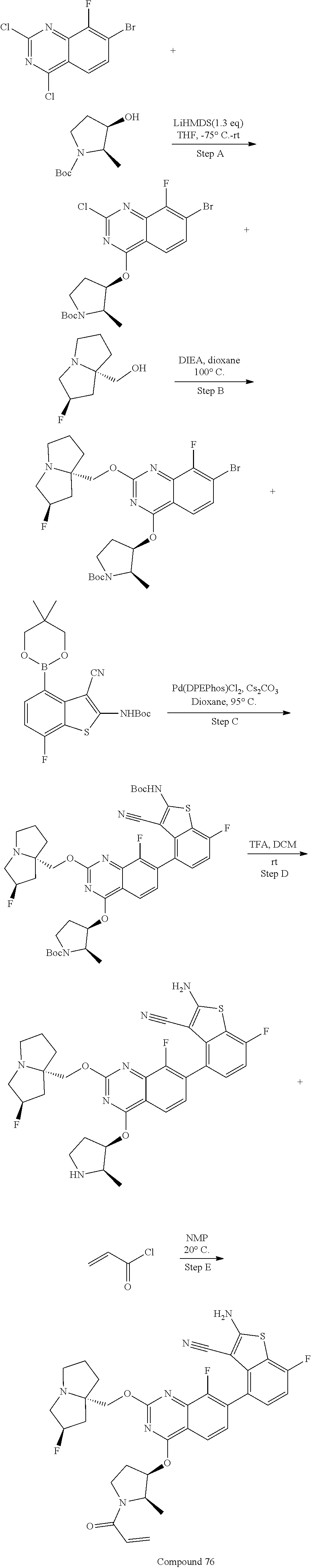

Step A: Preparation of tert-butyl (2R,3R)-3-((7-bromo-2-chloro-8-fluoroquinazolin-4-yl)oxy)-2-methylpyrrolidine-1-carboxylate: To a solution of tert-butyl (2R,3R)-3-hydroxy-2-methyl-pyrrolidine-1-carboxylate (106 mg, 0.53 mmol) in THF (3 mL) at −78° C. was added LiHMDS (0.45 mL, 0.44 mmol) dropwise under argon. The mixture was stirred at this temperature for 1 hour and then 7-bromo-2,4-dichloro-8-fluoro-quinazoline (120 mg, 0.41 mmol) in THF (3 mL) was added dropwise. Then the mixture was warmed to ambient temperature and kept at this temperature for 2 hours. The mixture was quenched with water and extracted with EA. The organics were washed with brine solution. The organics were then separated and dried (MgSO₄) before concentration to dryness. The crude was then purified by preparative-TLC (EA:PE=1:3) to give tert-butyl (2R,3R)-3-((7-bromo-2-chloro-8-fluoroquinazolin-4-yl)oxy)-2-methylpyrrolidine-1-carboxylate (153 mg, 81% yield) as solid.

Step B: Preparation of tert-butyl (2R,3R)-3-((7-bromo-8-fluoro-2-(((2R,7aS)-2-fluorotetrahydro-1H-pyrrolizin-7a (5H)-yl) methoxy)quinazolin-4-yl)oxy)-2-methylpyrrolidine-1-carboxylate: To a stirred solution of tert-butyl (2R,3R)-3-(7-bromo-2-chloro-8-fluoro-quinazolin-4-yl)oxy-2-methyl-pyrrolidine-1-carboxylate (153 mg, 0.332 mmol) in 1,4-dioxane (4 mL) were added [(2R,8S)-2-fluoro-1,2,3,5,6,7-hexahydropyrrolizin-8-yl]methanol (159 mg, 0.10 mmol) and DIEA (0.47 mL, 2.66 mmol). The mixture was heated to 95° C. and stirred at this temperature for 8 hours under argon. The mixture was cooled to room temperature and concentrated to dryness. The residue was purified by preparative-TLC (EA:PE=1:1) to give tert-butyl (2R,3R)-3-[7-bromo-8-fluoro-2-[[(2R,8S)-2-fluoro-1,2,3,5,6,7-hexahydropyrrolizin-8-yl]methoxy]quinazolin-4-yl]oxy-2-methyl-pyrrolidine-1-carboxylate (98 mg, 50% yield) as solid. LCMS ESI (+) m/z 583.2 (M+H).

Step C: Preparation of tert-butyl (2R,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a (5H)-yl) methoxy)quinazolin-4-yl)oxy)-2-methylpyrrolidine-1-carboxylate: To a stirred solution of tert-butyl (2R,3R)-3-[7-bromo-8-fluoro-2-[[(2R,8S)-2-fluoro-1,2,3,5,6,7-hexahydropyrrolizin-8-yl]methoxy]quinazolin-4-yl]oxy-2-methyl-pyrrolidine-1-carboxylate (98 mg, 0.17 mmol) in 1,4-dioxane (4 mL) were added tert-butyl N-[3-cyano-4-(5,5-dimethyl-1,3,2-dioxaborinan-2-yl)-7-fluoro-benzothiophen-2-yl]carbamate (204 mg, 0.50 mmol), Pd(DPEPhos)Cl₂ (12 mg, 0.017 mmol) and Cs₂CO₃ (109 mg, 0.34 mmol). The mixture was heated to 95° C. and stirred at this temperature for 8 hours under argon. The mixture was cooled to room temperature and diluted with EA, then filtered. The filtrate was concentrated and the residue was purified by preparative-TLC (MeOH:DCM=1:20) to give tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]quinazolin-4-yl]oxy-2-methyl-pyrrolidine-1-carboxylate (66 mg, 49% yield) as solid. LCMS ESI (+) m/z 795.3 (M+H).

Step D: Preparation of 2-amino-7-fluoro-4-(8-fluoro-2-(((2R,7aS)-2-fluorotetrahydro-1H-pyrrolizin-7a (5H)-yl)methoxy)-4-(((2R,3R)-2-methylpyrrolidin-3-yl)oxy) quinazolin-7-yl)benzo[b]thiophene-3-carbonitrile: To a stirred solution of tert-butyl (2R,3R)-3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]quinazolin-4-yl]oxy-2-methyl-pyrrolidine-1-carboxylate (33 mg, 0.042 mmol) in DCM (3 mL) was added TFA (1.0 mL, 13.0 mmol) at ambient temperature. The mixture was kept at ambient temperature for 4 hours. The mixture was concentrated, and the residue was dissolved in EtOAc (20 mL) and the organics washed with 10 mL saturated NaHCO₃ solution then 10 mL saturated brine solution. The organics were then separated and dried (MgSO₄) before concentration to dryness. The crude was then used in the next step without further purification. LCMS ESI (+) m/z 595.3 (M+H).

Step E: Preparation of 4-(4-(((2R,3R)-1-acryloyl-2-methylpyrrolidin-3-yl)oxy)-8-fluoro-2-(((2R,7aS)-2-fluorotetrahydro-1H-pyrrolizin-7a (5H)-yl) methoxy)quinazolin-7-yl)-2-amino-7-fluorobenzo[b]thiophene-3-carbonitrile: To a stirred solution of 2-amino-7-fluoro-4-[8-fluoro-2-[[(2R,8S)-2-fluoro-1,2,3,5,6,7-hexahydropyrrolizin-8-yl]methoxy]-4-[(2R,3R)-2-methylpyrrolidin-3-yl]oxy-quinazolin-7-yl]benzothiophene-3-carbonitrile (15 mg, 0.025 mmol) in NMP (2 mL) was added acryloyl chloride (0.0023 mL, 0.028 mmol) dropwise at ambient temperature. The mixture was stirred at ambient temperature for 1 hour under argon. The mixture was quenched with water (20 mL) and extracted with EA (15 mL×2). The organics were washed with 10 mL saturated brine solution. The organics were then separated and dried (MgSO₄) before concentration to dryness. The crude was then purified by RP preparative-HPLC to give 2-amino-7-fluoro-4-[8-fluoro-2-[[(2R,8S)-2-fluoro-1,2,3,5,6,7-hexahydropyrrolizin-8-yl]methoxy]-4-[(2R,3R)-2-methyl-1-prop-2-enoyl-pyrrolidin-3-yl]oxy-quinazolin-7-yl]benzothiophene-3-carbonitrile (5.4 mg, 32% yield) as solid. LCMS ESI (+) m/z 649.3 (M+H). ¹H NMR (400 MHZ, CD₃OD) δ 7.99 (d, J=8.36 Hz, 1H), 7.40 (t, J=6.96 Hz, 1H), 7.26 (t, J=6.92 Hz, 1H), 7.03 (t, J=8.88 Hz, 1H), 6.62-6.72 (m, 1H), 6.33 (t, J=17.76 Hz, 1H), 5.77-5.88 (m, 2H), 5.38 (d, J=53.88 Hz, 1H), 4.68-4.79 (m, 1H), 4.34-4.52 (m, 2H), 3.63-3.94 (m, 2H), 3.39-3.48 (m, 3H), 3.13 (s, 1H), 2.22-2.59 (m, 5H), 1.95-2.10 (m, 3H), 1.30-1.32 (m, 3H).

### Synthetic Example 13: Synthesis of 4-(4-(((2R,3R)-1-acryloyl-2-methylpyrrolidin-3-yl)oxy)-8-chloro-2-(((2R,7aS)-2-fluorotetrahydro-1H-pyrrolizin-7a (5H)-yl)methoxy)pyrido[4,3-d]pyrimidin-7-yl)-2-amino-7-fluorobenzo[b]thiophene-3-carbonitrile (Compound 106)

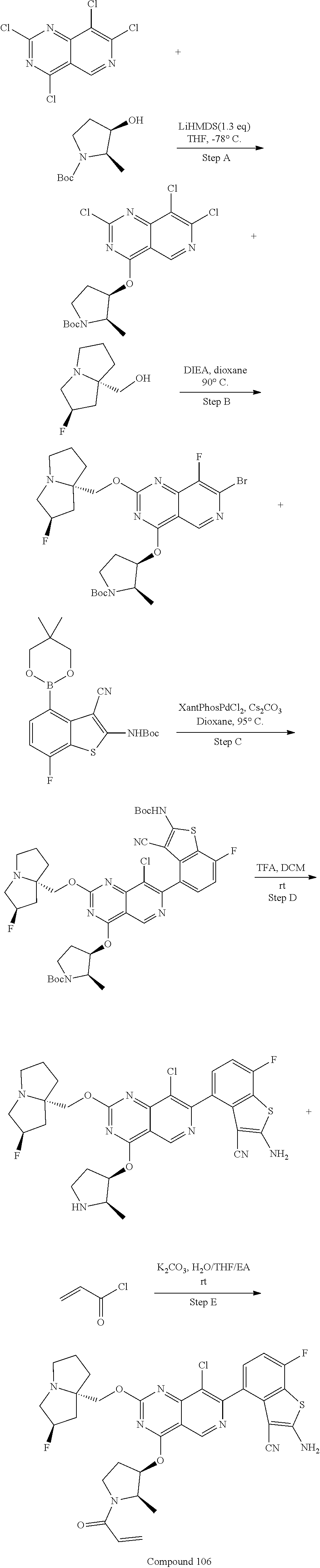

Step A: Preparation of tert-butyl (2R,3R)-2-methyl-3-((2,7,8-trichloropyrido[4,3-d]pyrimidin-4-yl)oxy)pyrrolidine-1-carboxylate: To a solution of 2,4,7,8-tetrachloropyrido[4,3-d]pyrimidine (150 mg, 0.56 mmol) in THF (1 mL) at −78° C. was added LiHMDS (76 mg, 0.56 mmol) under argon and the mixture was stirred at this temperature for 1 hour, then a solution of tert-butyl (2R,3R)-3-hydroxy-2-methyl-pyrrolidine-1-carboxylate (123 mg, 0.614 mmol) in THF (1 mL) was added dropwise. Then the mixture was warmed to room temperature and stirred at this temperature for another 1 hour. The mixture was quenched with water (10 mL) and extracted with EA. The organics were washed with brine and dried over Na₂SO₄ before concentration to dryness. The residue was purified by preparative-TLC to obtain tert-butyl (2R,3R)-2-methyl-3-(2,7,8-trichloropyrido[4,3-d]pyrimidine-4-yl)oxy-pyrrolidine-1-carboxylate (182 mg, 75% yield) as solid.

Step B: Preparation of tert-butyl (2R,3R)-3-((7-bromo-8-fluoro-2-(((2R,7aS)-2-fluorotetrahydro-1H-pyrrolizin-7a (5H)-yl)methoxy)pyrido[4,3-d]pyrimidin-4-yl)oxy)-2-methylpyrrolidine-1-carboxylate: To a stirred solution of [(2R,8S)-2-fluoro-1,2,3,5,6,7-hexahydropyrrolizin-8-yl]methanol (95 mg, 0.60 mmol) in 1,4-dioxane (1.3 mL) were added tert-butyl (2R,3R)-2-methyl-3-(2,7,8-trichloropyrido[4,3-d]pyrimidin-4-yl)oxy-pyrrolidine-1-carboxylate (130 mg, 0.30 mmol) and DIEA (0.16 mL, 0.90 mmol). The mixture was heated to 90° C. and stirred at this temperature for 4 hours under argon. The mixture was concentrated to dryness and the residue was purified by preparative-TLC (EA:PE=1:1) to give tert-butyl (2R,3R)-3-[7,8-dichloro-2-[[(2R,8S)-2-fluoro-1,2,3,5,6,7-hexahydropyrrolizin-8-yl]methoxy]pyrido[4,3-d]pyrimidin-4-yl]oxy-2-methyl-pyrrolidine-1-carboxylate (150 mg, 89% yield) as solid. LCMS ESI (+) m/z 556.0 (M+H).

Step C: Preparation of tert-butyl (2R,3R)-3-((7-(2-((tert-butoxycarbonyl)amino)-3-cyano-7-fluorobenzo[b]thiophen-4-yl)-8-chloro-2-(((2R,7aS)-2-fluorotetrahydro-1H-pyrrolizin-7a (5H)-yl) methoxy)pyrido[4,3-d]pyrimidin-4-yl)oxy)-2-methylpyrrolidine-1-carboxylate: To a solution of tert-butyl rac-(2R,3R)-3-[7,8-dichloro-2-[[rac-(2R,8S)-2-fluoro-1,2,3,5,6,7-hexahydropyrrolizin-8-yl]methoxy]pyrido[4,3-d]pyrimidin-4-yl]oxy-2-methyl-pyrrolidine-1-carboxylate (80 mg, 0.14 mmol) in 1,4-dioxane (2.4 mL) were added tert-butyl N-[3-cyano-4-(5,5-dimethyl-1,3,2-dioxaborinan-2-yl)-7-fluoro-benzothiophen-2-yl]carbamate (174 mg, 0.43 mmol), cesium carbonate (117 mg, 0.36 mmol) and XantPhosPdCl₂ (22 mg, 0.029 mmol). The mixture was heated to 95° C. and stirred at this temperature for 2 hrs. After cooled to ambient temperature, the reaction was diluted with EA (20 mL) and the organics washed with water, then brine solution. The organics were then separated and dried (MgSO₄) before concentration to dryness. The crude was then purified by preparative-TLC (PE:EA=1:1) to afford tert-butyl rac-(2R,3R)-3-[7-[2-(tert-butoxycarbonylamino)-3-cyano-7-fluoro-benzothiophen-4-yl]-8-chloro-2-[[rac-(2R,8S)-2-fluoro-1,2,3,5,6,7-hexahydropyrrolizin-8-yl]methoxy]pyrido[4,3-d]pyrimidin-4-yl]oxy-2-methyl-pyrrolidine-1-carboxylate (56 mg, 35% yield) as solid. LCMS ESI (+) m/z 812.0 (M+H).

Step D: Preparation of 2-amino-4-(8-chloro-2-(((2R,7aS)-2-fluorotetrahydro-1H-pyrrolizin-7a (5H)-yl)methoxy)-4-(((2R,3R)-2-methylpyrrolidin-3-yl)oxy)pyrido[4,3-d]pyrimidin-7-yl)-7-fluorobenzo[b]thiophene-3-carbonitrile: To a stirred solution of tert-butyl (2R,3R)-3-[7-[2-(tert-butoxycarbonylamino)-3-cyano-7-fluoro-benzothiophen-4-yl]-8-chloro-2-[[(2R,8S)-2-fluoro-1,2,3,5,6,7-hexahydropyrrolizin-8-yl]methoxy]pyrido[4,3-d]pyrimidin-4-yl]oxy-2-methyl-pyrrolidine-1-carboxylate (56 mg, 0.069 mmol) in DCM (1 mL) was added trifluoroacetic acid (0.50 mL, 6.49 mmol) at ambient temperature. The mixture was stirred at ambient temperature for 1 hours and then concentrated to dryness under vacuum to give 2-amino-4-[8-chloro-2-[[(2R,8S)-2-fluoro-1,2,3,5,6,7-hexahydropyrrolizin-8-yl]methoxy]-4-[(2R,3R)-2-methylpyrrolidin-3-yl]oxy-pyrido[4,3-d]pyrimidin-7-yl]-7-fluoro-benzothiophene-3-carbonitrile (42 mg, 99% yield) which was used in the next step without further purification. LCMS ESI (+) m/z 612.3 (M+H).

Step E: Preparation of 4-(4-(((2R,3R)-1-acryloyl-2-methylpyrrolidin-3-yl)oxy)-8-chloro-2-(((2R,7aS)-2-fluorotetrahydro-1H-pyrrolizin-7a (5H)-yl)methoxy)pyrido[4,3-d]pyrimidin-7-yl)-2-amino-7-fluorobenzo[b]thiophene-3-carbonitrile: To the mixture of K₂CO₃ (28 mg, 0.20 mmol) in water (1 mL) and ethyl acetate (1 mL) was added 2-amino-4-[8-chloro-2-[[(2R,8S)-2-fluoro-1,2,3,5,6,7-hexahydropyrrolizin-8-yl]methoxy]-4-[(2R,3R)-2-methylpyrrolidin-3-yl]oxy-pyrido[4,3-d]pyrimidin-7-yl]-7-fluoro-benzothiophene-3-carbonitrile (25 mg, 0.041 mmol) in THF (1 mL) dropwise, followed by a solution of acryloyl chloride (0.0033 mL, 0.041 mmol) in DCM (0.20 mL) at 0° C. 30 minutes later. The reaction mixture was then stirred at 0° C. for 30 minutes. The reaction was extracted with EtOAc. The organics were then separated and dried (Na₂SO₄) before concentration to dryness. The residue was purified by preparative-TLC (DCM/MeOH=10/1) to give 2-amino-4-[8-chloro-2-[[rac-(2R,8S)-2-fluoro-1,2,3,5,6,7-hexahydropyrrolizin-8-yl]methoxy]-4-[(2R,3R)-2-methyl-1-prop-2-enoyl-pyrrolidin-3-yl]oxy-pyrido[4,3-d]pyrimidin-7-yl]-7-fluoro-benzothiophene-3-carbonitrile (12 mg, 44% yield) as solid. LCMS ESI (+) m/z 666.2 (M+H). ¹H NMR (400 MHZ, CD₃OD) δ 9.28 (s, 1H), 7.32 (dd, J=8.3, 5.1 Hz, 1H), 7.00-7.09 (m, 1H), 6.61-6.73 (m, 1H), 6.28-6.39 (m, 1H), 5.86-5.96 (m, 1H), 5.74-5.83 (m, 1H), 5.32 (d, J=53.7 Hz, 1H), 4.35-4.80 (m, 4H), 3.65-3.98 (m, 2H), 3.20-3.29 (m, 2H), 2.99-3.10 (m, 1H), 2.16-2.61 (m, 5H), 1.87-2.10 (m, 3H), 1.27-1.38 (m, 3H).

A compound of present disclosure, such as a compound of a formula included in Table 2 or Table 3, may be synthesized according to one of the general routes outlined in Synthetic Examples 1-13 or by various other methods generally known in the art.

Table 2 include selected compounds of the present disclosure.

TABLE 2
Selected compounds of the present disclosure.

| Compound | | |
|---|---|---|
| No. | Structure | Analytical data |
| 1 | 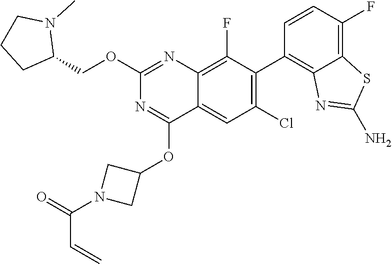 [SMILES: C=CC(=O)N1CC(Oc2nc(OC[C@@H]3CCCN3C)nc3c(F)c(-c4ccc(F)c5sc(N)nc45)c(Cl)cc23)C1] | LCMS m/z [M + 1]: 587.2 ¹HNMR (400 MHZ, CD₃OD) δ 8.15 (s, 1H), 7.22 (t, d = 8.8 Hz, 1H), 7.00 (t, J = 8.8 Hz, 1H), 6.25-6.45 (m, 2H), 5.71-5.81 (m, 2H), 4.54-4.74 (m, 5H), 4.25-4.32 (m, 1H), 3.46-3.48 (m, 2H), 2.80 (s, 3H), 2.20-2.30 (m, 1H), 1.90-2.05 (m, 4H). |
| 1a | 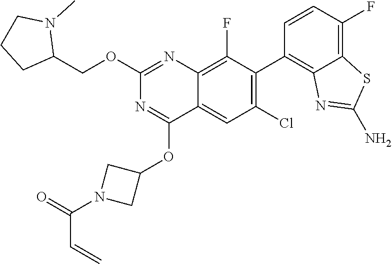 [SMILES: C=CC(=O)N1CC(Oc2nc(OCC3CCCN3C)nc3c(F)c(-c4ccc(F)c5sc(N)nc45)c(Cl)cc23)C1] | |
| 2 | 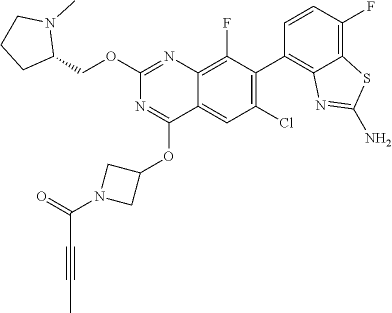 [SMILES: CC#CC(=O)N1CC(Oc2nc(OC[C@@H]3CCCN3C)nc3c(F)c(-c4ccc(F)c5sc(N)nc45)c(Cl)cc23)C1] | LCMS m/z [M + 1]: 599.2 ¹HNMR (400 MHz, CD₃OD) δ 8.14 (s, 1H), 7.19- 7.25 (m, 1H), 7.00 (t, J = 8.7 Hz, 1H), 5.68-5.75 (m, 1H), 4.67-4.80 (m, 2H), 4.52-4.64 (m, 3H), 4.45- 4.51 (m, 1H), 4.21-4.27 (m, 1H), 3.35-3.44 (m, 2H), 2.80 (s, 3H), 2.22-2.32 (m, 1H), 2.04 (s, 3H), 1.90- 2.01 (m, 3H). |
| 2a | 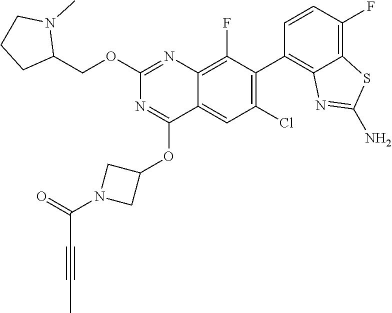 [SMILES: CC#CC(=O)N1CC(Oc2nc(OCC3CCCN3C)nc3c(F)c(-c4ccc(F)c5sc(N)nc45)c(Cl)cc23)C1] | |
| 3 | 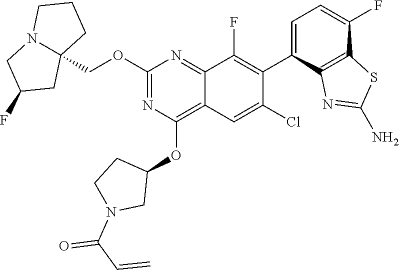 | LCMS m/z [M + 1]: 645.1 ¹HNMR (400 MHZ, CD₃OD) δ 8.03- 8.09 (m, 1H), 7.20-7.27 (m, 1H), 7.02 (t, J = 8.8 Hz, 1H), 6.56-6.75 (m, 1H), 6.28- 6.36 (m, 1H), 5.93-6.07 (m, 1H), 5.74-5.84 (m, 1H), 5.58 (d, J = 51.6 Hz, 1H), 4.70-4.78 (m, 2H), 4.04- 4.17 (m, 1H), 3.79-4.00 (m, 6H), 3.45-3.55 (m, 1H), 2.60-2.79 (m, 2H), 2.42-2.58 (m, 3H), 2.32- 2.40 (m, 2H), 2.14-2.23 (m, 1H). |
| 4 | 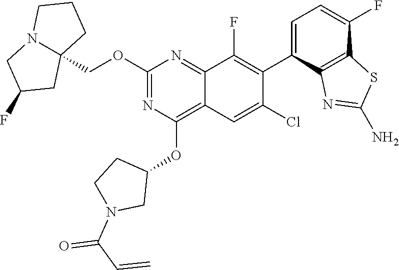 | LCMS m/z [M + 1]: 645.1 ¹HNMR (400 MHZ, CD₃OD) δ 8.05-8.06 (m, 1H), 7.21-7.24 (m, 1H), 7.01 (t, J = 8.8 Hz, 1H), 6.58- 6.74 (m, 1H), 6.30-6.34 (m, 1H), 5.81-5.95 (m, 1H), 5.65-5.78 (m, 1H), 5.58 (d, J = 51.6 Hz, 1H), 4.68- 4.86 (m, 2H), 4.09-4.15 (m, 1H), 3.86-4.06 (m, 6H), 3.49-3.53 (m, 1H), 2.36-2.77 (m, 8H). |
| 4a | 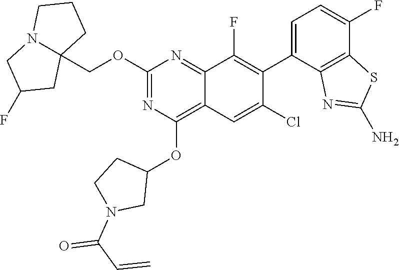 [SMILES: C=CC(=O)N1CCC(Oc2nc(OCC34CCCN3CC(F)C4)nc3c(F)c(-c4ccc(F)c5sc(N)nc45)c(Cl)cc23)C1] | |
| 5 | 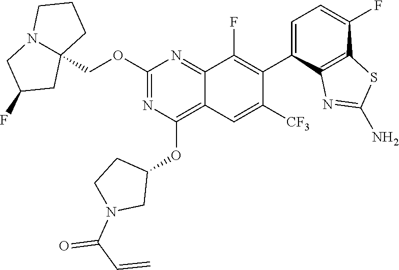 | LCMS m/z [M + 1]: 679.1 ¹HNMR (400 MHZ, CD₃OD): δ 8.31 (s, 1H), 7.19- 7.23 (m, 1H), 7.00 (t, J = 8.8 Hz, 1H), 6.58-6.74 (m, 1H), 6.30-6.35 (m, 1H), 5.99-6.06 (m, 1H), 5.76- 5.82 (m, 1H), 5.60 (d, J = 52.2 Hz, 1H), 4.71-4.82 (m, 1H), 3.73-4.17 (m, 7H), 3.46-3.53 (m, 1H), 2.17-2.78 (m, 8H). |
| 5a | 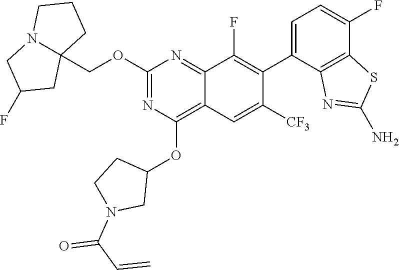 [SMILES: C=CC(=O)N1CCC(Oc2nc(OCC34CCCN3CC(F)C4)nc3c(F)c(-c4ccc(F)c5sc(N)nc45)c(C(F)(F)F)cc23)C1] | |
| 6 | 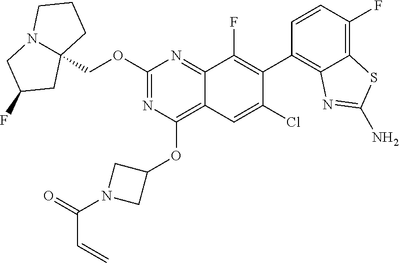 [SMILES: C=CC(=O)N1CC(Oc2nc(OC[C@@]34CCCN3C[C@H](F)C4)nc3c(F)c(-c4ccc(F)c5sc(N)nc45)c(Cl)cc23)C1] | LCMS m/z [M + 1]: 630.15 ¹HNMR (400 MHz, CD₃OD) δ 8.14 (s, 1H), 7.17- 7.23 (m, 1H), 7.02 (t, J = 8.8 Hz, 1H), 6.31-6.39 (m, 1H) , 6.23-6.30 (m, 1H), 5.71-5.82 (m, 1H), 5.64- 5.70 (m, 1H), 5.36-5.55 (m, 1H), 4.77-4.81 (m, 1H), 4.65 (s, 2H), 4.49-4.57 (m, 2H), 4.24-4.30 (m, 1H), 3.94-4.11 (m, 2H), 3.77-3.89 (m, 1H), 3.45-3.54 (m, 1H), 2.63-2.75 (m, 1H), 2.46-2.62 (m, 1H), 2.31- 2.43 (m, 3H), 2.01-2.11 (m, 1H). |
| 6a | 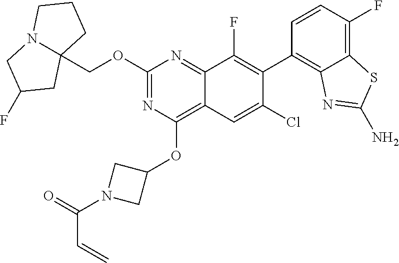 [SMILES: C=CC(=O)N1CC(Oc2nc(OCC34CCCN3CC(F)C4)nc3c(F)c(-c4ccc(F)c5sc(N)nc45)c(Cl)cc23)C1] | |
| 7 | 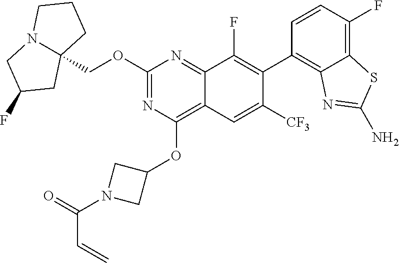 [SMILES: C=CC(=O)N1CC(Oc2nc(OC[C@@]34CCCN3C[C@H](F)C4)nc3c(F)c(-c4ccc(F)c5sc(N)nc45)c(C(F)(F)F)cc23)C1] | LCMS m/z [M + 1]: 665.2 ¹HNMR (400 MHZ, CD₃OD): δ 8.41 (s, 1H), 7.12- 7.21 (m, 1H), 6.99 (t, J = 8.8 Hz, 1H), 6.21-6.41 (m, 2H), 5.68-5.80 (m, 2H), 5.47 (dd, J = 52.4, 11.6 Hz, 1H), 4.68 (s, 2H), 4.49-4.55 (m, 2H), 4.26-4.31 (m, 1H), 3.98-3.13 (m, 3H), 3.79-3.90 (m, 1H), 3.51- 3.58 (m, 1H), 2.31-2.75 (m, 6H). |
| 7a | 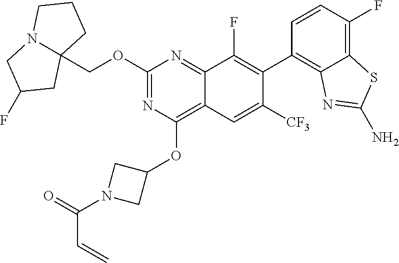 [SMILES: C=CC(=O)N1CC(Oc2nc(OCC34CCCN3CC(F)C4)nc3c(F)c(-c4ccc(F)c5sc(N)nc45)c(C(F)(F)F)cc23)C1] | |
| 8 | 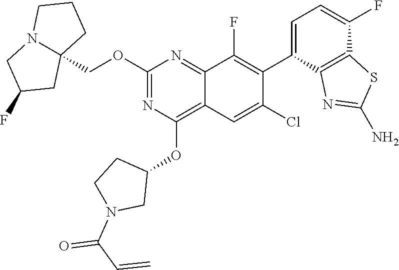 | LCMS m/z [M + 1]: 645.10 ¹HNMR (400 MHZ, CD₃OD) δ 8.05-8.06 (m, 1H), 7.21 (dd, J = 8.8, 5.2 Hz, 1H), 7.01 (t, J = 8.8 Hz, 1H), 6.6-6.8 (m, 1H), 6.30-6.34 (m, 1H), 5.81- 5.95 (m, 1H), 5.65-5.78 (m, 1H), 5.59 (d, J = 52.2 Hz, 1H), 4.68-4.78 (m, 2H), 4.09-4.15 (m, 1H), 3.86-4.03 (m, 6H), 3.43-3.56 (m, 1H), 2.33- 2.81 (m, 8H). |
| 9 | 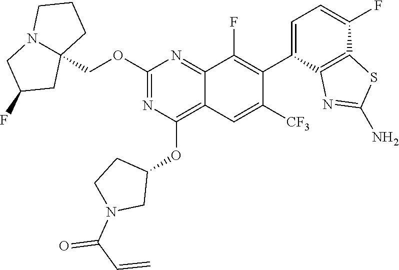 | LCMS m/z [M + 1]: 679.2 ¹HNMR (400 MHZ, CD₃OD) δ 8.31 (s, 1H), 7.19- 7.23 (m, 1H), 7.00 (t, J = 8.8 Hz, 1H), 6.58-6.74 (m, 1H), 6.30-6.35 (m, 1H), 6.03 (m, 1H), 5.76-5.82 (m, 1H), 5.60 (d, J = 52.2 Hz, 1H), 4.71-4.82 (m, 2H), 3.73-4.17 (m, 7H), 3.46-3.53 (m, 1H), 2.17-2.78 (m, 8H). |
| 10 | 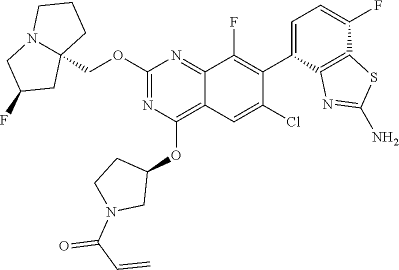 | LCMS m/z [M + 1]: 645.1 ¹HNMR (400 MHz, CD₃OD) δ 8.03-8.10 (m, 1H), 7.20-7.27 (m, 1H), 7.02 (t, J = 8.8 Hz, 1H), 6.56- 6.75 (m, 1H), 6.32 (d, J = 16.8 Hz, 1H), 5.93-6.07 (m, 1H), 5.78-5.85(m, 1H), 5.57 (d, J = 51.6 Hz, 1H), 4.70-4.79 (m, 2H), 4.05-4.17 (m, 1H), 3.88-4.04 (m, 6H), 3.45-3.55 (m, 1H), 2.61-2.82 (m, 2H), 2.42-2.58 (m, 3H), 2.31-2.40 (m, 2H), 2.14-2.25 (m, 1H). |
| 11 | 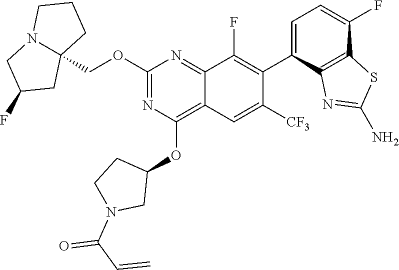 | LCMS m/z [M + 1]: 679.2 ¹H NMR (400 MHZ, CD₃OD) δ 8.27 (s, 1H), 7.12- 7.30 (m, 1H), 7.00 (t, J = 8.8 Hz, 1H), 6.58-6.81 (m, 1H), 6.29-6.41 (m, 1H), 5.88-6.15 (m, 1H), 5.74- 5.89 (m, 1H), 5.31 (d, J = 52 Hz, 1H), 4.28-4.48 (m, 2H), 3.75-4.21 (m, 4H), 3.15-3.31 (m, 3H), 2.98- 3.11 (m, 1H), 1.85-2.65 (m, 8H). |
| 12 | 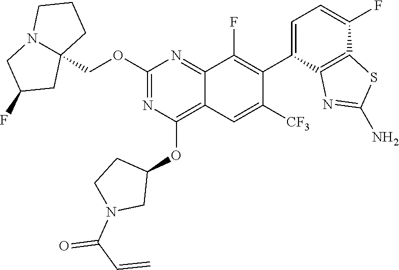 | LCMS m/z [M + 1]: 679.2 ¹HNMR (400 MHZ, CD₃OD) δ 8.27 (s, 1H), 7.12- 7.30 (m, 1H), 7.00 (t, J = 8.8 Hz, 1H), 6.58-6.81 (m, 1H), 6.29-6.41 (m, 1H), 5.88-6.15 (m, 1H), 5.74- 5.89 (m, 1H), 5.31 (d, J = 52 Hz, 1H), 4.28-4.48 (m, 2H), 3.75-4.21 (m, 4H), 3.15-3.31 (m, 3H), 2.98- 3.11 (m, 1H), 1.85-2.65 (m, 8H). |
| 13 | 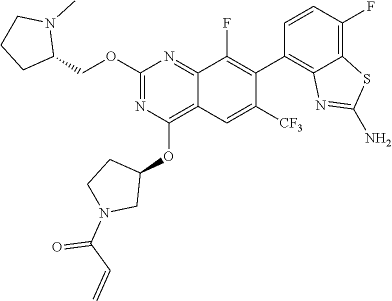 [SMILES: C=CC(=O)N1CC[C@@H](Oc2nc(OC[C@@H]3CCCN3C)nc3c(F)c(-c4ccc(F)c5sc(N)nc45)c(C(F)(F)F)cc23)C1] | LCMS m/z [M + 1]: 635.3 ¹HNMR (400 MHZ, CD₃OD) δ 8.30 (s, 1H), 7.16- 7.26 (m, 1H), 7.00 (t, J = 8.8 Hz, 1H), 6.57-6.73 (m, 1H), 6.32 (d, J = 15.2 Hz, 1H), 5.91-6.09 (m, 1H), 5.72-5.85 (m, 1H), 4.71-5.05 (m, 1H), 4.08-4.46 (m, 2H), 3.81-4.06 (m, 3H) 3.63-3.80 (m, 2H), 3.15 (s, 3H), 2.93-3.03 (m, 1H), 2.02-2.65 (m, 6H). |
| 13a | 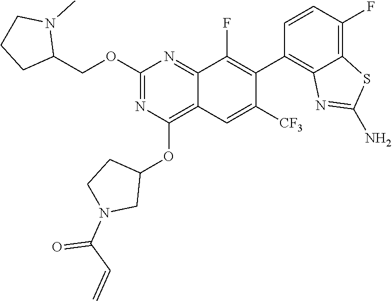 [SMILES: C=CC(=O)N1CCC(Oc2nc(OCC3CCCN3C)nc3c(F)c(-c4ccc(F)c5sc(N)nc45)c(C(F)(F)F)cc23)C1] | |
| 14 | 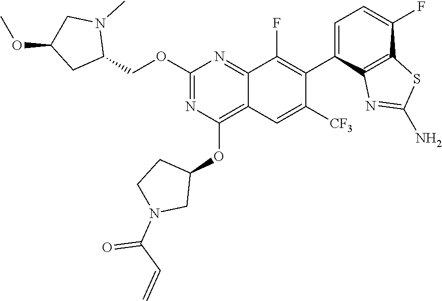 | LCMS m/z [M + 1]: 665.2 ¹HNMR (400 MHz, CD₃OD) δ 8.30 (s, 1H), 7.22 (s, 1H), 7.00 (t, J = 8.6 Hz, 1H), 6.57-6.76 (m, 1H), 6.32 (d, J = 16.8 Hz, 1H), 5.97-6.08(m, 1H), 5.73- 5.85 (m, 1H), 4.93-5.06 (m, 2H), 4.70-4.80 (m, 1H), 4.07-4.26 (m, 3H), 3.77-4.02 (m, 4H), 3.34-3.40(m, 3H), 3.11-3.21 (s, 3H), 2.41-2.66 (m, 3H), 2.14-2.25 (m, 1H). |
| 15 | 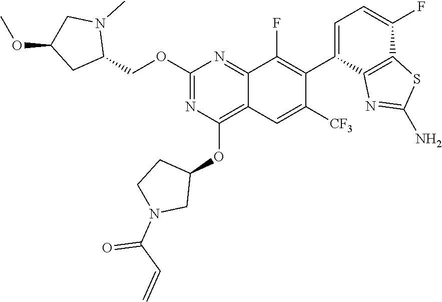 | LCMS m/z [M + 1]: 665.2 ¹HNMR (400 MHZ, CD₃OD) δ 8.30 (s, 1H), 7.22 (s, 1H), 7.00 (t, J = 8.6 Hz, 1H), 6.57-6.76 (m, 1H), 6.32 (d, J = 16.8 Hz, 1H), 5.97-6.08(m, 1H), 5.73- 5.85 (m, 1H), 4.93-5.06 (m, 2H), 4.70-4.80 (m, 1H), 4.07-4.26 (m, 3H), 3.77-4.02 (m, 4H), 3.34-3.40(m, 3H), 3.11-3.21 (s, 3H), 2.41-2.66 (m, 3H), 2.14-2.25 (m, 1H). |
| 15a | 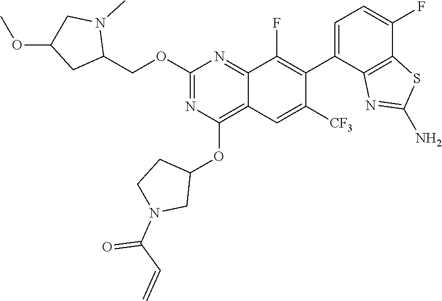 [SMILES: C=CC(=O)N1CCC(Oc2nc(OCC3CC(OC)CN3C)nc3c(F)c(-c4ccc(F)c5sc(N)nc45)c(C(F)(F)F)cc23)C1] | |
| 16 | 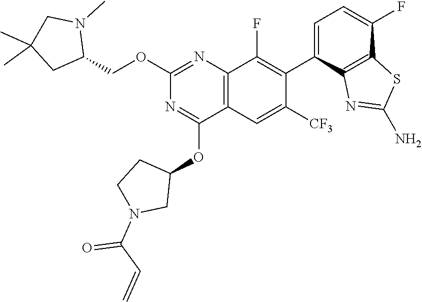 | LCMS m/z [M + 1]: 663.2 ¹HNMR (400 MHZ, CD₃OD) δ 8.31 (s, 1H), 7.19- 7.28 (m, 1H), 6.97-7.06 (m, 1H), 6.58-6.77 (m, 1H), 6.32 (d, J = 16.8 Hz, 1H), 5.95-6.04 (m, 1H), 5.75- 5.86 (m, 1H), 4.97-5.02 (m, 1H), 4.74-4.80 (m, 1H), 4.07-4.17 (m, 2H), 3.85-4.03 (m, 3H), 3.49-3.59(m, 1H), 3.09-3.18(m, 4H), 2.41-2.64 (m, 2H), 2.24- 2.31 (m, 1H), 1.96-2.05 (m, 1H), 1.29 (s, 3H), 1.27 (s, 3H). |
| 17 | 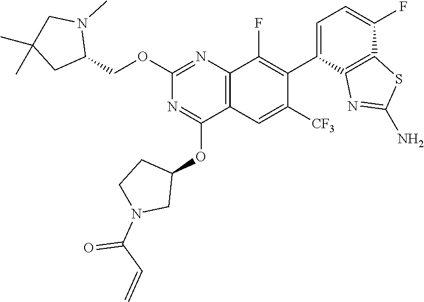 | LCMS m/z [M + 1]: 663.3 ¹HNMR (400 MHZ, CD₃OD) δ 8.31 (s, 1H), 7.19- 7.28 (m, 1H), 6.97-7.06 (m, 1H), 6.58-6.77 (m, 1H), 6.32 (d, J = 16.8 Hz, 1H), 5.95-6.04 (m, 1H), 5.75- 5.86 (m, 1H), 4.97-5.02 (m, 1H), 4.74-4.80 (m, 1H), 4.07-4.17 (m, 2H), 3.85-4.03 (m, 3H), 3.49-3.59(m, 1H), 3.09-3.18(m, 4H), 2.41-2.64 (m, 2H), 2.24- 2.31 (m, 1H), 1.96-2.05 (m, 1H), 1.29 (s, 3H), 1.27 (s, 3H). |
| 17a | 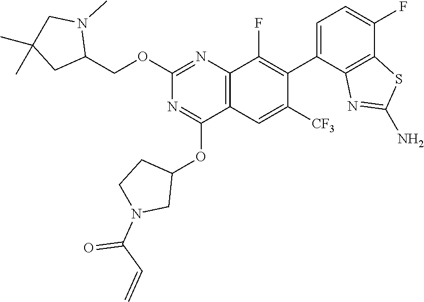 [SMILES: C=CC(=O)N1CCC(Oc2nc(OCC3CC(C)(C)CN3C)nc3c(F)c(-c4ccc(F)c5sc(N)nc45)c(C(F)(F)F)cc23)C1] | |
| 18 | 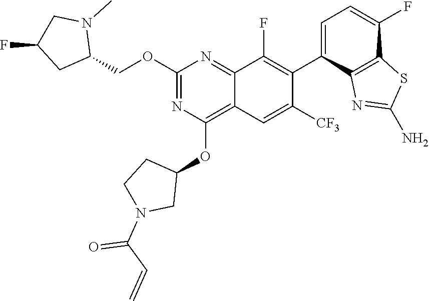 | LCMS m/z [M + 1]: 653.2 ¹HNMR (400 MHZ, CD₃OD) δ 8.30 (s, 1H), 7.15- 7.25 (m, 1H), 6.98 (t, J = 8.8 Hz, 1H), 6.57-6.75 (m, 1H), 6.32 (dd, J = 16.8, 1.8 Hz, 1H), 5.83-6.09 (m, 1H), 5.73-5.82 (m, 1H), 5.48(d, J = 52 Hz, 1H), 5.01-5.11 (m, 1H), 4.75-4.81 (m, 2H), 4.21-4.34 (m, 1H), 4.08-4.17 (m, 1H), 3.86-4.04 (m, 3H), 3.45- 3.81 (m, 1H), 3.19 (s, 3H), 2.31-2.75 (m, 4H). |
| 19 | 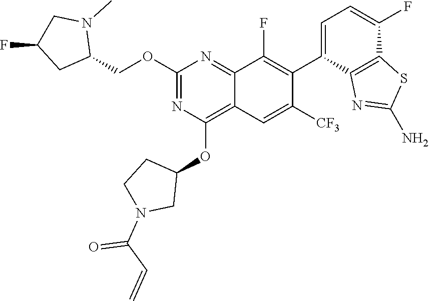 | LCMS m/z [M + 1]: 653.2 ¹HNMR (400 MHZ, CD₃OD) δ 8.31 (s, 1H), 7.15- 7.25 (m, 1H), 6.98 (t, J = 8.8 Hz, 1H), 6.57-6.75 (m, 1H), 6.32 (dd, J = 16.8, 1.8 Hz, 1H), 5.83-6.09 (m, 1H), 5.73-5.82 (m, 1H), 5.48(d, J = 52 Hz, 1H), 5.01-5.11 (m, 1H), 4.75-4.81 (m, 2H), 4.21-4.34 (m, 1H), 4.08-4.17 (m, 1H), 3.86-4.04 (m, 3H), 3.45- 3.81 (m, 1H), 3.19 (s, 3H), 2.31-2.75 (m, 4H). |
| 19a | 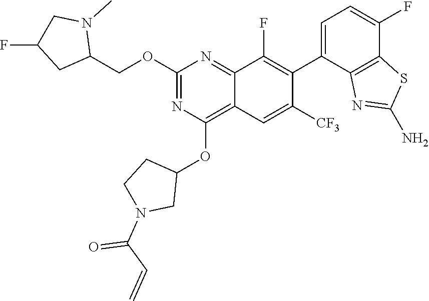 [SMILES: C=CC(=O)N1CCC(Oc2nc(OCC3CC(F)CN3C)nc3c(F)c(-c4ccc(F)c5sc(N)nc45)c(C(F)(F)F)cc23)C1] | |
| 20 | 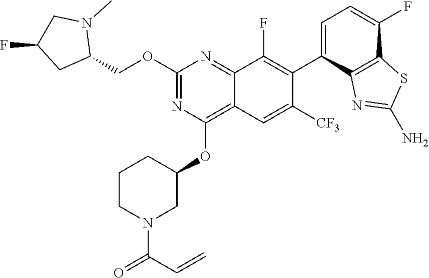 | LCMS m/z [M + 1]: 667.2 ¹HNMR (400 MHZ, CD₃OD) δ 8.19-8.22 (m, 1H), 7.17-7.21 (m, 1H), 6.98 (t, J = 8.8 Hz, 1H), 6.71- 6.88 (m, 1H) , 6.11-6.25 (m, 1H), 5.62-5.77 (m, 1H), 5.42-5.65 (m, 2H), 4.98-5.07 (m, 1H), 4.24- 4.28 (m, 2H), 4.04-4.14 (m, 1H), 3.86-3.94 (m, 2H), 3.43-3.67 (m, 2H), 3.19 (s, 3H), 2.65-2.75 (m, 1H), 1.68-2.48 (m, 6H). |
| 21 | 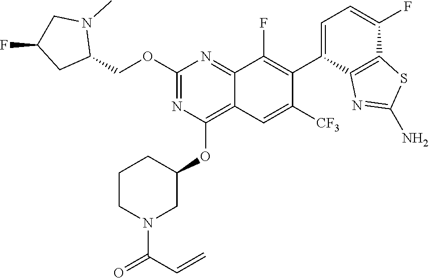 | LCMS m/z [M + 1]: 667.2 ¹HNMR (400 MHZ, CD₃OD) δ 8.19-8.22 (m, 1H), 7.17-7.20 (m, 1H), 6.97 (t, J = 8.8 Hz, 1H), 6.73- 6.88 (m, 1H) , 6.15-6.28 (m, 1H), 5.66-5.77 (m, 1H), 5.42-5.58 (m, 2H), 5.50-5.07 (m, 1H), 4.24- 4.28 (m, 2H), 4.04-4.14 (m, 1H), 3.86-3.94 (m, 2H),3.43-3.67 (m, 2H), 3.19 (s, 3H), 2.65-2.75 (m, 1H), 1.68-2.48 (m, 6H). |
| 21a | 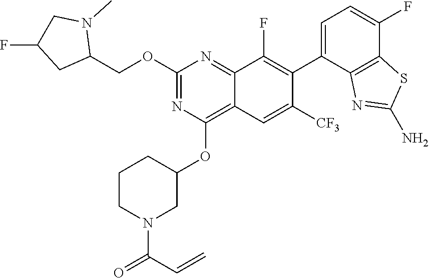 [SMILES: C=CC(=O)N1CCCC(Oc2nc(OCC3CC(F)CN3C)nc3c(F)c(-c4ccc(F)c5sc(N)nc45)c(C(F)(F)F)cc23)C1] | |
| 22 | 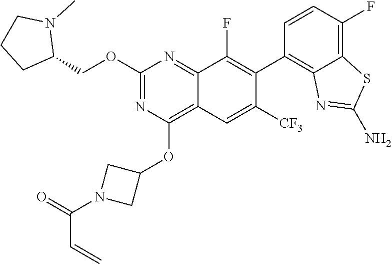 [SMILES: C=CC(=O)N1CC(Oc2nc(OC[C@@H]3CCCN3C)nc3c(F)c(-c4ccc(F)c5sc(N)nc45)c(C(F)(F)F)cc23)C1] | LCMS m/z [M + 1]: 621.2 ¹HNMR (400 MHZ, CD₃OD) δ 8.44 (s, 1H), 7.15- 7.29 (m, 1H), 6.92-7.07 (m, 1H), 6.24-6.48 (m, 2H), 5.72-5.87 (m, 2H), 4.88-5.09 (m, 2H), 4.53- 4.79 (m, 3H), 4.25-4.37 (m, 1H), 3.87-3.99 (m, 1H), 3.70-3.83 (m, 1H), 3.20-3.29 (m, 1H), 3.11 (s, 3H), 2.36-2.49 (m, 1H), 2.19-2.32 (m, 1H), 2.00-2.17 (m, 2H). |
| 22a | 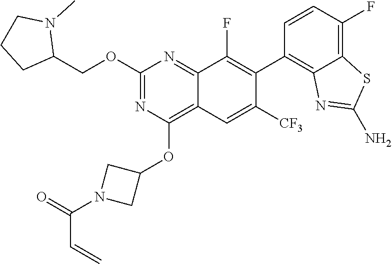 [SMILES: C=CC(=O)N1CC(Oc2nc(OCC3CCCN3C)nc3c(F)c(-c4ccc(F)c5sc(N)nc45)c(C(F)(F)F)cc23)C1] | |
| 23 | 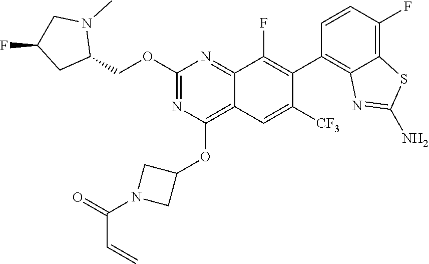 [SMILES: C=CC(=O)N1CC(Oc2nc(OC[C@@H]3C[C@@H](F)CN3C)nc3c(F)c(-c4ccc(F)c5sc(N)nc45)c(C(F)(F)F)cc23)C1] | LCMS m/z [M + 1]: 639.2 ¹HNMR (400 MHZ, CD₃OD) δ 8.45 (s, 1H), 7.16- 7.25 (m, 1H), 6.92-7.03 (m, 1H), 6.27-6.44 (m, 2H), 5.69-5.89 (m, 2H), 5.49 (d, J = 50.2 Hz, 1H), 4.97-5.10 (m, 1H), 4.88-4.98 (m, 1H), 4.73- 4.81 (m, 1H), 4.49-4.72 (m, 2H), 4.19-4.48 (m, 2H), 4.00-4.17 (m, 1H), 3.60-3.76 (m, 1H), 3.21 (s, 3H), 2.64-2.76 (m, 1H), 2.49-2.32 (m, 1H). |
| 23a | 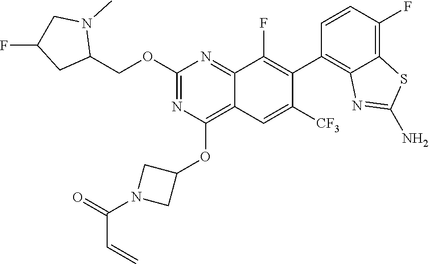 [SMILES: C=CC(=O)N1CC(Oc2nc(OCC3CC(F)CN3C)nc3c(F)c(-c4ccc(F)c5sc(N)nc45)c(C(F)(F)F)cc23)C1] | |
| 24 | 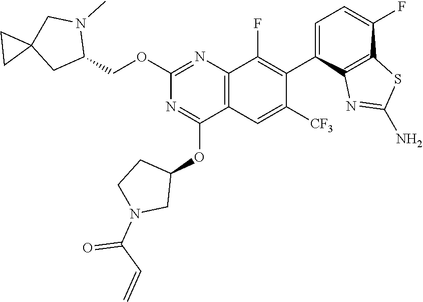 | LCMS m/z [M + 1]: 661.2 ¹HNMR(400 MHZ, CD₃OD) δ 8.31 (s, 1H), 7.16- 7.24 (m, 1H), 6.94-7.05 (m, 1H), 6.54-6.75 (m, 1H), 6.26-6.35 (m, H), 5.94-6.08 (m, 1H), 5.73-5.82 (m, 1H), 4.93-5.08 (m, 2H), 3.87-4.22 (m, 4H), 3.34-3.49 (m, 2H), 3.17 (s, 3H), 2.41-2.62 (m, 3H ), 1.98-2.08 (m, 1H), 1.23-1.28 (m, 1H), 0.72-0.89 (m, 4H). |
| 25 | 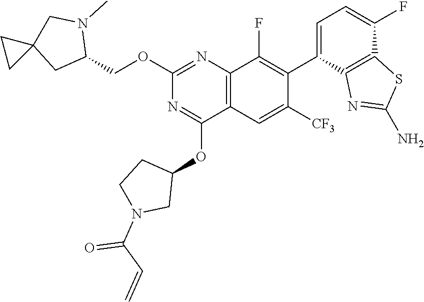 | LCMS m/z [M + 1]: 661.2 ¹HNMR (400 MHZ, CD₃OD) δ 8.34 (s, 1H), 7.17- 7.24 (m, 1H), 6.94-7.07 (m, 1H), 6.56-6.74 (m, 1H), 6.28-6.34 (m, H), 5.96-6.19 (m, 1H), 5.74-5.82 (m, 1H), 4.93-5.06 (m, 2H), 3.97-4.12 (m, 4H), 3.34-3.51 (m, 2H), 3.17 (s, 3H), 2.41-2.62 (m, 3H ), 1.97-2.08 (m, 1H), 1.21-1.32 (m, 1H), 0.73-0.91 (m, 4H) |
| 25a | 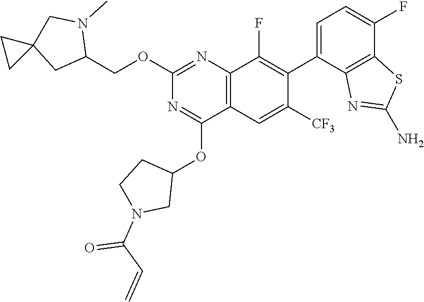 [SMILES: C=CC(=O)N1CCC(Oc2nc(OCC3CC4(CC4)CN3C)nc3c(F)c(-c4ccc(F)c5sc(N)nc45)c(C(F)(F)F)cc23)C1] | |
| 26 | 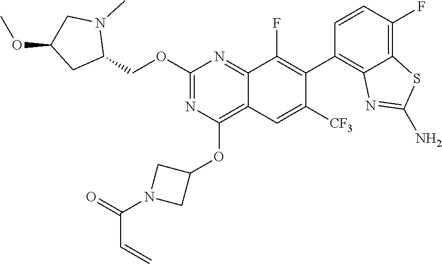 [SMILES: C=CC(=O)N1CC(Oc2nc(OC[C@@H]3C[C@@H](OC)CN3C)nc3c(F)c(-c4ccc(F)c5sc(N)nc45)c(C(F)(F)F)cc23)C1] | LCMS m/z [M + 1]: 651.2 ¹HNMR (400 MHZ, CD₃OD) δ 8.39 (s, 1H), 7.13- 7.27 (m, 1H), 6.89-7.04 (m, 1H), 6.22-6.47 (m, 2H), 5.71-5.84 (m, 2H), 4.84-4.90 (m, 1H), 4.46-4.72 (m, 4H), 4.32 (d, J = 11.9 Hz, 1H), 3.91-4.09 (m, 1H), 3.32-3.55 (m, 2H), 3.30 (s, 3H), 2.66-2.75 (m, 1H), 2.64 (s, 3H), 1.96-2.23 (m, 2H). |
| 26a | 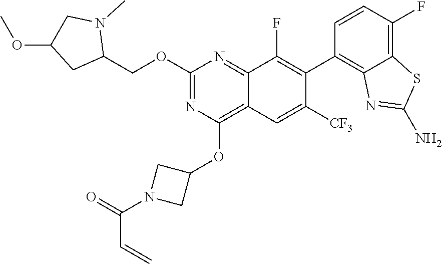 [SMILES: C=CC(=O)N1CC(Oc2nc(OCC3CC(OC)CN3C)nc3c(F)c(-c4ccc(F)c5sc(N)nc45)c(C(F)(F)F)cc23)C1] | |
| 27 | 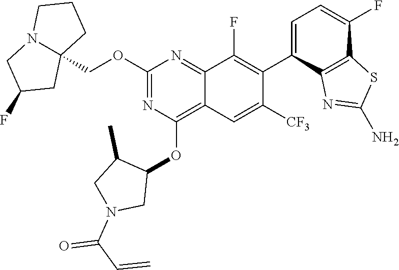 | LCMS m/z [M + 1]: 693.2 ¹H NMR (400 MHZ, CD₃OD) δ 8.21-8.28(m, 1H), 7.15-7.24 (m, 1H), 6.97 (t, J = 8.8 Hz, 1H), 6.55- 6.71 (m, 1H), 6.30 (dd, J = 16.8, 2.0 Hz, 1H), 5.91- 6.07 (m, 1H), 5.72-5.85 (m, 1H), 5.42 (d, J = 52 Hz, 1H), 5.11-5.22 (m, 1H), 4.33-4.49 (m, 2H), 3.91- 4.22 (m, 3H), 3.59-3.68 (m, 1H), 3.38-3.51 (m, 1H), 3.01-3.15 (m, 1H), 2.71-2.88 (m, 2H), 1.88-2.49 (m, 6H), 1.22-1.35(m, 3H). |
| 27a | 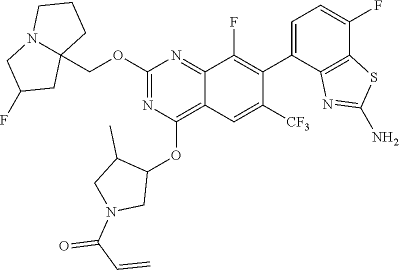 [SMILES: C=CC(=O)N1CC(C)C(Oc2nc(OCC34CCCN3CC(F)C4)nc3c(F)c(-c4ccc(F)c5sc(N)nc45)c(C(F)(F)F)cc23)C1] | |
| 28 | 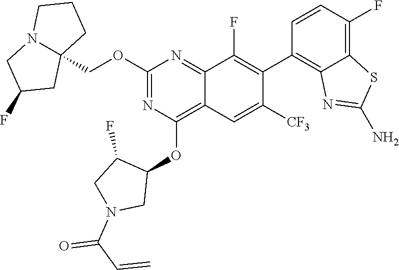 [SMILES: C=CC(=O)N1C[C@H](Oc2nc(OC[C@@]34CCCN3C[C@H](F)C4)nc3c(F)c(-c4ccc(F)c5sc(N)nc45)c(C(F)(F)F)cc23)[C@@H](F)C1] | LCMS m/z [M + 1]: 697.2 ¹HNMR (400 MHZ, CD₃OD) δ 8.22-8.29 (m, 1H), 7.12-7.23 (m, 1H), 6.97 (t, J = 8.8 Hz, 1H), 6.62- 6.72 (m, 1H), 6.35 (d, J = 16.7 Hz, 1H), 5.96-6.08 (m, 1H), 5.78-5.87 (m, 1H), 5.57 (dd, J = 48.6, 20.7 Hz, 1H), 5.35 (d, J = 55.2 Hz, 1H), 4.36-4.48 (m, 2H), 3.91-4.31(m, 4H), 3.33-3.42 (m, 3H), 3.06- 3.12 (m, 1H), 2.17-2.47 (m, 3H), 2.03 (m, 3H). |
| 28a | 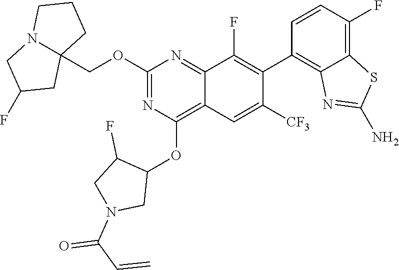 [SMILES: C=CC(=O)N1CC(F)C(Oc2nc(OCC34CCCN3CC(F)C4)nc3c(F)c(-c4ccc(F)c5sc(N)nc45)c(C(F)(F)F)cc23)C1] | |
| 29 | 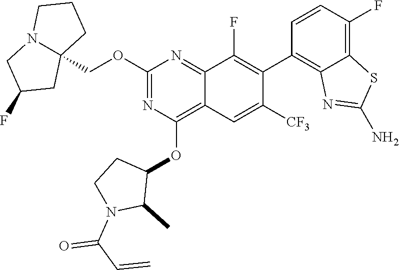 | LCMS m/z [M + 1]: 693.2 ¹H NMR (400 MHZ, CD₃OD) δ 8.27 (s, 1H), 7.18- 7.21 (m, 1H), 6.97 (t, J = 8.8 Hz, 1H), 6.58-6.74 (m, 1H), 6.29-6.34 (m, 1H), 5.99-6.06 (m, 1H), 5.75- 5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H). |
| 29a | 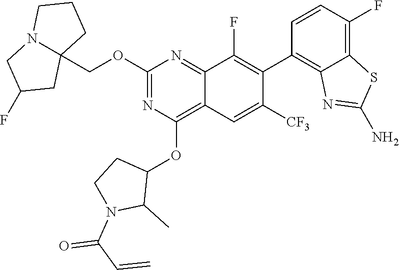 [SMILES: C=CC(=O)N1CCC(Oc2nc(OCC34CCCN3CC(F)C4)nc3c(F)c(-c4ccc(F)c5sc(N)nc45)c(C(F)(F)F)cc23)C1C] | |
| 30 | 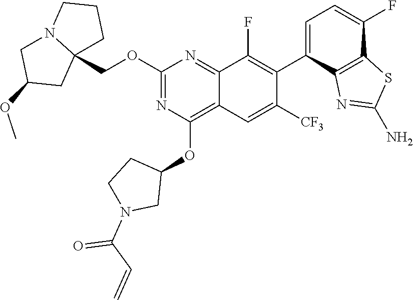 | LCMS m/z [M + 1]: 691.3 ¹HNMR (400 MHz, CD₃OD) δ 8.27 (s, 1H), 7.18- 7.21 (m, 1H), 6.97 (t, J = 8.8 Hz, 1H), 6.58-6.74 (m, 1H), 6.29-6.34 (m, 1H), 5.99-6.06 (m, 1H), 5.75- 5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H). |
| 31 | 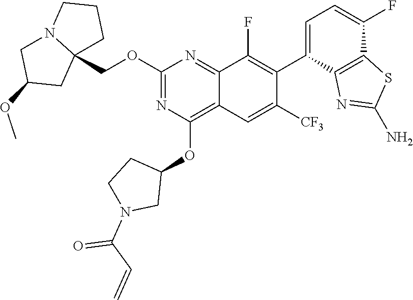 | LCMS m/z [M + 1]: 691.3 ¹HNMR (400 MHZ, CD₃OD) δ 8.27 (s, 1H), 7.18- 7.21 (m, 1H), 6.97 (t, J = 8.8 Hz, 1H), 6.58-6.74 (m, 1H), 6.29-6.34 (m, 1H), 5.99-6.06 (m, 1H), 5.75- 5.81 (m, 1H), 4.39-4.50 (m, 2H), 3.72-4.16 (m, 5H), 3.34-3.41 (m, 5H), 3.13-3.21 (m, 2H), 1.96-2.60 (m, 8H). |
| 31a | 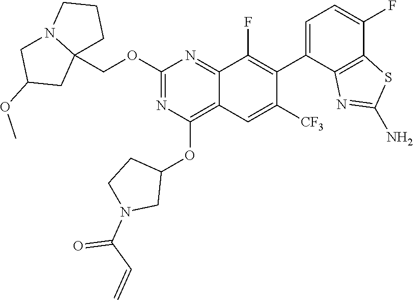 [SMILES: C=CC(=O)N1CCC(Oc2nc(OCC34CCCN3CC(OC)C4)nc3c(F)c(-c4ccc(F)c5sc(N)nc45)c(C(F)(F)F)cc23)C1] | |
| 32 | 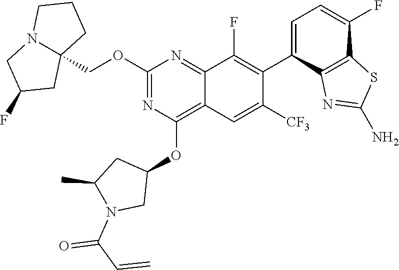 | LCMS m/z [M + 1]: 693.2 ¹HNMR (400 MHZ, CD₃OD) δ 8.27 (d, J = 6.9 Hz, 1H), 7.14-7.26 (m, 1H), 6.91-7.04 (m, 1H), 6.57- 6.77 (m, 1H), 6.26-6.39 (m, 1H), 5.97-6.05 (m, 1H), 5.72-5.84 (m, 1H), 5.35 (d, J = 53.2 Hz, 1H), 4.33-4.61 (m, 3H), 3.95-4.20 (m, 2H), 3.32- 3.52 (m, 3H), 3.03-3.16 (m, 1H), 2.52-2.84 (m, 1H), 2.12-2.53 (m, 4H), 1.82-2.11 (m, 3H), 1.51 (dd, J = 13.1, 6.6 Hz, 3H). |
| 33 | 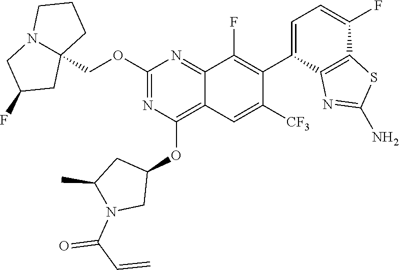 | LCMS m/z [M + 1]: 693.2 ¹HNMR (400 MHZ, CD₃OD) δ 8.26 (d, J = 6.2 Hz, 1H), 7.13-7.26 (m, 1H), 6.89-7.01 (m, 1H), 6.56- 6.79 (m, 1H), 6.27-6.38 (m, 1H), 5.96-6.06 (m, 1H), 5.72-5.84 (m, 1H), 5.33 (d, J = 53.8 Hz, 1H), 3.95-4.55 (m, 5H), 3.19-3.30 (m, 3H), 3.03- 3.14 (m, 1H), 2.52-2.80 (m, 1H), 2.13-2.49 (m, 4H), 1.77-2.12 (m, 3H), 1.52 (dd, J = 12.9, 6.6 Hz, 3H). |
| 33a | 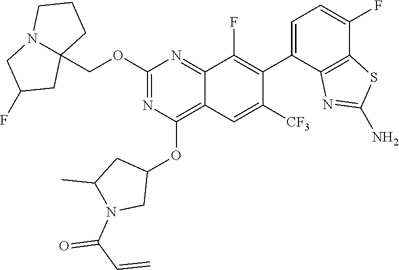 [SMILES: C=CC(=O)N1CC(Oc2nc(OCC34CCCN3CC(F)C4)nc3c(F)c(-c4ccc(F)c5sc(N)nc45)c(C(F)(F)F)cc23)CC1C] | |
| 34 | 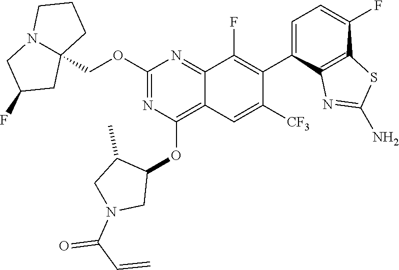 | LCMS m/z [M + 1]: 693.2 ¹HNMR (400 MHZ, CD₃OD) δ 8.27 (s, 1H), 7.17- 7.22 (m, 1H), 6.97 (t, d = 8.8 Hz,1H), 6.58-6.72 (m, 1H), 6.31-6.37 (m, 1H), 5.73-5.82 (m, 1H), 5.58- 5.69 (m, 1H), 5.17-5.42 (m, 1H), 4.55-4.61 (m, 1H), 4.31-4.52 (m, 2H), 4.04-4.21 (m, 1H), 3.79-4.01 (m, 1H ), 3.31-3.61 (m, 4H), 3.12-3.22 (m, 1H), 2.73- 2.93 (m, 1H), 1.89-2.48 (m, 6H), 1.21-1.27 (m, 3H). |
| 35 | 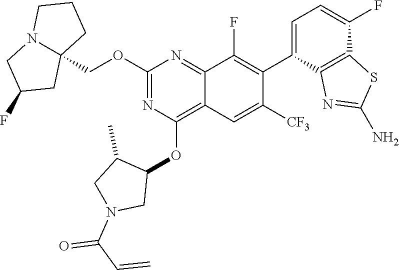 | LCMS m/z [M + 1]: 693.2 ¹HNMR (400 MHZ, CD₃OD) δ 8.28 (s, 1H), 7.14- 7.22 (m, 1H), 6.97 (t, d = 8.8 Hz, 1H), 6.58-6.73 (m, 1H), 6.24-6.35 (m, 1H), 5.72-5.81 (m, 1H), 5.56- 5.68 (m, 1H), 5.17-5.24 (m, 1H), 4.48-4.62 (m, 1H), 4.31-4.56 (m, 2H), 4.03-4.21 (m, 1H), 3.79-4.01 (m, 1H ), 3.38-3.57 (m, 4H), 3.17-3.22 (m, 1H), 2.86- 2.94 (m, 1H), 1.93-2.49 (m, 6H), 1.17-1.26 (m, 3H). |
| 36 | 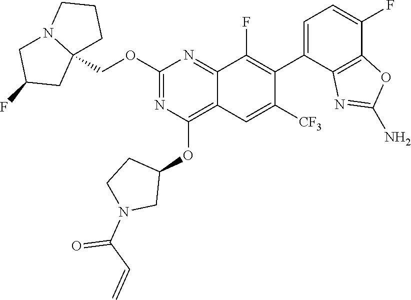 [SMILES: C=CC(=O)N1CC[C@@H](Oc2nc(OC[C@@]34CCCN3C[C@H](F)C4)nc3c(F)c(-c4ccc(F)c5oc(N)nc45)c(C(F)(F)F)cc23)C1] | LCMS m/z [M + 1]: 663.3 ¹HNMR (400 MHZ, CD₃OD): δ 8.27 (s, 1H), 7.05- 7.08 (m, 1H), 6.96 (t, J = 8.8 Hz, 1H), 6.58-6.73 (m, 1H), 6.29-6.33 (m, 1H), 5.99-6.02 (m, 1H), 5.75- 5.81 (m, 1H), 5.38 (d, J = 52.6 Hz, 1H), 4.38-4.50 (m, 2H), 3.72-4.16 (m, 4H), 3.37-3.48 (m, 2H), 3.08-3.17 (m, 1H), 1.97-2.55 (m, 9H). |
| 36a | 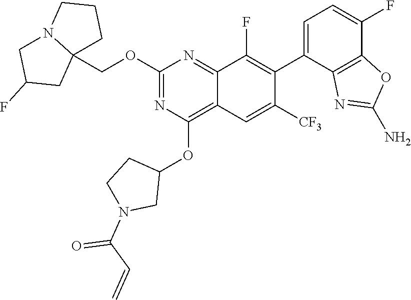 [SMILES: C=CC(=O)N1CCC(Oc2nc(OCC34CCCN3CC(F)C4)nc3c(F)c(-c4ccc(F)c5oc(N)nc45)c(C(F)(F)F)cc23)C1] | |
| 37 | 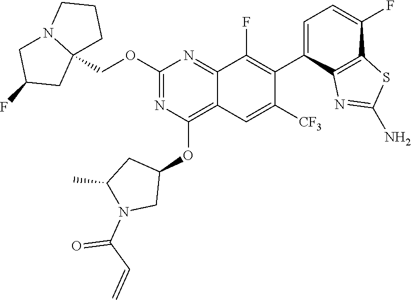 | LCMS m/z [M + 1]: 693.2 ¹HNMR (400 MHZ, CD₃OD) δ 8.20 (d, J = 8.8 Hz, 1H), 7.13-7.21 (m, 1H), 6.96 (t, J = 8.8 Hz, 1H), 6.54-6.81 (m, 1H), 6.21-6.37 (m, 1H), 5.51-5.98 (m, 1H), 5.71-5.85 (m, 1H), 5.32 (d, J = 52.8 Hz, 1H), 5.17 (s, 1H), 4.49-4.28 (m, 2H), 3.89-4.20 (m, 2H), 2.98-3.24 (m, 3H), 2.62-2.81 (m, 1H), 1.85-2.45 (m, 8H), 1.41 (d, J = 6.4 Hz, 3H). |
| 38 | 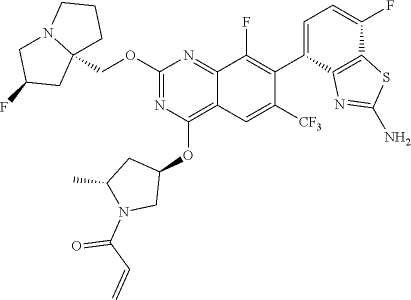 | LCMS m/z [M + 1]: 693.2 ¹HNMR (400 MHZ, CD₃OD) δ 8.21 (d, J = 10.8 Hz, 1H), 7.13-7.21 (m, 1H), 6.96 (t, J = 8.8 Hz, 1H), 6.54-6.81 (m, 1H), 6.21-6.37 (m, 1H), 5.51-5.98 (m, 1H), 5.71-5.85 (m, 1H), 5.32 (d, J = 52.8 Hz, 1H), 5.17 (s, 1H), 4.49-4.28 (m, 2H), 3.89-4.20 (m, 2H), 2.98-3.24 (m, 3H), 2.62-2.81 (m, 1H), 1.85-2.45 (m, 8H), 1.38-1.42 (m, 3H). |
| 38a | 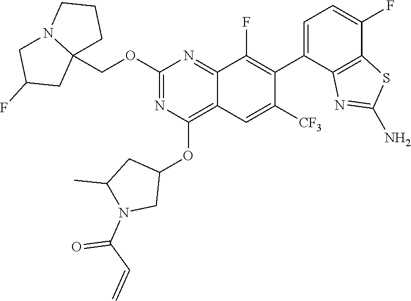 [SMILES: C=CC(=O)N1CC(Oc2nc(OCC34CCCN3CC(F)C4)nc3c(F)c(-c4ccc(F)c5sc(N)nc45)c(C(F)(F)F)cc23)CC1C] | |
| 39 | 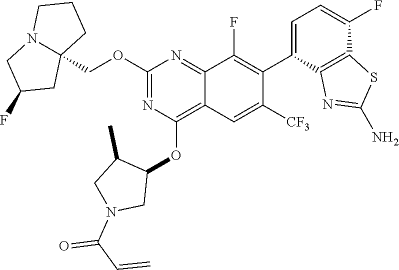 | LCMS m/z [M + 1]: 693.2 ¹HNMR (400 MHz, CD₃OD) δ 8.21-8.28(m, 1H), 7.15-7.24 (m, 1H), 6.97 (t, J = 8.8 Hz, 1H), 6.55- 6.71 (m, 1H), 6.30 (d, J = 16.8 Hz, 1H), 5.91-6.07 (m, 1H), 5.72-5.85 (m, 1H), 5.42 (d, J = 52 Hz, 1H), 5.11-5.22 (m, 1H), 4.33-4.49 (m, 2H), 3.91-4.22 (m, 3H), 3.59-3.68 (m, 1H), 3.38-3.51 (m, 1H), 3.01- 3.15 (m, 1H), 2.71-2.88 (m, 2H), 1.88-2.49 (m, 6H), 1.19-1.31(m, 3H). |
| 40 | 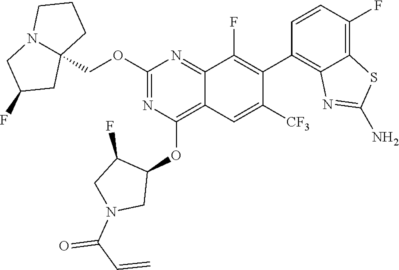 [SMILES: C=CC(=O)N1C[C@@H](F)[C@@H](Oc2nc(OC[C@@]34CCCN3C[C@H](F)C4)nc3c(F)c(-c4ccc(F)c5sc(N)nc45)c(C(F)(F)F)cc23)C1] | LCMS m/z [M + 1]: 697.2 ¹HNMR (400 MHZ, CD₃OD) δ 8.51 (s, 1H), 8.33 (s, 1H), 7.18-7.24 (m, 1H), 6.97 (t, J = 8.8 Hz, 1H), 6.59-6.72 (m, 1H), 6.36 (d, J = 16.4 Hz, 1H), 5.89- 6.02 (m, 2H), 5.66 (dd, J = 54.4, 4.0 Hz, 1H), 5.33 (d, J = 54 Hz, 1H), 5.09-5.21 (m, 1H), 4.30-4.51 (m, 3H), 3.94-4.19 (m, 3H), 3.01-3.28 (m, 3H), 1.85- 2.43 (m, 6H). |
| 41 | 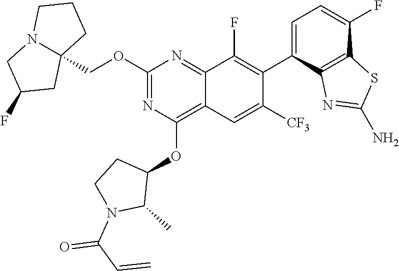 | LCMS m/z [M + 1]: 693.2 ¹HNMR (400 MHZ, CD₃OD) δ 8.21 (d, J = 7.4 Hz, 1H), 7.13-7.26 (m, 1H), 6.87-7.04 (m, 1H), 6.56- 6.77 (m, 1H), 6.20-6.39 (m, 1H), 5.57-5.87 (m, 2H), 5.33 (d, J = 54.0 Hz, 1H), 4.47-4.64 (m, 1H), 4.21-4.46 (m, 2H), 3.77-4.07 (m, 2H), 3.18- 3.30 (m, 3H), 2.99-3.14 (m, 1H), 1.84-2.77 (m, 8H), 1.42 (t, J = 6.9 Hz, 3H). |
| 42 | 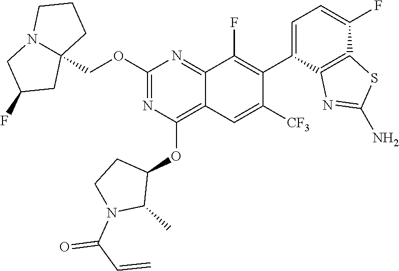 | LCMS m/z [M + 1]: 693.2 ¹HNMR (400 MHZ, CD₃OD) δ 8.21 (d, J = 8.4 Hz, 1H), 7.11-7.26 (m, 1H), 6.89-7.02 (m, 1H), 6.58- 6.76 (m, 1H), 6.22-6.42 (m, 1H), 5.56-5.84 (m, 2H), 5.33 (d, J = 53.9 Hz, 1H), 4.48-4.62 (m, 1H), 4.21-4.46 (m, 2H), 3.76-4.03 (m, 2H), 3.31- 3.43 (m, 1H), 3.18-3.29 (m, 2H), 2.97-3.10 (m, 1H), 2.43-2.76 (m, 2H), 2.11-2.39 (m, 3H), 1.85- 2.07 (m, 3H), 1.43 (t, J = 6.8 Hz, 3H). |
| 43 | 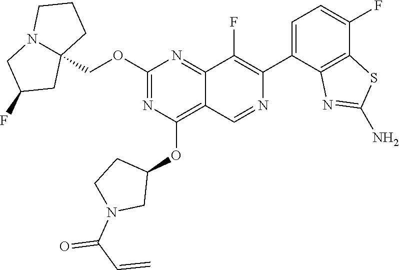 [SMILES: C=CC(=O)N1CC[C@@H](Oc2nc(OC[C@@]34CCCN3C[C@H](F)C4)nc3c(F)c(-c4ccc(F)c5sc(N)nc45)ncc23)C1] | LCMS m/z [M + 1]: 612.46 ¹HNMR (300 MHz, CD₃OD) 9.13 (d, J = 1.5 Hz, 1H), 7.46 (dd, J = 8.5, 5.5 Hz, 1H), 7.02 (t, J = 8.8 Hz, 1H), 6.65 (ddd, J = 24.2, 16.8, 10.4 Hz, 1H), 6.31 (dt, J = 16.8, 2.1 Hz, 1H), 6.03 (d, J = 14.8 Hz, 1H), 5.78 (ddd, J = 10.2, 8.0, 2.0 Hz, 1H), 5.33 (d, J = 53.8 Hz, 1H), 4.45-4.30 (m, 2H), 4.14- 3.67 (m, 4H), 3.39-3.13 (m, 3H), 3.05 (td, J = 9.4, 5.3 Hz, 1H), 2.60-2.29 (m, 3H), 2.28-2.10 (m, 2H), 2.09-1.85 (m, 3H) |
| 43a | 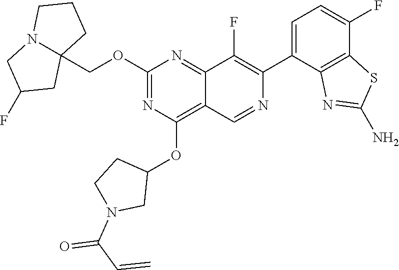 [SMILES: C=CC(=O)N1CCC(Oc2nc(OCC34CCCN3CC(F)C4)nc3c(F)c(-c4ccc(F)c5sc(N)nc45)ncc23)C1] | |
| 44 | 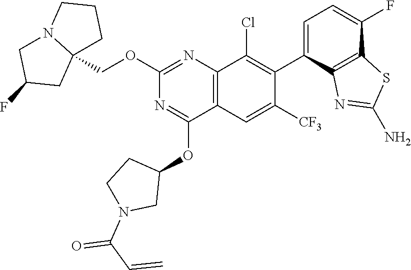 | LCMS m/z [M + 1]: 695.3 |
| 44a | 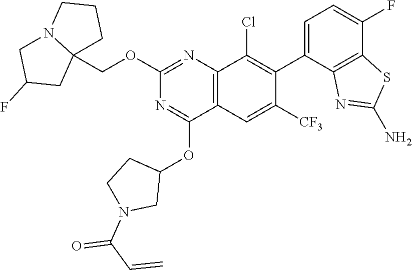 [SMILES: C=CC(=O)N1CCC(Oc2nc(OCC34CCCN3CC(F)C4)nc3c(Cl)c(-c4ccc(F)c5sc(N)nc45)c(C(F)(F)F)cc23)C1] | |
| 45 | 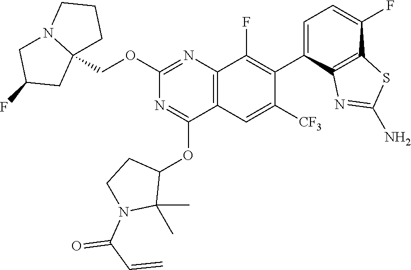 | LCMS m/z [M + 1]: 707.4 |
| 45a | 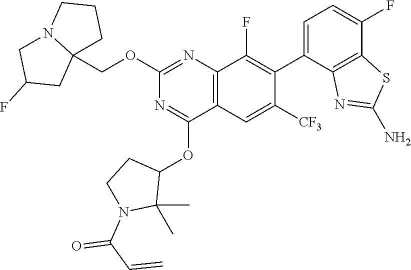 [SMILES: C=CC(=O)N1CCC(Oc2nc(OCC34CCCN3CC(F)C4)nc3c(F)c(-c4ccc(F)c5sc(N)nc45)c(C(F)(F)F)cc23)C1(C)C] | |
| 46 | 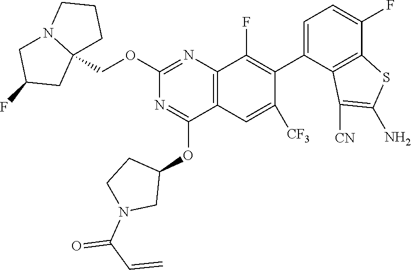 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M+1]: 703.2 ¹HNMR (400 MHZ, CD₃OD) δ 8.28 (d, J = 3.6 Hz, 1H), 7.15-7.27 (m, 1H), 6.99 (t, J = 9.2 Hz, 1H), 6.58-6.77 (m, 1H), 6.33 (d, J = 16.8 Hz, 1H), 5.96- 6.08 (m, 1H), 5.72-5.83 (m, 1H), 5.36 (d, J = 53.2 Hz, 1H), 4.32-4.39 (m, 2H), 3.81-4.08 (m, 4H), 3.58-3.79 (m, 1H), 3.38-3.56(m, 2H), 3.08-3.13 (m, 1H), 1.91-2.50 (m, 8H). |
| 47 | 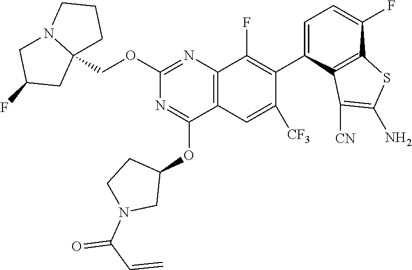 | |
| 48 | 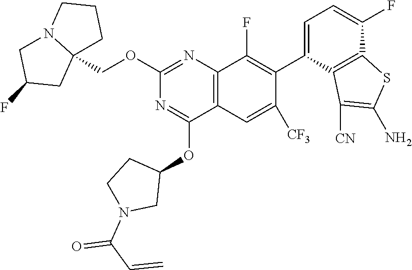 | |
| 48a | 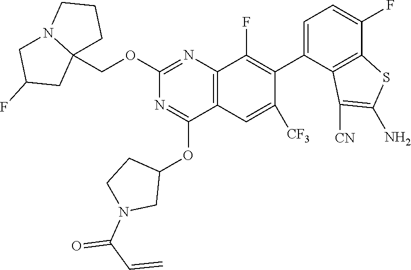 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1] | |
| 49 | 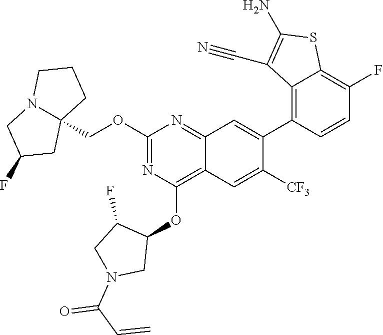 [SMILES: C=CC(=O)N1C[C@H](Oc2nc(OC[C@@]34CCCN3C[C@H](F)C4)nc3cc(-c4ccc(F)c5sc(N)c(C#N)c45)c(C(F)(F)F)cc23)[C@@H](F)C1] | LCMS m/z [M + 1]: 703.3 ¹HNMR (400 MHZ, CD₃OD) δ 8.42-8.44 (m, 1H), 7.64 (s, 1H), 7.16-7.19 (m, 1H), 6.97 (t, J = 9.2 Hz, 1H), 6.63-6.71 (m, 1H), 6.32-6.37 (m, 1H), 5.95- 6.06 (m, 1H), 5.79-5.84 (m, 1H), 5.48-5.65 (m, 1H), 5.36 (d, J = 53.2 Hz, 1H), 4.35-4.45 (m, 2H), 3.91- 4.28 (m, 4H), 3.33-3.38 (m, 3H), 3.08-3.13 (m, 1H), 1.96-2.42 (m, 6H). |
| 50 | 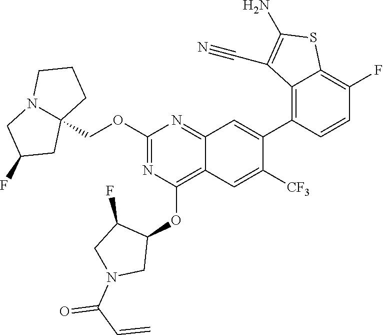 [SMILES: C=CC(=O)N1C[C@@H](F)[C@@H](Oc2nc(OC[C@@]34CCCN3C[C@H](F)C4)nc3cc(-c4ccc(F)c5sc(N)c(C#N)c45)c(C(F)(F)F)cc23)C1] | LCMS m/z [M + 1]: 703.3 ¹H NMR (400 MHZ, CD₃OD) δ 8.50 (s, 1H), 7.65 (s, 1H), 7.18-7.20 (m, 1H), 6.98 (t, J = 8.8 Hz, 1H), 6.61-6.70 (m, 1H), 6.33-6.38 (m, 1H), 5.86-5.95 (m, 1H), 5.81-5.86 (m, 1H), 5.56-5.70 (m, 1H), 5.33 (d, J = 54.4 Hz, 1H), 4.60 (s, 1H), 4.29-4.50 (m, 3H), 3.72-4.15 (m, 3H), 3.25-3.27 (m, 2H), 3.06-3.13 (m, 1H), 1.93-2.37 (m, 6H). |
| 50a | 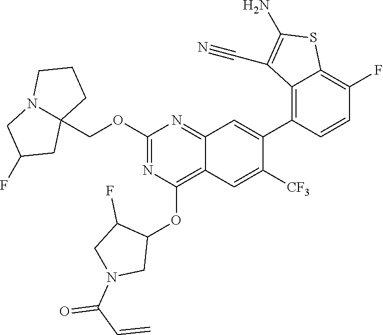 [SMILES: C=CC(=O)N1CC(F)C(Oc2nc(OCC34CCCN3CC(F)C4)nc3cc(-c4ccc(F)c5sc(N)c(C#N)c45)c(C(F)(F)F)cc23)C1] | |
| 51 | 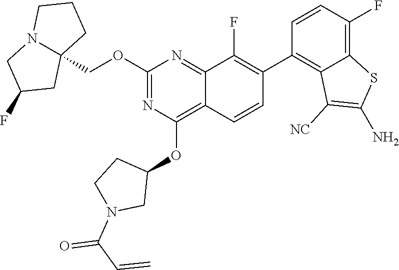 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)C1] | LCMS m/z [M + 1]: 635.3 ¹HNMR (400 MHZ, CD₃OD) δ 7.95-8.00 (m, 1H), 7.43 (t, J = 6.8 Hz, 1H), 7.26 (dd, J = 8.0, 4.8Hz, 1H), 7.03 (t, J = 8.8 Hz, 1H), 6.66-6.72(m, 1H), 6.28-6.34 (m, 1H), 6.00 (d, J= 16.8 Hz, 1H), 5.75-5.80 (m, 1H), 5.57 (d, J= 52 Hz, 1H), 4.80-4.84 (m, 1H),4.67-4.74 (m, 1H), 4.01-4.08 (m, 1H), 3.71-3.98 (m, 6H), 3.45-3.54 (m, 1H), 2.34-2.82 (m, 7H), 2.14-2.18 (m, 1H). |
| 51a | 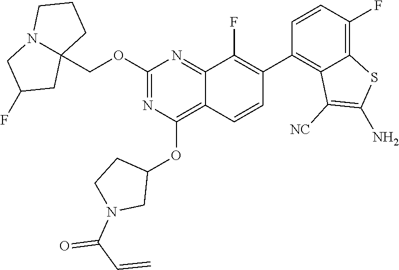 [SMILES: C=CC(=O)N1CCC(Oc2nc(OCC34CCCN3CC(F)C4)nc3c(F)c(-c4ccc(F)c5sc(N)c(C#N)c45)ccc23)C1] | |
| 52 | 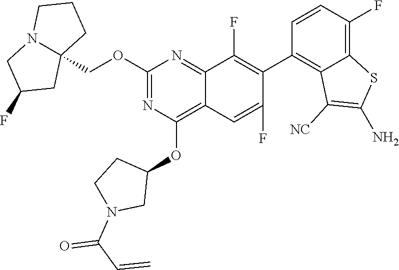 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(F)cc23)C1] | LCMS m/z [M + 1]: 653.2 ¹H NMR (400 MHZ, CD₃OD) δ 7.65-7.69 (m, 1H), 7.26-7.27 (m, 1H), 7.02 (t, J = 8.4 Hz, 1H), 6.5- 6.72 (m, 1H), 6.28-6.32 (m, 1H), 5.90-6.00 (m, 1H), 5.74-5.79 (m, 1H), 5.38 (d, J = 53.2 Hz, 1H), 4.37-4.45 (m, 2H), 3.92-4.09 (m, 3H), 3.41-3.47 (m, 3H), 3.12-3.16 (m, 2H), 2.22- 2.52 (m, 6H), 2.03-2.10 (m, 2H). |
| 52a | 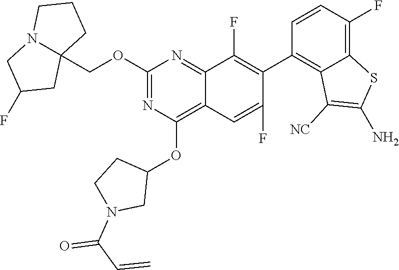 [SMILES: C=CC(=O)N1CCC(Oc2nc(OCC34CCCN3CC(F)C4)nc3c(F)c(-c4ccc(F)c5sc(N)c(C#N)c45)c(F)cc23)C1] | |
| 53 | 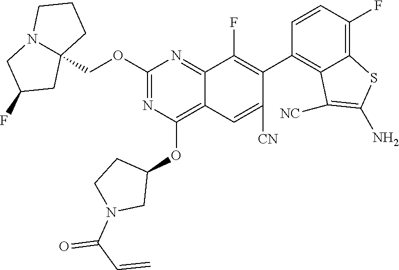 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C#N)cc23)C1] | |
| 53a | 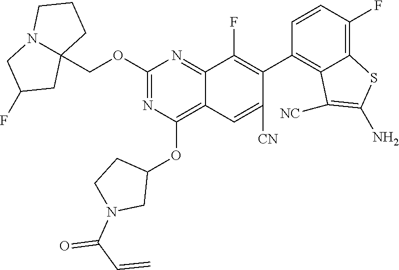 [SMILES: C=CC(=O)N1CCC(Oc2nc(OCC34CCCN3CC(F)C4)nc3c(F)c(-c4ccc(F)c5sc(N)c(C#N)c45)c(C#N)cc23)C1] | |
| 54 | 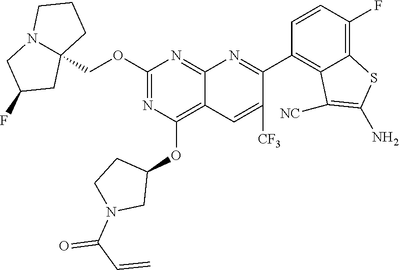 [SMILES: C=CC(=O)N1CC[C@@H](Oc2nc(OC[C@@]34CCCN3C[C@H](F)C4)nc3nc(-c4ccc(F)c5sc(N)c(C#N)c45)c(C(F)(F)F)cc23)C1] | |
| 54a | 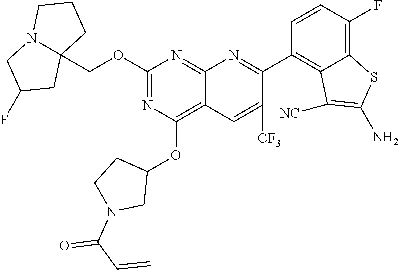 [SMILES: C=CC(=O)N1CCC(Oc2nc(OCC34CCCN3CC(F)C4)nc3nc(-c4ccc(F)c5sc(N)c(C#N)c45)c(C(F)(F)F)cc23)C1] | |
| 55 | 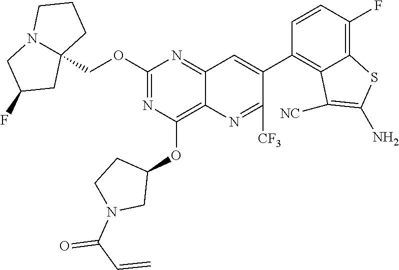 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C(F)(F)F)nc23)C1] | LCMS m/z [M + 1]: 686.1 ¹HNMR(400 MHz,CD₃OD) δ 8.51 ( s,1H), 8.10 (s, 1H), 7.24 (s, 1H), 7.03 (t, 1H), 6.61-6.75 (m, 2H), 6.34 (s, 0.5H), 6.30 (s, 0.5H), 6.07 (s, 0.5H), 6.05 (s, 0.5H), 5.77-5.82 (m, 1H), 5.50-5.51 (m, 1H), 4.51-4.63 (m, 2H), 4.16 (s, 1H), 3.98-4.04 (m, 2H), 3.50- 3.63 (m, 4H), 2.05-2.58 (m, 9H). |
| 55a | 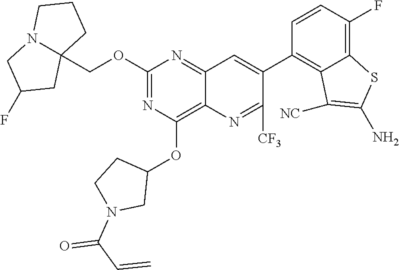 [SMILES: C=CC(=O)N1CCC(Oc2nc(OCC34CCCN3CC(F)C4)nc3cc(-c4ccc(F)c5sc(N)c(C#N)c45)c(C(F)(F)F)nc23)C1] | |
| 56 | 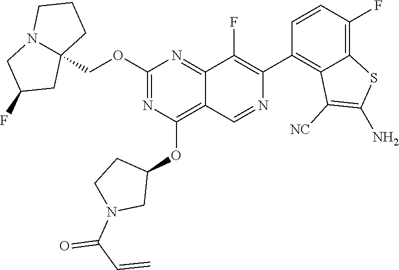 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ncc23)C1] | LCMS m/z [M + 1]: 636.2 ¹H NMR (400 MHZ, CD₃OD) δ 9.16 (s, 1H), 7.40- 7.42 (m, 1H), 7.06 (t, J = 8.0 Hz, 1H), 6.62-6.69 (m, 1H), 6.28-6.33 (m, 1H), 5.77- 6.11 (m, 2H), 5.30 (d, J = 54.8 Hz, 1H), 4.47- 4.61 (m, 2H), 3.44-3.97 (m, 7H), 3.12-3.14 (m, 1H), 1.96-2.42 (m, 8H). |
| 56a | 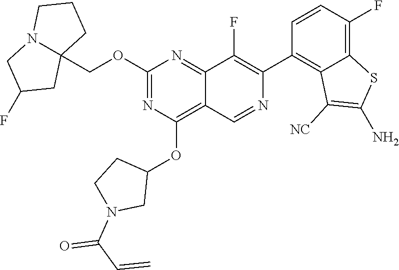 [SMILES: C=CC(=O)N1CCC(Oc2nc(OCC34CCCN3CC(F)C4)nc3c(F)c(-c4ccc(F)c5sc(N)c(C#N)c45)ncc23)C1] | |
| 57 | 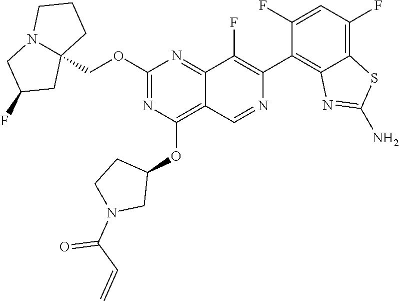 [SMILES: C=CC(=O)N1CC[C@@H](Oc2nc(OC[C@@]34CCCN3C[C@H](F)C4)nc3c(F)c(-c4c(F)cc(F)c5sc(N)nc45)ncc23)C1] | LCMS m/z [M + 1]: 630.3 ¹HNMR (400 MHZ, CD₃OD) δ 9.17 (d, J = 2.7 Hz, 1H), 6.91 (t, J = 8.8 Hz, 1H), 6.65 (m, 1H), 6.31 (d, J = 16.8 Hz, 1H), 6.04 (d, J = 24 Hz, 1H), 5.78 (t, J = 12 Hz, 1H), 5.35(d, J = 53.6 Hz, 1H), 4.35- 4.60(m, 3H), 3.63-4.12 (m, 4H), 3.43-3.51(m, 2H), 3.03-3.16 (m, 1H), 1.86-2.66 (m, 8H). |
| 57a | 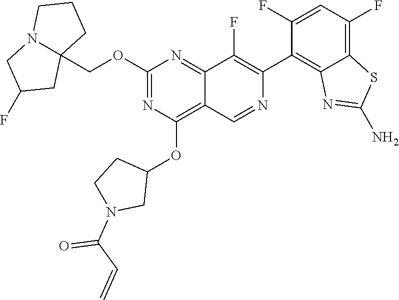 [SMILES: C=CC(=O)N1CCC(Oc2nc(OCC34CCCN3CC(F)C4)nc3c(F)c(-c4c(F)cc(F)c5sc(N)nc45)ncc23)C1] | |
| 58 | 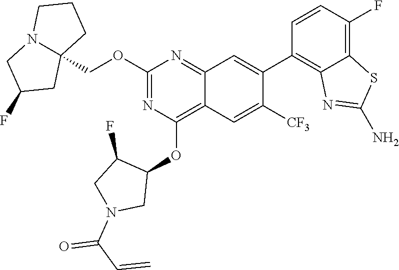 [SMILES: C=CC(=O)N1C[C@@H](F)[C@@H](Oc2nc(OC[C@@]34CCCN3C[C@H](F)C4)nc3cc(-c4ccc(F)c5sc(N)nc45)c(C(F)(F)F)cc23)C1] | LCMS m/z [M + 1]: 679.3 ¹HNMR (400 MHZ, CD₃OD) δ 8.51 (s, 1H), 7.67 (s, 1H), 7.17-7.20 (m, 1H), 6.93 (t, J = 8.4 Hz, 1H), 6.61-6.71 (m, 1H), 6.36 (d, J = 16.8 Hz, 1H), 5.84- 5.99 (m, 1H), 5.81-5.82(m, 1H), 5.65 (d, J = 51.6 Hz, 1H), 5.39 (d, J = 52.4 Hz, 1H), 4.34-4.50 (m, 3H), 3.74-4.15 (m, 3H), 3.40-3.50 (m, 3H), 3.13- 3.21 (m, 1H), 1.99-2.50 (m, 6H). |
| 59 | 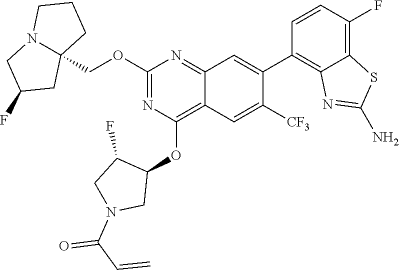 [SMILES: C=CC(=O)N1C[C@H](Oc2nc(OC[C@@]34CCCN3C[C@H](F)C4)nc3cc(-c4ccc(F)c5sc(N)nc45)c(C(F)(F)F)cc23)[C@@H](F)C1] | LCMS m/z [M + 1]: 679.3 ¹HNMR (400 MHZ, CD₃OD) δ 8.43 (d, J = 5.2 Hz, 1H), 7.65 (s, 1H), 7.15-7.18 (m, 1H), 6.92 (t, J = 8.8 Hz, 1H), 6.62-6.71 (m, 1H), 6.35 (d, J = 16.8 Hz, 1H), 5.96-6.05 (m, 1H), 5.80-5.84 (m, 1H), 5.48- 5.66 (m, 1H), 5.37 (d, J = 53.6 Hz, 1H), 4.35-4.45 (m, 2H), 3.94-4.28 (m, 4H), 3.35-3.42 (m, 3H), 3.08-3.16 (m, 1H), 1.96-2.43 (m, 6H). |
| 59a | 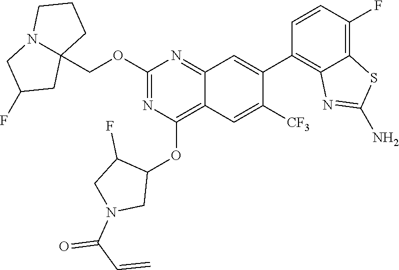 [SMILES: C=CC(=O)N1CC(F)C(Oc2nc(OCC34CCCN3CC(F)C4)nc3cc(-c4ccc(F)c5sc(N)nc45)c(C(F)(F)F)cc23)C1] | |
| 60 | 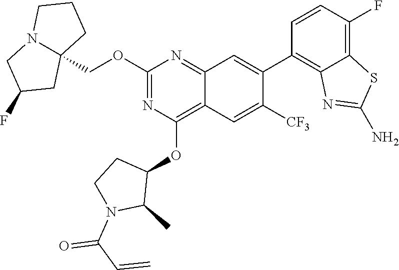 [SMILES: C=CC(=O)N1CC[C@@H](Oc2nc(OC[C@@]34CCCN3C[C@H](F)C4)nc3cc(-c4ccc(F)c5sc(N)nc45)c(C(F)(F)F)cc23)[C@H]1C] | LCMS m/z [M + 1]: 675.2 ¹HNMR (400 MHZ, CD₃OD) δ 8.48 (s, 1H), 7.65 (s, 1H), 7.19 (t, J = 8.4, 5.6 Hz, 1H), 6.93 (t, J = 9.2 Hz, 1H), 6.59-6.75 (m, 1H), 6.25-6.41 (m, 1H), 5.74- 5.95 (m, 2H), 5.41 (d, J = 50.8 Hz, 1H), 4.68-4.81 (m, 1H), 4.20-4.46 (m, 2H), 3.61-3.99 (m, 3H), 3.26-3.41 (m, 2H), 3.02-3.18 (m, 1H), 1.82-2.68 (m, 8H), 1.28 (dd, J = 6.8, 2.8 Hz, 3H). |
| 60a | 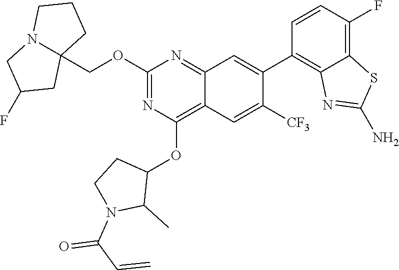 [SMILES: C=CC(=O)N1CCC(Oc2nc(OCC34CCCN3CC(F)C4)nc3cc(-c4ccc(F)c5sc(N)nc45)c(C(F)(F)F)cc23)C1C] | |
| 61 | 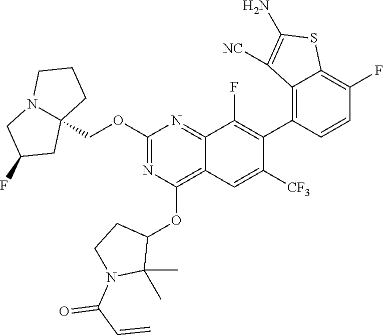 [SMILES: C=CC(=O)N1CCC(Oc2nc(OC[C@@]34CCCN3C[C@H](F)C4)nc3c(F)c(-c4ccc(F)c5sc(N)c(C#N)c45)c(C(F)(F)F)cc23)C1(C)C] | LCMS m/z [M + 1]: 731.3 ¹HNMR (400 MHZ, CD₃OD): δ 8.25 (s, 1H), 7.19- 7.24 (m, 1H), 7.02 (t, J = 8.8 Hz, 1H), 6.64-6.71 (m, 1H), 6.23-6.28 (m, 1H), 5.67-5.75 (m, 2H), 5.34 (d, J = 53.6 Hz, 1H), 4.34-4.46 (m, 2H), 3.87-3.97 (m, 2H), 3.27-3.35 (m, 3H), 3.05-3.13 (m, 1H), 1.95- 2.66 (m, 8H), 1.68-1.72 (m, 3H), 1.63 (s, 3H). |
| 61a | 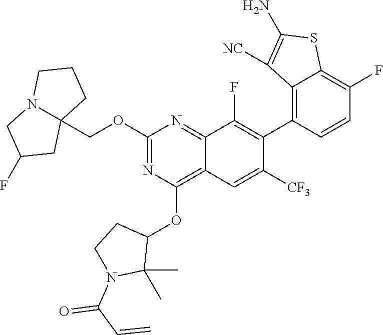 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1(C)C] | |
| 62 | 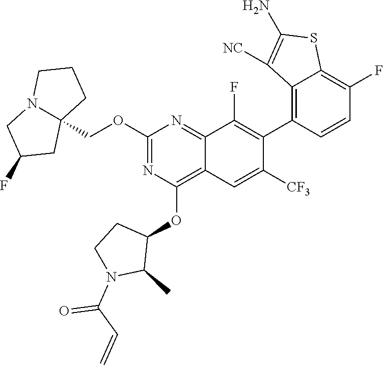 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 717.3 ¹HNMR (400 MHZ, CD₃OD) δ 8.31 (s, 1H), 7.21- 7.29 (m, 1H), 7.04 (t, J = 9.2 Hz, 1H), 6.67-6.77 (m, 1H), 6.35 (t, J = 16.8 Hz, 1H), 5.75-5.94 (m, 2H), 5.34 (d, J = 54.4 Hz , 1H), 4.70-4.83 (m, 1H), 4.28-4.51 (m, 2H), 3.62-3.98 (m, 2H), 3.22-3.29 (m, 1H), 3.07-3.12 (m, 1H), 2.16-2.64 (m, 5 H), 1.85-2.01 (m, 3H), 1.29-1.35 (m, 3H), 0.71-0.96 (m, 3H). |
| 63 | 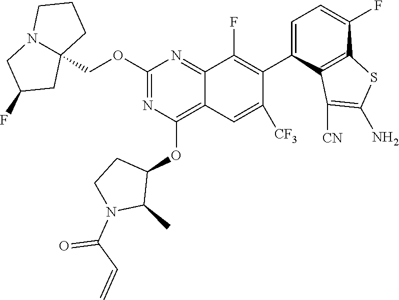 | LCMS m/z [M + 1]: 717.3 ¹HNMR (400 MHz, CD₃OD) δ 8.28 (s, 1H), 7.20- 7.26 (m, 1H), 6.98-7.06 (m, 1H), 6.62-6.73 (m, 1H), 6.27-6.40 (m, 1H), 5.75-5.90 (m, 2H), 5.30 (d, J = 54.8 Hz, 1H), 4.70-4.83 (m, 1H), 4.29-4.41 (m, 2H), 3.66-3.95 (m, 2H), 2.99-3.25 (m, 4H), 1.85-2.64 (m, 8H), 1.27-1.35 (m, 3H). |
| 64 | 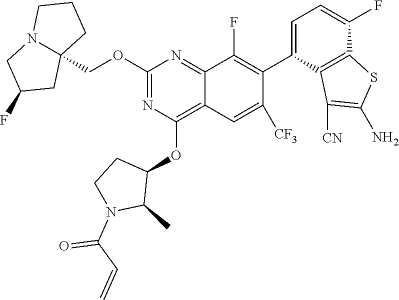 | LCMS m/z [M + 1]: 717.4 ¹HNMR (400 MHZ, CD₃OD) δ 8.28 (s, 1H), 7.20- 7.26 (m, 1H), 6.98-7.06 (m, 1H), 6.62-6.73 (m, 1H), 6.27-6.40 (m, 1H), 5.75-5.90 (m, 2H), 5.30 (d, J = 54.8 Hz, 1H), 4.70-4.83 (m, 1H), 4.29-4.41 (m, 2H), 3.66-3.95 (m, 2H), 2.99-3.25 (m, 4H), 1.85-2.64 (m, 8H), 1.27-1.35 (m, 3H). |
| 64a | 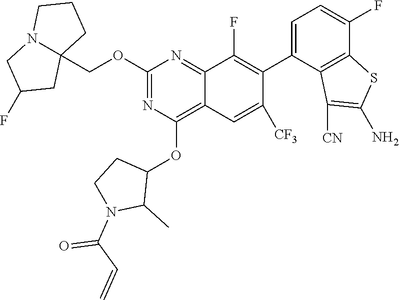 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1C] | |
| 65 | 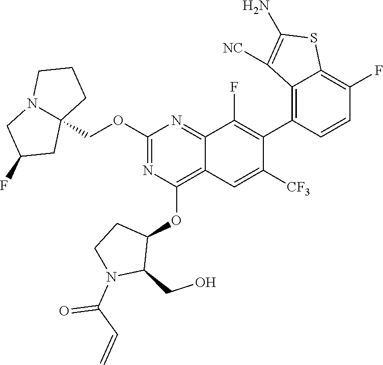 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@H]1CO] | LCMS m/z [M + 1]: 733.3 ¹HNMR (400 MHZ, CD₃OD) δ 8.36 (d, J = 8.0 Hz, 1H), 7.18-7.24 (m, 1H), 7.01 (t, J = 8.8 Hz, 1H), 6.39-6.43 (m, 1H), 6.18-6.25 (m, 1H), 5.90-5.92 (m, 1H), 5.35 (d, J = 52.8 Hz, 1H), 5.02-5.08 (m, 1H), 4.78-4.80 (m, 1H), 4.59-4.63 (m, 2H), 4.36-4.45 (m, 4H), 4.09-4.13 (m, 1H), 3.21-3.55 (m, 2H), 3.09- 3.17 (m, 1H), 1.97-2.45 (m, 8H). |
| 65a | 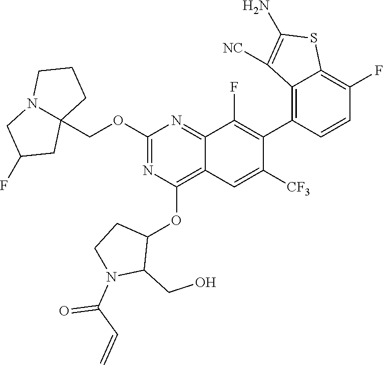 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1CO] | |
| 66 | 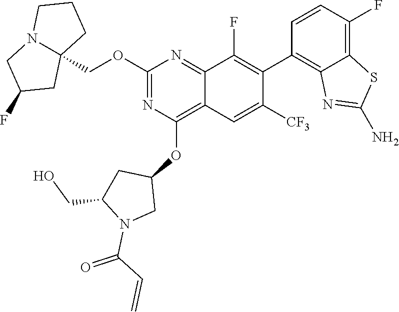 [SMILES: C=CC(=O)N1C[C@H](Oc2nc(OC[C@@]34CCCN3C[C@H](F)C4)nc3c(F)c(-c4ccc(F)c5sc(N)nc45)c(C(F)(F)F)cc23)C[C@H]1CO] | LCMS m/z [M + 1]: 709.3 ¹HNMR (400 MHZ, CD₃OD) δ 8.29 (d, J = 9.6 Hz, 1H), 7.17-7.22 (m, 1H), 6.97 (t, J = 8.8 Hz, 1H), 6.83 (dd, J = 16.8, 6.4 Hz, 1H), 6.60-6.68 (m, 1H), 6.29-6.37 (m, 1H), 5.96-6.03 (m, 1H), 5.76-5.81 (m, 1H), 5.32 (d, J = 52.8 Hz, 1H), 4.23-4.47 (m, 3H), 4.06-4.12 (m, 1H), 3.96-4.02 (m, 1H), 3.83-3.91(m, 1H), 3.70-3.78 (m, 1H), 3.56-3.64 (m, 1H), 3.21- 3.25 (m, 1H), 3.01-3.08 (m, 1H), 2.53-2.65 (m, 2H), 2.13-2.38 (m, 3H) 1.87-2.04 (m, 3H). |
| 66a | 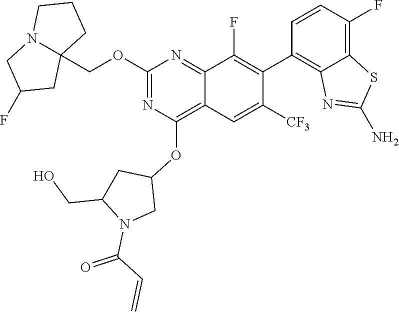 [SMILES: C=CC(=O)N1CC(Oc2nc(OCC34CCCN3CC(F)C4)nc3c(F)c(-c4ccc(F)c5sc(N)nc45)c(C(F)(F)F)cc23)CC1CO] | |
| 67 | 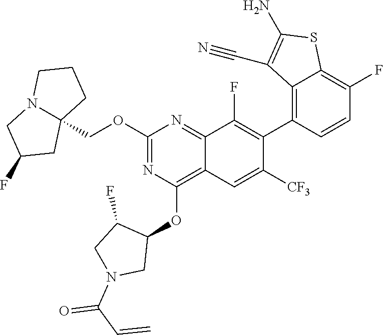 [SMILES: C=CC(=O)N1C[C@H](Oc2nc(OC[C@@]34CCCN3C[C@H](F)C4)nc3c(F)c(-c4ccc(F)c5sc(N)c(C#N)c45)c(C(F)(F)F)cc23)[C@@H](F)C1] | LCMS m/z [M + 1]: 721.2 ¹HNMR(400 MHZ, CD₃OD) δ 8.24-8.28 (m, 1H), 7.15-7.26 (m, 1H), 7.02 (t, J = 9.2 Hz, 1H), 6.61- 6.72 (m, 1H), 6.32-6.38 (m, 1H), 5.93-6.08 (m, 1H), 5.77-5.85 (m, 1H), 5.45-5.67 (m, 1H), 5.36 (d, J = 54.4 Hz, 1H), 4.36-4.64 (m, 3H), 3.91-4.30 (m, 4H), 3.34-3.45 (m, 3H), 3.05-3.16 (m, 1H), 2.16-2.47 (m, 3H), 1.90-2.15 (m, 3H). |
| 67a | 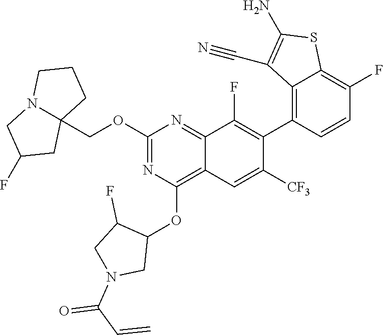 [SMILES: C=CC(=O)N1CC(F)C(Oc2nc(OCC34CCCN3CC(F)C4)nc3c(F)c(-c4ccc(F)c5sc(N)c(C#N)c45)c(C(F)(F)F)cc23)C1] | |
| 68 | 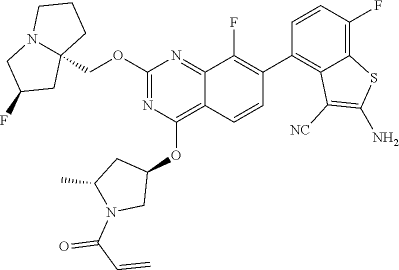 [SMILES: C=CC(=O)N1C[C@H](Oc2nc(OC[C@@]34CCCN3C[C@H](F)C4)nc3c(F)c(-c4ccc(F)c5sc(N)c(C#N)c45)ccc23)C[C@H]1C] | LCMS m/z [M + 1]: 649.4 ¹HNMR (400 MHZ, CD₃OD) δ 7.84-7.88 (m, 1H), 7.32-7.39 (m, 1H), 7.21-7.25 (m, 1H), 7.01 (t, J = 8.8 Hz, 1H), 6.54-6.76 (m, 1H), 6.19-6.40 (m, 1H), 5.85-6.02 (m, 1H), 5.68-5.85 (m, 1H), 5.38 (d, J = 52.0 Hz, 1H), 4.35-4.77 (m, 3H), 3.90-4.16 (m, 2H), 3.33-3.54 (m, 3H), 3.08-3.23 (m, 1 H), 2.59- 2.71 (m, 1H), 1.96-2.47 (m, 7H), 1.32-1.48 (m, 3H). |
| 68a | 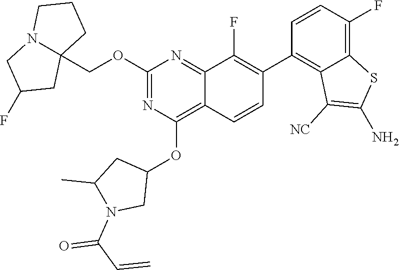 [SMILES: C=CC(=O)N1CC(Oc2nc(OCC34CCCN3CC(F)C4)nc3c(F)c(-c4ccc(F)c5sc(N)c(C#N)c45)ccc23)CC1C] | |
| 69 | 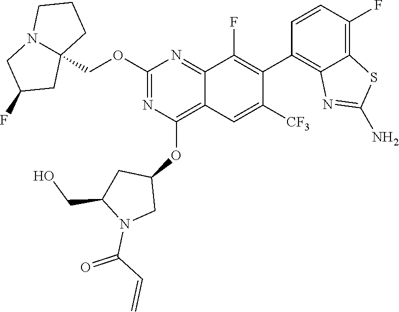 [SMILES: C=CC(=O)N1C[C@H](Oc2nc(OC[C@@]34CCCN3C[C@H](F)C4)nc3c(F)c(-c4ccc(F)c5sc(N)nc45)c(C(F)(F)F)cc23)C[C@@H]1CO] | LCMS m/z [M + 1]: 709.2 ¹HNMR(400 MHZ, CD₃OD) δ 8.20-8.28 (m, 1H), 7.12-7.22 (m, 1H), 6.97 (t, J = 9.2 Hz, 1H), 6.58- 6.87 (m, 1H), 6.22-6.37 (m, 1H), 5.91-6.07 (m, 1H), 5.71-5.82 (m, 1H), 5.42 (d, J = 55.6 Hz, 1H), 4.36- 4.65 (m, 3H), 3.90-4.25 (m, 4H), 3.61-3.79 (m, 1H), 3.35-3.54 (m, 3H), 3.09-3.22 (m, 1H), 2.51-2.69 (m, 2H), 2.18-2.49 (m, 3H), 1.90-2.15 (m, 3H). |
| 70 | 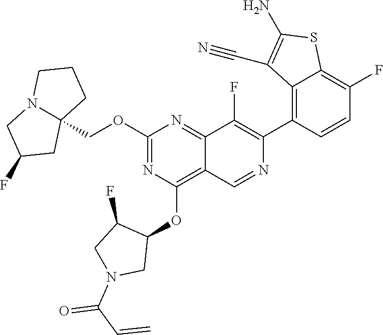 [SMILES: C=CC(=O)N1C[C@@H](F)[C@@H](Oc2nc(OC[C@@]34CCCN3C[C@H](F)C4)nc3c(F)c(-c4ccc(F)c5sc(N)c(C#N)c45)ncc23)C1] | LCMS m/z [M + 1]: 654.2 ¹HNMR (400 MHZ, CD₃OD) δ 9.26 (s, 1H), 8.39 (s, 1H), 7.39-7.44 (m, 1H), 7.07 (t, J = 9.08 Hz, 1H), 6.61-6.69 (m, 1H), 6.33-6.37 (m, 1H), 5.81-5.99 (m, 1H), 5.39-5.71 (m, 2H), 4.30-4.63 (m, 3H), 3.93- 4.15 (m, 2H), 3.55-3.76 (m, 3H), 2.00-2.73 (m, 8H). |
| 70a | 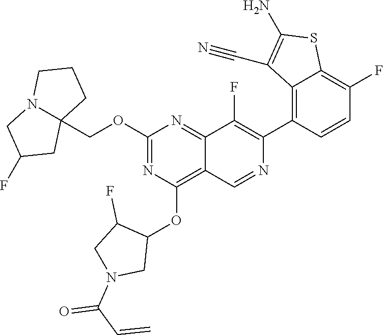 [SMILES: C=CC(=O)N1CC(F)C(Oc2nc(OCC34CCCN3CC(F)C4)nc3c(F)c(-c4ccc(F)c5sc(N)c(C#N)c45)ncc23)C1] | |
| 71 | 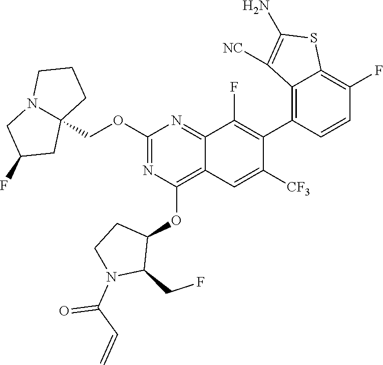 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@H]1CF] | LCMS m/z [M + 1]: 735.2 ¹HNMR (400 MHZ, CD₃OD) δ 8.28-8.30 (m, 1H), 7.20-7.23 (m, 1H), 7.02 (t, J = 8.84 Hz, 1H), 6.61- 6.72 (m, 1H), 6.31-6.35 (m, 1H), 5.79-5.83 (m, 1H), 5.29-5.52 (m, 2H), 4.72-4.83 (m, 4H), 4.39-4.51 (m, 2H), 3.80-3.95 (m, 2H), 3.41-3.47 (m, 2H), 3.12- 3.18 (m, 1H), 2.10-2.59 (m, 8H). |
| 71a | 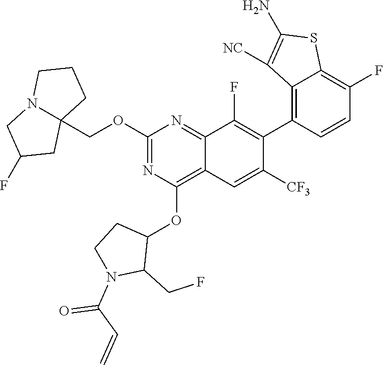 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1CF] | |

Table 3 include selected compounds of the present disclosure.

TABLE 3
Selected compounds of the present disclosure.

| Compound | | |
|---|---|---|
| No. | Structure | Analytical data |
| 73 | 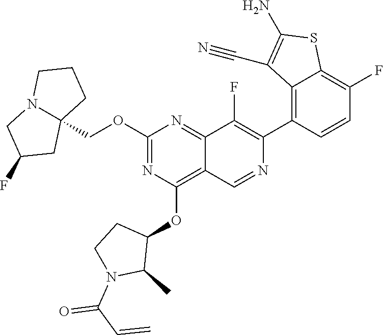 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ncc23)[C@H]1C] | LCMS m/z [M + 1]: 650.2 ¹H NMR (400 MHz, CD₃OD) δ 9.22 (s, 1H), 7.40- 7.43 (m, 1H), 7.06 (t, J = 8.0 Hz, 1H), 6.62- 6.69 (m, 1H), 6.28-6.33 (m, 1H), 5.77-5.90 (m, 2H), 5.30 (d, J = 54.8 Hz, 1H), 4.44-4.70 (m, 3H), 3.38-3.81 (m, 5H), 3.13-3.14 (m, 1H), 2.08-2.52 (m, 8H), 1.33 (d, J = 6.4 Hz, 3H). |
| 73a | 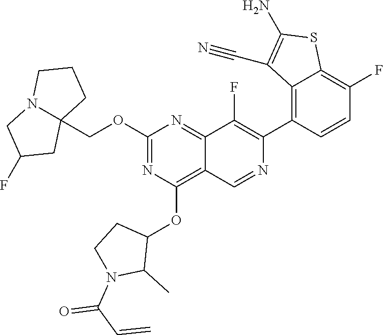 [SMILES: C=CC(=O)N1CCC(Oc2nc(OCC34CCCN3CC(F)C4)nc3c(F)c(-c4ccc(F)c5sc(N)c(C#N)c45)ncc23)C1C] | |
| 75 | 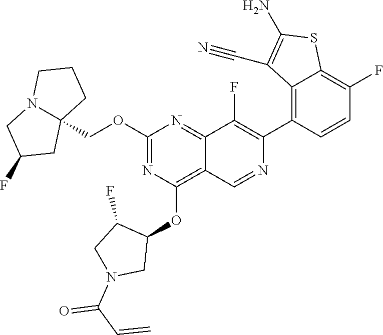 [SMILES: C=CC(=O)N1C[C@H](Oc2nc(OC[C@@]34CCCN3C[C@H](F)C4)nc3c(F)c(-c4ccc(F)c5sc(N)c(C#N)c45)ncc23)[C@@H](F)C1] | LCMS m/z [M + 1]: 654.3 ¹H NMR (400 MHz, CD₃OD) δ 9.23-9.26 (m, 1H), 7.42-7.46 (m, 1H), 7.10 (t, J = 12.0 Hz, 1H), 6.65- 6.75 (m, 1H), 6.25-6.30 (m, 1H), 6.00-6.12 (m, 1H), 5.78-5.82 (m, 1H), 5.56-5.75 (m, 2H), 4.75-4.82 (m, 2H), 3.80-4.25 (m, 7H), 3.48-3.58 (m, 1H), 2.56-2.72 (m, 2H), 2.30-2.50 (m, 3H), 2.18-2.25 (m, 1H). |
| 76 | 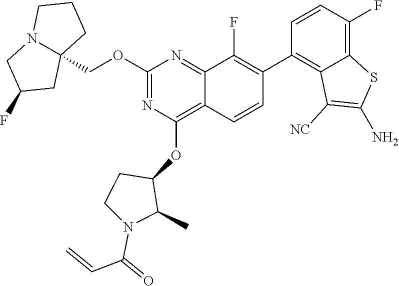 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@H]1C] | LCMS m/z [M + 1]: 649.3 ¹H NMR (400 MHz, CD₃OD) δ 7.99 (d, J = 8.36 Hz, 1H), 7.40 (t, J = 6.96 Hz, 1H), 7.26 (t, J = 6.92 Hz, 1H), 7.03 (t, J = 8.88 Hz, 1H), 6.62-6.72 (m, 1H), 6.33 (t, J = 17.76 Hz, 1H), 5.77-5.88 (m, 2H), 5.38 (d, J = 53.88 Hz, 1H), 4.68-4.79 (m, 1H), 4.34- 4.52 (m, 2H), 3.63-3.94 (m, 2H), 3.39-3.48 (m, 3H), 3.13 (s, 1H), 2.22-2.59 (m, 5H), 1.95-2.10 (m, 3H), 1.30-1.32 (m, 3H) |
| 76a | 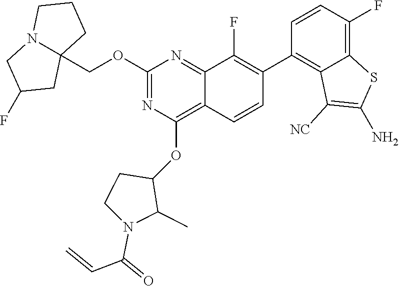 [SMILES: C=CC(=O)N1CCC(Oc2nc(OCC34CCCN3CC(F)C4)nc3c(F)c(-c4ccc(F)c5sc(N)c(C#N)c45)ccc23)C1C] | |
| 77 | 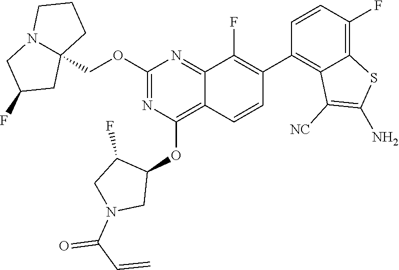 [SMILES: C=CC(=O)N1C[C@H](Oc2nc(OC[C@@]34CCCN3C[C@H](F)C4)nc3c(F)c(-c4ccc(F)c5sc(N)c(C#N)c45)ccc23)[C@@H](F)C1] | LCMS m/z [M + 1]: 653.2 ¹H NMR (400 MHz CD₃OD) δ 7.90-7.92 (m, 1H), 7.37-7.40 (m, 1H), 7.22-7.25 (m, 1H), 7.00-7.04 (m, 1H), 6.63-6.65 (m, 1H), 6.32-6.36 (m, 1H), 5.78-6.09 (m, 2H), 5.49-5.83 (m, 2H), 4.47-4.57 (m, 2H), 3.90-4.29 (m, 4H), 3.47-3.56 (m, 3H), 3.21-3.23 (m, 1H), 2.03-2.45 (m, 6H). |
| 77a | 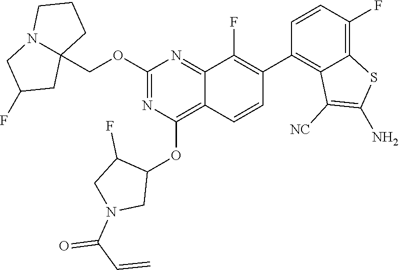 [SMILES: C=CC(=O)N1CC(F)C(Oc2nc(OCC34CCCN3CC(F)C4)nc3c(F)c(-c4ccc(F)c5sc(N)c(C#N)c45)ccc23)C1] | |
| 78 | 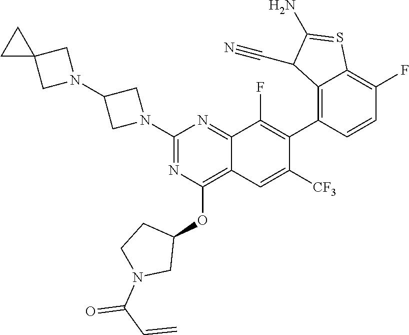 | LCMS m/z [M + 1]: 682.3 ¹H NMR (400 MHz CD₃OD) δ 8.09 (s, 1H), 7.16- 7.17 (m, 1H), 6.99 (t, J = 9.08 Hz, 1H), 6.58-6.72 (m, 1H), 6.29-6.33 (m, 1H), 5.88-5.96 (m, 1H), 5.75-5.79 (m, 1H), 4.40-4.42 (m, 2H), 3.76-4.09 (m, 2H), 3.80-3.95 (m, 7H), 3.65-3.69 (m, 4H), 2.36- 2.52 (m, 2H), 0.66-0.67 (m, 4H). |
| 78a | 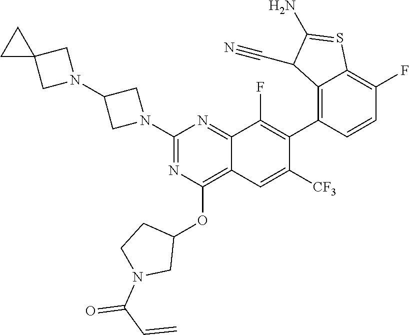 | |
| 79 | 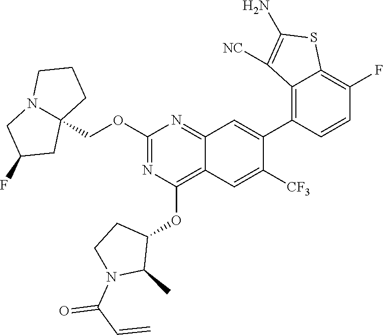 | LCMS m/z [M + 1]: 717.3 ¹H NMR (400 MHz, CD₃OD) δ 8.21-8.24 (m, 1H), 7.20 (s, 1H), 7.01 (t, J = 8.92 Hz, 1H), 6.63-6.73 (m, 1H), 6.32 (t, J = 14.8 Hz, 1H), 5.60-5.79 (m, 2H), 5.39 (d, J = 50.28 Hz, 1H), 4.41-4.52 (m, 2H), 3.83- 3.99 (m, 2H), 3.35-3.47 (m, 3H), 3.12-3.17 (m, 2H), 2.23-2.67 (m, 5H), 2.01-2.10 (m, 3H), 1.41-1.43 (m, 3H). |
| 79a | 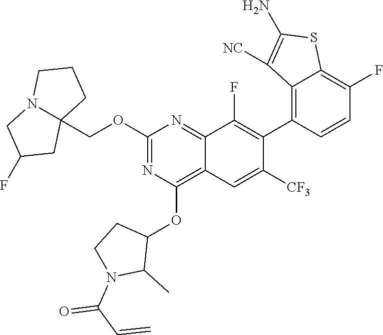 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1C] | |
| 80 | 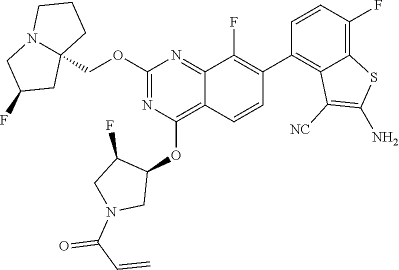 [SMILES: C=CC(=O)N1C[C@@H](F)[C@@H](Oc2nc(OC[C@@]34CCCN3C[C@H](F)C4)nc3c(F)c(-c4ccc(F)c5sc(N)c(C#N)c45)ccc23)C1] | LCMS m/z [M + 1]:653.3 ¹H NMR (400 MHz, CD₃OD) δ 8.00- 8.04 (m, 1H), 7.37-7.49 (m, 1H), 7.26- 7.33 (m, 1H), 7.00-7.16 (m, 1H), 6.62- 6.76 (m, 1H), 6.27-6.36 (m, 1H), 5.63-6.00 (m, 2H), 5.55-5.76 (m, 1H), 5.28-5.50 (m, 1H), 4.26-4.70 (m, 3H), 3.75-4.13 (m, 3H), 3.49- 3.70 (m, 3H), 3.22-3.26 (m, 1H), 2.30- 2.56 (m, 3H), 2.12-2.25 (m, 2H), 2.00-2.14 (m, 1H). |
| 82 | 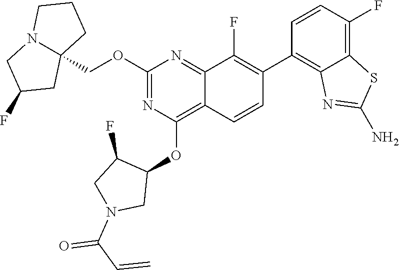 [SMILES: C=CC(=O)N1C[C@@H](F)[C@@H](Oc2nc(OC[C@@]34CCCN3C[C@H](F)C4)nc3c(F)c(-c4ccc(F)c5sc(N)nc45)ccc23)C1] | LCMS m/z [M + 1]: 629.2 ¹H NMR (400 MHz, CD₃OD) δ 7.96 (s, 1H), 7.52- 7.63 (m, 1H), 7.30-7.41 (m, 1H), 6.98 (t, J = 8.8 Hz, 1H), 6.56-6.69 (m, 1H), 6.28-6.40 (m, 1H), 5.77-5.98 (m, 2H), 5.52-5.72 (m, 1H), 5.38 (d, J = 53.2 Hz, 1H), 4.27-4.54 (m, 3H), 3.73-4.14 (m, 3H), 3.34-3.56 (m, 3H), 3.09-3.20 (m, 1H), 2.20- 2.52 (m, 3H), 1.95-2.14 (m, 3H). |
| 82a | 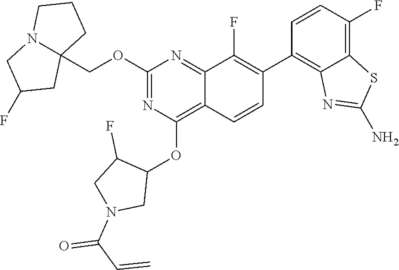 [SMILES: C=CC(=O)N1CC(F)C(Oc2nc(OCC34CCCN3CC(F)C4)nc3c(F)c(-c4ccc(F)c5sc(N)nc45)ccc23)C1] | |
| 83 | 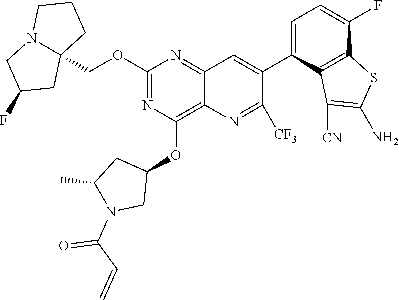 | LCMS m/z [M + 1]: 700.1 ¹H NMR (400 MHz CD₃OD) δ 8.05 (s, 1H), 7.21- 7.27 (m, 1H), 7.01 (t, 1H), 8.57-8.79 (m, 1H), 6.23- 6.37 (m, 1H), 5.98 (s, 1H), 5.71-5.84 (m, 1H), 5.40 (s, 0.5H), 5.27 (s, 0.5H), 4.48-4.65 (m, 1H), 4.29-4.43 (m, 2H), 3.95-4.23 (m, 2H), 3.24 (m, 2H), 3.02-3.10 (m, 1H), 2.70-2.86 (m, 1H), 1.85- 2.40 (m, 8H), 1.42 (t, 3H). |
| 84 | 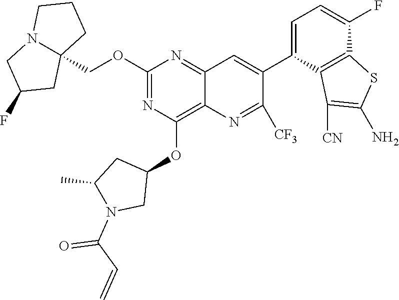 | LCMS m/z [M + 1]: 700.1 ¹H NMR (400 MHz, CD₃OD) δ 8.05 (s, 1H), 7.20- 7.26 (m, 1H), 7.01 (t, 1H), 8.58-8.79 (m, 1H), 6.26- 6.36 (m, 1H), 5.99 (s, 1H), 5.73-5.84 (m, 1H), 5.39 (s, 0.5H), 5.26 (s, 0.5H), 4.48-4.61 (m, 1H), 4.30- 4.40 (m, 2H), 4.02-4.19 (m, 2H), 3.24 (d, 2H), 3.01- 3.10 (m, 1H), 2.67-2.83 (m, 1H), 1.88-2.43 (m, 8H), 1.42 (t, 3H). |
| 84a | 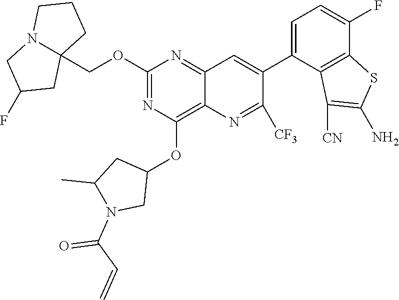 [SMILES: C=CC(=O)N1CC(Oc2nc(OCC34CCCN3CC(F)C4)nc3cc(-c4ccc(F)c5sc(N)c(C#N)c45)c(C(F)(F)F)nc23)CC1C] | |
| 85 | 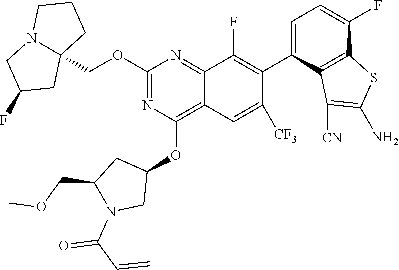 | LCMS m/z [M + 1]: 747.1 ¹H NMR (400 MHz CD₃OD) δ 8.28 (d, 1H), 7.234 (m, 1H), 7.037 (t, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 4.26-4.44 (m, 3H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10- 2.42 (m, 4H), 1.91-2.08 (m, 3H). |
| 86 | 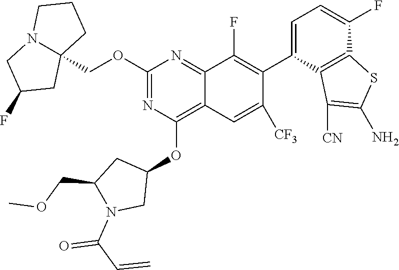 | LCMS m/z [M + 1]: 747.1 ¹H NMR (400 MHz, CD₃OD) δ 8.26-8.34 (m, 1H), 7.21-7.32 (m, 1H), 7.04 (t, 1H), 6.77-6.92 (m, 0.5H), 6.55-6.68 (m, 0.5H), 6.33 (d, 1H), 5.92-6.09 (m, 1H), 5.71-5.85 (m, 1H), 5.41 (s, 0.5H), 5.27 (s, 0.5H), 4.52-4.66 (m, 1H), 4.20-4.48 (m, 3H), 3.83- 3.94 (m, 1H), 3.67-3.81 (m, 1H), 3.57-3.66 (m, 1H), 3.34-3.47 (m, 3H), 3.10-3.32 (m, 3H), 2.54- 2.73 (m, 2H), 2.08-2.53 (m, 4H), 1.87-2.07 (m, 3H). |
| 86a | 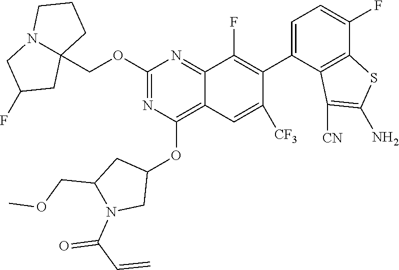 [SMILES: C=CC(=O)N1CC(Oc2nc(OCC34CCCN3CC(F)C4)nc3c(F)c(-c4ccc(F)c5sc(N)c(C#N)c45)c(C(F)(F)F)cc23)CC1COC] | |
| 87 | 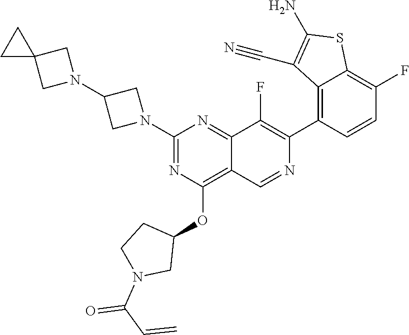 [SMILES: C=CC(=O)N1CC[C@@H](Oc2nc(N3CC(N4CC5(CC5)C4)C3)nc3c(F)c(-c4ccc(F)c5sc(N)c(C#N)c45)ncc23)C1] | LCMS m/z [M + 1]: 615.3 ¹H NMR (400 MHz CD₃OD) δ 8.90 (s, 1H), 7.31- 7.47 (m, 1H), 7.01-7.06 (m, 1H), 6.58-6.71 (m, 1H), 6.30 (d, J = 16.8 Hz, 1H), 5.71-5.98 (m, 2H), 4.35- 4.45 (m, 2H), 3.66-4.08 (m, 7H), 3.50-3.62 (m, 4H), 2.27-2.58 (m, 2H), 0.55-0.70 (m, 4H). |
| 87a | 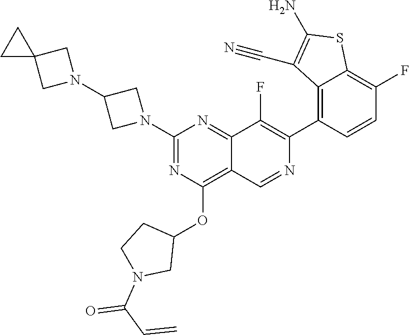 [SMILES: C=CC(=O)N1CCC(Oc2nc(N3CC(N4CC5(CC5)C4)C3)nc3c(F)c(-c4ccc(F)c5sc(N)c(C#N)c45)ncc23)C1] | |
| 88 | 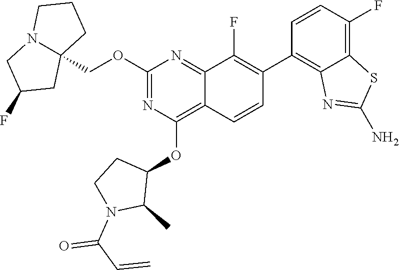 [SMILES: C=CC(=O)N1CC[C@@H](Oc2nc(OC[C@@]34CCCN3C[C@H](F)C4)nc3c(F)c(-c4ccc(F)c5sc(N)nc45)ccc23)[C@H]1C] | LCMS m/z [M + 1]: 625.2 ¹HNMR(400 MHz, CD₃OD): δ 7.93-7.96 (m, 1H), 7.57-7.60 (m, 1H), 7.34-7.38 (m, 1H), 6.98 (t, J = 8.38 Hz, 1H), 6.61-6.70 (m, 1H), 6.28-6.37 (m, 1H), 5.77-5.85 (m,2H), 5.35-5.51 (m, 1H), 4.44-4.75 (m, 3H), 3.48-3.93 (m, 5H), 3.18-3.27 (m, 1H), 2.03- 2.58 (m,8H), 1.26 (d, J = 6.96 Hz, 3H). |
| 88a | 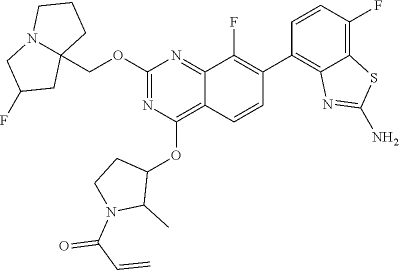 [SMILES: C=CC(=O)N1CCC(Oc2nc(OCC34CCCN3CC(F)C4)nc3c(F)c(-c4ccc(F)c5sc(N)nc45)ccc23)C1C] | |
| 89 | 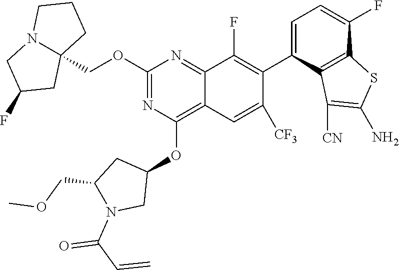 | LCMS m/z [M + 1]: 747.1 ¹H NMR (400 MHz CD₃OD) δ 8.23 (s, 1H), 7.21- 7.24 (m, 1H), 7.00-7.05 (m, 1H), 6.79-6.81 (m, 0.5H), 6.61-6.68 (m, 0.5H), 6.29-6.37 (m, 1H), 5.96-6.02 (m, 1H), 5.76-5.82 (m, 1H), 5.29-5.42 (m, 2H), 4.60-4.65 (m, 1H), 4.34-4.38 (m, 1H), 4.43- 4.45 (m, 1H), 4.11-4.24 (m, 1H), 3.83-3.92 (m, 1H), 3.49-3.58 (m, 2H), 3.41 (s, 3H), 3.08 (s, 1H), 2.56- 2.70 (m, 2H), 2.19-2.39 (m, 4H), 2.04-2.05 (m, 3H), 1.62 (s, 1H). |
| 90 | 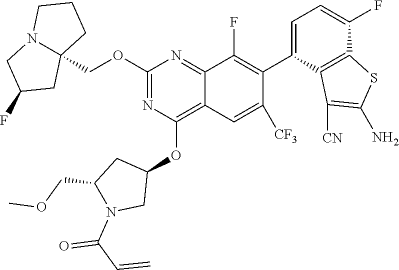 | LCMS m/z [M + 1]: 747.2 ¹H NMR (400 MHz, CD₃OD) δ 8.23 (s, 1H), 7.21- 7.22 (m, 1H), 7.01-7.05 (m, 1H), 6.79-6.81 (m, 0.5H), 6.61-6.68 (m, 0.5H), 6.29-6.33 (m, 1H), 6.01 (s, 1H), 5.76-5.79 (m, 1H), 5.27-5.41 (m, 2H), 4.62- 4.71 (m, 1H), 4.37-4.43 (m, 2H), 4.12-4.14 (m, 1H), 3.81-3.83 (m, 1H), 4.55-4.65 (m, 2H), 3.41 (s, 3H), 3.06 (s, 1H), 2.59-2.65 (m, 2H), 2.04-2.37 (m, 7H), 1.62 (s, 1H). |
| 91 | 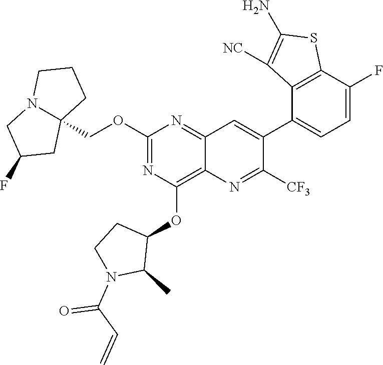 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C(F)(F)F)nc23)[C@H]1C] | LCMS m/z [M + 1]: 700.1 ¹H NMR (400 MHz CD₃OD) δ 8.06 (s, 1H), 7.20- 7.29 (m, 1H), 7.02 (t, 1H), 6.64-6.75 (m, 1H), 6.25- 6.41 (m, 1H), 5.75-5.92 (m, 2H), 5.38 (s, 0.5H), 5.25 (s, 0.5H), 4.71-4.85 (m, 1H), 4.22-4.43 (m, 2H), 3.65-3.98 (m, 2H), 3.15-3.30 (m, 3H), 2.95- 3.08 (m, 1H), 2.45-2.67 (m, 2H), 1.85-2.39 (m, 6H), 1.32-1.37 (t, 3H). |
| 91a | 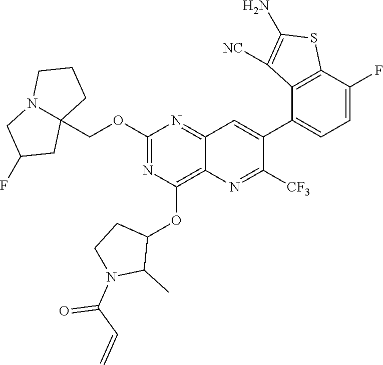 [SMILES: C=CC(=O)N1CCC(Oc2nc(OCC34CCCN3CC(F)C4)nc3cc(-c4ccc(F)c5sc(N)c(C#N)c45)c(C(F)(F)F)nc23)C1C] | |
| 93 | 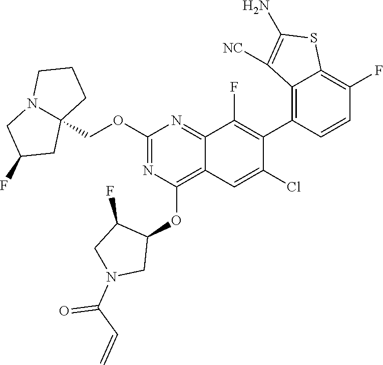 [SMILES: C=CC(=O)N1C[C@@H](F)[C@@H](Oc2nc(OC[C@@]34CCCN3C[C@H](F)C4)nc3c(F)c(-c4ccc(F)c5sc(N)c(C#N)c45)c(Cl)cc23)C1] | LCMS m/z [M + 1]: 687.2 ¹H NMR (400 MHz, CD₃OD) δ 8.06 (s, 1H), 7.20- 7.22 (m, 1H), 7.04 (t, J = 8.8 Hz, 1H), 6.62-6.67 (m, 1H), 6.33-6.37 (m, 1H), 5.77-5.90 (m, 2H), 5.30- 5.83 (m, 2H), 4.46-4.86 (m, 3H), 3.94-4.15 (m, 3H), 3.13-3.41 (m, 4H), 2.03-2.37 (m, 6H). |
| 93a | 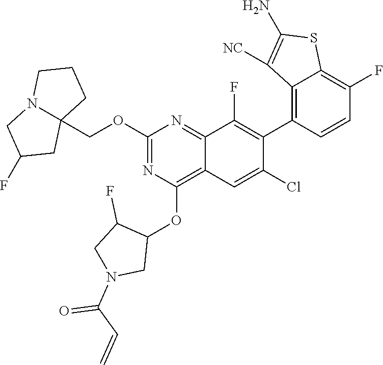 [SMILES: C=CC(=O)N1CC(F)C(Oc2nc(OCC34CCCN3CC(F)C4)nc3c(F)c(-c4ccc(F)c5sc(N)c(C#N)c45)c(Cl)cc23)C1] | |
| 94 | 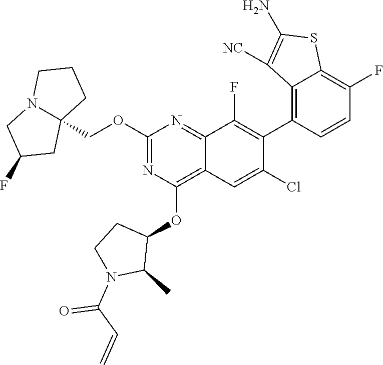 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 683.2 ¹H NMR (400 MHz, CD₃OD) δ 8.02 (s, 1H), 7.20- 7.22 (m, 1H), 7.04 (t, J = 8.8 Hz, 1H), 6.62-6.67 (m, 1H), 6.33-6.37 (m, 1H), 5.77-5.84 (m, 2H), 5.38 (d, J = 51.78 Hz, 1H), 4.33-4.86 (m, 4H), 3.78-3.95 (m, 2H), 3.07-3.30 (m, 3H), 1.93-2.51 (m, 8H), 1.29- 1.32 (m, 3H). |
| 94a | 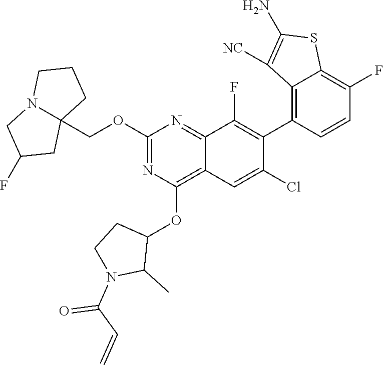 [SMILES: C=CC(=O)N1CCC(Oc2nc(OCC34CCCN3CC(F)C4)nc3c(F)c(-c4ccc(F)c5sc(N)c(C#N)c45)c(Cl)cc23)C1C] | |
| 95 | 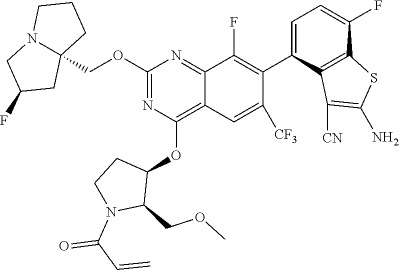 | LCMS m/z [M + 1]: 747.2 ¹HNMR (400 MHz, CD₃OD): δ 8.34 (s, 1H), 7.21- 7.24 (m, 1H), 7.02 (t, J = 9.04 Hz, 1H), 6.64-6.76 (m, 1H), 6.30-6.34 (m, 1H), 5.94-5.97 (m, 1H), 5.77-5.82 (m, 1H), 5.39 (d, J = 50.28 Hz, 1H), 4.39- 4.77 (m, 3H), 3.37-3.89 (m, 6 H), 3.13-3.25 (m, 5H), 1.98-2.60 (m, 8H). |
| 96 | 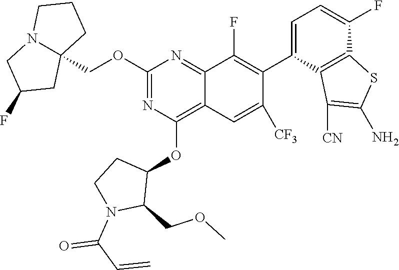 | LCMS m/z [M + 1]: 747.3 ¹HNMR (400 MHz, CD₃OD): δ 8.33 (d, J = 6.69 Hz, 1H), 7.20-7.23 (m, 1H), 7.02 (t, J = 8.94 Hz, 1H), 6.64-6.76 (m, 1H), 6.30-6.34 (m, 1H), 5.94- 5.99 (m, 1H), 5.77-5.82 (m, 1H), 5.35 (d, J = 53.32 Hz, 1H), 4.36-4.76 (m, 3H),3.37-3.89 (m, 6 H), 3.09-3.22 (m, 5H), 1.95-2.60 (m, 8H). |
| 96a | 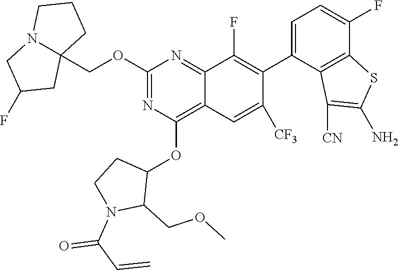 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1COC] | |
| 97 | 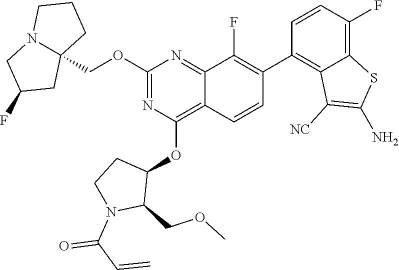 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@H]1COC] | LCMS m/z [M + 1]: 679.3 ¹HNMR (400 MHz, CD₃OD) δ 7.94-7.97 (m, 1H), 7.36-7.41 (m, 1H), 7.23-7.27 (m, 1H), 7.02 (t, J = 8.94 Hz, 1H), 6.61-6.78 (m, 1H), 6.26-6.31 (m, 1H), 5.70-5.78 (m, 2H), 5.38 (d, J = 51.78Hz, 1H), 4.68- 4.78 (m, 2H), 4.43-4.57 (m, 2H), 3.37-3.83 (m, 6H), 3.21-3.26 (m, 3H), 3.11-3.13 (m, 1H), 1.99-2.52 (m, 8H). |
| 97a | 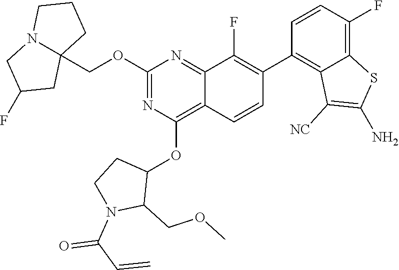 [SMILES: C=CC(=O)N1CCC(Oc2nc(OCC34CCCN3CC(F)C4)nc3c(F)c(-c4ccc(F)c5sc(N)c(C#N)c45)ccc23)C1COC] | |
| 98 | 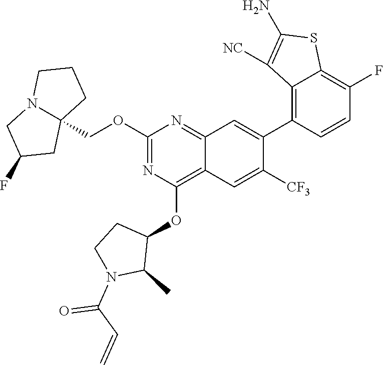 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C(F)(F)F)cc23)[C@H]1C] | LCMS m/z [M + 1]: 699.2 ¹H NMR (400 MHz, CD₃OD) δ 8.51 (s, 1H), 7.69 (s, 1H), 7.15-7.25 (m, 1H), 6.99 (t, J = 9.0 Hz, 1H), 6.53-6.72 (m, 1H), 6.34 (t, J = 15.8 Hz, 1H), 5.77- 5.90 (m, 2H), 5.60 (d, J = 51.4 Hz, 1H), 4.56-4.78 (m, 3H), 3.45-4.01 (m, 6H), 2.19-2.79 (m, 8H), 1.26-1.35 (m, 3H). |
| 98a | 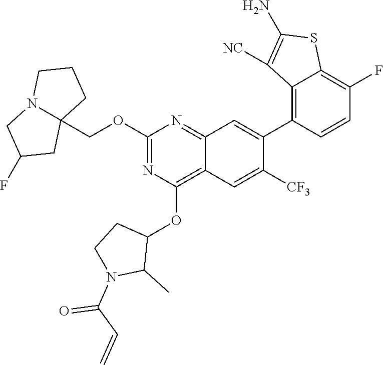 [SMILES: C=CC(=O)N1CCC(Oc2nc(OCC34CCCN3CC(F)C4)nc3cc(-c4ccc(F)c5sc(N)c(C#N)c45)c(C(F)(F)F)cc23)C1C] | |
| 99 | 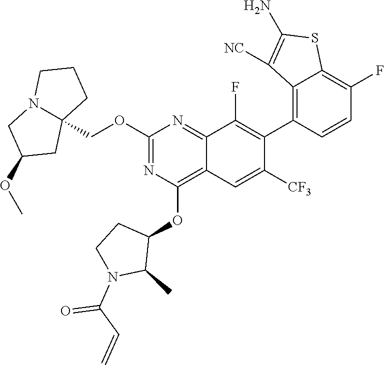 [SMILES: C=CC(=O)N1CC[C@@H](Oc2nc(OC[C@@]34CCCN3C[C@H](OC)C4)nc3c(F)c(-c4ccc(F)c5sc(N)c(C#N)c45)c(C(F)(F)F)cc23)[C@H]1C] | LCMS m/z [M + 1]: 729.3 ¹H NMR (400 MHz, CD₃OD) δ 8.31 (s, 1H), 7.20- 7.22 (m, 1H), 7.02 (t, J = 8.8 Hz, 1H), 6.65-6.69 (m, 1H), 6.29-6.33 (m, 1H), 5.80-5.86 (m, 2H), 4.49- 4.86 (m, 3H), 4.17-4.19 (m, 1H), 3.45-3.80 (m, 7H), 2.00-2.56 (m, 8H), 1.28-1.33 (m, 5H). |
| 99a | 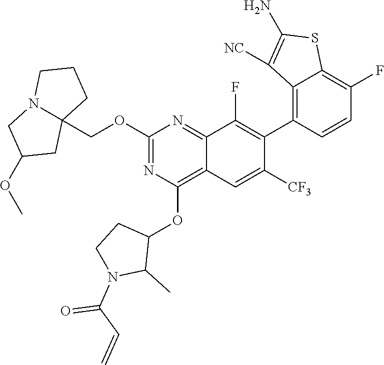 [SMILES: C=CC(=O)N1CCC(Oc2nc(OCC34CCCN3CC(OC)C4)nc3c(F)c(-c4ccc(F)c5sc(N)c(C#N)c45)c(C(F)(F)F)cc23)C1C] | |
| 100 | 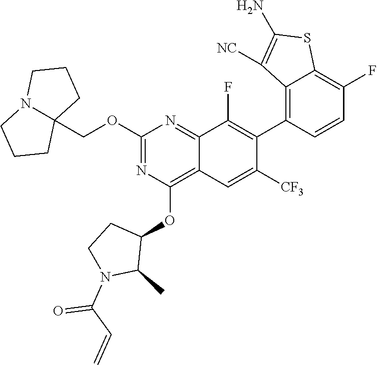 [SMILES: C=CC(=O)N1CC[C@@H](Oc2nc(OCC34CCCN3CCC4)nc3c(F)c(-c4ccc(F)c5sc(N)c(C#N)c45)c(C(F)(F)F)cc23)[C@H]1C] | LCMS m/z [M + 1]: 699.2 ¹H NMR (400 MHz, CD₃OD) δ 8.32 (s, 1H), 7.20- 7.22 (m, 1H), 7.03 (t, J = 8.4 Hz, 1H), 6.65-6.69 (m, 1H), 6.29-6.33 (m, 1H), 5.80-5.87 (m, 2H), 4.59- 4.86 (m, 3H), 3.47-3.92 (m, 6H), 3.12-3.22 (m, 3H), 2.00-2.26 (m, 7H), 1.28-1.33 (m, 3H). |
| 100a | 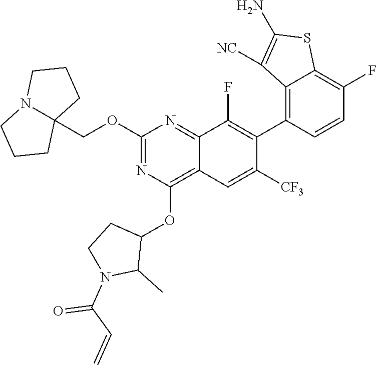 [SMILES: C=CC(=O)N1CCC(Oc2nc(OCC34CCCN3CCC4)nc3c(F)c(-c4ccc(F)c5sc(N)c(C#N)c45)c(C(F)(F)F)cc23)C1C] | |
| 101 | 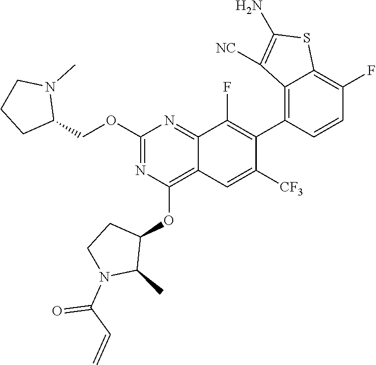 [SMILES: C=CC(=O)N1CC[C@@H](Oc2nc(OC[C@@H]3CCCN3C)nc3c(F)c(-c4ccc(F)c5sc(N)c(C#N)c45)c(C(F)(F)F)cc23)[C@H]1C] | LCMS m/z [M + 1]: 673.2 ¹H NMR (400 MHz, CD₃OD) δ 8.30 (s, 1H), 7.22- 7.24 (m, 1H), 7.02 (t, J = 8.4 Hz, 1H), 6.65-6.69 (m, 1H), 6.29-6.33 (m, 1H), 5.77-5.86 (m, 2H), 4.59- 4.86 (m, 3H), 3.47-3.93 (m, 4H), 2.51-2.75 (m, 7H), 1.98-2.22 (m, 3H), 1.28-1.33 (m, 3H). |
| 101a | 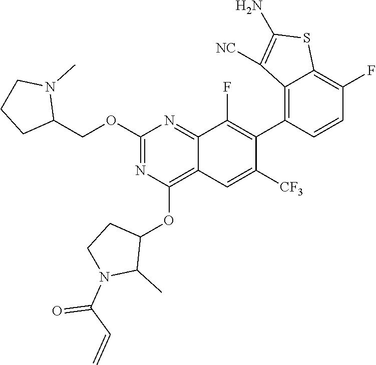 [SMILES: C=CC(=O)N1CCC(Oc2nc(OCC3CCCN3C)nc3c(F)c(-c4ccc(F)c5sc(N)c(C#N)c45)c(C(F)(F)F)cc23)C1C] | |
| 102 | 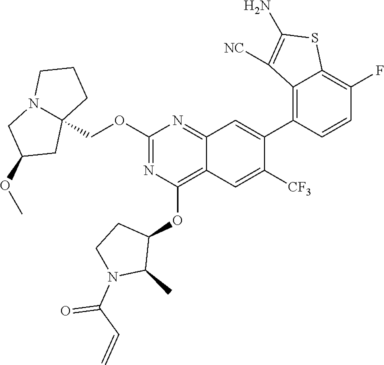 [SMILES: C=CC(=O)N1CC[C@@H](Oc2nc(OC[C@@]34CCCN3C[C@H](OC)C4)nc3cc(-c4ccc(F)c5sc(N)c(C#N)c45)c(C(F)(F)F)cc23)[C@H]1C] | LCMS m/z [M + 1]: 711.3 ¹H NMR (400 MHz, CD₃OD) δ 8.49 (s, 1H), 7.66 (s, 1H), 7.15-7.25 (m, 1H), 6.98 (t, J = 8.8 Hz, 1H), 6.63-6.73 (m, 1H), 6.33 (t, J = 17.0 Hz, 1H), 5.77- 5.85 (m, 2H) , 4.75-4.80 (m, 2H), 4.44-4.56 (m, 2H), 4.15-4.25 (m, 1H), 3.37-3.92 (m, 8H), 2.03- 2.54 (m, 8H), 1.21-1.39 (m, 3H). |
| 102a | 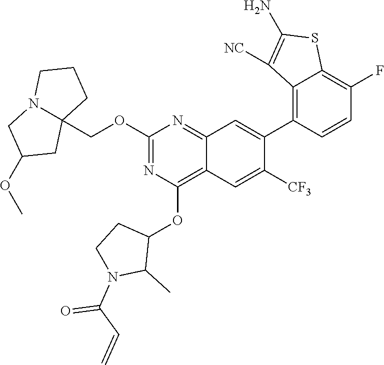 [SMILES: C=CC(=O)N1CCC(Oc2nc(OCC34CCCN3CC(OC)C4)nc3cc(-c4ccc(F)c5sc(N)c(C#N)c45)c(C(F)(F)F)cc23)C1C] | |
| 103 | 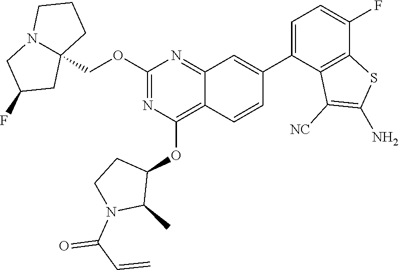 [SMILES: C=CC(=O)N1CC[C@@H](Oc2nc(OC[C@@]34CCCN3C[C@H](F)C4)nc3cc(-c4ccc(F)c5sc(N)c(C#N)c45)ccc23)[C@H]1C] | LCMS m/z [M + 1]: 631.2 ¹H NMR (400 MHz, CD₃OD) δ 8.17 (d, J = 8.2 Hz, 1H), 7.69 (s, 1H), 7.49 (d, J = 8.4 Hz, 1H), 7.22- 7.25 (m, 1H), 6.99 (t, J = 8.8 Hz, 1H), 6.63-6.73 (m, 1H), 6.33 (t, J = 17.0 Hz, 1H), 5.77-5.86 (m, 2H) , 5.45 (d, J = 55.4 Hz, 1H), 4.67-4.79 (m, 1H), 4.36- 4.50 (m, 2H), 3.62-3.94 (m, 2H), 3.37-3.58 (m, 3H), 3.13-3.23 (m, 1H), 1.89-2.57 (m, 8H), 1.29 (d, J = 6.6 Hz, 3H). |
| 103a | 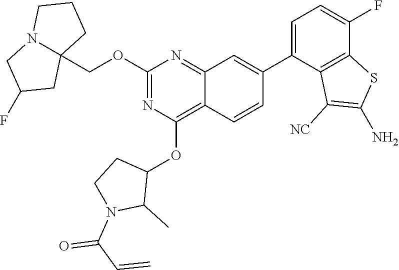 [SMILES: C=CC(=O)N1CCC(Oc2nc(OCC34CCCN3CC(F)C4)nc3cc(-c4ccc(F)c5sc(N)c(C#N)c45)ccc23)C1C] | |
| 104 | 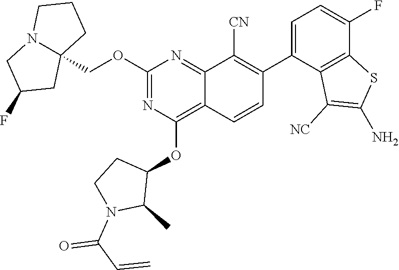 [SMILES: C=CC(=O)N1CC[C@@H](Oc2nc(OC[C@@]34CCCN3C[C@H](F)C4)nc3c(C#N)c(-c4ccc(F)c5sc(N)c(C#N)c45)ccc23)[C@H]1C] | LCMS m/z [M + 1]: 656.0 ¹H NMR (400 MHz, CD₃OD) δ 8.33 (d, J = 8.2 Hz, 1H), 7.47 (d, J = 8.3 Hz, 1H), 7.18-7.25 (m, 1H), 6.97 (t, J = 8.8 Hz, 1H), 6.53-6.63 (m, 1H), 6.20- 6.30 (m, 1H), 5.65-5.80 (m, 2H), 5.30 (d, J = 55.7 Hz, 2H), 4.56-4.64 (m, 1H), 4.37-4.53 (m, 3H), 3.59-3.86 (m, 3H), 3.33-3.43 (m, 1H), 3.04 (t, J = 6.5 Hz, 1H), 2.17-2.46 (m, 5H), 1.89-2.06 (m, 3H), 1.22 (dd, J = 12.3, 6.6 Hz, 3H). |
| 104a | 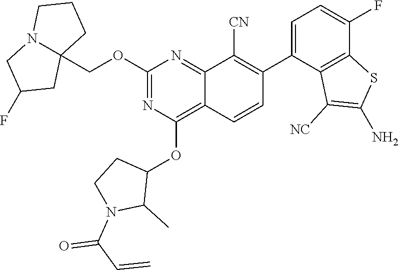 [SMILES: C=CC(=O)N1CCC(Oc2nc(OCC34CCCN3CC(F)C4)nc3c(C#N)c(-c4ccc(F)c5sc(N)c(C#N)c45)ccc23)C1C] | |
| 105 | 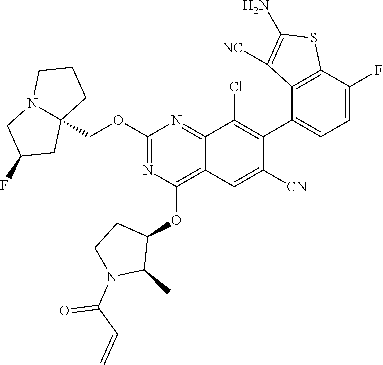 [SMILES: C=CC(=O)N1CC[C@@H](Oc2nc(OC[C@@]34CCCN3C[C@H](F)C4)nc3c(Cl)c(-c4ccc(F)c5sc(N)c(C#N)c45)c(C#N)cc23)[C@H]1C] | LCMS m/z [M + 1]: 690.1 ¹H NMR (400 MHz, CD₃OD) δ 8.47 (d, J = 2.7 Hz, 1H), 7.13-7.20 (m, 1H), 6.99 (t, J = 8.9 Hz, 1H), 6.53-6.64 (m, 1H), 6.18-6.29 (m, 1H), 5.66-5.82 (m, 2H), 5.26 (d, J = 54.5 Hz, 1H), 4.60-4.70 (m, 1H), 4.29-4.52 (m, 3H), 3.79-3.91 (m, 1H), 3.51- 3.77 (m, 2H), 3.26-3.32 (m, 1H), 2.97-3.05 (m, 1H), 2.04-2.54 (m, 5H), 1.84-2.01 (m, 3H), 1.17- 1.28 (m, 3H). |
| 105a | 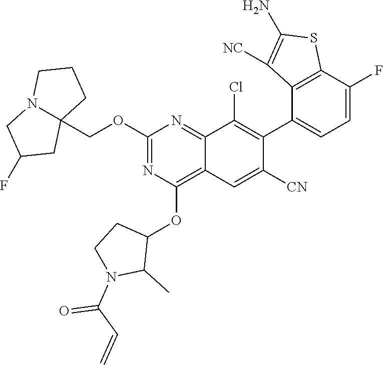 [SMILES: C=CC(=O)N1CCC(Oc2nc(OCC34CCCN3CC(F)C4)nc3c(Cl)c(-c4ccc(F)c5sc(N)c(C#N)c45)c(C#N)cc23)C1C] | |
| 106 | 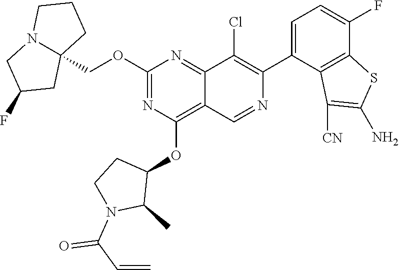 [SMILES: C=CC(=O)N1CC[C@@H](Oc2nc(OC[C@@]34CCCN3C[C@H](F)C4)nc3c(Cl)c(-c4ccc(F)c5sc(N)c(C#N)c45)ncc23)[C@H]1C] | LCMS m/z [M + 1]: 666.2 ¹H NMR (400 MHz, CD₃OD) δ 9.28 (s, 1H), 7.32 (dd, J = 8.3, 5.1 Hz, 1H), 7.00-7.09 (m, 1H), 6.61- 6.73 (m, 1H), 6.28-6.39 (m, 1H), 5.86-5.96 (m, 1H), 5.74-5.83 (m, 1H), 5.32 (d, J = 53.7 Hz, 1H), 4.35-4.80 (m, 4H), 3.65-3.98 (m, 2H), 3.20-3.29 (m, 2H), 2.99-3.10 (m, 1H), 2.16-2.61 (m, 5H), 1.87-2.10 (m, 3H), 1.27-1.38 (m, 3H). |
| 106a | 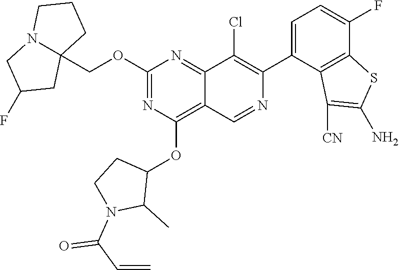 [SMILES: C=CC(=O)N1CCC(Oc2nc(OCC34CCCN3CC(F)C4)nc3c(Cl)c(-c4ccc(F)c5sc(N)c(C#N)c45)ncc23)C1C] | |
| 107 | 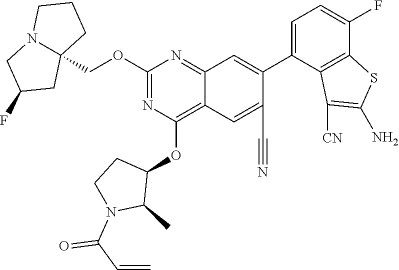 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C#N)cc23)[C@H]1C] | LCMS m/z [M + 1]: 656.3 ¹H NMR (400 MHz, CD₃OD) δ 8.60 (s, 1H), 7.74 (s, 1H), 7.26-7.32 (m, 1H), 7.05 (t, J = 8.9 Hz, 1H), 6.63-6.72 (m, 1H), 6.27-6.38 (m, 1H), 5.77-5.89 (m, 2H), 5.35 (d, J = 51.6 Hz, 1H), 4.69-4.77 (m, 1H), 4.57-4.63 (m, 1H), 4.35-4.46 (m, 2H), 3.89- 3.98 (m, 1H), 3.64-3.84 (m, 2H), 3.35-3.42 (m, 1H), 3.09-3.15 (m, 1H), 2.20-2.57 (m, 5H), 1.95- 2.09 (m, 3H), 1.27-1.35 (m, 3H). |
| 107a | 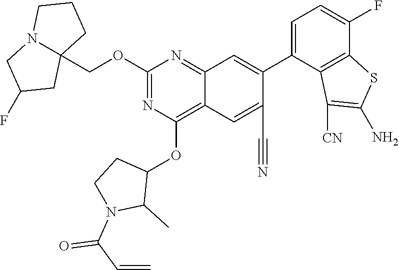 [SMILES: C=CC(=O)N1CCC(Oc2nc(OCC34CCCN3CC(F)C4)nc3cc(-c4ccc(F)c5sc(N)c(C#N)c45)c(C#N)cc23)C1C] | |
| 108 | 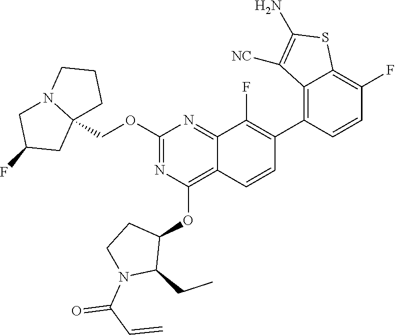 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@H]1CC] | LCMS m/z [M + 1]: 663.0 ¹H NMR (400 MHz, CD₃OD) δ 8.00 (d, J = 8.3 Hz, 1H), 7.43-7.46 (m, 1H), 7.27-7.30 (m, 1H), 7.01- 7.10 (m, 1H), 6.65-6.75 (m, 1H), 6.33-6.36 (m, 1H), 5.93-6.04 (m, 1H), 5.76-5.85 (m, 1H), 5.40 (d, J = 53.8 Hz, 1H), 4.40-4.63 (m, 3H), 3.63- 3.92 (m, 2H), 3.35-3.57 (m, 3H), 3.12-3.22 (m, 1H), 2.23-2.60 (m, 5H), 1.75-2.14 (m, 5H), 0.90-1.03 (m, 3H). |
| 108a | 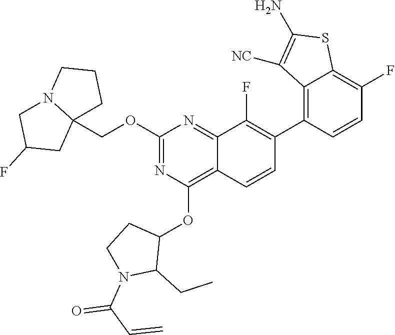 [SMILES: C=CC(=O)N1CCC(Oc2nc(OCC34CCCN3CC(F)C4)nc3c(F)c(-c4ccc(F)c5sc(N)c(C#N)c45)ccc23)C1CC] | |
| 109 | 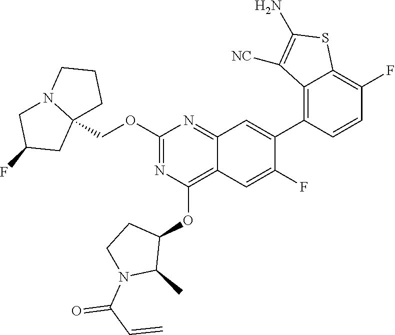 [SMILES: C=CC(=O)N1CC[C@@H](Oc2nc(OC[C@@]34CCCN3C[C@H](F)C4)nc3cc(-c4ccc(F)c5sc(N)c(C#N)c45)c(F)cc23)[C@H]1C] | LCMS m/z [M + 1]: 649.0 ¹H NMR (400 MHz, CD₃OD) δ 7.80 (d, J = 9.0 Hz, 1H), 7.68 (d, J = 6.5 Hz, 1H), 7.22-7.28 (m, 1H), 7.01 (t, J = 8.9 Hz, 1H), 6.60-6.73 (m, 1H), 6.28- 6.37 (m, 1H), 5.86-5.76 (m, 2H), 5.33 (d, J = 55.6 Hz, 1H), 4.68-4.79 (m, 1H), 4.24-4.39 (m, 2H), 3.66-3.94 (m, 2H), 3.32-3.38 (m, 1H), 3.23-3.26 (m, 2H), 3.02-3.11(m, 1H), 2.61-2.16 (m, 5H), 1.86- 2.08 (m, 3H), 1.24-1.36 (m, 3H). |
| 109a | 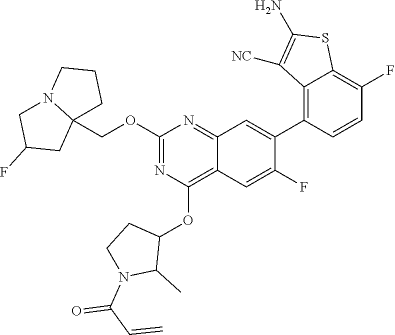 [SMILES: C=CC(=O)N1CCC(Oc2nc(OCC34CCCN3CC(F)C4)nc3cc(-c4ccc(F)c5sc(N)c(C#N)c45)c(F)cc23)C1C] | |
| 110 | 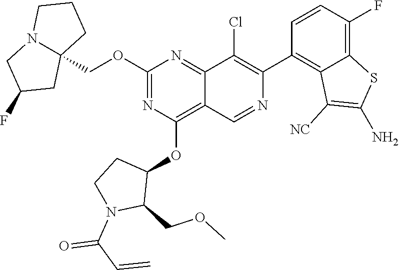 [SMILES: C=CC(=O)N1CC[C@@H](Oc2nc(OC[C@@]34CCCN3C[C@H](F)C4)nc3c(Cl)c(-c4ccc(F)c5sc(N)c(C#N)c45)ncc23)[C@H]1COC] | LCMS m/z [M + 1]: 696.3 ¹H NMR (400 MHz CD₃OD) δ 9.31 (s, 1H), 7.31- 7.34 (m, 1H), 7.05 (t, J = 9.18 Hz, 1H), 6.63-6.70 (m, 1H), 6.30-6.35 (m, 1H), 5.97-6.02 (m, 1H), 5.76-5.81 (m, 1H), 5.40 (d, J = 51.98 Hz, 1H), 4.50- 4.76 (m, 4H), 3.45-3.90 (m, 7H), 3.17 (s, 3H), 2.00- 2.59 (m, 8H). |
| 110a | 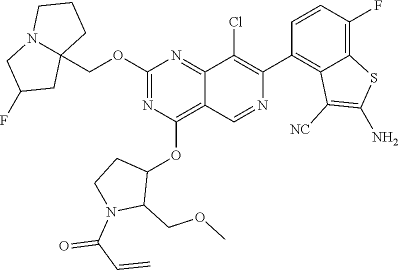 [SMILES: C=CC(=O)N1CCC(Oc2nc(OCC34CCCN3CC(F)C4)nc3c(Cl)c(-c4ccc(F)c5sc(N)c(C#N)c45)ncc23)C1COC] | |
| 111 | 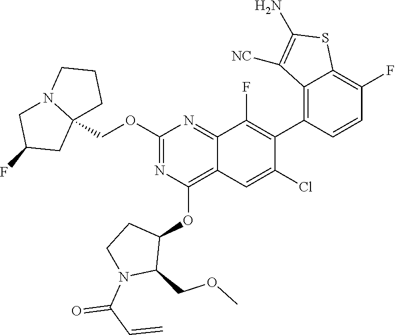 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(Cl)cc23)[C@H]1COC] | LCMS m/z [M + 1]: 713.2 ¹H NMR (400 MHz, CD₃OD) δ 8.13 (s, 1H), 7.20- 7.24 (m, 1H), 7.08 (t, J = 8.9 Hz, 1H), 6.65-6.75 (m, 1H), 6.33-6.40 (m, 1H), 6.01-5.93 (m, 1H), 5.79-5.85 (m, 1H), 5.61 (d, J = 51.8 Hz, 1H), 4.70- 4.80 (m, 3H), 4.05-3.72 (m, 6H), 3.56-3.68 (m, 1H), 3.47-3.57 (m, 1H), 3.22-3.27 (m, 1H), 3.15 (s, 2H), 2.37-2.77 (m, 7H), 2.14-2.26 (m, 1H). |
| 111a | 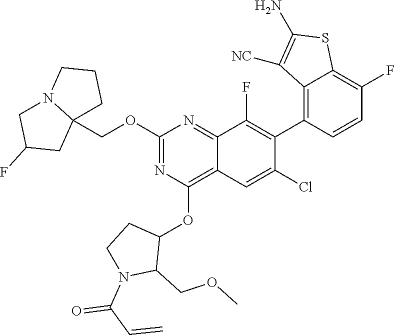 [SMILES: C=CC(=O)N1CCC(Oc2nc(OCC34CCCN3CC(F)C4)nc3c(F)c(-c4ccc(F)c5sc(N)c(C#N)c45)c(Cl)cc23)C1COC] | |
| 112 | 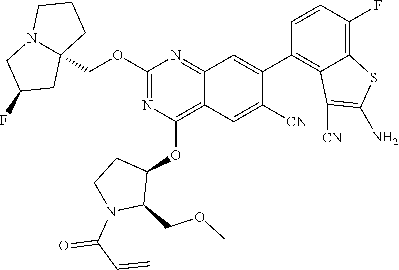 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C#N)cc23)[C@H]1COC] | LCMS m/z [M + 1]: 686.0 ¹H NMR (400 MHz, CD₃OD) δ 8.61 (s, 1H), 7.66 (s, 1H), 7.26-7.30 (m, 1H), 7.01-7.06 (m, 1H), 6.63- 6.73 (m, 1H), 6.29-6.34 (m, 1H), 5.93-5.98 (m, 1H), 5.75-5.81 (m, 1H), 5.35 (d, J = 54.2 Hz, 1H), 4.58- 4.77 (m, 2H), 4.25-4.42 (m, 2H), 3.54-3.93 (m, 4H), 3.06-3.27 (m, 6H), 1.93-2.57 (m, 8H). |
| 112a | 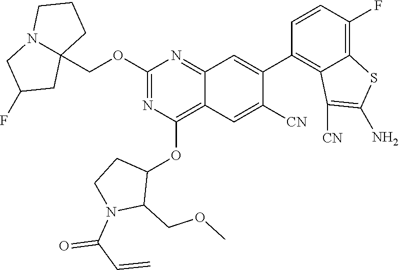 [SMILES: C=CC(=O)N1CCC(Oc2nc(OCC34CCCN3CC(F)C4)nc3cc(-c4ccc(F)c5sc(N)c(C#N)c45)c(C#N)cc23)C1COC] | |
| 113 | 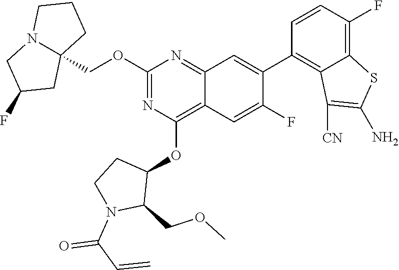 [SMILES: C=CC(=O)N1CC[C@@H](Oc2nc(OC[C@@]34CCCN3C[C@H](F)C4)nc3cc(-c4ccc(F)c5sc(N)c(C#N)c45)c(F)cc23)[C@H]1COC] | LCMS m/z [M + 1]: 679.0 ¹HNMR (400 MHz CD₃OD) δ 7.77-7.80 (m, 1H), 7.67-7.72 (m, 1H), 7.23-7.27 (m, 1H), 7.01 (t, J = 8.84 Hz, 1H), 6.61-6.79 (m, 1H), 6.27-6.31 (m, 1H), 5.67-5.78 (m, 2H), 5.40 (d, J = 52.44 Hz, 1H), 4.68- 4.76 (m, 2H), 4.48-4.56 (m, 2H), 3.34-3.82 (m, 6H), 3.21-3.25 (m, 3H), 3.13-3.15 (m, 1H), 1.99-2.46 (m, 8H). |
| 113a | 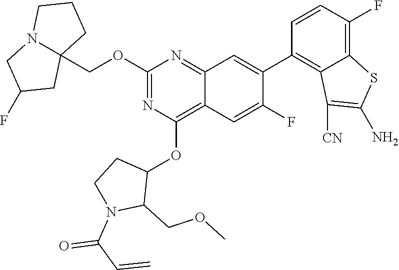 [SMILES: C=CC(=O)N1CCC(Oc2nc(OCC34CCCN3CC(F)C4)nc3cc(-c4ccc(F)c5sc(N)c(C#N)c45)c(F)cc23)C1COC] | |
| 114 | 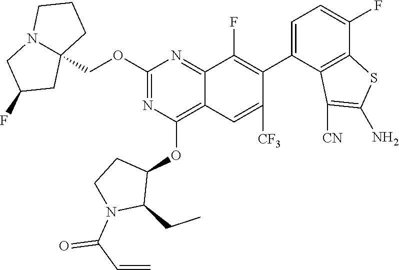 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@H]1CC] | LCMS m/z [M + 1]: 731.2 ¹H NMR (400 MHz, CD₃OD) δ 8.28 (s, 1H), 7.22- 7.23 (m, 1H), 7.00-7.04 (m, 1H), 6.67-6.71 (m, 1H), 6.33-6.64 (m,1H), 5.95-6.28 (m, 1H), 5.78-5.81 (m, 1H), 5.32-5.49 (m, 2H), 4.36-4.42 (m, 3H), 3.84- 3.87 (m, 2H), 3.06-3.37 (m, 3H), 1.96-2.45 (m, 10H), 1.28-1.30 (m, 3H). |
| 115 | 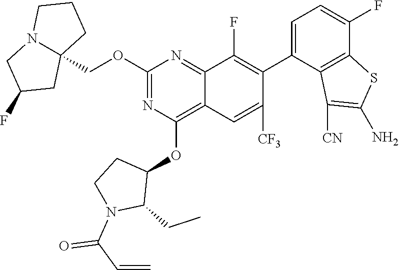 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@@H]1CC] | LCMS m/z [M + 1]: 731.2 ¹H NMR (400 MHz, CD₃OD) δ 8.53 (s, 1H), 8.17- 8.24 (m, 1H), 7.17-7.24 (m, 1H), 7.01 (t, J = 8.9 Hz, 1H), 6.58-6.74 (m, 1H), 6.27-6.37 (m, 1H), 5.73- 5.90 (m, 2H), 5.34 (d, J = 54.3 Hz, 1H), 4.59 (s, 1H), 4.33-4.49 (m, 3H), 3.74-4.02 (m, 2H), 3.32- 3.37 (m, 1H), 3.23-3.28 (m, 1H), 3.02-3.15 (m, 1H), 2.45-2.66 (m, 2H), 2.14-2.40 (m, 3H), 1.60-2.07 (m, 5H), 1.14-1.24 (m, 3H). |
| 115a | 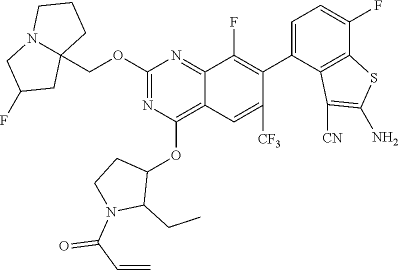 [SMILES: C=CC(=O)N1CCC(Oc2nc(OCC34CCCN3CC(F)C4)nc3c(F)c(-c4ccc(F)c5sc(N)c(C#N)c45)c(C(F)(F)F)cc23)C1CC] | |
| 116 | 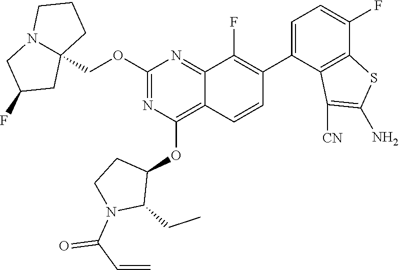 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@@H]1CC] | LCMS m/z [M + 1]: 663.3 ¹H NMR (400 MHz, CD₃OD) δ 8.53 (s, 1H), 7.83- 7.90 (m, 1H), 7.30-7.37 (m, 1H), 7.19-7.25 (m, 1H), 7.00 (t, J = 8.9 Hz, 1H), 6.56-6.73 (m, 1H), 6.26-6.35 (m, 1H), 5.71-5.88 (m, 2H), 5.35 (d, J = 55.0 Hz, 1H), 4.29-4.46 (m, 3H), 3.73-3.97 (m, 2H), 3.32-3.45 (m, 2H), 3.26-3.30 (m, 1H), 3.0-3.16 (m, 1H), 2.15-2.65 (m, 5H), 1.55-2.11 (m, 5H), 1.12- 1.24 (m, 3H). |
| 117 | 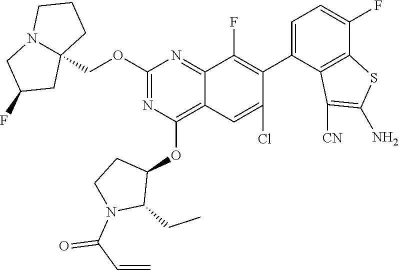 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(Cl)cc23)[C@@H]1CC] | LCMS m/z [M + 1]: 697.2 ¹H NMR (400 MHz, CD₃OD) δ 7.91-7.93(m, 1H), 7.16-7.21 (m, 1H), 7.02 (t, J = 8.76 Hz, 1H), 6.56- 6.72 (m, 1H), 6.27-6.34 (m, 1H), 5.72-5.84 (m, 2H), 5.30 (d, J = 53.3 Hz, 1H), 4.27-4.41 (m, 3H), 3.74- 3.97 (m, 2H), 3.17-3.26 (m, 3H), 3.00-3.03 (m, 1H), 2.12-2.61 (m, 5H), 1.53-2.03 (m, 5H), 1.14- 1.21 (m, 3H). |
| 117a | 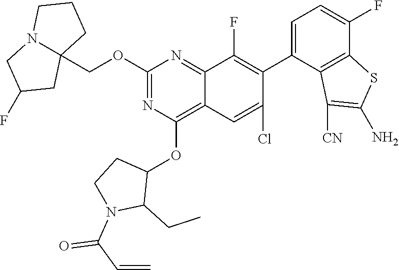 [SMILES: C=CC(=O)N1CCC(Oc2nc(OCC34CCCN3CC(F)C4)nc3c(F)c(-c4ccc(F)c5sc(N)c(C#N)c45)c(Cl)cc23)C1CC] | |
| 118 | 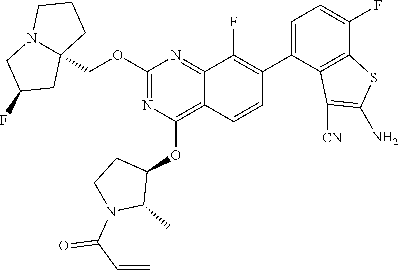 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@@H]1C] | LCMS m/z [M + 1]: 649.3 ¹H NMR (400 MHz, CD₃OD): δ 7.93-8.01 (m, 1H), 7.44 (t, J = 8.4 Hz, 1H), 7.24-7.28 (m, 1H), 7.04 (t, J = 8.8, 1H), 6.58-6.74 (m, 1H), 6.24-6.38 (m, 1H), 5.47-5.83 (m, 3H), 4.65-4.78 (m, 1H), 4.54 (t, J = 6.7 Hz, 1H), 3.80-4.10 (m, 5H), 3.43-3.57 (m, 1H), 2.07-2.86 (m, 9H), 1.34-1.47 (m, 3H). |
| 119 | 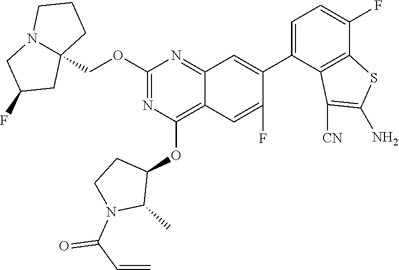 [SMILES: C=CC(=O)N1CC[C@@H](Oc2nc(OC[C@@]34CCCN3C[C@H](F)C4)nc3cc(-c4ccc(F)c5sc(N)c(C#N)c45)c(F)cc23)[C@@H]1C] | LCMS m/z [M + 1]: 649.2 ¹H NMR (400 MHz, CD₃OD): δ 7.75-7.84 (m, 1H), 7.71 (d, J = 5.6 Hz, 1H), 7.21-7.26 (m, 1H), 7.01 (t, J = 9.2 Hz, 1H), 6.60-6.74 (m, 1H), 6.25-6.40 (m, 1H), 5.74-5.79 (m, 1H), 5.68 (s, 1H), 5.53 (d, J = 53.3 Hz, 1H), 4.65-4.78 (m, 2H), 4.51-4.54(m, 1H), 3.81-4.05 (m, 5H), 3.46-3.53 (m, 1H), 2.30-2.82 (m, 7H), 2.21 (s, 1H), 1.34-1.47 (m, 3H). |
| 120 | 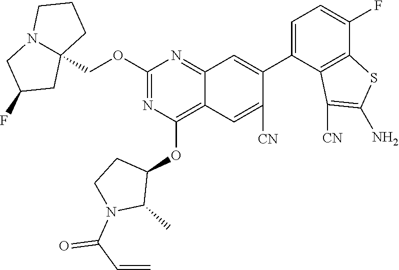 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C#N)cc23)[C@@H]1C] | LCMS m/z [M + 1]: 656.3 ¹H NMR (400 MHz, CD₃OD) δ 8.46-8.52 (m, 1H), 7.41-7.80 (m, 2H), 7.18-7.32 (m, 1H), 7.03 (t, J = 9.08 Hz, 1H), 6.61-6.72 (m, 1H), 6.24-6.36 (m, 1H), 5.56-5.80 (m, 2H), 5.35 (d, J = 53.6 Hz, 1H), 4.47- 4.59 (m, 3H), 4.29-4.43 (m, 2H), 3.75-4.03 (m, 2H), 3.06-3.13 (m, 1H), 1.95-2.63 (m, 8H), 1.25-1.47 (m, 3H). |
| 121 | 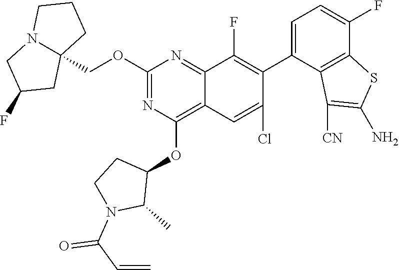 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(Cl)cc23)[C@@H]1C] | LCMS m/z [M + 1]: 683.3 ¹H NMR (400 MHz, CD₃OD) δ 8.04-8.10 (m, 1H), 7.18-7.25 (m, 1H), 6.95-7.06 (m, 1H), 6.61-6.72 (m, 1H), 6.29-6.37 (m, 1H), 5.76-5.84 (m, 3H), 4.72-4.92 (m, 2H), 4.55-4.63 (m, 1H), 4.20-4.30 (m, 1H), 3.91-4.03 (m, 5H), 3.51-3.62 (m, 1H), 2.10-2.88 (m, 7H), 1.47-1.52 (m, 3H). |
| 122 | 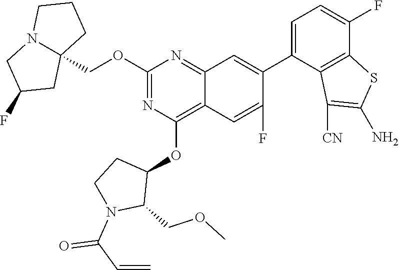 [SMILES: C=CC(=O)N1CC[C@@H](Oc2nc(OC[C@@]34CCCN3C[C@H](F)C4)nc3cc(-c4ccc(F)c5sc(N)c(C#N)c45)c(F)cc23)[C@@H]1COC] | LCMS m/z [M + 1]: 679.3 ¹H NMR (400 MHz, CD₃OD) δ 7.70-7.77 (m, 1H), 7.65 (d, J = 6.56 Hz, 1H), 7.21-7.26 (m, 1H), 7.00 (t, J = 8 Hz, 1H), 6.66-6.74 (m, 1H), 6.31-6.33 (m, 1H), 5.72-5.90 (m, 2H), 5.35 (d, J = 54.6 Hz, 1H), 4.56-4.59 (m, 3H), 4.28-4.38 (m , 2H), 3.59- 3.96 (m, 4H), 3.42-3.43 (m , 4H), 3.07-3.08 (m, 1H), 1.95-2.70 (m, 8H). |
| 123 | 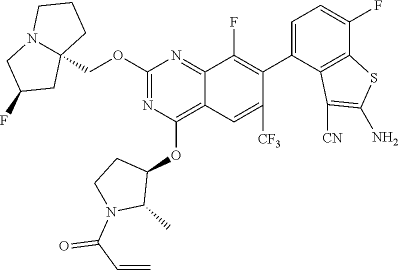 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@@H]1C] | LCMS m/z [M + 1]: 717.2 ¹HNMR (400 MHz, CD₃OD) δ 8.18-8.22 (m, 1H), 7.20-7.26 (m, 1H), 6.98-7.03 (m, 1H), 6.63-6.75 (m, 1H), 6.30-6.35(m, 1H), 5.55- 5.84 (m, 2H), 5.24-5.44 (m, 1H), 4.50-4.68 (m, 2H), 4.38-4.49 (m, 2H), 3.78- 4.04 (m, 2H), 3.34-3.58 (m, 2H), 3.05-3.18 (m,1H), 1.92-2.72 (m, 8H), 1.36- 1.52 (m, 3H). |
| 124 | 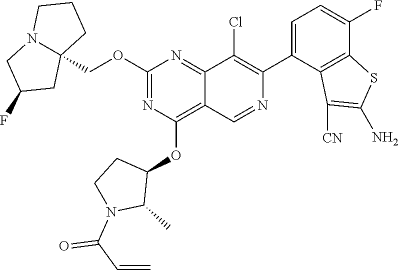 [SMILES: C=CC(=O)N1CC[C@@H](Oc2nc(OC[C@@]34CCCN3C[C@H](F)C4)nc3c(Cl)c(-c4ccc(F)c5sc(N)c(C#N)c45)ncc23)[C@@H]1C] | LCMS m/z [M + 1]: 666.2 ¹H NMR (400 MHz, CD₃OD) δ 9.19-9.26 (m, 1H), 7.29-7.32 (m, 1H), 7.03 (t, J = 9.0 Hz, 1H), 6.61- 6.71 (m, 1H), 6.27-6.35 (m, 1H), 5.62-5.78 (m, 2H), 5.33 (d, J = 53.2 Hz, 1H), 4.40-4.54 (m, 4H), 3.80- 4.01 (m, 2H), 3.33-3.38 (m, 1H), 3.06-3.13 (m, 1H), 1.95-2.69 (m, 9H), 1.36-1.45 (m, 3H). |
| 125 | 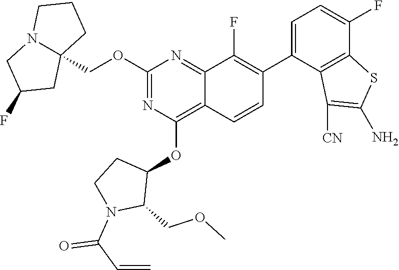 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)ccc23)[C@@H]1COC] | LCMS m/z [M + 1]: 679.3 ¹H NMR (400 MHz, CD₃OD) δ 7.73-7.82 (m, 1H), 7.18-7.26 (m, 1H), 7.06-7.17 (m, 1H), 6.90 (t, J = 8.8 Hz, 1H), 6.59 (dd, J = 10.2, 17.4 Hz, 1H), 6.22 (d, J = 16.8 Hz, 1H), 5.57-5.88 (m, 2H), 5.25 (d, J = 53.9 Hz, 1H), 4.38-4.51 (m, 2H), 4.18-4.36 (m, 3H), 3.48-3.85 (m, 4H), 3.28-3.37 (m, 3H), 2.89-2.98 (m, 1H), 2.02-2.63 (m, 5H), 1.76-2.01 (m, 3H). |
| 126 | 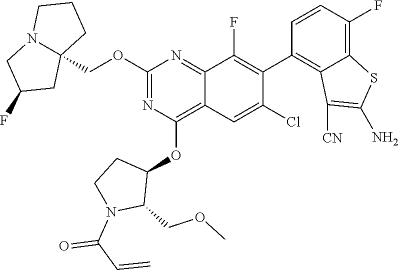 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(Cl)cc23)[C@@H]1COC] | LCMS m/z [M + 1]: 713.2 ¹H NMR (400 MHz, CD₃OD) δ 7.94-7.97 (m, 1H), 7.18-7.21 (m, 1H), 7.03 (t, J = 9.16 Hz, 1H), 6.67- 6.74 (m, 1H), 6.32 (d, J = 16.72 Hz, 1H), 5.73-5.91 (m, 2H), 5.34 (d, J = 54.8 Hz, 1H), 4.59 (s, 3H), 4.31-4.41 (m, 2H), 3.59-3.99 (m, 4H), 3.41-3.43 (m, 3H), 3.05-3.26 (m, 2H), 1.94-2.68 (m, 8H). |
| 127 | 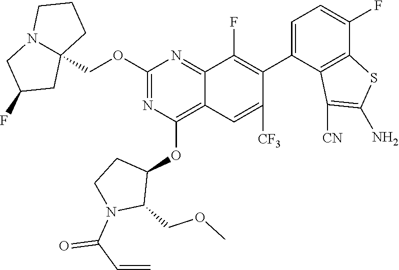 [SMILES: C=CC(=O)N1CC[C@@H](Oc2nc(OC[C@@]34CCCN3C[C@H](F)C4)nc3c(F)c(-c4ccc(F)c5sc(N)c(C#N)c45)c(C(F)(F)F)cc23)[C@@H]1COC] | LCMS m/z [M + 1]: 747.3 ¹H NMR (400 MHz, CD₃OD) δ 8.53 (s, 1H), 8.20- 8.25 (m, 1H), 7.18-7.24 (m, 1H), 7.01 (t, J = 8.9 Hz, 1H), 6.67-6.75 (m, 1H), 6.29-6.36 (m, 1H), 5.87-5.96 (m, 1H), 5.73-5.83 (m, 1H), 5.34 (d, J = 54.2 Hz, 1H), 4.58-4.68 (m, 3H), 4.33-4.47 (m, 2H), 3.63-3.97 (m, 4H), 3.40-3.46 (m, 3H), 3.24-3.28 (m, 1H), 3.03-3.13 (m, 1H), 2.56-2.73 (m, 1H), 2.17- 2.51 (m, 4H), 1.92-2.10 (m, 3H). |
| 128 | 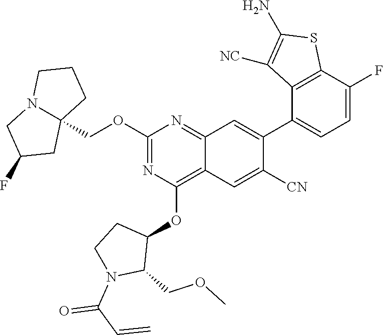 [SMILES: C=CC(=O)N1CC[C@@H](Oc2nc(OC[C@@]34CCCN3C[C@H](F)C4)nc3cc(-c4ccc(F)c5sc(N)c(C#N)c45)c(C#N)cc23)[C@@H]1COC] | LCMS m/z [M + 1]: 686.2 ¹H NMR (400 MHz, CD₃OD) δ 8.52-8.56 (m, 1H), 7.70 (s, 1H), 7.24-7.29 (m, 1H), 7.03 (t, J = 8.9 Hz, 1H), 6.67-6.75 (m, 1H), 6.29-6.36 (m, 1H), 5.85- 5.94 (m, 1H), 5.73-5.82 (m, 1H), 5.33 (d, J = 55.1 Hz, 1H), 4.57-4.66 (m, 2H), 4.30-4.41 (m, 2H), 3.88-4.02 (m, 2H), 3.59-3.80 (m, 3H), 3.40-3.45 (m, 3H), 3.19-3.25 (m, 1H), 3.01-3.09 (m, 1H), 2.46- 2.68 (m, 2H), 2.13-2.31 (m, 3H), 1.92-2.06 (m, 3H). |
| 129 | 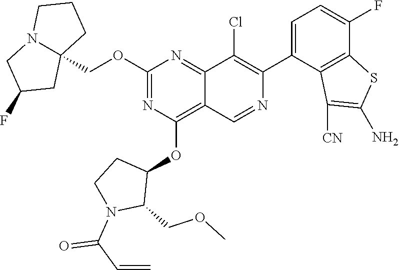 [SMILES: C=CC(=O)N1CC[C@@H](Oc2nc(OC[C@@]34CCCN3C[C@H](F)C4)nc3c(Cl)c(-c4ccc(F)c5sc(N)c(C#N)c45)ncc23)[C@@H]1COC] | LCMS m/z [M + 1]: 696.3 ¹H NMR (400 MHz, CD₃OD) δ 9.23 (d, J = 10.5 Hz, 1H), 7.31 (dd, J = 8.4, 5.1 Hz, 1H), 7.01-7.07 (m, 1H), 6.66-6.74 (m, 1H), 6.29-6.36 (m, 1H), 5.90-5.97 (m, 1H), 5.74-5.82 (m, 1H), 5.45 (d, J = 52.4 Hz, 1H), 4.56-4.65 (m, 4H), 3.92-3.97 (m, 1H), 3.75-3.82 (m, 1H), 3.67-3.74 (m, 1H), 3.51-3.62 (m, 3H), 3.40-3.45 (m, 3H), 3.20-3.26 (m, 1H), 2.40- 2.65 (m, 4H), 2.09-2.36 (m, 4H). |

### Biological Example 1: Covalent Cysteine 12 Modification Analysis Using Matrix Assisted Laser Desorption Ionization-Time of Flight Mass Spectrometry (MALDI-TOF MS)

Reaction: 1 μM of GppNHp-, GTP-, or GDP-loaded KRAS4b (amino acids 1-169) G12C/C118S protein (produced in-house by Protein Expression Laboratory, FNLCR/Leidos Biomed) in 20 mM 4-(2-hydroxyethyl)-1-piperazineethanesulfonic acid (HEPES) buffer containing 150 mM NaCl, 1 mM MgCl₂, pH 7.3 was prepared freshly before assay. Ten-μL aliquots of protein were dispensed onto low volume 384-well plate, then 375 nL DMSO, and 25 nL of tested compounds from 5 mM DMSO stocks were added to appropriate wells using ECHO 555 acoustic liquid handler (Labcyte Inc.). For each reaction/assay, three blanks were prepared by mixing 10 μL of protein solution with 400 nL DMSO. The contents of the wells were carefully mixed by aspiration, and then each plate was sealed with an adhesive cover, centrifuged at 2000 g for 1 minute, and kept in the dark at room temperature until 5 min, 15 min, 30 min, or 2 h collections.

Target pretreatment: Before each assay MALDI target (Bruker MPT 384 ground steel BC) was pre-treated by pipetting on each spot 0.75 μL of saturated sinapinic acid in acetonitrile (ACN). This step significantly improves uniformity of sample crystallization across the plate resulting in enhanced sensitivity.

Sample preparation: At collection time point, 2 μL of reaction mixtures were pipetted out into 20 μL MALDI matrix solution (saturated solution of sinapinic acid in 1:1 ACN:water solution containing 0.75% trifluoroacetic acid (TFA)) deposited on 384 well polypropylene plate. Resulting solution was mixed by aspiration, centrifuged at 2000 g for 1 minute, then 1 μL aliquots were dispensed on pre-treated MALDI target using Beckman Coulter Biomek FXP 96/384-Span-8 Laboratory Automation Workstation. Finally, the MALDI target was dried under mild vacuum to produce spots with fine crystalline structure.

Measurements: MALDI-TOF measurements were performed on a Bruker Daltonics rapiflex Tissuetyper TOF-TOF mass spectrometer using linear mode and mass range from 18.6 to 21.6 kDa. Detector gain was set to 3.3× (483 V), sample rate to 5 GS/s, real time smoothing to medium (175 MHz), laser smart beam pattern was set to: “MS Thin Layer M5”, and the laser frequency was 10000 Hz. Spectra were automatically collected using custom Auto Xecute method. Laser power was auto adjusted using fuzzy control. The peak selection range was set to be between 19000 and 21500 Da. Peak evaluation uses half width parameter set to be smaller than 40 Da for processed spectrograms (centroid peak detection; smoothed by SavitzkyGolay algorithm using 7 m/z width and 2 cycles; baseline was subtracted using median algorithm with flatness 1 and median level 0.01). Fuzzy control used Proteins/Oligonucleotides protocol with minimum half width 1/10 times above threshold. Up to 40000 satisfactory shots were collected in 10000 shot steps. Dynamic termination was implemented to finish data collection when peak signal/noise ratio was reaching value of 50.

Spectra processing: Spectra were smoothed by SavitzkyGolay algorithm using 7 m/z width and three cycles. Centroid peak detection algorithm was used with signal to noise threshold set to 6, relative intensity threshold 4%, peak width 10 m/z and median baseline subtraction using flatness of 1 and median level of 0.01. Peak intensity and area under the peak were evaluated and recorded for all peaks between 19,300 Da and 21,550 Da.

Calculation of percent modification: Percent modification was calculated as a ratio of peak height for protein modified by compound to sum of peak height of remaining protein plus peak height for protein modified by compound. If multiple modifications were observed each was calculated as a ratio of peak height for given modification versus sum of peak heights for all observed protein species. Data reported in Table 5 are from 15 minutes reaction time point.

### Biological Example 2: Disrupting KRAS G12C-Effector Binding (Protein: Protein Interaction HTRF Assay)

A protein: protein (PPI) interaction Homogeneous Time Resolved Fluorescence (HTRF) assay was used to determine the effectiveness of compounds of the present disclosure in disrupting KRAS G12C protein and effector (RAF1 or PIK3CA) binding.

The HTRF assay used the following reagents and proteins: 50 nM Avi-KRAS G12C Q25A (1-169) GppNHp/3×FLAG-PI3K CA (157-299); 50 nM Avi-KRAS G12C (1-169) GppNHp/RAF1 RBD-3×FLAG (52-151); 35 nM Avi-PI3K RBD-3×FLAG; Assay Buffer: 50 mM Tris pH 7.5, 100 mM NaCl, 5 mM MgCl₂, 0.1% BSA, 0.01% Tween 20, 10% DMSO; Bead Buffer: 50 mM Tris pH 7.5, 0.01% Tween 20; Assay volume: 20 μL (384 well plate-low volume format); and Compound titration: 30-0.02 μM, 3× dilution series.

The HTRF assay employed the following protocol:

Compounds were dispensed in assay plate (384-well, Grenier Bione #784075) using Echo (model 555) with dose response settings: 200 nL final volume, titration from 100 μM as a 10-point dilution series. KRAS proteins were prepared in assay buffer, and dispensed on plates, 5 μL per well, then incubated for 1 h at room temperature, with 700 rpm shaking. RAF1 RBD and PI3K RBD CA were prepared in assay buffer, dispensed onto plates, 5 μL per well, and then incubated for 1 h at room temperature, with 700 rpm shaking. Reagent mix was then prepared and dispensed on plates, 10 μL per well, and then incubated for 1 h at room temperature, with 700 rpm shaking.

Plates were analyzed on an Envision plate reader using the following setting: Excitation 320 nm, Bandwidth 75 nm; Emission 615 nm, Bandwidth 85 nm; Gain 100%; Flashes 100; Lag 60 μs. Data was reported as percentage of activity, with DMSO as 100%. Data was plotted and analyzed using Prism 8. Table 4 summarizes parameters used with the Envision plate reader.

TABLE 4
Envision plate reader settings.

| | |
|---|---|
| Top Mirror | TRF LANCE/DELFIA dual enh (D400/630) |
| Excitation Filter | UV2 (TRF) 320 (Bandwidth: 75 nm) |
| Emission Filter | APC 665 (Bandwidth: 75 nm) |
| 2^{nd} Emission Filter | Europium 615 (Bandwidth: 85 nM) |
| Cycle | 2000 |
| Gain (%) | 100 |
| Number of Flashes | 100 |
| Delay (μs) | 60 |
| Total time of windows (μs) | 400 |
| | Flash Lamp |

### Biological Example 3: Cell-Based pERK HTRF Assay

pERK assays (Perkin Elmer) were used to determine the effectiveness of compounds of the present disclosure in disrupting KRAS G12C protein/effector signaling in cells.

On Day 1, cells (NCI-H358) were seeded into 96-well plates at 4×10⁴ cells/well in complete growth media (RPMI, 10% FBS).

On Day 2, cells were treated with compounds at 0.25% DMSO. The source plate was created with compounds diluted in media at 5-fold the final assay concentration. The compounds were run in a 9-point concentration curve starting at 75 μM, with a 3-fold dilution between concentrations. 20 μL was transferred onto the cell plates (final volume in wells was 100 μL). Plates were harvested after 30 min incubation by aspirating media and adding kit-supplied 1× supplemented lysis buffer to all wells (75 μl per well). Plates were then placed on a plate shaker and incubated at 850 rpm for an additional 30 min.

Antibody mixture solution was prepared by diluting aliquoted d2 and Eu Cryptate antibodies 1:20 in kit supplied detection buffer, then mixed the diluted antibodies solutions (1:1 v:v). 4 μL of this solution was then added to a 384-well detection plate (Perkin Elmer; 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for Imin) and allowed to incubate between 4 and 24 h at room temperature. Maximum signal is reached after 4 h incubation time and remains stable over a period of 24 hours. Therefore, readings can be made between 4 and 24 h of incubation. Plates were centrifuged again (524 g for Imin) and analyzed on the En Vision plate reader using the following settings: Excitation 320 nm, Bandwidth 75 nm; Emission 615 nm, Bandwidth 85 nm; Gain 100%; Flashes 100; Lag 60 μs.

The percent of modification of GppNHp-, GTP-, or GDP-loaded KRAS G12C by MALDI-TOF MS, biochemical Raf1 RBD-KRAS G12C-GppNHp disruption assay IC₅₀, and pERK inhibition IC₅₀ of selected compounds described herein are shown in Table 5. For percent of modification at 15 minutes (MALDI-TOF MS) of GppNHp-, GTP-, or GDP-loaded KRAS G12C: A: percent of modification ≥70%; B: 50≤percent of modification <70%; C: 10%≤modification <50%; D: percent of modification <10%. For Raf1 RBD-KRAS G12C-GppNHp disruption assay: A: IC₅₀≤0.5 μM; B: 0.5 μM<IC₅₀≤5 μM; C: 5 μM<IC₅₀≤20 μM; D: IC₅₀>20 μM. For pERK inhibition in H358 cell assay: A: IC₅₀≤0.1 μM; B: 0.1 μM<IC₅₀≤1 μM; C: IC₅₀>1 μM. Blanks in the table represent that compound was not tested in the indicated assay.

TABLE 5
Biological characterization of selected compounds of the present disclosure.

| | GppNHp- | GTP-KRAS | GDP-KRAS | Raf1- | |
|---|---|---|---|---|---|
| | KRAS G12C | G12C | G12C | GppNHp- | pERK in |
| Compound | percent of | percent of | percent of | KRAS G12C | NCI-H358 |
| No. | modification | modification | modification | disruption | cell |
| 1 | C | | A | B | B |
| 2 | D | | B | B | B |
| 3 | A | | A | A | A |
| 4 | D | | D | D | C |
| 5 | D | | D | C | C |
| 6 | D | | D | D | C |
| 7 | D | | D | D | C |
| 8 | D | | C | B | C |
| 9 | D | | C | B | B |
| 10 | D | | D | B | B |
| 11 | A | | A | A | A |
| 12 | D | | D | B | B |
| 13 | B | | A | A | B |
| 14 | B | | A | A | A |
| 15 | D | | D | D | C |
| 16 | C | | A | B | B |
| 17 | D | | D | D | C |
| 18 | C | | A | A | A |
| 19 | D | | D | C | C |
| 20 | D | | B | C | C |
| 21 | D | | D | D | C |
| 22 | C | | A | A | A |
| 23 | C | | B | B | B |
| 24 | B | | A | A | A |
| 25 | D | | D | C | C |
| 26 | C | | A | B | A |
| 27 | C | | A | A | A |
| 28 | A | | A | A | A |
| 29 | A | | A | A | A |
| 30 | A | | A | A | A |
| 31 | D | | D | B | C |
| 32 | C | | A | A | A |
| 33 | D | | D | C | C |
| 34 | B | | A | A | A |
| 35 | D | | C | B | B |
| 36 | C | | C | A | A |
| 37 | A | | A | A | A |
| 38 | D | | D | B | B |
| 39 | C | | C | B | B |
| 40 | A | | A | A | A |
| 41 | C | | A | A | B |
| 42 | D | | D | D | C |
| 43 | C | | A | B | C |
| 44 | A | B | A | A | A |
| 45 | C | C | B | A | A |
| 46 | B | B | B | A | A |
| 49 | B | C | C | A | A |
| 50 | A | B | B | A | A |
| 51 | A | A | A | A | A |
| 52 | D | D | D | C | C |
| 55 | C | D | C | A | A |
| 56 | A | B | A | A | A |
| 57 | C | D | A | B | B |
| 58 | A | B | A | A | A |
| 59 | B | C | A | A | A |
| 60 | B | C | A | A | A |
| 61 | C | C | C | A | A |
| 62 | B | C | B | A | A |
| 63 | A | A | A | A | A |
| 64 | D | D | D | D | C |
| 65 | A | B | B | A | A |
| 66 | A | C | A | A | A |
| 67 | B | B | B | A | A |
| 68 | A | C | A | A | B |
| 69 | A | C | A | A | A |
| 70 | A | B | A | A | A |
| 71 | C | D | C | A | A |
| 73 | A | A | A | A | A |
| 75 | A | A | A | A | A |
| 76 | A | A | A | A | A |
| 77 | A | A | A | A | A |
| 78 | C | D | C | A | A |
| 79 | D | D | D | B | B |
| 80 | A | A | A | A | A |
| 82 | B | C | A | A | A |
| 83 | B | C | A | A | A |
| 84 | C | C | C | A | A |
| 85 | C | C | C | A | A |
| 86 | D | D | D | C | C |
| 87 | C | D | A | B | B |
| 88 | C | C | C | A | B |
| 89 | B | B | A | A | A |
| 90 | D | D | D | D | C |
| 91 | B | C | A | A | A |
| 93 | A | B | A | A | A |
| 94 | A | B | A | A | A |
| 95 | A | A | A | A | A |
| 96 | D | D | D | C | B |
| 97 | A | A | A | A | A |
| 98 | B | C | B | A | B |
| 99 | B | B | B | A | A |
| 100 | C | C | B | A | B |
| 101 | B | C | A | A | A |
| 102 | A | A | A | A | A |
| 103 | B | C | A | A | A |
| 104 | C | C | A | A | B |
| 105 | A | B | B | A | A |
| 106 | A | A | A | A | A |
| 107 | A | C | A | A | A |
| 108 | C | C | A | A | A |
| 109 | B | C | A | | A |
| 110 | A | A | A | A | A |
| 111 | D | D | C | B | A |
| 112 | A | C | A | A | A |
| 113 | D | D | C | D | C |
| 114 | C | C | C | A | A |
| 115 | D | D | C | B | B |
| 116 | D | D | C | D | C |
| 117 | D | D | C | D | C |
| 118 | D | D | A | B | C |
| 119 | D | D | B | C | C |
| 120 | D | D | B | C | B |
| 121 | C | D | C | A | B |
| 122 | D | D | C | D | C |
| 123 | C | D | B | A | A |
| 124 | D | D | B | B | B |
| 125 | D | D | C | D | C |
| 126 | D | D | A | B | B |
| 127 | D | D | B | A | B |
| 128 | D | D | C | D | C |
| 129 | A | C | B | A | B |

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that methods and structures within the scope of these claims and their equivalents be covered thereby.

1. A compound according to Formula I: 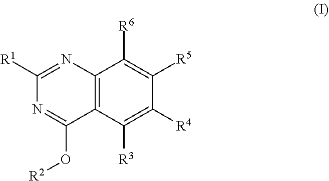
or a salt (e.g., pharmaceutically acceptable salt) thereof, wherein:
R¹ is selected from —OR⁷, 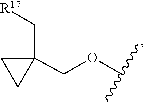
and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is substituted with one or more E and 0-4 R⁸;
R³ is selected from H, —OR¹⁰, and C₁₋₆alkyl, wherein any C₁₋₆alkyl is unsubstituted or substituted with one or more R¹¹;
R⁴ is selected from H, halogen, —CN, —OR¹², and C₁₋₆alkyl, wherein any C₁₋₆alkyl is unsubstituted or substituted with one or more R¹¹;
R⁵ is a bicyclic heteroaryl substituted with one or more R⁹;
R⁶ is selected from halogen, —OR¹², —CN, and H;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is independently selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
each E is independently selected from 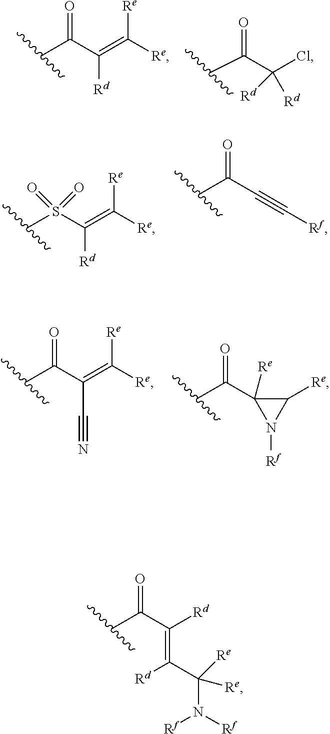
and —CN;
each R^{a} and R^{b} is independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} and R^{e} is independently selected from halogen, C₁₋₆ alkyl, and H; and
each R^{f} is independently selected from C₁₋₆ alkyl and H.

2. The compound of claim 1, wherein R² is a 4-6 membered heterocycle containing one nitrogen atom, wherein the heterocycle is substituted with one or more E and 0-4 R⁸.

3. The compound of claim 2, wherein R² is a heterocycle selected from azetidine, pyrrolidine, and piperidine, wherein the heterocycle is substituted with one or more E and 0-4 R⁸.

4. The compound of any one of claims 1-3, wherein R² is substituted with one E and 0-4 R⁸.

5. The compound of claim 4, wherein R² is substituted with one E and 1-4 R⁸.

6. The compound of claim 5, wherein the 1-4 R⁸ are each independently selected from halogen and C₁₋₆alkyl, wherein any C₁₋₆alkyl is unsubstituted.

7. The compound of any one of claims 1-6, wherein each E is independently selected from:

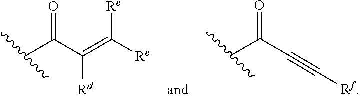

8. The compound of claim 1, wherein the compound is a compound according to Formula IA: 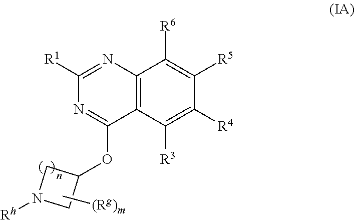
or a salt (e.g., pharmaceutically acceptable salt) thereof, wherein:
n is 1, 2, or 3;
m is 0, 1, 2, 3, or 4;
R^{h} and each R^{g} is independently selected from E, halogen, and C₁₋₆alkyl, wherein any C₁₋₆alkyl is unsubstituted or substituted with one or more R¹¹, and R^{h} or at least one R^{g} is E.

9. The compound of claim 8, wherein the compound is a compound according to Formula IA1: 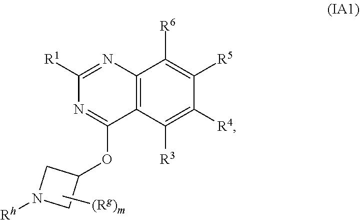
or a salt (e.g., pharmaceutically acceptable salt) thereof.

10. The compound of claim 8, wherein the compound is a compound according to Formula IA2: 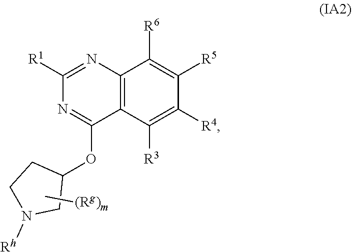
or a salt (e.g., pharmaceutically acceptable salt) thereof.

11. The compound of claim 9, wherein the compound is a compound according to Formula IA3: 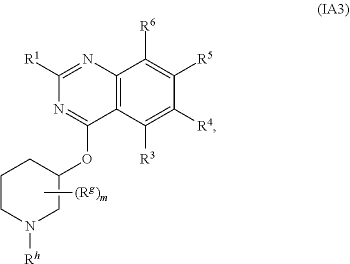
or a salt (e.g., pharmaceutically acceptable salt) thereof.

12. The compound of any one of claims 8-11, wherein R^{h} is E.

13. The compound of claim 12, wherein R^{h} is:

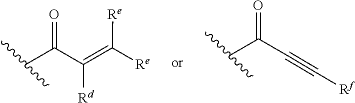

14. The compound of claim 13, wherein R^{h} is 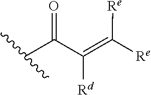
and each R^{d} and R^{e} is H.

15. The compound of any one of claims 8-14, wherein m is 1 or 2, and each R^{g} is independently selected from halogen and C₁₋₆alkyl, wherein any C₁₋₆alkyl is unsubstituted.

16. The compound of any one of claims 8-14, wherein m is 0.

17. The compound of any one of claims 1-16, wherein R¹ is —OR⁷.

18. The compound of claim 17, wherein R¹ is: 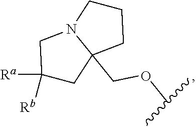
wherein R^{a} and R^{b} are each independently selected from halogen, C₁₋₆alkyl, —OR¹², and H, wherein any C₁₋₆alkyl is unsubstituted or is substituted with one or more R¹³.

19. The compound of claim 18, wherein R^{b} is H.

20. The compound of claim 18, wherein R¹ is selected from:

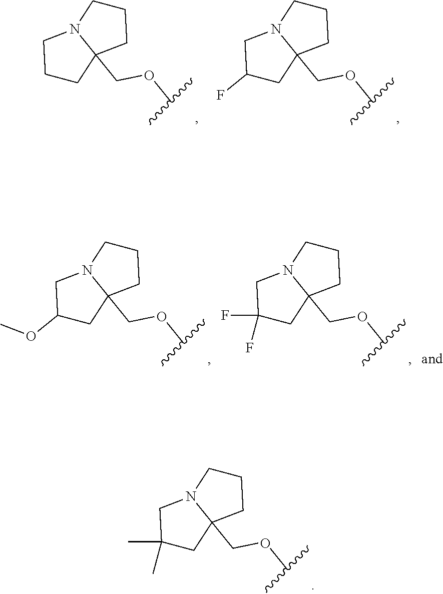 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

21. The compound of claim 17, wherein R¹ is selected from: 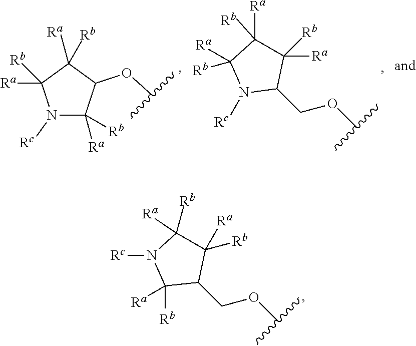
wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, wherein any C₁₋₆ alkyl or 3-6 membered carbocycle or heterocycle is unsubstituted or is substituted with one or more R¹³.

22. The compound of claim 21, wherein R¹ is selected from:

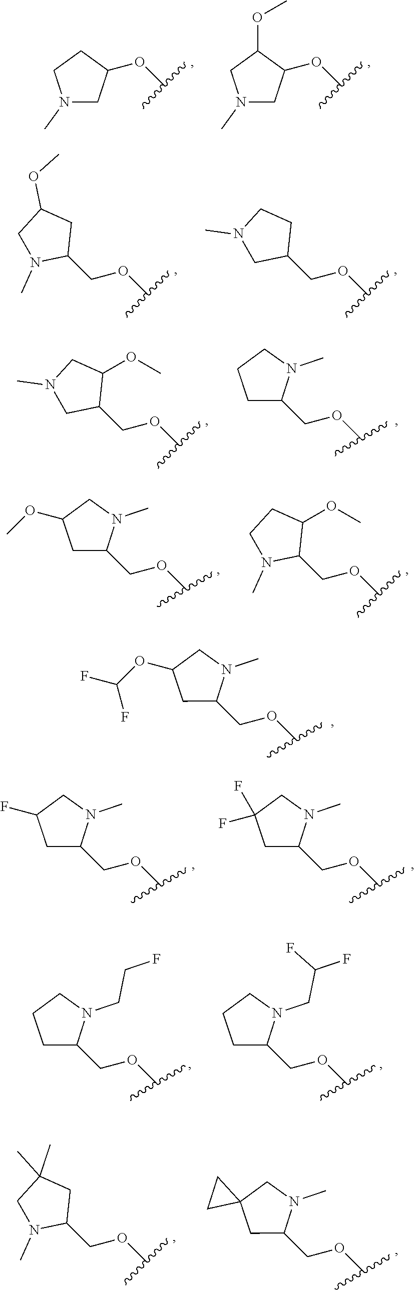

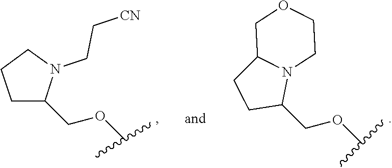 [SMILES: *OCC1CCCN1CCC#N ; *OCC1CCC2COCCN12]

23. The compound of any one of claims 1-16, wherein R¹ is

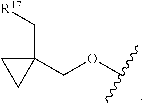

24. The compound of claim 23, wherein R¹ is selected from:

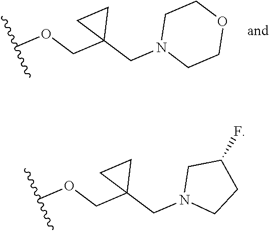 [SMILES: *OCC1(CN2CCOCC2)CC1 ; *OCC1(CN2CC[C@@H](F)C2)CC1]

25. The compound of any one of claims 1-16, wherein R¹ is a 4-6 membered heterocycle comprising a nitrogen atom, wherein the heterocycle is unsubstituted or substituted with one or more R¹⁵.

26. The compound of claim 25, wherein R¹ is selected from:

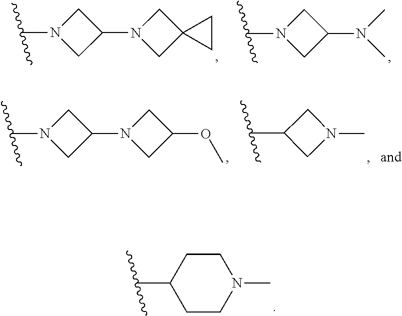

27. The compound of claim 1, wherein the compound is a compound according to Formula IB: 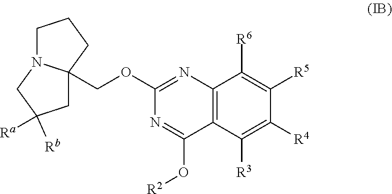
or a salt (e.g., pharmaceutically acceptable salt) thereof.

28. The compound of claim 27, wherein R^{a} is a halogen.

29. The compound of claim 27, wherein R^{a} is a —OR¹².

30. The compound of any one of claims 27-29, wherein R^{b} is H.

31. The compound of any one of claims 27-30, wherein R² is a 4-6 membered heterocycle containing one nitrogen atom, wherein the heterocycle is substituted with one or more E and 0-4 R⁸.

32. The compound of claim 31, wherein R² is a heterocycle selected from azetidine, pyrrolidine, and piperidine, wherein the heterocycle is substituted with one or more E and 0-4 R⁸.

33. The compound of any one of claims 27-32, wherein R² is substituted with one E and 0-4 R⁸.

34. The compound of claim 33, wherein R² is substituted with one E and 1-4 R⁸.

35. The compound of claim 34, wherein the 1-4 R⁸ are each independently selected from halogen and C₁₋₆alkyl, wherein any C₁₋₆alkyl is unsubstituted.

36. The compound of any one of claims 27-35, wherein each E is independently selected from:

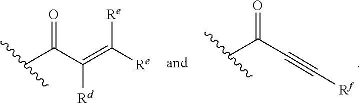

37. The compound of claim 36, wherein R² is substituted with one E having the structure: 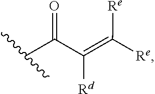
wherein each R^{d} and R^{e} is H.

38. The compound of any one of claims 1-37, wherein R³ is H.

39. The compound of any one of claims 1-37, wherein R³ is selected from —OR¹⁰ and C₁₋₆alkyl, wherein any C₁₋₆alkyl is unsubstituted or substituted with one or more R¹¹.

40. The compound of any one of claims 1-39, wherein R⁴ is H.

41. The compound of any one of claims 1-39, wherein R⁴ is selected from halogen, —CN, —OR¹², and C₁₋₆alkyl, wherein any C₁₋₆alkyl is unsubstituted or substituted with one or more R¹¹.

42. The compound of claim 41, wherein R⁴ is —CN.

43. The compound of claim 41, wherein R⁴ is a halogen.

44. The compound of claim 41, wherein R⁴ is C₁₋₆alkyl that is substituted with one or more R¹¹.

45. The compound of claim 44, wherein R⁴ is C₁₋₆alkyl that is substituted with one or more halogens or —CN.

46. The compound of claim 45, wherein R⁴ is selected from —CF₂H, —CF₃, —CF₂CH₃, and —CH₂CN.

47. The compound of claim 46, wherein R⁴ is —CF₃.

48. The compound of any one of claims 1-47, wherein R⁶ is selected from halogen, —CN, and H.

49. The compound of claim 48, wherein R⁶ is a halogen.

50. The compound of claim 48, wherein R⁶ is —CN.

51. The compound of claim 48, wherein R⁶ is H.

52. The compound of any one of claims 1-51, wherein R⁵ is: 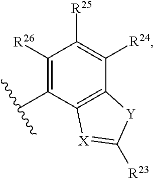
wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

53. The compound of any one of claims 1-52, wherein R⁵ is selected from: 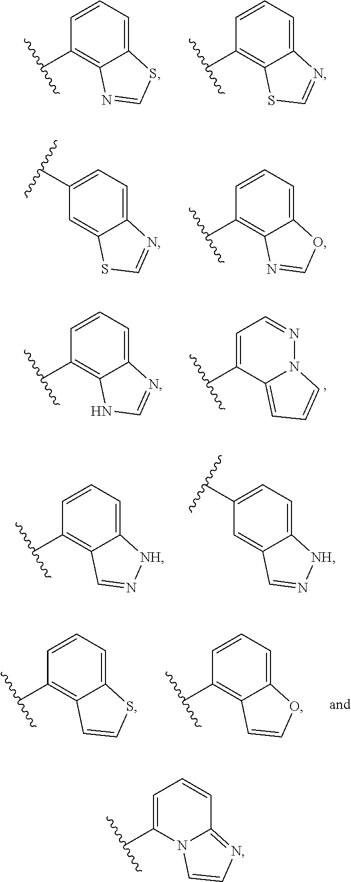
any of which is substituted with one or more R⁹.

54. The compound of any one of claims 1-53, wherein R⁵ is selected from:

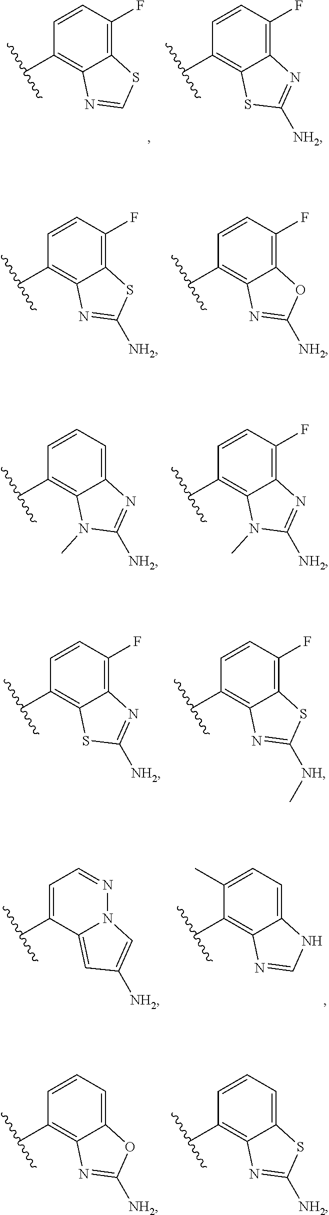

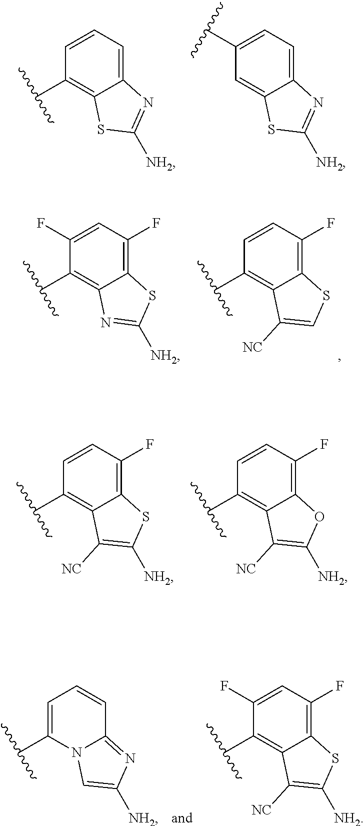

55. The compound of claim 54, wherein R⁵ is selected from:

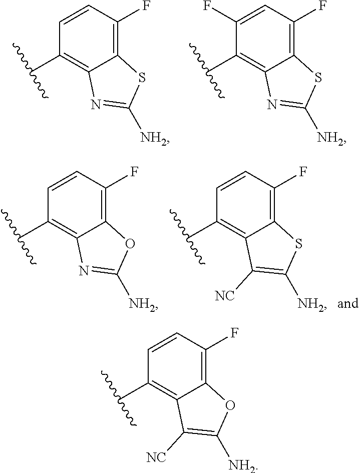

56. The compound of claim 1, wherein R² is a heterocycle selected from azetidine, pyrrolidine, and piperidine, wherein the heterocycle is substituted with one or more E and 0-4 R⁸; R³ is H; R¹ is selected from: 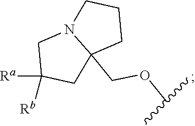
and R⁵ is selected from:

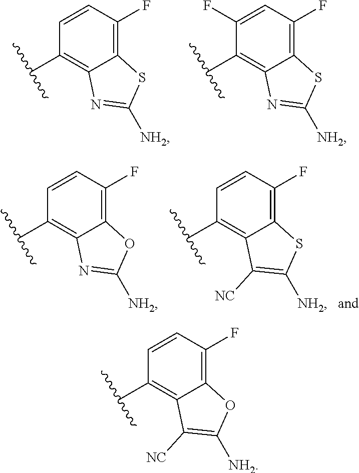

57. The compound of claim 56, wherein R² is substituted with one E having the structure: 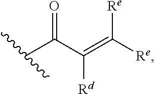
wherein each R^{d} and R^{e} is H.

58. The compound of claim 56 or 57, wherein R⁴ is —CF₃.

59. The compound of any one of claims 56-58, wherein R⁶ is selected from halogen and H.

60. A compound according to Formula II: 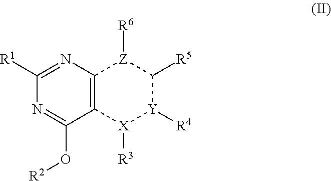
or a salt (e.g., pharmaceutically acceptable salt) thereof, wherein:
each dashed line represents a single or double bond;
X, Y, and Z are selected from N and C, wherein one and only one of X, Y, and Z is N; 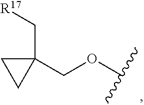
R¹ is selected from —OR⁷, and a 4-6 membered heterocycle comprising a nitrogen atom, wherein the heterocycle is unsubstituted or substituted with one or more R¹⁵;
R² is a 4-6 membered heterocycle containing one or more nitrogen atoms, wherein the heterocycle is substituted with one or more E and 0-4 R⁸;
when X is C, R³ is selected from H, —OR¹⁰, and C₁₋₆alkyl, wherein any C₁₋₆alkyl is unsubstituted or substituted with one or more R¹¹, and when X is N, R³ is absent;
when Y is C, R⁴ is selected from H, halogen, —CN, —OR¹², and C₁₋₆alkyl, wherein any C₁₋₆alkyl is unsubstituted or substituted with one or more R¹¹, and when Y is N, R⁴ is absent;
R⁵ is a bicyclic heteroaryl substituted with one or more R⁹;
when Z is C, R⁶ is selected from halogen, —OR¹², —CN, and H, and when Z is N, R⁶ is absent;
R⁷ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl;
each R⁸ is selected from halogen and C₁₋₆alkyl, wherein any C₁₋₆alkyl is unsubstituted or substituted with one or more R¹¹;
each R⁹ is independently selected from halogen, —N(R¹²)₂, —CN, and C₁₋₆alkyl, wherein any C₁₋₆alkyl is unsubstituted or substituted with one or more R¹³;
each R¹⁰ is independently selected from C₁₋₆ alkyl, C₂₋₆ alkenyl, and H;
each R¹¹ is independently selected from halogen, —OR¹², and —CN;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, C₁₋₆alkyl, —OR¹⁴, and a 3-6 membered heterocycle, wherein any C₁₋₆alkyl is unsubstituted or substituted with one or more R¹³, and any heterocycle is unsubstituted or substituted with one or more R¹⁶;
each R¹⁶ is independently selected from —OH, —OC₁₋₆alkyl, —CN, —NH₂, —NHC₁₋₆alkyl, and halogen;
R¹⁷ is a 3-6 membered heterocycle including one or more heteroatoms selected from N, O, and S, wherein the heterocycle is unsubstituted or substituted with one or more R¹⁸;
each R¹⁸ is independently selected from C₁₋₆alkyl and halogen;
each E is independently selected from 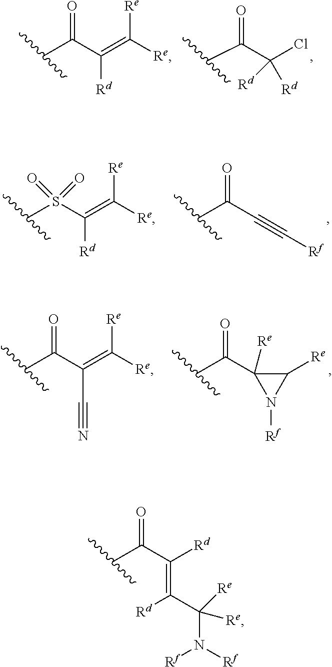
and —CN;
R^{a} and R^{b} are each independently selected from halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} and R^{e} are independently selected from halogen, C₁₋₆ alkyl, and H; and
each R^{f} is independently selected from C₁₋₆ alkyl and H.

61. The compound of claim 60, wherein X is N, Y and Z are C, and the compound is a compound according to Formula IIA: 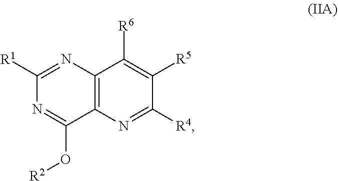
or a salt (e.g., pharmaceutically acceptable salt) thereof.

62. The compound of claim 61, wherein R⁴ is H.

63. The compound of claim 61, wherein R⁴ is selected from halogen, —CN, —OR¹², and C₁₋₆alkyl, wherein any C₁₋₆alkyl is unsubstituted or substituted with one or more R¹¹.

64. The compound of claim 63, wherein R⁴ is halogen (e.g., Cl or F).

65. The compound of claim 63, wherein R⁴ is C₁₋₆alkyl that is substituted with one or more halogens or —CN.

66. The compound of claim 65, wherein R⁴ is selected from —CF₂H, —CF₃, —CF₂CH₃, and —CH₂CN.

67. The compound of claim 66, wherein R⁴ is —CF₃.

68. The compound of any one of claims 61-67, wherein R⁶ is selected from halogen, —CN, and H.

69. The compound of claim 68, wherein R⁶ is halogen (e.g., Cl or F).

70. The compound of claim 68, wherein R⁶ is H.

71. The compound of claim 60, wherein Y is N, X and Z are C, and the compound is a compound according to Formula IIB: 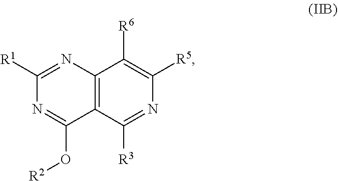
or a salt (e.g., pharmaceutically acceptable salt) thereof.

72. The compound of claim 71, wherein R³ is H.

73. The compound of claim 71, wherein R³ is selected from —OR¹⁰ and C₁₋₆alkyl, wherein any C₁₋₆alkyl is unsubstituted or substituted with one or more R¹¹.

74. The compound of any one of claims 71-73, wherein R⁶ is selected from halogen, —CN, and H.

75. The compound of claim 74, wherein R⁶ is a halogen (e.g., Cl or F).

76. The compound of claim 74, wherein R⁶ is H.

77. The compound of claim 60, wherein Z is N, X and Y are C, and the compound is a compound according to Formula IIC: 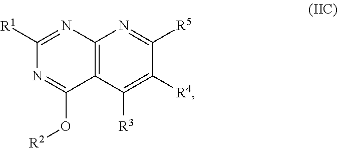
or a salt (e.g., pharmaceutically acceptable salt) thereof.

78. The compound of claim 77, wherein R³ is H.

79. The compound of claim 77, wherein R³ is selected from —OR¹⁰ and C₁₋₆alkyl, wherein any C₁₋₆alkyl is unsubstituted or substituted with one or more R¹¹.

80. The compound of any one of claims 77-79, wherein R⁴ is H.

81. The compound of any one of claims 77-79, wherein R⁴ is selected from halogen, —CN, —OR¹², and C₁₋₆alkyl, wherein any C₁₋₆alkyl is unsubstituted or substituted with one or more R¹¹.

82. The compound of claim 81, wherein R⁴ is halogen (e.g., Cl or F).

83. The compound of claim 81, wherein R⁴ is C₁₋₆alkyl that is substituted with one or more halogens or —CN.

84. The compound of claim 83, wherein R⁴ is selected from —CF₂H, —CF₃, —CF₂CH₃, and —CH₂CN.

85. The compound of claim 84, wherein R⁴ is —CF₃.

86. The compound of any one of claims 60-85, wherein R² is a 4-6 membered heterocycle containing one nitrogen atom, wherein the heterocycle is substituted with one or more E and 0-4 R⁸.

87. The compound of claim 86, wherein R² is a heterocycle selected from azetidine, pyrrolidine, and piperidine, wherein the heterocycle is substituted with one or more E and 0-4 R⁸.

88. The compound of any one of claims 60-87, wherein R² is substituted with one E and 0-4 R⁸.

89. The compound of claim 88, wherein R² is substituted with one E and 1-4 R⁸.

90. The compound of claim 89, wherein the 1-4 R⁸ are each independently selected from halogen and C₁₋₆alkyl, wherein any C₁₋₆alkyl is unsubstituted.

91. The compound of any one of claims 60-90, wherein each E is independently selected from:

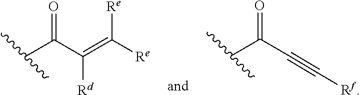

92. The compound of claim 91, wherein R² is substituted with one E that is 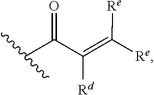
wherein each R^{d} and R^{e} is H.

93. The compound of any one of claims 60-92, wherein R¹ is —OR⁷.

94. The compound of claim 93, wherein R¹ is 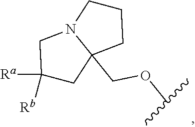
wherein R^{a} and R^{b} are each independently selected from halogen, C₁₋₆alkyl, —OR¹², and H, wherein any C₁₋₆alkyl is unsubstituted or is substituted with one or more R¹³.

95. The compound of claim 94, wherein R^{b} is H.

96. The compound of claim 94, wherein R¹ is selected from:

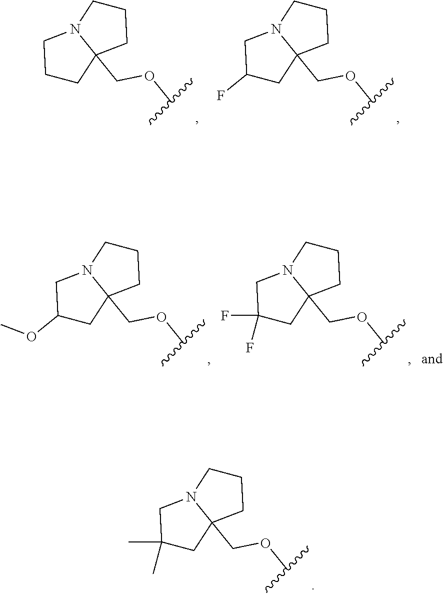 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

97. The compound of claim 93, wherein R¹ is selected from: 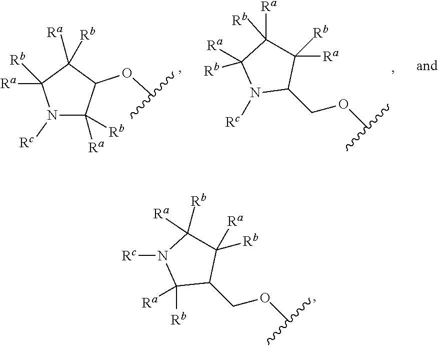
wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, wherein any C₁₋₆ alkyl or 3-6 membered carbocycle or heterocycle is unsubstituted or is substituted with one or more R¹³.

98. The compound of claim 97, wherein R¹ is selected from:

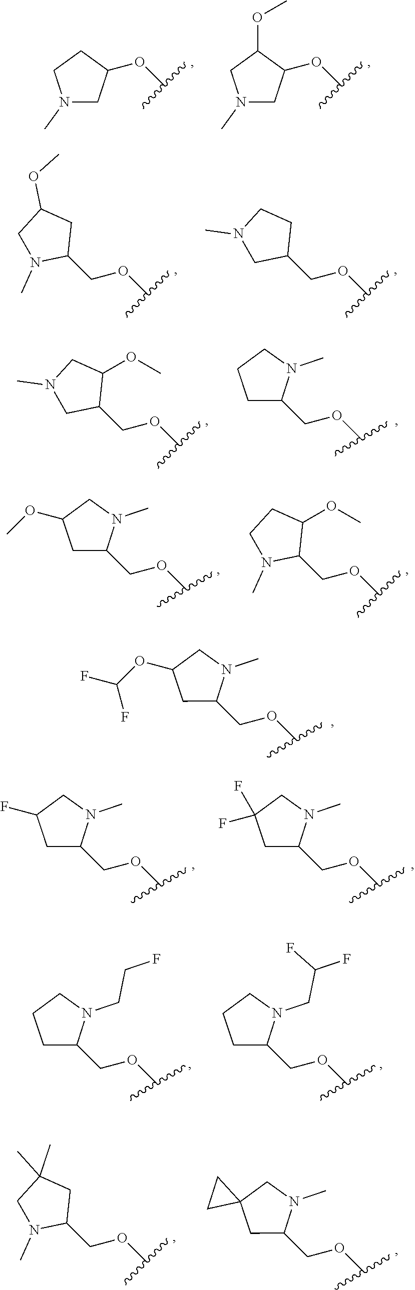

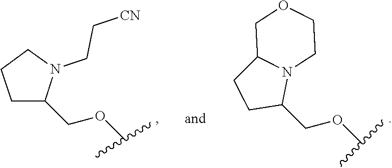 [SMILES: *OCC1CCCN1CCC#N ; *OCC1CCC2COCCN12]

99. The compound of any one of claims 60-92, wherein R¹ is

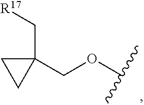

100. The compound of claim 99, wherein R¹ is selected from:

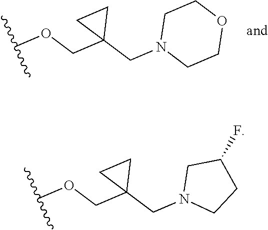 [SMILES: *OCC1(CN2CCOCC2)CC1 ; *OCC1(CN2CC[C@@H](F)C2)CC1]

101. The compound of any one of claims 60-92, wherein R¹ is a 4-6 membered heterocycle comprising a nitrogen atom, wherein the heterocycle is unsubstituted or substituted with one or more R¹⁵.

102. The compound of claim 101, wherein R¹ is selected from:

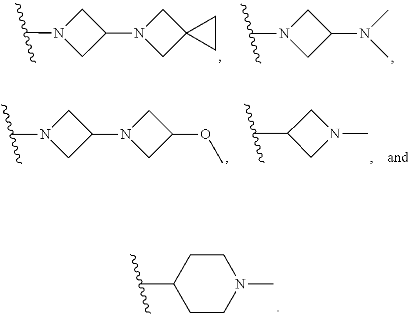

103. The compound of any one of claims 60-102, wherein R⁵ is: 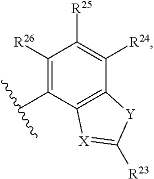
wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

104. The compound of claim 103, wherein R⁵ is selected from: 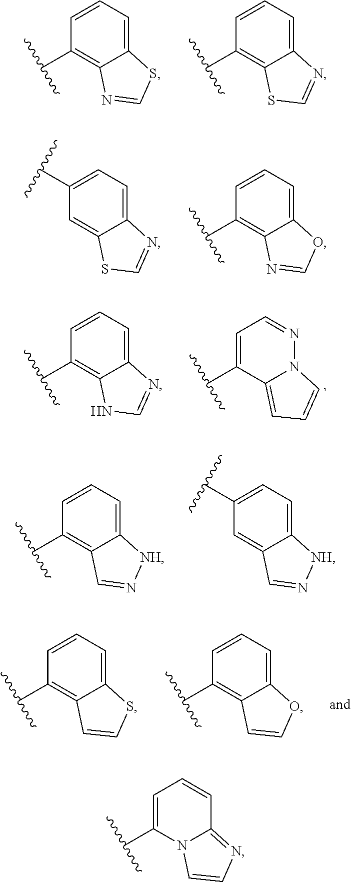
any of which is substituted with one or more R⁹.

105. The compound of claim 103 or 104, wherein R⁵ is selected from:

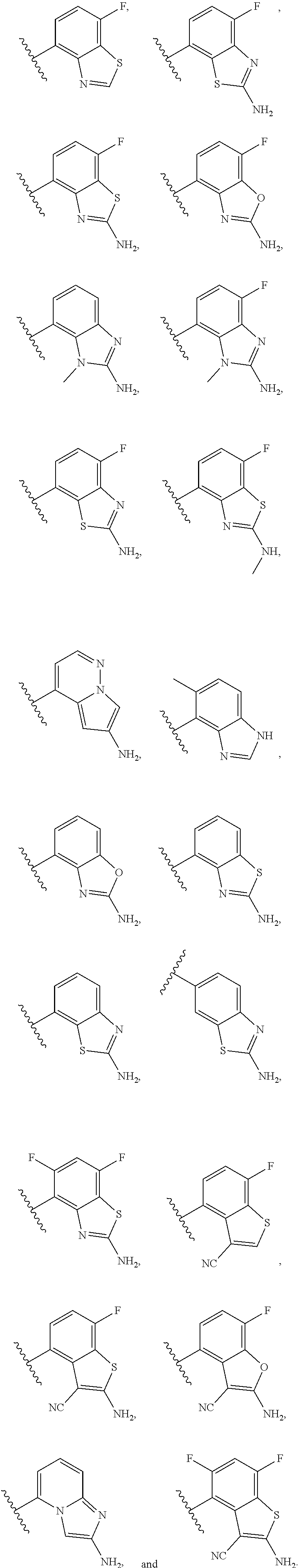

106. The compound of claim 105, wherein R⁵ is selected from:

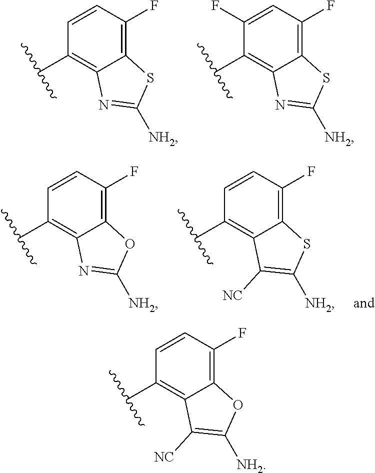

107. A compound shown in Table 2, or a salt (e.g., pharmaceutically acceptable salt) thereof.

108. A compound shown in Table 3, or a salt (e.g., pharmaceutically acceptable salt) thereof.

109. A pharmaceutical composition comprising a compound of any one of claims 1-108, or a salt (e.g., pharmaceutically acceptable salt) thereof, and a pharmaceutically acceptable excipient.

110. A compound of any one of claims 1-108, or a salt (e.g., pharmaceutically acceptable salt) thereof, for use as a medicament.

111. The compound of claim 110, wherein the medicament is useful in the prevention or treatment of a disease, disorder, or condition ameliorated by the inhibition of KRAS having a G12C mutation.

112. The compound of claim 110 or 111, wherein the medicament is useful in the prevention or treatment of a cancer.

113. The compound of claim 112, wherein the cancer is selected from the group consisting of pancreatic cancer, colorectal cancer, and lung cancer.

114. A compound of any one of claims 1-108, or a salt (e.g., pharmaceutically acceptable salt) thereof, for use in the treatment of a disease, disorder, or condition.

115. The compound of claim 114, wherein the disease, disorder, or condition is a cancer.

116. The compound of claim 115, wherein the cancer is selected from the group consisting of pancreatic cancer, colorectal cancer, and lung cancer.

117. The compound of any one of claims 114-116, wherein the compound is used in the treatment of a disease, disorder, or condition in a subject in need thereof.

118. A compound of any one of claims 1-108, or a salt (e.g., pharmaceutically acceptable salt) thereof, for use in the manufacture of a medicament.

119. The compound of claim 118, wherein the medicament is useful in the prevention or treatment of a disease, disorder, or condition ameliorated by the inhibition of KRAS having a G12C mutation.

120. The compound of claim 118 or 119, wherein the medicament is useful in the treatment of a cancer.

121. The compound of claim 120, wherein the cancer is selected from the group consisting of pancreatic cancer, colorectal cancer, and lung cancer.

122. A method, comprising administering a therapeutically effective amount of a compound of any one of claims 1-108, or a salt (e.g., pharmaceutically acceptable salt) thereof, to a subject in need thereof.

123. The method of claim 122, wherein the subject has a disease, disorder, or condition ameliorated by the inhibition of KRAS having a G12C mutation.

124. The method of claim 122 or 123, wherein the subject has a cancer.

125. The method of claim 124, wherein the subject was previously diagnosed with the cancer.

126. The method of claim 124, wherein the subject has previously undergone a treatment regimen for the cancer.

127. The method of claim 124, wherein the subject has previously entered remission from the cancer.

128. The method of any one of claims 124-127, wherein the cancer is selected from the group consisting of pancreatic cancer, colorectal cancer, and lung cancer.

129. The method of any one of claims 122-128, wherein the compound, or the salt thereof, is administered in combination with an additional therapeutic agent.

130. The use of a compound of any one of claims 1-108 or a salt (e.g., pharmaceutically acceptable salt) thereof, for the manufacture of a medicament for the treatment of a cancer.

131. The use of claim 130, wherein the cancer is selected from the group consisting of pancreatic cancer, colorectal cancer, and lung cancer.

132. A method, comprising contacting a KRAS protein with a compound of any one of claims 1-108, or a salt (e.g., pharmaceutically acceptable salt) thereof.

133. The method of claim 132, wherein contacting the KRAS protein with the compound modulates KRAS.

134. The method of claim 132 or 133, wherein the KRAS protein has a G12C mutation.

135. The method of any one of claims 132-134, wherein the KRAS protein is in an active (GTP-bound) state.

136. The method of any one of claims 132-134, wherein the KRAS protein is in an inactive (GDP-bound) state.

137. The method of any one of claims 132-136, wherein the KRAS protein is located within a cell.

138. The method of claim 137, wherein the cell is located within a subject.

139. The method of claim 138, wherein the subject is a human.

140. The method of claim 138 or 139, wherein the subject has a cancer.

141. The method of claim 140, wherein the cancer is selected from the group consisting of pancreatic cancer, colorectal cancer, and lung cancer.

142. A method of inhibiting the function of a KRAS protein having a G12C mutation, comprising contacting the KRAS protein with a compound of any one of claims 1-108, or a salt (e.g., pharmaceutically acceptable salt) thereof.

143. The method of claim 142, wherein the KRAS protein is in an active (GTP-bound) state.

144. The method of claim 142, wherein the KRAS protein is in an inactive (GDP-bound) state.

145. The method of any one of claims 142-144, wherein the KRAS protein is located within a cell.

146. The method of claim 145, wherein the cell is located within a subject.

147. The method of claim 146, wherein the subject is a human.

148. The method of claim 146 or 147, wherein the subject has a cancer.

149. The method of claim 148, wherein the cancer is selected from the group consisting of pancreatic cancer, colorectal cancer, and lung cancer.

150. A compound of any one of claims 1-108, or a salt (e.g., pharmaceutically acceptable salt) thereof, wherein the compound:
(i) demonstrates modification of ≥70%, 50%≤modification <70%, or 10%≤modification <50% of GppNHp-, GTP-, or GDP-loaded KRAS G12C in the assay of Biological Example 1 (e.g., a Matrix-Assisted Laser Desorption Ionization-Time of Flight Mass Spectrometry (MALDI-TOF MS) analysis of covalent modification of Cys12 in GppNHp-, GTP-, or GDP-loaded KRAS4b (amino acids 1-169) G12C/C118S);
(ii) has IC₅₀≤0.5 μM, 0.5 μM<IC₅₀≤5 μM, or 5 μM<IC₅₀≤20 μM in the assay of Biological Example 2 (e.g., a protein:protein interaction (PPI) Homogenous Time Resoled Fluorescence (HTRF) analysis of Avi-KRAS G12C Q25A (amino acids 1-169) GppNHp/3×FLAG-PI3K CA (157-299), Avi-KRAS G12C (1-169) GppNHp/RAF1 RBD-3×FLAG (52-151)); and/or
(iii) has IC₅₀≤0.1 μM or 0.1 μM<IC₅₀≤1 μM in the assay of Biological Example 3 (e.g., cell-based pERK).

151. The compound of claim 150, wherein the compound is capable of reversibly binding the KRAS protein.

152. The compound of claim 150, wherein the compound is capable of irreversibly binding the KRAS protein.
